(19) United States
(12) Patent Application Publication
(10) Pub. No.: US 2024/0294508 A1
(43) Pub. Date: Sep. 5, 2024

(54) RIP1 MODULATORS INCLUDING AZETIDINE CYCLIC UREAS, PREPARATIONS, AND USES THEREOF

(71) Applicant: SIRONAX LTD., Grand Cayman (KY)

(72) Inventors: Zhaolan ZHANG, Beijing (CN); Zhiyuan ZHANG, Beijing (CN); Yaning SU, Beijing (CN); Yanping XU, Beijing (CN)

(21) Appl. No.: 18/561,308

(22) PCT Filed: May 16, 2022
(86) PCT No.: PCT/CN2022/092907

§ 371 Date: Nov. 16, 2023

(30) Foreign Application Priority Data
May 20, 2021 (WO) PCT/CN2021/094944

(51) Int. Cl. C07D 403/14; A61K 31/4439; A61K 31/444; A61K 31/506; C07D 401/14; C07D 405/14; C07D 413/14; C07D 417/14

(52) U.S. Cl. CPC C07D 403/14; A61K 31/4439; A61K 31/444; A61K 31/506; C07D 401/14; C07D 405/14; C07D 413/14; C07D 417/14

(57) ABSTRACT

Provided herein are compounds of Formula Ia and Ib, compositions comprising the same, and methods of using the same, including use in treating various diseases and conditions, including those mediated by receptor-interacting protein 1 (RIP1) signaling

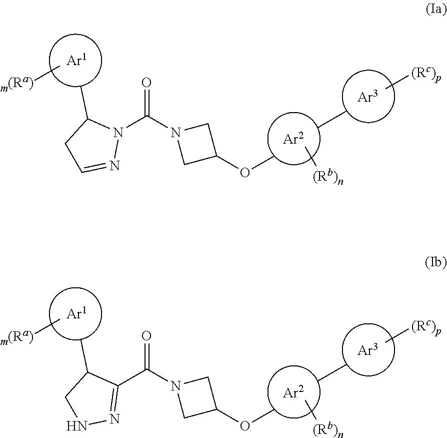

### FIELD OF THE DISCLOSURE

The present disclosure relates to compounds that modulate the receptor-interacting protein 1 (RIP1), compositions comprising the compounds, methods of preparing the compounds, and methods of using the compounds to treat various diseases or conditions, e.g., mediated by RIP1.

### BACKGROUND OF THE DISCLOSURE

Necroptosis, an important form of programmed cell death (PCD), is a highly regulated caspase-independent type of cell death that plays a critical role in many necrotic cell diseases, manifested in various pathological forms of cell death, including ischemic brain injury, neurodegenerative diseases, viral infections, and peripheral autoimmune diseases. (Dunai, et al., December 2011, Pathol. Oncol. Res.: POR 17 (4): 791-800. J. Med. Chem. 2020, 63, 4, 1490-1510. Nature Reviews Drug Discovery, 19, 553-571(2020)). Tumor necrosis factor alpha (TNF-α)-induced NF-κB activation plays a central role in the immune system and inflammatory responses.

Receptor-interacting protein 1 (RIP1) is a multi-functional signal transducer involved in mediating nuclear factor κB (NF-κB) activation, apoptosis, and necroptosis. The kinase activity of RIP1 is critically involved in mediating necroptosis, a caspase-independent pathway of necrotic cell death. (Holler et al. Nat Immunol 2000; 1: 489-495; Degterev et al. Nat Chem Biol 2008; 4: 313-321). RIP1 can contribute to D-1 immunotherapy resistance (e.g., Manguso et al., 2017 Nature 547, 413-418) and can act as a checkpoint kinase governing tumor immunity (e.g., Wang et al, Cancer Cell 34, 757-774, Nov. 12, 2018). RIP1 has emerged as a promising therapeutic target for the treatment of a wide range of human neurodegenerative, autoimmune, and inflammatory diseases, such as psoriasis, rheumatoid arthritis, and ulcerative colitis (Pharmacol. Res. Perspect. 2017, 5, e00365, PNAS May 14, 2019 116 (20) 9714-9722), as well for CNS indications such as ALS and Alzheimer's disease. (Nat. Rev. Neurosci. 2019, 20, 19-33).

Certain compounds for modulating necrosis or necroptosis are disclosed in U.S. Pat. Nos. 9,974,762, 10,092,529, 6,756,394, 8,278,344, U.S. Patent Publication No. 20120122889, U.S. Patent Publication No. 20090099242, U.S. Patent Publication No. 20100317701, U.S. Patent Publication No. 20110144169, U.S. Patent Publication No. 20030083386, U.S. Patent Publication No. 201200309795, WO2009023272, WO2010075290, WO2010075561, WO2012125544, and WO 2020/103884.

### SUMMARY OF THE DISCLOSURE

One aspect of this disclosure provides a compound selected from compounds of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, a tautomer thereof, a hydrate or stereoisomer of the compound or the tautomer, or a pharmaceutically acceptable salt of the foregoing, which can be employed in the treatment of various diseases or conditions, such as diseases or conditions mediated by receptor-interacting protein 1 (RIP1). For example, disclosed herein is a compound of one of the following structural Formulae Ia and Ib:

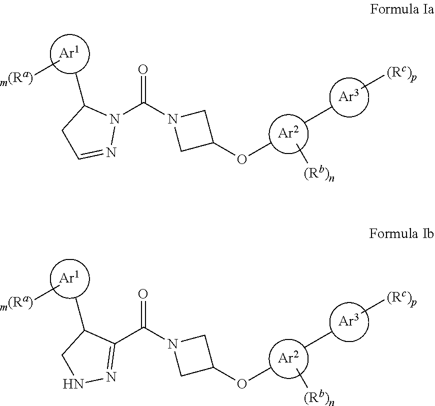

a tautomer thereof, a hydrate or stereoisomer of the compound or the tautomer, or a pharmaceutically acceptable salt of the foregoing, wherein:
Ar¹ is phenyl, C₅-C₆ cycloalkyl, 5- to 6-membered heteroaryl, or 5- to 6-membered heterocyclyl,
Ar² is phenyl, C₅-C₆ cycloalkyl, 5- to 6-membered heteroaryl, or 5- to 6-membered heterocyclyl, provided that when

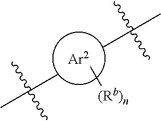

in formula Ia is

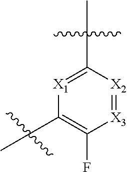

wherein X₁, X₂, and X₃ are C; or X₁ is N, X₂ and X₃ are C; or X₂ is N, X₁ and X₃ are C; or X₁ and X₂ are N, and X₃ is C; or X₁ and X₂ are C, and X₃ is N,

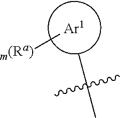

cannot be

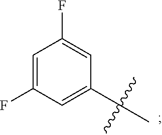 [SMILES: *c1cc(F)cc(F)c1]

Ar³ is phenyl, C₅-C₆ cycloalkyl, 5- to 6-membered heteroaryl, or 5- to 6-membered heterocyclyl;
R^{a}, for each occurrence, is independently selected from halogen, CN, C₁-C₃ alkyl, and OH;
R^{b}, for each occurrence, is independently selected from halogen, CN, C₁-C₃ alkyl, and OH;
R^{c}, for each occurrence, is independently selected from halogen, cyano, C₁-C₆ alkyl, C₃-C₆ cycloalkyl, 3- to 6-membered heterocyclyl, C₂-C₆ alkenyl, C₁-C₆ alkoxy, —C(═O)(C₁-C₆ alkyl), —C(═O)(C₃-C₆ cycloalkyl), —C(═O)(3- to 6-membered heterocyclyl), ═O, —NO₂, —C(═O)OR^{s}, —C(═O)NR^{p}R^{q}, —NR^{p}R^{q}, —NR^{p}C(═O)R^{s}, —NR^{p}C(═O)OR^{s}, —NR^{p}C(═O)NR^{q}R^{r}, —NR^{p}S(═O)_{w}R^{s}, —OR^{s}, —OC(═O)R^{s}, —OC(═O)OR^{s}, —OC(═O)NR^{p}R^{q}, —S(═O)_{w}R^{s}, and —S(═O)_{w}NR^{p}R^{q}; wherein
the C₁-C₆ alkyl, C₃-C₆ cycloalkyl, 3- to 6-membered heterocyclyl, C₂-C₆ alkenyl, and C₁-C₆ alkoxy of R^{c}, the C₁-C₆ alkyl of —C(═O)(C₁-C₆ alkyl), the C₃-C₆ cycloalkyl of —C(═O)(C₃-C₆ cycloalkyl), and the 3- to 6-membered heterocyclyl of —C(═O)(3- to 6-membered heterocyclyl) are each optionally substituted with 1 to 3 groups selected from halogen, cyano, —C(═O)R^{s}, —C(═O)OR^{s}, —C(═O)NR^{p}R^{q}, —NR^{p}R^{q}, —NR^{p}C(═O)R^{s}, —NR^{p}C(═O)OR^{s}, —NR^{p}C(═O)NR^{q}R^{r}, —NR^{p}S(═O)R^{s}, —OR^{s}, —OC(═O)R^{s}, —OC(═O)OR^{s}, —OC(═O)NR^{p}R^{q}, —S(═O)_{w}R^{s}, —S(═O)_{w}NR^{p}R^{q}, C₃-C₆ cycloalkyl, and 3- to 6-membered heterocyclyl; wherein
R^{p}, R^{q}, R^{r}, and R^{s}, for each occurrence, are each independently selected from hydrogen, OH, NH₂, C₁-C₄ alkyl, —C(═O)(C₁-C₄ alkyl), C₃-C₆ cycloalkyl, and 3- to 6-membered heterocyclyl; wherein
the C₁-C₄ alkyl, C₃-C₆ cycloalkyl, and 3- to 6-membered heterocyclyl of any one of R^{p}, R^{q}, R^{r}, and R^{s} are optionally substituted with 1 to 3 groups selected from halogen, cyano, —OH, C₁-C₆ alkyl, —O(C₁-C₆ alkyl), —C(═O)N(C₁-C₆ alkyl)(C₁-C₆ alkyl), —C(═O)NH(C₁-C₆ alkyl), —C(═O)(3- to 6-membered heterocyclyl), —C(═O)(C₃-C₆ cycloalkyl), C₃-C₆ cycloalkyl, phenyl, and 3- to 6-membered heterocyclyl; and wherein
w is an integer selected from 0, 1 and 2;
m and p are each an integer independently selected from 0, 1, 2, and 3; and
n is selected from 0, 1, and 2.

In one aspect of the disclosure, the compounds of Formulae Ia and Ib are selected from Compounds 1 to 169 shown below, a tautomer thereof, a hydrate or stereoisomer of the compound or the tautomer, or a pharmaceutically acceptable salt of the foregoing.

In some embodiments, the disclosure provides pharmaceutical compositions comprising a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, a tautomer thereof, a hydrate or stereoisomer of the compound or the tautomer, or a pharmaceutically acceptable salt of the foregoing, and a pharmaceutically acceptable carrier. In some embodiments, the pharmaceutical compositions may comprise a compound selected from Compounds 1 to 169 shown below, a tautomer thereof, a hydrate or stereoisomer of the compound or the tautomer, or a pharmaceutically acceptable salt of the foregoing, and a pharmaceutically acceptable carrier. These compositions may further comprise an additional active pharmaceutical agent.

Another aspect of the disclosure provides methods of treating a disease or condition, comprising administering to a subject in need thereof, a therapeutically effective amount of a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, a tautomer thereof, a hydrate or stereoisomer of the compound or the tautomer, or a pharmaceutically acceptable salt of the foregoing, or a pharmaceutical composition comprising any of the foregoing, wherein the disease or condition is selected from an inflammatory disease, an immune disease (e.g., an autoimmune disease), an allergic disease, transplant rejection, a necrotic cell disease, a neurodegenerative disease, a central nervous system (CNS) disease, an ocular disease, an infectious disease, and a malignancy.

A further aspect of the disclosure provides methods of treating a disease or condition mediated by RIP1, comprising administering to a subject in need thereof, a therapeutically effective amount of a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, a tautomer thereof, a hydrate or stereoisomer of the compound or the tautomer, or a pharmaceutically acceptable salt of the foregoing, or a pharmaceutical composition comprising any of the foregoing.

In some embodiments, the methods of treatment comprise administering to a subject in need thereof, a compound selected from Compounds 1 to 169 shown below, a tautomer thereof, a hydrate or stereoisomer of the compound or the tautomer, or a pharmaceutically acceptable salt of the foregoing, or a pharmaceutical composition comprising any of the foregoing.

In some embodiments, the methods of treatment comprise administration of an additional active pharmaceutical agent to the subject in need thereof, either in the same pharmaceutical composition as a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, a tautomer thereof, a hydrate or stereoisomer of the compound or the tautomer, or a pharmaceutically acceptable salt of the foregoing, or in a separate composition. In some embodiments, the methods of treatment comprise administering a compound selected from Compounds 1 to 169 shown below, a tautomer thereof, a hydrate or stereoisomer of the compound or the tautomer, or a pharmaceutically acceptable salt of the foregoing with an additional active pharmaceutical agent either in the same pharmaceutical composition or in a separate composition. When administered as a separate dosage form, the additional therapeutic agent may be administered prior to, at the same time as, or following administration of the compound, tautomer, hydrate, stereoisomer, or a pharmaceutically acceptable salt disclosed herein.

Also disclosed herein are methods of mediating, e.g., inhibiting, RIP1, comprising contacting the RIP1 protein or a fragment thereof with a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, a tautomer thereof, a hydrate or stereoisomer of the compound or the tautomer, or a pharmaceutically acceptable salt of the foregoing, or a pharmaceutical composition comprising any of the foregoing. In some embodiments, the methods of inhibiting RIP1 comprise contacting the RIP1 protein or a fragment thereof with a compound selected from Compounds 1 to 169 shown below, a tautomer thereof, a hydrate or stereoisomer of the compound or the tautomer, or a pharmaceutically acceptable salt of the foregoing, or a pharmaceutical composition comprising any of the foregoing.

### DETAILED DESCRIPTION OF THE DISCLOSURE

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “alkyl” refers to a hydrocarbon group selected from linear and branched saturated hydrocarbon groups of 1-18, or 1-12, 1-6, or 1-3 carbon atoms. Examples of the alkyl group include methyl, ethyl,1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of the alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

Lower alkyl means 1-8, preferably 1-6, more preferably 1-4 carbon atoms, e.g., 1-3 carbon atoms, and lower alkenyl or alkynyl means 2-8, 2-6 or 2-4 carbon atoms.

The term “alkenyl” refers to a hydrocarbon group selected from linear and branched hydrocarbon groups comprising at least one C═C double bond and of 2-18, or 2-12, or 2-6 carbon atoms. Examples of the alkenyl group may be selected from ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups.

The term “alkynyl” refers to a hydrocarbon group selected from linear and branched hydrocarbon group, comprising at least one C≡C triple bond and of 2-18, or 2-12, or 2-6 carbon atoms. Examples of the alkynyl group include ethynyl, 1-propynyl, 2-propynyl (propargyl), 1-butynyl, 2-butynyl, and 3-butynyl groups. Lower alkyl means 1-8, preferably 1-6, more preferably 1-4 carbon atoms; lower alkenyl or alkynyl means 2-8, 2-6 or 2-4 carbon atoms.

The term “heteroalkyl” as used herein refers to an alkyl group, as defined herein, in which one or more of the constituent carbon atoms have been replaced by nitrogen, oxygen, or sulfur, e.g., CH₃CH₂OH, CH₃CH₂OC₂H₅, CH₃CH₂SH, CH₃CH₂SC₂H₅, CH₃CH₂NH₂, CH₃CH₂NHC₂H₅, etc. In some embodiments, in addition to the replacement of one or more of the constituent carbon atoms by nitrogen, oxygen, or sulfur, a heteroalkyl group is further optionally substituted as defined herein.

The term “cycloalkyl” refers to a hydrocarbon group selected from saturated and partially unsaturated cyclic hydrocarbon groups, comprising monocyclic and polycyclic (e.g., bicyclic and tricyclic) groups. For example, the cycloalkyl group may be of 3-12, or 3-8, or 3-6, or 3-4, or 5-6 carbon atoms. Even further for example, the cycloalkyl group may be a monocyclic group of 3-12, or 3-8, or 3-6, or 5-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e., partially unsaturated), but is not fully conjugated, and is not aromatic, as aromatic is defined herein.

The term “heterocyclic” or “heterocycle” or “heterocyclyl” refers to a ring selected from 4- to 12-membered, e.g., 3- to 6-membered, or 3 to 5-membered, or 4- to 5-membered, or 5- to 6-membered, monocyclic, bicyclic and tricyclic, saturated and partially unsaturated rings comprising at least one carbon atoms in addition to 1, 2, 3 or 4 heteroatoms, selected from oxygen, sulfur, and nitrogen. “Heterocycle” also refers to a 5- to 7-membered heterocyclic ring comprising at least one heteroatom selected from N, O, and S fused with 5-, 6-, and/or 7-membered cycloalkyl, carbocyclic aromatic or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e., partially unsaturated). The heterocycle may be substituted with oxo. The point of the attachment may be carbon or heteroatom in the heterocyclic ring. A heterocycle is not a heteroaryl as defined herein.

Examples of the heterocycle include, but not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl and 1,1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicyclic aryl ring such as 7 to 12 membered bicyclic aryl ring systems as mentioned above, a fused tricyclic aryl ring such as 10 to 15 membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The term “heteroatom” means one or more of oxygen, sulfur, nitrogen, and phosphorus, including, any oxidized form of nitrogen or sulfur; the quaternized form of any basic nitrogen or a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains one or more double or triple bonds.

The term “alkoxy” as used herein, refers to an alkyl group, as defined above, wherein one carbon of the alkyl group is replaced by an oxygen atom, provided that the oxygen atom is linked between two carbon atoms.

The term “halogen” includes F, Cl, Br, and I, i.e., fluoro, chloro, bromo, and iodo, respectively.

As used herein, a “cyano” or “nitrile” group refers to —C≡N.

As used herein, an “aromatic ring” refers to a carbocyclic or heterocyclic ring that contains conjugated, planar ring systems with delocalized pi electron orbitals comprised of [4n+2] p orbital electrons, wherein n is an integer of 0 to 6. A “non-aromatic” ring refers to a carbocyclic or heterocyclic that does not meet the requirements set forth above for an aromatic ring, and can be either completely or partially saturated. Non-limiting examples of aromatic rings include aryl and heteroaryl rings that are further defined as follows.

The term “aryl” herein refers to a group selected from: 5- and 6-membered carbocyclic aromatic rings, for example, phenyl; bicyclic ring systems such as 7-12 membered bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group is selected from 5- and 6-membered carbocyclic aromatic rings fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “heteroaryl” refers to a group selected from: 5- to 7-membered, e.g., 5- to 6-membered, aromatic, monocyclic rings comprising 1, 2, 3 or 4 heteroatoms selected from N, O, and S, with the remaining ring atoms being carbon; 8- to 12-membered bicyclic rings comprising 1, 2, 3 or 4 heteroatoms, selected from N, O, and S, with the remaining ring atoms being carbon and wherein at least one ring is aromatic and at least one heteroatom is present in the aromatic ring; and 11- to 14-membered tricyclic rings comprising 1, 2, 3 or 4 heteroatoms, selected from N, O, and S, with the remaining ring atoms being carbon and wherein at least one ring is aromatic and at least one heteroatom is present in an aromatic ring.

For example, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

Some of the compounds may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

The compounds, tautomers, hydrates, or pharmaceutically acceptable salts of the disclosure may contain an asymmetric center and may thus exist as enantiomers. For example, where the compounds possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included in this disclosure. All stereoisomers of the compounds, tautomers, hydrates, and pharmaceutically acceptable salts thereof are intended to be included. Unless specifically mentioned otherwise, reference to one isomer applies to any of the possible isomers. Whenever the isomeric composition is unspecified, all possible isomers are included.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents. Racemic mixtures of chiral compounds of the disclosure can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions.

The term “substantially pure” in the context of stereoisomers means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

Unless otherwise indicated, structures depicted herein are also meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the disclosure. Unless otherwise stated, all tautomeric forms of the compounds of the disclosure are within the scope of the disclosure.

The disclosure provides pharmaceutically acceptable salts of the disclosed compounds, tautomers, hydrates, and stereoisomers. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n-COOH, wherein n is selected from 0 to 4. Similarly, examples of pharmaceutically acceptable cations include, but are not limited to, sodium, potassium, calcium, aluminum, lithium, and ammonium. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

If a compound is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The compounds, tautomers, hydrates, stereoisomers, and pharmaceutically acceptable salts of the disclosure may also contain unnatural proportions of atomic isotopes at one or more of the atoms that constitute such compounds, such as deuterium, e.g., —CD₃, CD₂H or CDH₂ in place of methyl. For example, the compounds may be radiolabeled with radioactive isotopes, such as for example tritium (³H), iodine-125 (¹²⁵I) or carbon-14 (¹⁴C). All isotopic variations of the compounds of the disclosure, whether radioactive or not, are intended to be encompassed within the scope of the disclosure.

As used herein, “optionally substituted” is interchangeable with the phrase “substituted or unsubstituted.” In general, the term “substituted,” refers to the replacement of a hydrogen radical in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

In some embodiments, substituents are selected from optionally substituted heteroatom and optionally substituted, optionally hetero-, optionally cyclic C₁-C₁₈ hydrocarbyl, particularly wherein the optionally substituted, optionally hetero-, optionally cyclic C₁-C₁₈ hydrocarbyl is optionally-substituted, optionally hetero-, optionally cyclic alkyl, alkenyl or alkynyl, or optionally-substituted, optionally hetero-aryl; and/or the optionally substituted heteroatom is halogen, optionally substituted hydroxyl (such as alkoxy, aryloxy), optionally substituted acyl (such as formyl, alkanoyl, carbamoyl, carboxyl, amido), optionally substituted amino (such as amino, alkylamino, dialkylamino, amido, sulfamidyl), optionally substituted thiol (such as mercapto, alkylthiol, aryl thiol), optionally substituted sulfinyl or sulfonyl (such as alkylsulfinyl, arylsulfinyl, alkyl sulfonyl, arylsulfonyl), nitro, or cyano.

In some embodiments, substituents are selected from: halogen, —R′, —OR′, ═O, ═NR′, ═N—OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR′—SO₂NR′″, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R, —CN and —NO₂, —N₃, —CH(Ph)₂, perfluoro(C₁-C₄)alkoxy and perfluoro(C₁-C₄)alkyl, in a number ranging from zero to three, with those groups having zero, one or two substituents being particularly preferred. R′, R″ and R′″ each independently refer to hydrogen, unsubstituted (C₁-C₈)alkyl and heteroalkyl, (C₁-C₈)alkyl and heteroalkyl substituted with one to three halogens, unsubstituted aryl, aryl substituted with one to three halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl, “alkyl” includes groups such as trihaloalkyl (e.g., —CF₃ and —CH₂CF₃), and when the aryl group is 1,2,3,4-tetrahydronaphthalene, it may be substituted with a substituted or unsubstituted (C₃-C₇)spirocycloalkyl group. The (C₃-C₇)spirocycloalkyl group may be substituted in the same manner as defined herein for “cycloalkyl.”

In some embodiments, substituents are selected from: halogen, —R′, —OR′, ═O, —NR′R″, —SR′, —SiR′R″R′″, —OC(O)R′, —C(O)R′, —CO2R′, —CONR′R″, —OC(O)NR′R″, —NR″C(O)R′, —NR″CO₂R′, —NR′—SO2NR″R′″, —S(O)R′, —SO₂R′, —SO₂NR′R″, —NR″SO₂R, —CN and —NO₂, perfluoro(C₁-C₄)alkoxy and perfluoro(C1-C4)alkyl, where R′ and R″ are as defined above.

In some embodiments, substituents are independently substituted or unsubstituted heteroatom, substituted or unsubstituted, 0-3 heteroatom C₁-C₆ alkyl, C₁-C₃ alkyl, or C₁-C₂ alkyl, substituted or unsubstituted, 0-3 heteroatom C₂-C₆ alkenyl, substituted or unsubstituted, 0-3 heteroatom C₂-C₆ alkynyl, or substituted or unsubstituted, 0-3 heteroatom C₆-C₁₄ aryl, or C₅-C₆ aryl, wherein each heteroatom is independently oxygen, phosphorus, sulfur, or nitrogen.

In some embodiments, substituents are independently aldehyde, aldimine, alkanoyloxy, alkoxy, alkoxycarbonyl, alkyloxy, alkyl, alkenyl, alkynyl, amine, azo, halogen, carbamoyl, carbonyl, carboxamido, carboxyl, cyanyl, ester, haloformyl, hydroperoxyl, hydroxyl, imine, isocyanide, iscyante, N-tert-butoxycarbonyl, nitrate, nitrile, nitrite, nitro, nitroso, phosphate, phosphono, sulfide, sulfonyl, sulfo, sulfhydryl, thiol, thiocyanyl, trifluoromethyl or trifluromethyl ether (OCF3).

Preferred substituents are disclosed herein and exemplified in the tables, structures, examples, and claims, and may be applied across different compounds of this disclosure. For example, substituents of a given compound may be combinatorically used with other compounds. It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art may apply techniques most likely to achieve the desired separation.

Non-limiting examples of suitable solvents that may be used in this disclosure include water, methanol (MeOH), ethanol (EtOH), dichloromethane or methylene chloride (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

The term “subject” refers to an animal including a human.

The term “therapeutically effective amount” refers to the amount of a compound that produces the desired effect for which it is administered (e.g., improvement in a disease or condition, lessening the severity of a disease or condition, and/or reducing progression of a disease or condition, a disease or condition selected from an inflammatory disease, an immune disease (e.g., an autoimmune disease), an allergic disease, transplant rejection, a necrotic cell disease (e.g., a disease associated with necroptosis), a neurodegenerative disease, a central nervous system (CNS) disease, ischemic brain injury, an ocular disease, an infectious disease, and a malignancy, including those mediated by receptor-interacting protein 1 (RIP1) signaling; a disease or condition selected from ulcerative colitis, Crohn's disease, psoriasis, rheumatoid arthritis, amyotrophic lateral sclerosis (ALS), Alzheimer's disease, and a viral infection, including those mediated by RIP1 signaling; a disease or condition mediated by RIP1 signaling. The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, curing a disease or condition or a symptom thereof, lower risk of a disease or condition, a disease or condition selected from an inflammatory disease, an immune disease (e.g., an autoimmune disease), an allergic disease, transplant rejection, a necrotic cell disease, a neurodegenerative disease, a central nervous system (CNS) disease, ischemic brain injury, an ocular disease, an infectious disease, and a malignancy, including those mediated by receptor-interacting protein 1 (RIP1) signaling; a disease or condition selected from ulcerative colitis, Crohn's disease, psoriasis, rheumatoid arthritis, amyotrophic lateral sclerosis (ALS), Alzheimer's disease, and a viral infection, including those mediated by receptor-interacting protein 1 (RIP1) signaling; a disease or condition mediated by RIP1 signaling. Improvements in or lessening the severity of any of these symptoms can be assessed according to methods and techniques known in the art.

The terms “about” and “approximately,” when used in connection with a number such as a percentage include the number as specified, and a range of the number (e.g., a range of percentages) that is recognized by one of ordinary skill in the art.

### II. Compounds and Compositions

In a first embodiment, a compound of this disclosure is a compound of any one of the following structural formulae Ia and Ib:

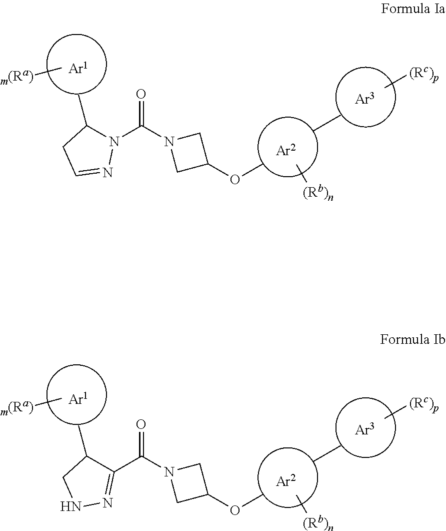

a tautomer thereof, a hydrate or stereoisomer of the compound or the tautomer, or a pharmaceutically acceptable salt of the foregoing, wherein:
Ar¹ is phenyl, C₅-C₆ cycloalkyl, 5- to 6-membered heteroaryl, or 5- to 6-membered heterocyclyl,
Ar² is phenyl, C₅-C₆ cycloalkyl, 5- to 6-membered heteroaryl, or 5- to 6-membered heterocyclyl, provided that when

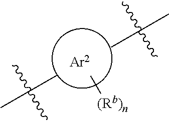

in formula Ia is

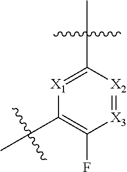

wherein X₁, X₂, and X₃ are C; or X₁ is N, X₂ and X₃ are C; or X₂ is N, X₁ and X₃ are C; or X₁ and X₂ are N, and X₃ is C; or X₁ and X₂ are C, and X₃ is N,

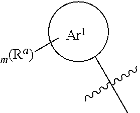

cannot be

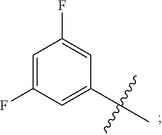 [SMILES: *c1cc(F)cc(F)c1]

Ar³ is phenyl, C₅-C₆ cycloalkyl, 5- to 6-membered heteroaryl, or 5- to 6-membered heterocyclyl;
R^{a}, for each occurrence, is independently selected from halogen, CN, C₁-C₃ alkyl, and OH;
R^{b}, for each occurrence, is independently selected from halogen, CN, C₁-C₃ alkyl, and OH;
R^{c}, for each occurrence, is independently selected from halogen, cyano, C₁-C₆ alkyl, C₃-C₆ cycloalkyl, 3- to 6-membered heterocyclyl, C₂-C₆ alkenyl, C₁-C₆ alkoxy, —C(═O)(C₁-C₆ alkyl), —C(═O)(C₃-C₆ cycloalkyl), —C(═O) (3- to 6-membered heterocyclyl), ═O, —NO₂, —C(═O)OR^{s}, —C(═O)NR^{p}R^{q}, —NR^{p}R^{q}, —NR^{p}C(═O)R^{s}, —NR^{p}C(═O)OR^{s}, —NR^{p}C(═O)NR^{q}R^{r}, —NR^{p}S(═O)_{w}R^{s}, —OR^{s}, —OC(═O)R^{s}, —OC(═O)OR^{s}, —OC(═O)NR^{p}R^{q}, —S(═O)_{w}R^{s}, and —S(═O)_{w}NR^{p}R^{q}; wherein
the C₁-C₆ alkyl, C₃-C₆ cycloalkyl, 3- to 6-membered heterocyclyl, C₂-C₆ alkenyl, and C₁-C₆ alkoxy of R^{c}, the C₁-C₆ alkyl of —C(═O)(C₁-C₆ alkyl), the C₃-C₆ cycloalkyl of —C(═O)(C₃-C₆ cycloalkyl), and the 3- to 6-membered heterocyclyl of —C(═O)(3- to 6-membered heterocyclyl) are each optionally substituted with 1 to 3 groups selected from halogen, cyano, —C(═O)R^{s}, —C(═O)OR^{s}, —C(═O)NR^{p}R^{q}, —NR^{p}R^{q}, —NR^{p}C(═O)R^{s}, —NR^{p}C(═O)OR^{s}, —NR^{p}C(═O)NR^{q}R^{r}, —NR^{p}S(═O)R^{s}, —OR^{s}, —OC(═O)R^{s}, —OC(═O)OR^{s}, —OC(═O)NR^{p}R^{q}, —S(═O)_{w}R^{s}, —S(═O)_{w}NR^{p}R^{q}, C₃-C₆ cycloalkyl, and 3- to 6-membered heterocyclyl; wherein
R^{p}, R^{q}, R^{r}, and R^{s}, for each occurrence, are each independently selected from hydrogen, OH, NH₂, C₁-C₄ alkyl, —C(═O)(C₁-C₄ alkyl), C₃-C₆ cycloalkyl, and 3- to 6-membered heterocyclyl; wherein
the C₁-C₄ alkyl, C₃-C₆ cycloalkyl, and 3- to 6-membered heterocyclyl of any one of R^{p}, R^{q}, R^{r}, and R^{s} are optionally substituted with 1 to 3 groups selected from halogen, cyano, —OH, C₁-C₆ alkyl, —O(C₁-C₆ alkyl), —C(═O)N(C₁-C₆ alkyl)(C₁-C₆ alkyl), —C(═O)NH(C₁-C₆ alkyl), —C(═O)(3- to 6-membered heterocyclyl), —C(═O)(C₃-C₆ cycloalkyl), C₃-C₆ cycloalkyl, phenyl, and 3- to 6-membered heterocyclyl; and wherein
w is an integer selected from 0, 1 and 2;
m and p are each an integer independently selected from 0, 1, 2, and 3; and
n is selected from 0, 1, and 2.

Combinations of substituents as disclosed herein are those that result in the formation of stable or chemically feasible compounds. For abbreviation or according to common practice, certain hydrogen atoms attached to a certain atom (e.g., a carbon atom C or a nitrogen atom N) are not specifically spelled out in a chemical structure, formula, or notation; hydrogen atoms are deemed to be present to the extent the valences of the certain atom (e.g., C or N) are completed. Certain hydrogen atoms shown in a chemical structure, formula, or notation, e.g., in Ar¹ or Ar², may be optionally substituted, e.g., by an R^{a} or R^{c}.

In a second embodiment, in a compound, tautomer, a hydrate or stereoisomer of the compound or the tautomer, or pharmaceutically acceptable salt of this disclosure, Ar¹ is phenyl or 5- to 6-membered heteroaryl, Ar² is phenyl or 6-membered heteroaryl, and Ar³ is 5- to 6-membered heteroaryl; and all other variables not specifically defined herein are as defined in the preceding embodiment.

In a third embodiment, a compound of the disclosure is of the following structural formula IIa:

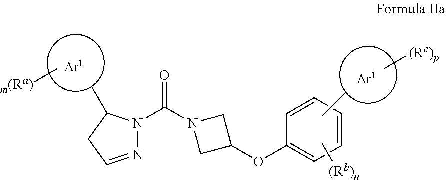

a tautomer thereof, a hydrate or stereoisomer of the compound or the tautomer, or a pharmaceutically acceptable salt of the foregoing; and all other variables not specifically defined herein are as defined in any one of the preceding embodiments.

In a fourth embodiment, a compound of the disclosure is of the following structural formula IIb:

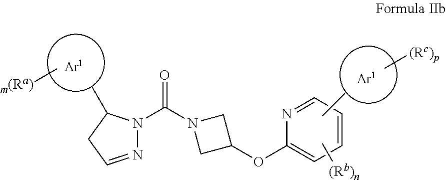

a tautomer thereof, a hydrate or stereoisomer of the compound or the tautomer, or a pharmaceutically acceptable salt of the foregoing; and all other variables not specifically defined herein are as defined in any one of the appropriate preceding embodiments.

In a fifth embodiment, a compound of the disclosure is of the following structural formula IIc:

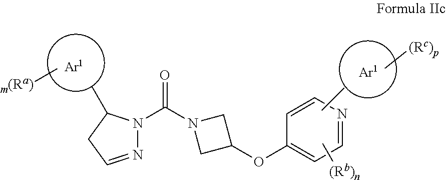

a tautomer thereof, a hydrate or stereoisomer of the compound or the tautomer, or a pharmaceutically acceptable salt of the foregoing; and all other variables not specifically defined herein are as defined in any one of the appropriate preceding embodiments.

In a sixth embodiment, a compound of the disclosure is of the following structural formula IId:

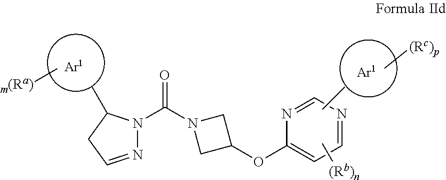

a tautomer thereof, a hydrate or stereoisomer of the compound or the tautomer, or a pharmaceutically acceptable salt of the foregoing; and all other variables not specifically defined herein are as defined in any one of the appropriate preceding embodiments.

In a seventh embodiment, a compound of the disclosure is of the following structural formula IIe:

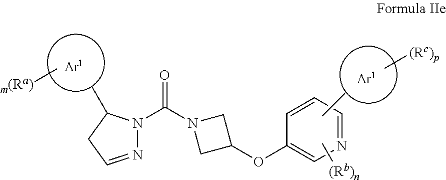

a tautomer thereof, a hydrate or stereoisomer of the compound or the tautomer, or a pharmaceutically acceptable salt of the foregoing; and all other variables not specifically defined herein are as defined in any one of the appropriate preceding embodiments.

In an eighth embodiment, a compound of the disclosure is of the following structural formula IIf:

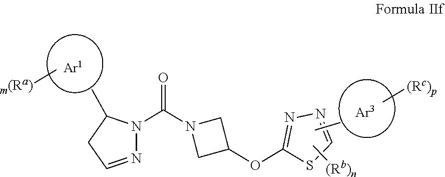

a tautomer thereof, a hydrate or stereoisomer of the compound or the tautomer, or a pharmaceutically acceptable salt of the foregoing; and all other variables not specifically defined herein are as defined in any one of the appropriate preceding embodiments.

In a ninth embodiment, a compound of the disclosure is of the following structural formula IIg:

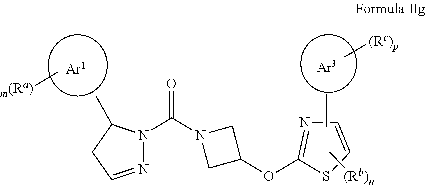

a tautomer thereof, a hydrate or stereoisomer of the compound or the tautomer, or a pharmaceutically acceptable salt of the foregoing; and all other variables not specifically defined herein are as defined in any one of the appropriate preceding embodiments.

In a tenth embodiment, a compound of the disclosure is of the following structural formula IIh:

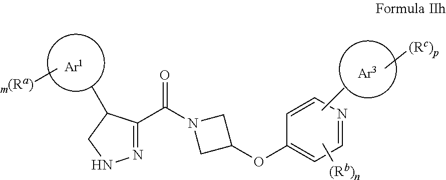

a tautomer thereof, a hydrate or stereoisomer of the compound or the tautomer, or a pharmaceutically acceptable salt of the foregoing; and all other variables not specifically defined herein are as defined in any one of the appropriate preceding embodiments.

In an eleventh embodiment, a compound of the disclosure is of one of the following structural formulae IIIa-1 and IIIa-2:

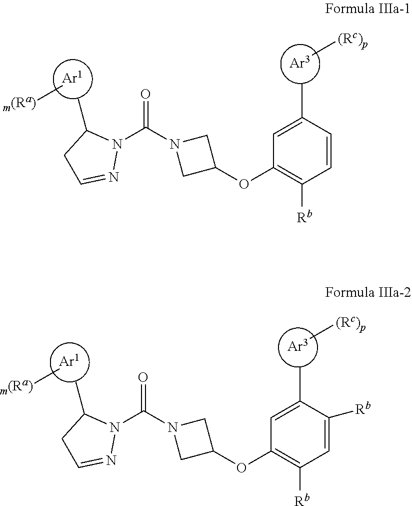

a tautomer thereof, a hydrate or stereoisomer of the compound or the tautomer, or a pharmaceutically acceptable salt of the foregoing, wherein R^{b}, for each occurrence, is independently selected from F and Cl; and all other variables not specifically defined herein are as defined in any one of the appropriate preceding embodiments.

In a twelfth embodiment, a compound of the disclosure is of one of the following structural formulae IIIb-1 and IIIb-2:

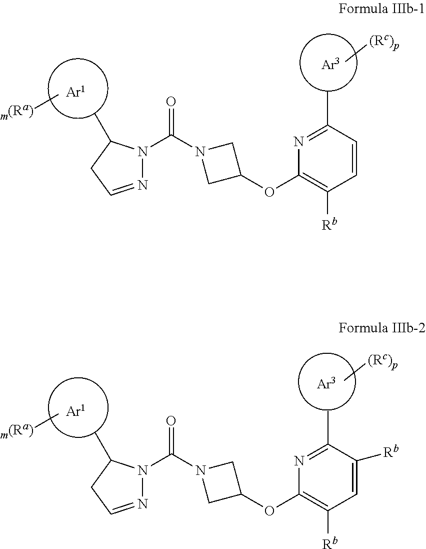

a tautomer thereof, a hydrate or stereoisomer of the compound or the tautomer, or a pharmaceutically acceptable salt of the foregoing, R^{b}, for each occurrence, is independently selected from F and Cl; and all other variables not specifically defined herein are as defined in any one of the appropriate preceding embodiments.

In a thirteenth embodiment, a compound of the disclosure is of the following structural formula IIIc-1:

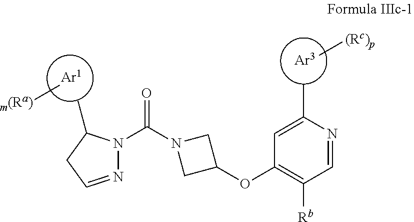

a tautomer thereof, a hydrate or stereoisomer of the compound or the tautomer, or a pharmaceutically acceptable salt of the foregoing, wherein R^{b} is selected from F and Cl; and all other variables not specifically defined herein are as defined in any one of the appropriate preceding embodiments.

In a fourteenth embodiment, a compound of the disclosure is of the following structural formula IIId-1:

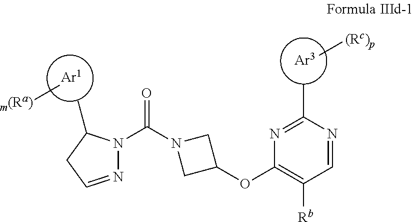

a tautomer thereof, a hydrate or stereoisomer of the compound or the tautomer, or a pharmaceutically acceptable salt of the foregoing, wherein R^{b} is selected from F and Cl; and all other variables not specifically defined herein are as defined in any one of the appropriate preceding embodiments.

In a fifteenth embodiment, a compound of the disclosure is of the following structural formula IIIe-1:

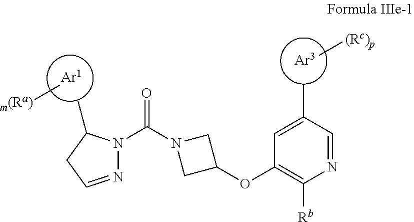

a tautomer thereof, a hydrate or stereoisomer of the compound or the tautomer, or a pharmaceutically acceptable salt of the foregoing, wherein R^{b} is selected from F and Cl; and all other variables not specifically defined herein are as defined in any one of the appropriate preceding embodiments.

In a sixteenth embodiment, a compound of the disclosure is of the following structural formula IIIf-1:

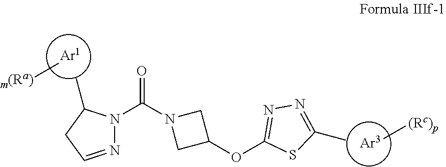

a tautomer thereof, a hydrate or stereoisomer of the compound or the tautomer, or a pharmaceutically acceptable salt of the foregoing; and all other variables not specifically defined herein are as defined in any one of the appropriate preceding embodiments.

In a seventeenth embodiment, a compound of the disclosure is of one of the following structural formulae IIIg-1 and IIIg-2:

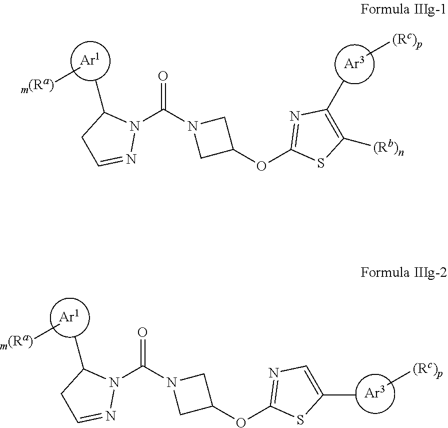

a tautomer thereof, a hydrate or stereoisomer of the compound or the tautomer, or a pharmaceutically acceptable salt of the foregoing, wherein R^{b} is selected from CN and Cl; and all other variables not specifically defined herein are as defined in any one of the appropriate preceding embodiments.

In an eighteenth embodiment, a compound of the disclosure is of the following structural formula IIIh-1:

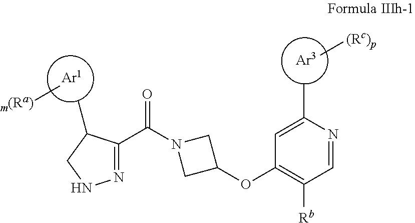

a tautomer thereof, a hydrate or stereoisomer of the compound or the tautomer, or a pharmaceutically acceptable salt of the foregoing, wherein R^{b} is selected from F and Cl; and all other variables not specifically defined herein are as defined in any one of the appropriate preceding embodiments.

In a nineteenth embodiment, in a compound, tautomer, a hydrate or stereoisomer of the compound or the tautomer, or pharmaceutically acceptable salt of this disclosure,

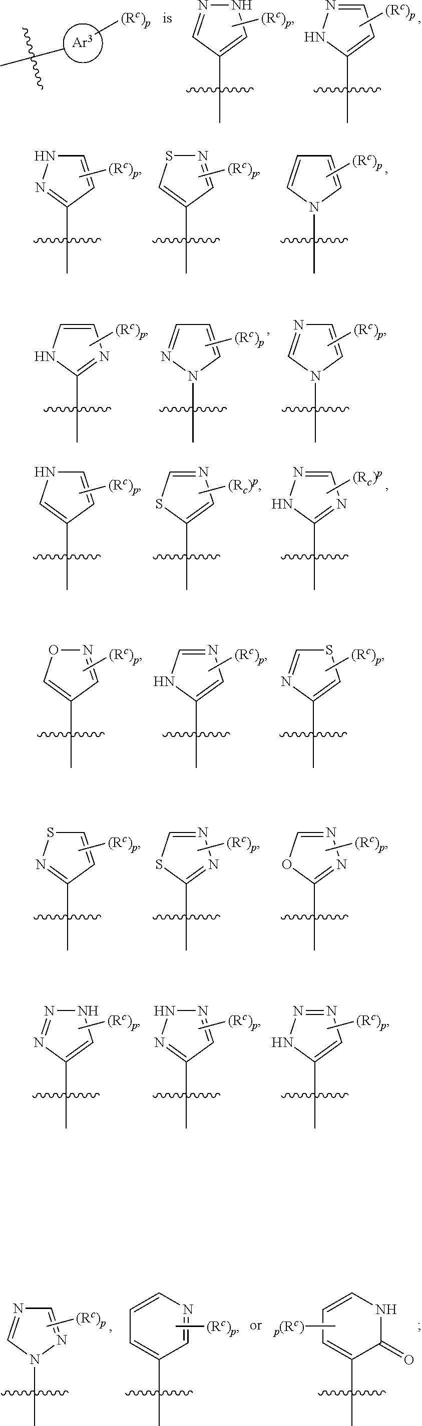

and all other variables not specifically defined herein are as defined in any one of the appropriate preceding embodiments.

In a twentieth embodiment, in a compound, tautomer, a hydrate or stereoisomer of the compound or the tautomer, or pharmaceutically acceptable salt of this disclosure,

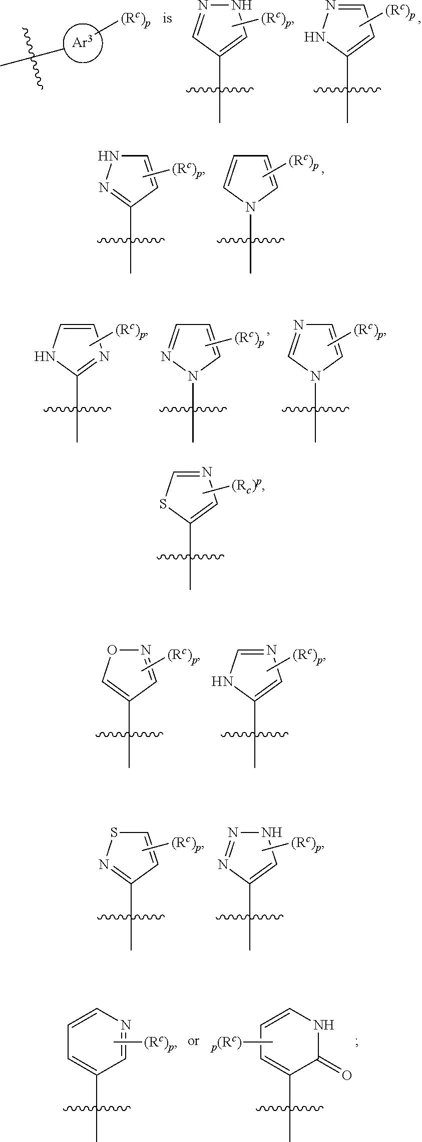

and all other variables not specifically defined herein are as defined in any one of the appropriate preceding embodiments.

In a twenty-first embodiment, in a compound, tautomer, a hydrate or stereoisomer of the compound or the tautomer, or pharmaceutically acceptable salt of this disclosure,

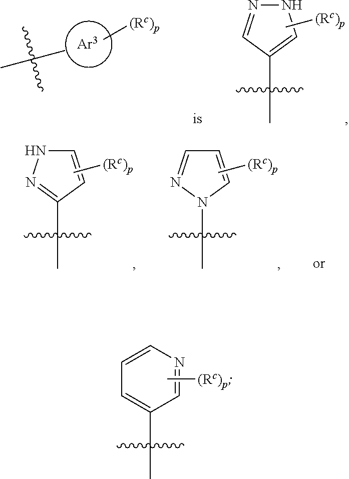

and all other variables not specifically defined herein are as defined in any one of the appropriate preceding embodiments.

In a twenty-second embodiment, in a compound, tautomer, a hydrate or stereoisomer of the compound or the tautomer, or pharmaceutically acceptable salt of this disclosure,

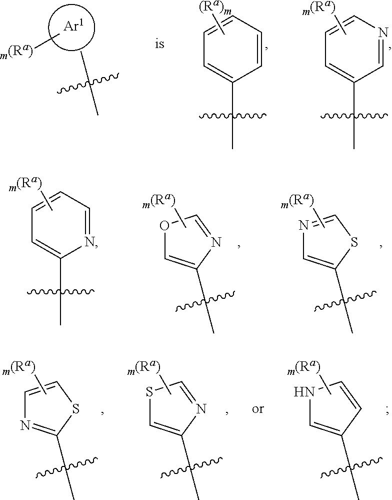

and all other variables not specifically defined herein are as defined in any one of the appropriate preceding embodiments.

In a twenty-third embodiment, in a compound, tautomer, a hydrate or stereoisomer of the compound or the tautomer, or pharmaceutically acceptable salt of this disclosure, R^{a}, for each occurrence, is independently selected from F, Cl, CN, C₁-C₃ alkyl, and OH; and all other variables not specifically defined herein are as defined in any one of the appropriate preceding embodiments.

In a twenty-fourth embodiment, in a compound, tautomer, a hydrate or stereoisomer of the compound or the tautomer, or pharmaceutically acceptable salt of this disclosure, R^{a}, for each occurrence, is independently selected from F, Cl, CN, and methyl; and all other variables not specifically defined herein are as defined in any one of the appropriate preceding embodiments.

In a twenty-fifth embodiment, in a compound, tautomer, a hydrate or stereoisomer of the compound or the tautomer, or pharmaceutically acceptable salt of this disclosure, m is 1 or 2; and all other variables not specifically defined herein are as defined in any one of the appropriate preceding embodiments.

In a twenty-sixth embodiment, in a compound, tautomer, a hydrate or stereoisomer of the compound or the tautomer, or pharmaceutically acceptable salt of this disclosure, R^{c}, for each occurrence, is independently selected from halogen; CN; ═O;
—C(═O)OR^{s}, wherein R^{s} is H or C₁-C₃ alkyl;
C₁-C₃ alkyl, optionally substituted with 1 to 3 groups selected from OH, NH₂, cyano, halogen, C₁-C₃ alkoxyl, 3- to 4-membered cycloalkyl;
—C(═O)NR^{p}R^{q}, wherein R^{p} and R^{q} each are independently selected from H, OH, 3- to 4-membered cycloalkyl, and 4- to 6-membered heterocyclyl;
—NR^{p}R^{q}, wherein R^{p} and R^{q} each are independently selected from H, OH, —C(═O)CH₃, and C₁-C₃ alkyl;
—S(═O)_{w}R^{s}, wherein R^{s} is selected from C₁-C₃ alkyl and wherein w is 0 or 2; and —S(═O)_{w}NR^{p}R^{q}, wherein R^{p} and R^{q} each are independently selected from H and C₁-C₃ alkyl and wherein w is 2;
and all other variables not specifically defined herein are as defined in any one of the appropriate preceding embodiments.

In a twenty-seventh embodiment, in a compound, tautomer, a hydrate or stereoisomer of the compound or the tautomer, or pharmaceutically acceptable salt of this disclosure, R^{c}, for each occurrence, is independently selected from methyl, Cl, CN, ethyl, —C(═O)NH₂, —CH₂CH₂OCH₃, —CH₂C(═O)NH₂, —NH₂, F, —S(═O)₂CH₂CH₃,

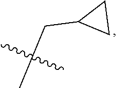 [SMILES: *CC1CC1]

—C(═O)NHCH₂CH₂OH,

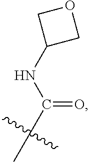 [SMILES: *C(=O)NC1COC1]

### —C(═O)NHCH₃,

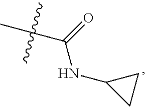 [SMILES: *C(=O)NC1CC1]

—C(═O)OH, —C(═O)NHCH₂CH₃, and —S(═O)₂NH₂; and all other variables not specifically defined herein are as defined in any one of the appropriate preceding embodiments.

In a twenty-eighth embodiment, in a compound, tautomer, a hydrate or stereoisomer of the compound or the tautomer, or pharmaceutically acceptable salt of this disclosure, R^{c}, for each occurrence, is independently selected from CN, methyl, ethyl, F, Cl, and —C(═O)NH₂; and all other variables not specifically defined herein are as defined in any one of the preceding embodiments.

In a twenty-ninth embodiment, in a compound, tautomer, a hydrate or stereoisomer of the compound or the tautomer, or pharmaceutically acceptable salt of this disclosure, p is 1, 2, or 3; and all other variables not specifically defined herein are as defined in any one of the appropriate preceding embodiments.

In a thirtieth embodiment, a compound of the disclosure is of the following structural formula IVa:

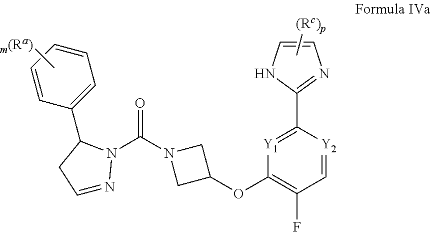

a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

In a thirty-first embodiment, a compound of the disclosure is of the following structural formula IVb:

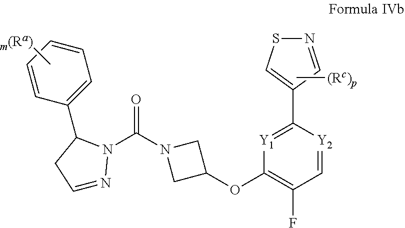

a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

In a thirty-second embodiment, a compound of the disclosure is of the following structural formula IVc:

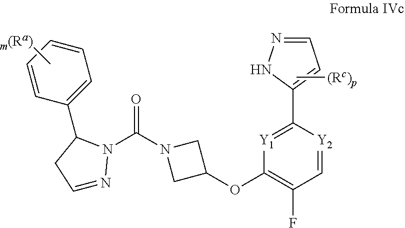

a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

In a thirty-third embodiment, a compound of the disclosure is of the following structural formula IVd:

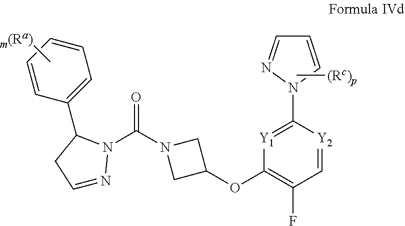

a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

In a thirty-fourth embodiment, a compound of the disclosure is of the following structural formula IVe:

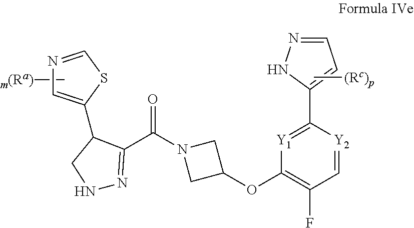

a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

In a thirty-fifth embodiment, a compound of the disclosure is of the following structural formula IVf:

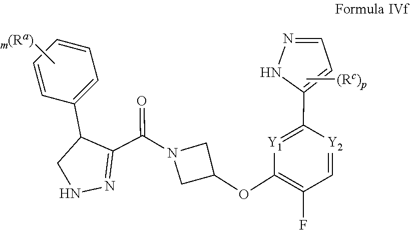

a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

In a thirty-sixth embodiment, a compound of the disclosure is of the following structural formula IVg:

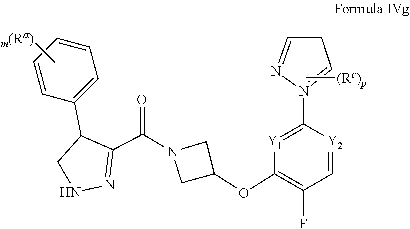

a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

In certain embodiments, the at least one compound of the disclosure is selected from Compounds 1 to 169 depicted in Table 1, a tautomer thereof, a hydrate or stereoisomer of the compound or the tautomer, or a pharmaceutically acceptable salt of the foregoing.

TABLE 1
Compounds 1 to 169

| | |
|---|---|
| 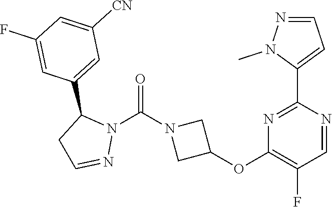 [SMILES: Cn1nccc1-c1ncc(F)c(OC2CN(C(=O)N3N=CC[C@H]3c3cc(F)cc(C#N)c3)C2)n1] | 1 |
| 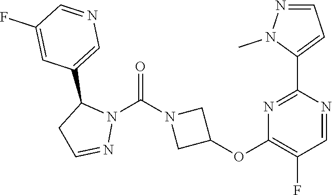 [SMILES: Cn1nccc1-c1ncc(F)c(OC2CN(C(=O)N3N=CC[C@H]3c3cncc(F)c3)C2)n1] | 2 |
| 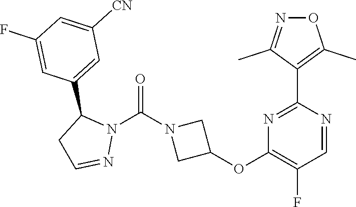 [SMILES: Cc1noc(C)c1-c1ncc(F)c(OC2CN(C(=O)N3N=CC[C@H]3c3cc(F)cc(C#N)c3)C2)n1] | 3 |
| 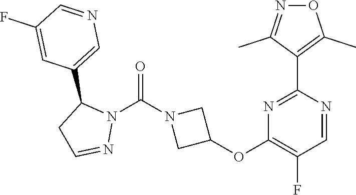 [SMILES: Cc1noc(C)c1-c1ncc(F)c(OC2CN(C(=O)N3N=CC[C@H]3c3cncc(F)c3)C2)n1] | 4 |
| 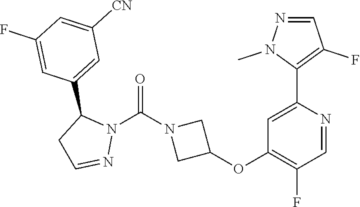 [SMILES: Cn1ncc(F)c1-c1cc(OC2CN(C(=O)N3N=CC[C@H]3c3cc(F)cc(C#N)c3)C2)c(F)cn1] | 5 |
| 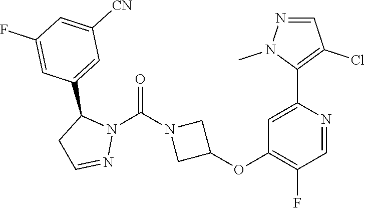 [SMILES: Cn1ncc(Cl)c1-c1cc(OC2CN(C(=O)N3N=CC[C@H]3c3cc(F)cc(C#N)c3)C2)c(F)cn1] | 6 |
| 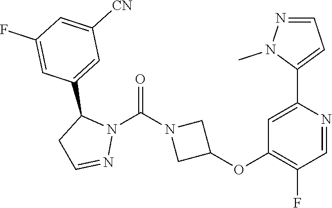 [SMILES: Cn1nccc1-c1cc(OC2CN(C(=O)N3N=CC[C@H]3c3cc(F)cc(C#N)c3)C2)c(F)cn1] | 7 |
| 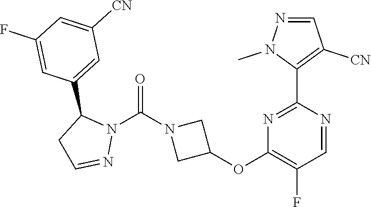 [SMILES: Cn1ncc(C#N)c1-c1ncc(F)c(OC2CN(C(=O)N3N=CC[C@H]3c3cc(F)cc(C#N)c3)C2)n1] | 8 |
| 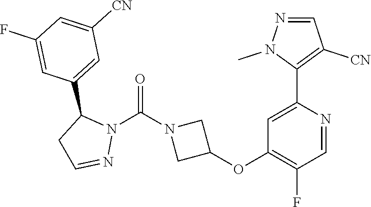 [SMILES: Cn1ncc(C#N)c1-c1cc(OC2CN(C(=O)N3N=CC[C@H]3c3cc(F)cc(C#N)c3)C2)c(F)cn1] | 9 |
| 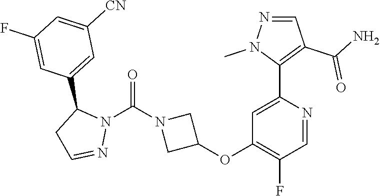 [SMILES: Cn1ncc(C(N)=O)c1-c1cc(OC2CN(C(=O)N3N=CC[C@H]3c3cc(F)cc(C#N)c3)C2)c(F)cn1] | 10 |
| 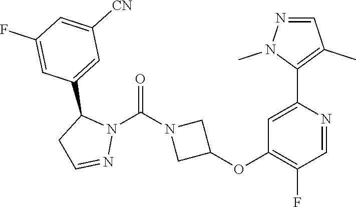 [SMILES: Cc1cnn(C)c1-c1cc(OC2CN(C(=O)N3N=CC[C@H]3c3cc(F)cc(C#N)c3)C2)c(F)cn1] | 11 |
| 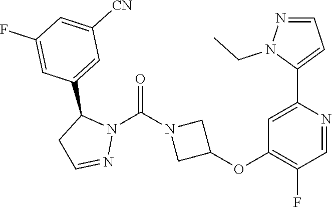 [SMILES: CCn1nccc1-c1cc(OC2CN(C(=O)N3N=CC[C@H]3c3cc(F)cc(C#N)c3)C2)c(F)cn1] | 12 |
| 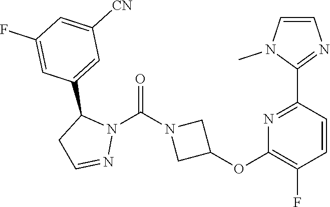 [SMILES: Cn1ccnc1-c1ccc(F)c(OC2CN(C(=O)N3N=CC[C@H]3c3cc(F)cc(C#N)c3)C2)n1] | 13 |
| 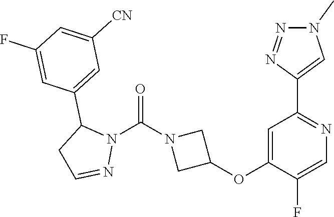 [SMILES: Cn1cc(-c2cc(OC3CN(C(=O)N4N=CCC4c4cc(F)cc(C#N)c4)C3)c(F)cn2)nn1] | 14 |
| 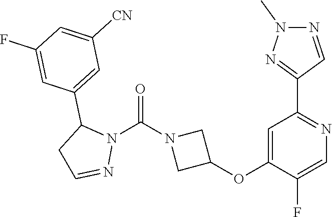 [SMILES: Cn1ncc(-c2cc(OC3CN(C(=O)N4N=CCC4c4cc(F)cc(C#N)c4)C3)c(F)cn2)n1] | 15 |
| 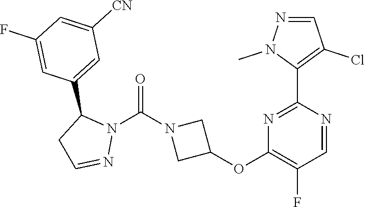 [SMILES: Cn1ncc(Cl)c1-c1ncc(F)c(OC2CN(C(=O)N3N=CC[C@H]3c3cc(F)cc(C#N)c3)C2)n1] | 16 |
| 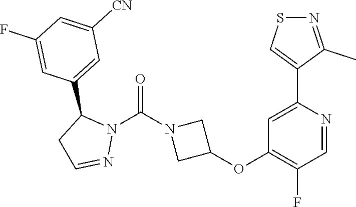 [SMILES: Cc1nscc1-c1cc(OC2CN(C(=O)N3N=CC[C@H]3c3cc(F)cc(C#N)c3)C2)c(F)cn1] | 17 |
| 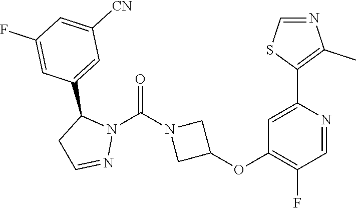 [SMILES: Cc1ncsc1-c1cc(OC2CN(C(=O)N3N=CC[C@H]3c3cc(F)cc(C#N)c3)C2)c(F)cn1] | 18 |
| 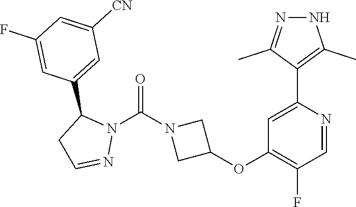 [SMILES: Cc1n[nH]c(C)c1-c1cc(OC2CN(C(=O)N3N=CC[C@H]3c3cc(F)cc(C#N)c3)C2)c(F)cn1] | 19 |
| 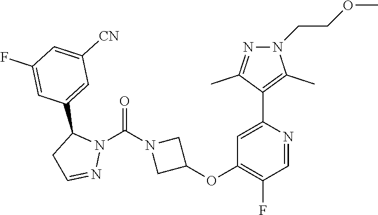 [SMILES: COCCn1nc(C)c(-c2cc(OC3CN(C(=O)N4N=CC[C@H]4c4cc(F)cc(C#N)c4)C3)c(F)cn2)c1C] | 20 |
| 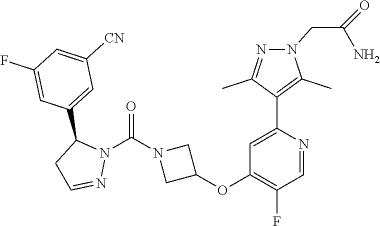 [SMILES: Cc1nn(CC(N)=O)c(C)c1-c1cc(OC2CN(C(=O)N3N=CC[C@H]3c3cc(F)cc(C#N)c3)C2)c(F)cn1] | 21 |
| 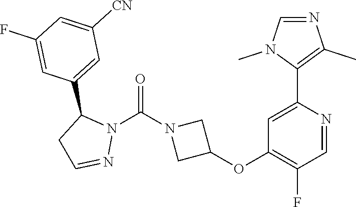 [SMILES: Cc1ncn(C)c1-c1cc(OC2CN(C(=O)N3N=CC[C@H]3c3cc(F)cc(C#N)c3)C2)c(F)cn1] | 22 |
| 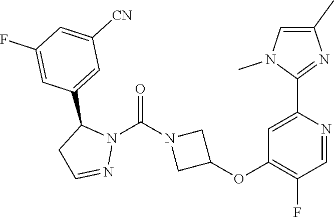 [SMILES: Cc1cn(C)c(-c2cc(OC3CN(C(=O)N4N=CC[C@H]4c4cc(F)cc(C#N)c4)C3)c(F)cn2)n1] | 23 |
| 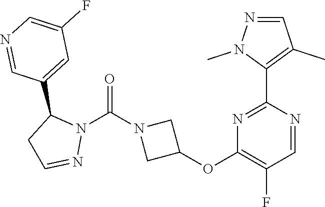 [SMILES: Cc1cnn(C)c1-c1ncc(F)c(OC2CN(C(=O)N3N=CC[C@H]3c3cncc(F)c3)C2)n1] | 24 |
| 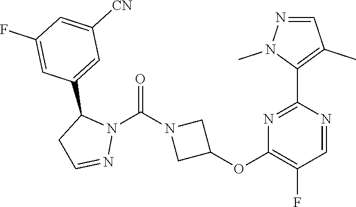 [SMILES: Cc1cnn(C)c1-c1ncc(F)c(OC2CN(C(=O)N3N=CC[C@H]3c3cc(F)cc(C#N)c3)C2)n1] | 25 |
| 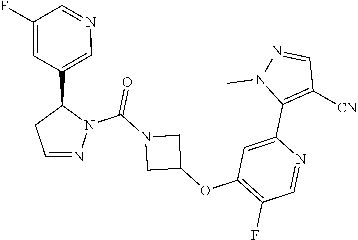 [SMILES: Cn1ncc(C#N)c1-c1cc(OC2CN(C(=O)N3N=CC[C@H]3c3cncc(F)c3)C2)c(F)cn1] | 26 |
| 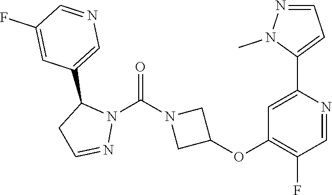 [SMILES: Cn1nccc1-c1cc(OC2CN(C(=O)N3N=CC[C@H]3c3cncc(F)c3)C2)c(F)cn1] | 27 |
| 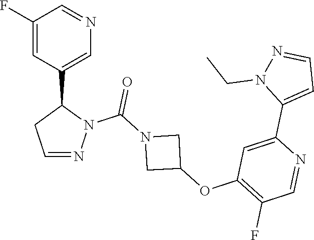 [SMILES: CCn1nccc1-c1cc(OC2CN(C(=O)N3N=CC[C@H]3c3cncc(F)c3)C2)c(F)cn1] | 28 |
| 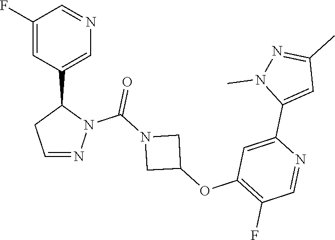 [SMILES: Cc1cc(-c2cc(OC3CN(C(=O)N4N=CC[C@H]4c4cncc(F)c4)C3)c(F)cn2)n(C)n1] | 29 |
| 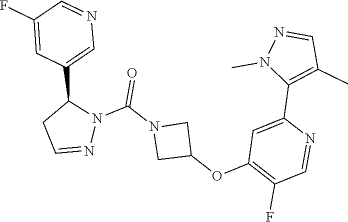 [SMILES: Cc1cnn(C)c1-c1cc(OC2CN(C(=O)N3N=CC[C@H]3c3cncc(F)c3)C2)c(F)cn1] | 30 |
| 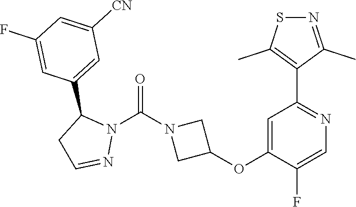 [SMILES: Cc1nsc(C)c1-c1cc(OC2CN(C(=O)N3N=CC[C@H]3c3cc(F)cc(C#N)c3)C2)c(F)cn1] | 31 |
| 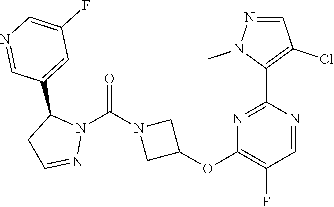 [SMILES: Cn1ncc(Cl)c1-c1ncc(F)c(OC2CN(C(=O)N3N=CC[C@H]3c3cncc(F)c3)C2)n1] | 32 |
| 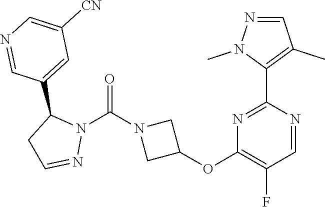 [SMILES: Cc1cnn(C)c1-c1ncc(F)c(OC2CN(C(=O)N3N=CC[C@H]3c3cncc(C#N)c3)C2)n1] | 33 |
| 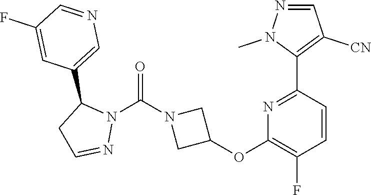 [SMILES: Cn1ncc(C#N)c1-c1ccc(F)c(OC2CN(C(=O)N3N=CC[C@H]3c3cncc(F)c3)C2)n1] | 34 |
| 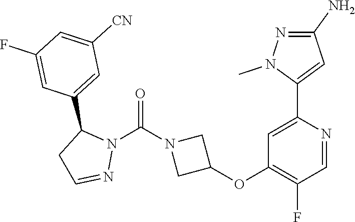 [SMILES: Cn1nc(N)cc1-c1cc(OC2CN(C(=O)N3N=CC[C@H]3c3cc(F)cc(C#N)c3)C2)c(F)cn1] | 35 |
| 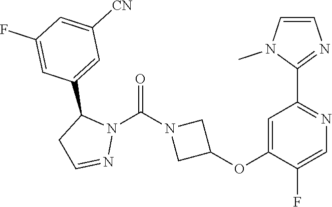 [SMILES: Cn1ccnc1-c1cc(OC2CN(C(=O)N3N=CC[C@H]3c3cc(F)cc(C#N)c3)C2)c(F)cn1] | 36 |
| 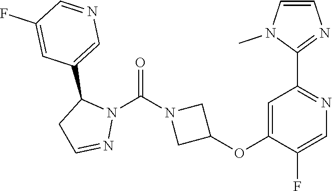 [SMILES: Cn1ccnc1-c1cc(OC2CN(C(=O)N3N=CC[C@H]3c3cncc(F)c3)C2)c(F)cn1] | 37 |
| 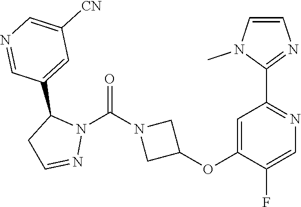 [SMILES: Cn1ccnc1-c1cc(OC2CN(C(=O)N3N=CC[C@H]3c3cncc(C#N)c3)C2)c(F)cn1] | 38 |
| 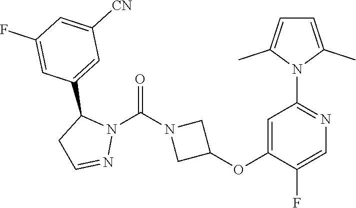 [SMILES: Cc1ccc(C)n1-c1cc(OC2CN(C(=O)N3N=CC[C@H]3c3cc(F)cc(C#N)c3)C2)c(F)cn1] | 39 |
| 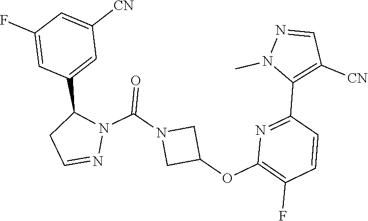 [SMILES: Cn1ncc(C#N)c1-c1ccc(F)c(OC2CN(C(=O)N3N=CC[C@H]3c3cc(F)cc(C#N)c3)C2)n1] | 40 |
| 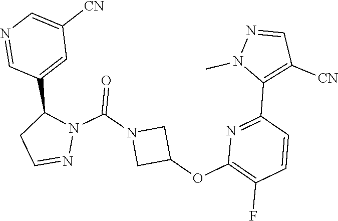 [SMILES: Cn1ncc(C#N)c1-c1ccc(F)c(OC2CN(C(=O)N3N=CC[C@H]3c3cncc(C#N)c3)C2)n1] | 41 |
| 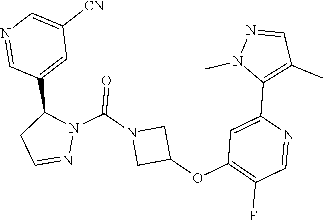 [SMILES: Cc1cnn(C)c1-c1cc(OC2CN(C(=O)N3N=CC[C@H]3c3cncc(C#N)c3)C2)c(F)cn1] | 42 |
| 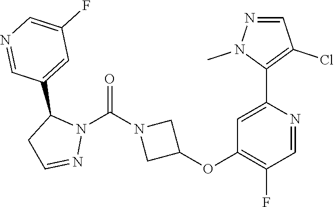 [SMILES: Cn1ncc(Cl)c1-c1cc(OC2CN(C(=O)N3N=CC[C@H]3c3cncc(F)c3)C2)c(F)cn1] | 43 |
| 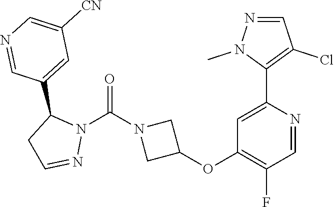 [SMILES: Cn1ncc(Cl)c1-c1cc(OC2CN(C(=O)N3N=CC[C@H]3c3cncc(C#N)c3)C2)c(F)cn1] | 44 |
| 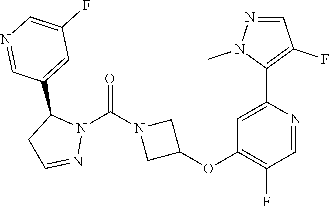 [SMILES: Cn1ncc(F)c1-c1cc(OC2CN(C(=O)N3N=CC[C@H]3c3cncc(F)c3)C2)c(F)cn1] | 45 |
| 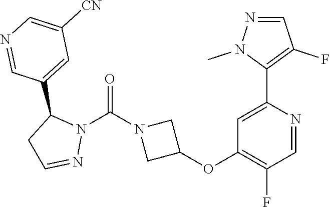 [SMILES: Cn1ncc(F)c1-c1cc(OC2CN(C(=O)N3N=CC[C@H]3c3cncc(C#N)c3)C2)c(F)cn1] | 46 |
| 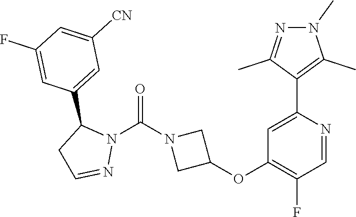 [SMILES: Cc1nn(C)c(C)c1-c1cc(OC2CN(C(=O)N3N=CC[C@H]3c3cc(F)cc(C#N)c3)C2)c(F)cn1] | 47 |
| 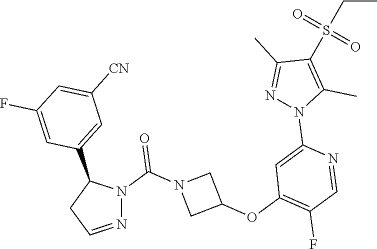 [SMILES: CCS(=O)(=O)c1c(C)nn(-c2cc(OC3CN(C(=O)N4N=CC[C@H]4c4cc(F)cc(C#N)c4)C3)c(F)cn2)c1C] | 48 |
| 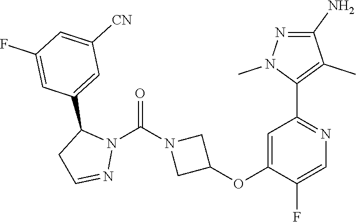 [SMILES: Cc1c(N)nn(C)c1-c1cc(OC2CN(C(=O)N3N=CC[C@H]3c3cc(F)cc(C#N)c3)C2)c(F)cn1] | 49 |
| 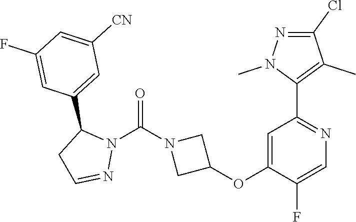 [SMILES: Cc1c(Cl)nn(C)c1-c1cc(OC2CN(C(=O)N3N=CC[C@H]3c3cc(F)cc(C#N)c3)C2)c(F)cn1] | 50 |
| 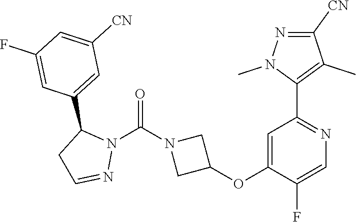 [SMILES: Cc1c(C#N)nn(C)c1-c1cc(OC2CN(C(=O)N3N=CC[C@H]3c3cc(F)cc(C#N)c3)C2)c(F)cn1] | 51 |
| 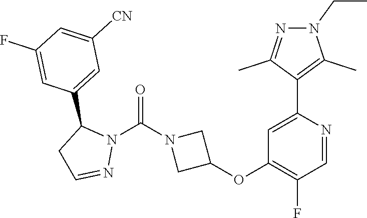 [SMILES: CCn1nc(C)c(-c2cc(OC3CN(C(=O)N4N=CC[C@H]4c4cc(F)cc(C#N)c4)C3)c(F)cn2)c1C] | 52 |
| 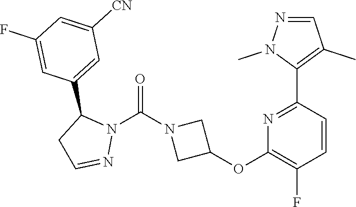 [SMILES: Cc1cnn(C)c1-c1ccc(F)c(OC2CN(C(=O)N3N=CC[C@H]3c3cc(F)cc(C#N)c3)C2)n1] | 53 |
| 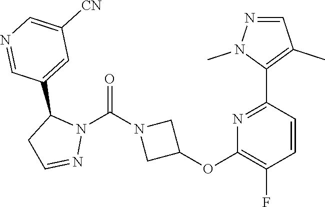 [SMILES: Cc1cnn(C)c1-c1ccc(F)c(OC2CN(C(=O)N3N=CC[C@H]3c3cncc(C#N)c3)C2)n1] | 54 |
| 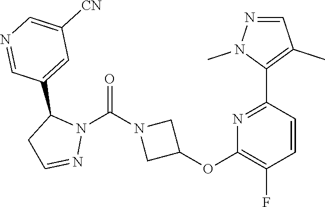 [SMILES: Cc1cnn(C)c1-c1ccc(F)c(OC2CN(C(=O)N3N=CC[C@H]3c3cncc(C#N)c3)C2)n1] | 55 |
| 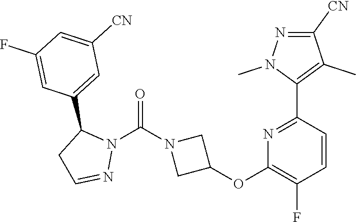 [SMILES: Cc1c(C#N)nn(C)c1-c1ccc(F)c(OC2CN(C(=O)N3N=CC[C@H]3c3cc(F)cc(C#N)c3)C2)n1] | 56 |
| 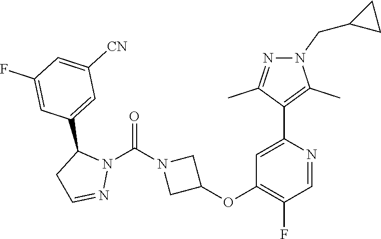 [SMILES: Cc1nn(CC2CC2)c(C)c1-c1cc(OC2CN(C(=O)N3N=CC[C@H]3c3cc(F)cc(C#N)c3)C2)c(F)cn1] | 57 |
| 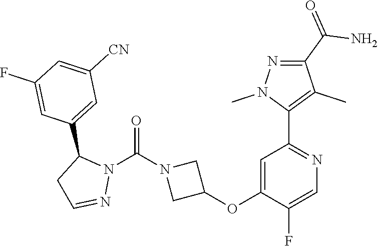 [SMILES: Cc1c(C(N)=O)nn(C)c1-c1cc(OC2CN(C(=O)N3N=CC[C@H]3c3cc(F)cc(C#N)c3)C2)c(F)cn1] | 58 |
| 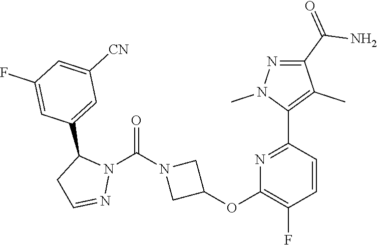 [SMILES: Cc1c(C(N)=O)nn(C)c1-c1ccc(F)c(OC2CN(C(=O)N3N=CC[C@H]3c3cc(F)cc(C#N)c3)C2)n1] | 59 |
| 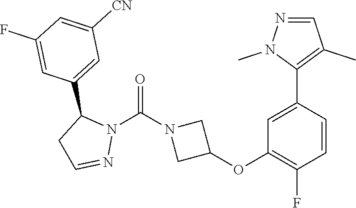 [SMILES: Cc1cnn(C)c1-c1ccc(F)c(OC2CN(C(=O)N3N=CC[C@H]3c3cc(F)cc(C#N)c3)C2)c1] | 60 |
| 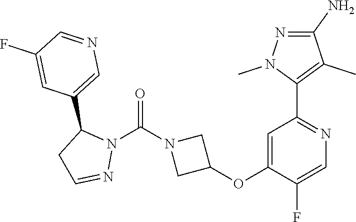 [SMILES: Cc1c(N)nn(C)c1-c1cc(OC2CN(C(=O)N3N=CC[C@H]3c3cncc(F)c3)C2)c(F)cn1] | 61 |
| 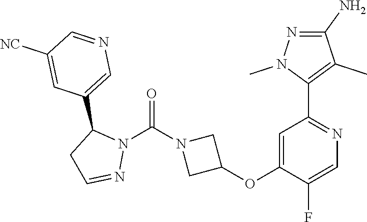 [SMILES: Cc1c(N)nn(C)c1-c1cc(OC2CN(C(=O)N3N=CC[C@H]3c3cncc(C#N)c3)C2)c(F)cn1] | 62 |
| 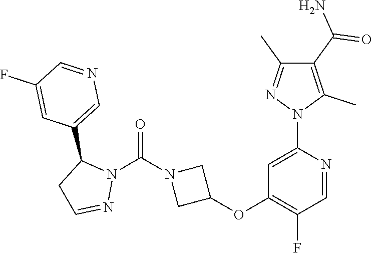 [SMILES: Cc1nn(-c2cc(OC3CN(C(=O)N4N=CC[C@H]4c4cncc(F)c4)C3)c(F)cn2)c(C)c1C(N)=O] | 63 |
| 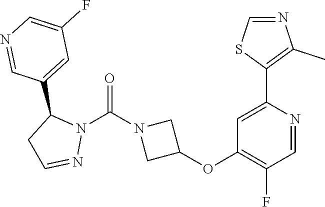 [SMILES: Cc1ncsc1-c1cc(OC2CN(C(=O)N3N=CC[C@H]3c3cncc(F)c3)C2)c(F)cn1] | 64 |
| 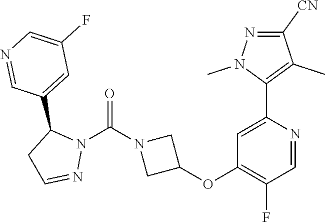 [SMILES: Cc1c(C#N)nn(C)c1-c1cc(OC2CN(C(=O)N3N=CC[C@H]3c3cncc(F)c3)C2)c(F)cn1] | 65 |
| 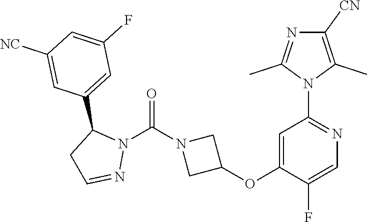 [SMILES: Cc1nc(C#N)c(C)n1-c1cc(OC2CN(C(=O)N3N=CC[C@H]3c3cc(F)cc(C#N)c3)C2)c(F)cn1] | 66 |
| 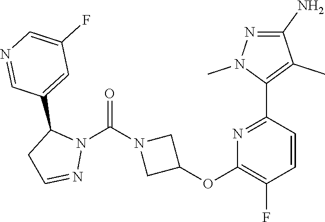 [SMILES: Cc1c(N)nn(C)c1-c1ccc(F)c(OC2CN(C(=O)N3N=CC[C@H]3c3cncc(F)c3)C2)n1] | 67 |
| 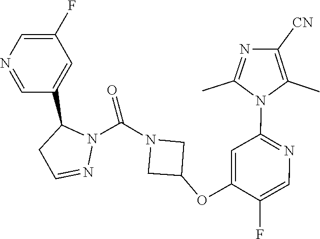 [SMILES: Cc1nc(C#N)c(C)n1-c1cc(OC2CN(C(=O)N3N=CC[C@H]3c3cncc(F)c3)C2)c(F)cn1] | 68 |
| 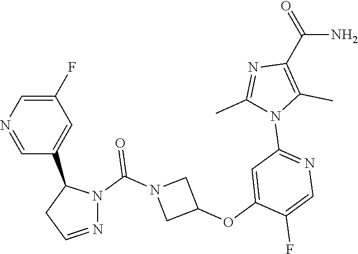 [SMILES: Cc1nc(C(N)=O)c(C)n1-c1cc(OC2CN(C(=O)N3N=CC[C@H]3c3cncc(F)c3)C2)c(F)cn1] | 69 |
| 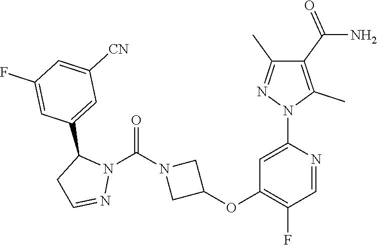 [SMILES: Cc1nn(-c2cc(OC3CN(C(=O)N4N=CC[C@H]4c4cc(F)cc(C#N)c4)C3)c(F)cn2)c(C)c1C(N)=O] | 70 |
| 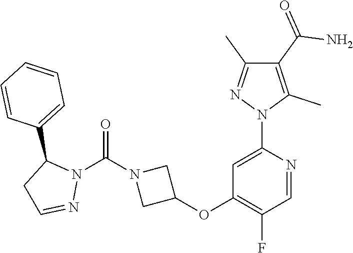 [SMILES: Cc1nn(-c2cc(OC3CN(C(=O)N4N=CC[C@H]4c4ccccc4)C3)c(F)cn2)c(C)c1C(N)=O] | 71 |
| 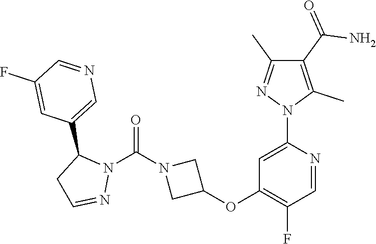 [SMILES: Cc1nn(-c2cc(OC3CN(C(=O)N4N=CC[C@H]4c4cncc(F)c4)C3)c(F)cn2)c(C)c1C(N)=O] | 72 |
| 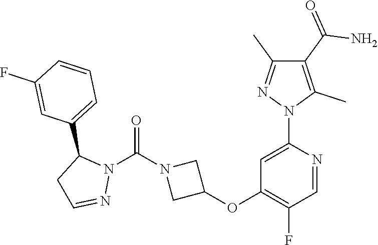 [SMILES: Cc1nn(-c2cc(OC3CN(C(=O)N4N=CC[C@H]4c4cccc(F)c4)C3)c(F)cn2)c(C)c1C(N)=O] | 73 |
| 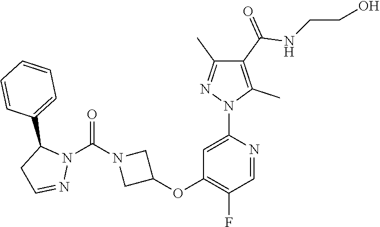 [SMILES: Cc1nn(-c2cc(OC3CN(C(=O)N4N=CC[C@H]4c4ccccc4)C3)c(F)cn2)c(C)c1C(=O)NCCO] | 74 |
| 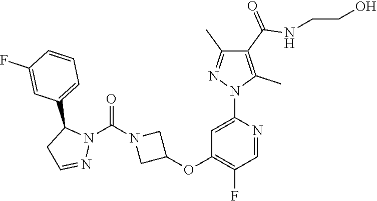 [SMILES: Cc1nn(-c2cc(OC3CN(C(=O)N4N=CC[C@H]4c4cccc(F)c4)C3)c(F)cn2)c(C)c1C(=O)NCCO] | 75 |
| 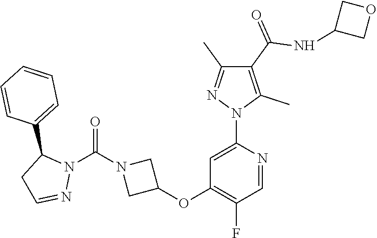 [SMILES: Cc1nn(-c2cc(OC3CN(C(=O)N4N=CC[C@H]4c4ccccc4)C3)c(F)cn2)c(C)c1C(=O)NC1COC1] | 76 |
| 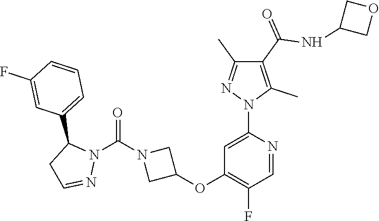 [SMILES: Cc1nn(-c2cc(OC3CN(C(=O)N4N=CC[C@H]4c4cccc(F)c4)C3)c(F)cn2)c(C)c1C(=O)NC1COC1] | 77 |
| 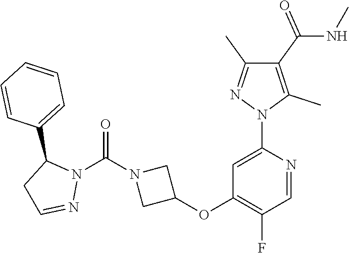 [SMILES: CNC(=O)c1c(C)nn(-c2cc(OC3CN(C(=O)N4N=CC[C@H]4c4ccccc4)C3)c(F)cn2)c1C] | 78 |
| 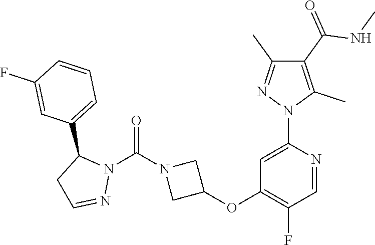 [SMILES: CNC(=O)c1c(C)nn(-c2cc(OC3CN(C(=O)N4N=CC[C@H]4c4cccc(F)c4)C3)c(F)cn2)c1C] | 79 |
| 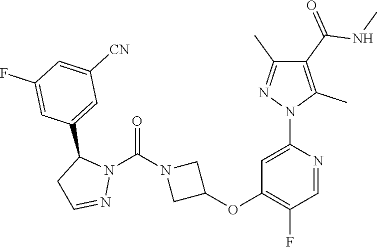 [SMILES: CNC(=O)c1c(C)nn(-c2cc(OC3CN(C(=O)N4N=CC[C@H]4c4cc(F)cc(C#N)c4)C3)c(F)cn2)c1C] | 80 |
| 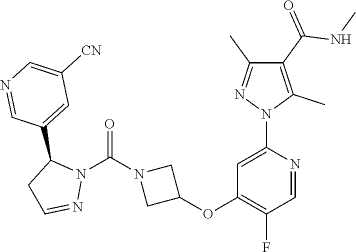 [SMILES: CNC(=O)c1c(C)nn(-c2cc(OC3CN(C(=O)N4N=CC[C@H]4c4cncc(C#N)c4)C3)c(F)cn2)c1C] | 81 |
| 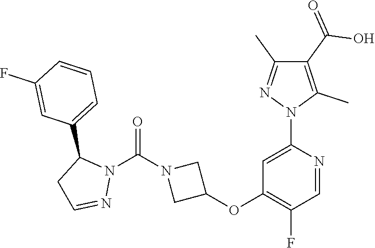 [SMILES: Cc1nn(-c2cc(OC3CN(C(=O)N4N=CC[C@H]4c4cccc(F)c4)C3)c(F)cn2)c(C)c1C(=O)O] | 82 |
| 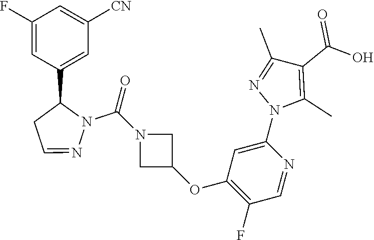 [SMILES: Cc1nn(-c2cc(OC3CN(C(=O)N4N=CC[C@H]4c4cc(F)cc(C#N)c4)C3)c(F)cn2)c(C)c1C(=O)O] | 83 |
| 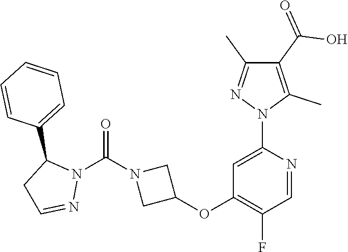 [SMILES: Cc1nn(-c2cc(OC3CN(C(=O)N4N=CC[C@H]4c4ccccc4)C3)c(F)cn2)c(C)c1C(=O)O] | 84 |
| 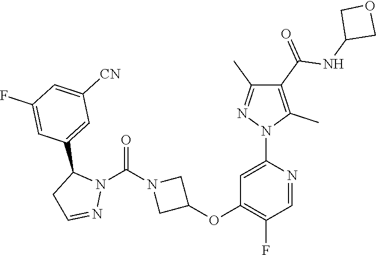 [SMILES: Cc1nn(-c2cc(OC3CN(C(=O)N4N=CC[C@H]4c4cc(F)cc(C#N)c4)C3)c(F)cn2)c(C)c1C(=O)NC1COC1] | 85 |
| 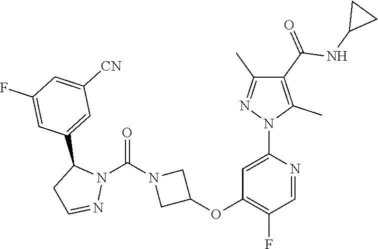 [SMILES: Cc1nn(-c2cc(OC3CN(C(=O)N4N=CC[C@H]4c4cc(F)cc(C#N)c4)C3)c(F)cn2)c(C)c1C(=O)NC1CC1] | 86 |
| 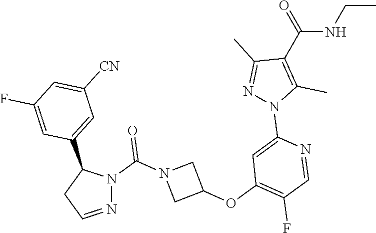 [SMILES: CCNC(=O)c1c(C)nn(-c2cc(OC3CN(C(=O)N4N=CC[C@H]4c4cc(F)cc(C#N)c4)C3)c(F)cn2)c1C] | 87 |
| 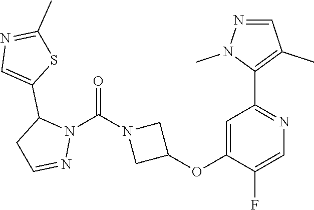 [SMILES: Cc1ncc(C2CC=NN2C(=O)N2CC(Oc3cc(-c4c(C)cnn4C)ncc3F)C2)s1] | 88 |
| 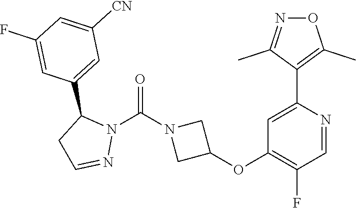 [SMILES: Cc1noc(C)c1-c1cc(OC2CN(C(=O)N3N=CC[C@H]3c3cc(F)cc(C#N)c3)C2)c(F)cn1] | 89 |
| 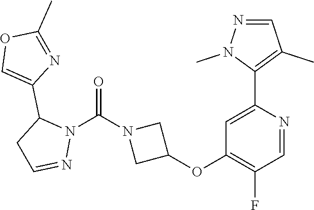 [SMILES: Cc1nc(C2CC=NN2C(=O)N2CC(Oc3cc(-c4c(C)cnn4C)ncc3F)C2)co1] | 90 |
| 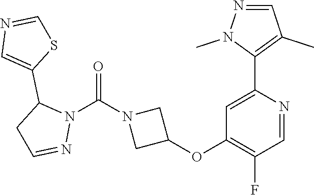 [SMILES: Cc1cnn(C)c1-c1cc(OC2CN(C(=O)N3N=CCC3c3cncs3)C2)c(F)cn1] | 91 |
| 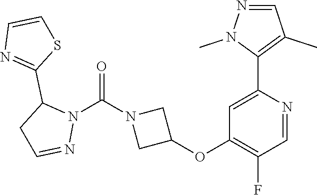 [SMILES: Cc1cnn(C)c1-c1cc(OC2CN(C(=O)N3N=CCC3c3nccs3)C2)c(F)cn1] | 92 |
| 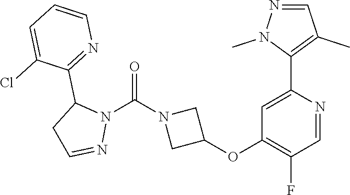 [SMILES: Cc1cnn(C)c1-c1cc(OC2CN(C(=O)N3N=CCC3c3ncccc3Cl)C2)c(F)cn1] | 93 |
| 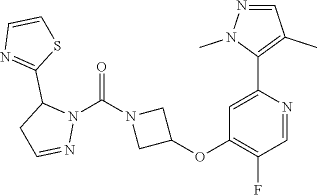 [SMILES: Cc1cnn(C)c1-c1cc(OC2CN(C(=O)N3N=CCC3c3nccs3)C2)c(F)cn1] | 94 |
| 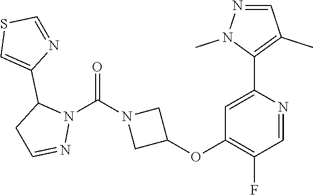 [SMILES: Cc1cnn(C)c1-c1cc(OC2CN(C(=O)N3N=CCC3c3cscn3)C2)c(F)cn1] | 95 |
| 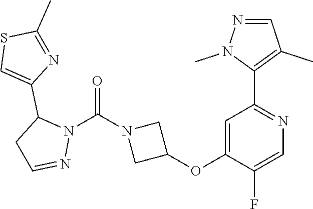 [SMILES: Cc1nc(C2CC=NN2C(=O)N2CC(Oc3cc(-c4c(C)cnn4C)ncc3F)C2)cs1] | 96 |
| 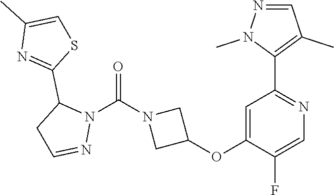 | 97 |
| 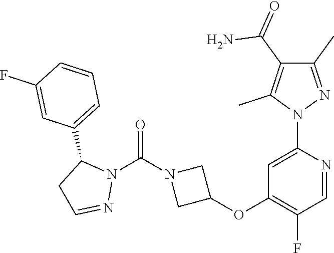 [SMILES: Cc1nn(-c2cc(OC3CN(C(=O)N4N=CC[C@@H]4c4cccc(F)c4)C3)c(F)cn2)c(C)c1C(N)=O] | 98 |
| 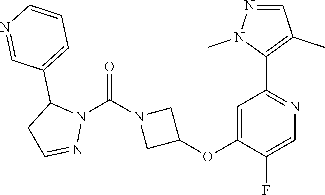 [SMILES: Cc1cnn(C)c1-c1cc(OC2CN(C(=O)N3N=CCC3c3cccnc3)C2)c(F)cn1] | 99 |
| 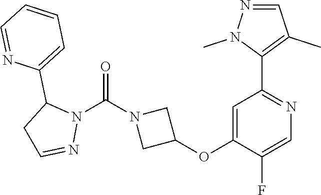 [SMILES: Cc1cnn(C)c1-c1cc(OC2CN(C(=O)N3N=CCC3c3ccccn3)C2)c(F)cn1] | 100 |
| 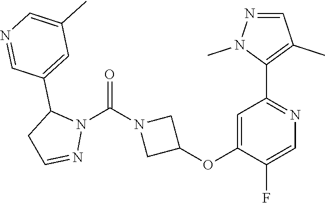 [SMILES: Cc1cncc(C2CC=NN2C(=O)N2CC(Oc3cc(-c4c(C)cnn4C)ncc3F)C2)c1] | 101 |
| 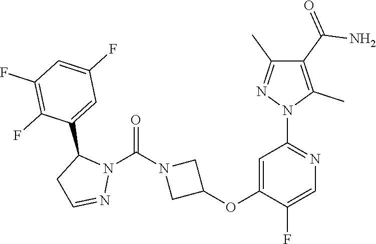 [SMILES: Cc1nn(-c2cc(OC3CN(C(=O)N4N=CC[C@H]4c4cc(F)cc(F)c4F)C3)c(F)cn2)c(C)c1C(N)=O] | 102 |
| 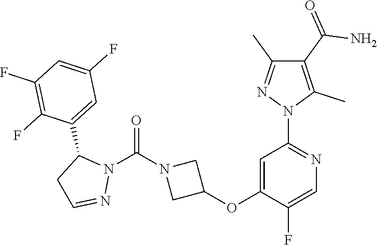 [SMILES: Cc1nn(-c2cc(OC3CN(C(=O)N4N=CC[C@@H]4c4cc(F)cc(F)c4F)C3)c(F)cn2)c(C)c1C(N)=O] | 103 |
| 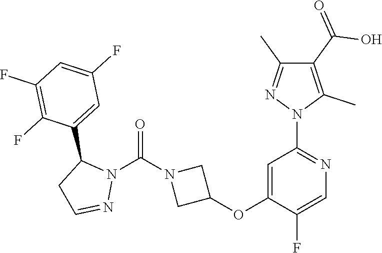 [SMILES: Cc1nn(-c2cc(OC3CN(C(=O)N4N=CC[C@H]4c4cc(F)cc(F)c4F)C3)c(F)cn2)c(C)c1C(=O)O] | 104 |
| 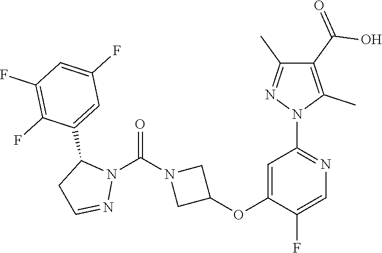 [SMILES: Cc1nn(-c2cc(OC3CN(C(=O)N4N=CC[C@@H]4c4cc(F)cc(F)c4F)C3)c(F)cn2)c(C)c1C(=O)O] | 105 |
| 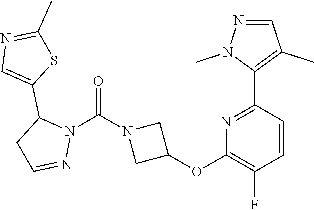 [SMILES: Cc1ncc(C2CC=NN2C(=O)N2CC(Oc3nc(-c4c(C)cnn4C)ccc3F)C2)s1] | 106 |
| 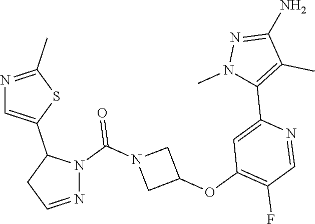 [SMILES: Cc1ncc(C2CC=NN2C(=O)N2CC(Oc3cc(-c4c(C)c(N)nn4C)ncc3F)C2)s1] | 107 |
| 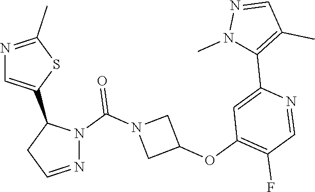 [SMILES: Cc1ncc([C@@H]2CC=NN2C(=O)N2CC(Oc3cc(-c4c(C)cnn4C)ncc3F)C2)s1] | 108 |
| 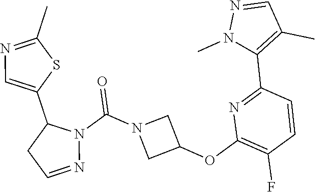 [SMILES: Cc1ncc(C2CC=NN2C(=O)N2CC(Oc3nc(-c4c(C)cnn4C)ccc3F)C2)s1] | 109 |
| 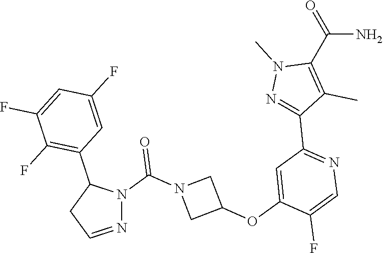 [SMILES: Cc1c(-c2cc(OC3CN(C(=O)N4N=CCC4c4cc(F)cc(F)c4F)C3)c(F)cn2)nn(C)c1C(N)=O] | 110 |
| 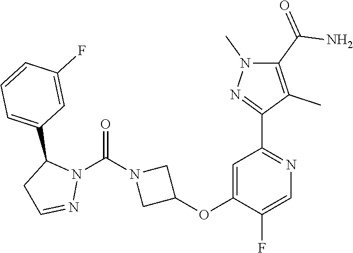 [SMILES: Cc1c(-c2cc(OC3CN(C(=O)N4N=CC[C@H]4c4cccc(F)c4)C3)c(F)cn2)nn(C)c1C(N)=O] | 111 |
| 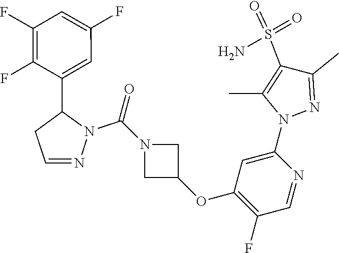 [SMILES: Cc1nn(-c2cc(OC3CN(C(=O)N4N=CCC4c4cc(F)cc(F)c4F)C3)c(F)cn2)c(C)c1S(N)(=O)=O] | 112 |
| 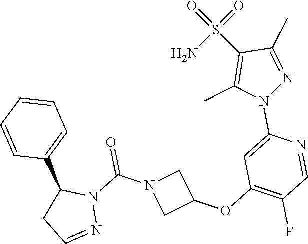 [SMILES: Cc1nn(-c2cc(OC3CN(C(=O)N4N=CC[C@H]4c4ccccc4)C3)c(F)cn2)c(C)c1S(N)(=O)=O] | 113 |
| 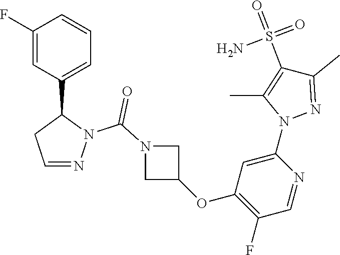 [SMILES: Cc1nn(-c2cc(OC3CN(C(=O)N4N=CC[C@H]4c4cccc(F)c4)C3)c(F)cn2)c(C)c1S(N)(=O)=O] | 114 |
| 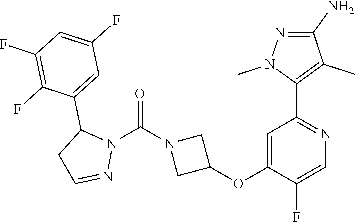 [SMILES: Cc1c(N)nn(C)c1-c1cc(OC2CN(C(=O)N3N=CCC3c3cc(F)cc(F)c3F)C2)c(F)cn1] | 115 |
| 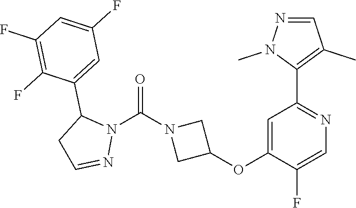 [SMILES: Cc1cnn(C)c1-c1cc(OC2CN(C(=O)N3N=CCC3c3cc(F)cc(F)c3F)C2)c(F)cn1] | 116 |
| 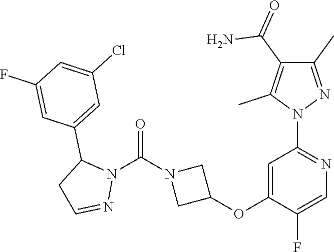 [SMILES: Cc1nn(-c2cc(OC3CN(C(=O)N4N=CCC4c4cc(F)cc(Cl)c4)C3)c(F)cn2)c(C)c1C(N)=O] | 117 |
| 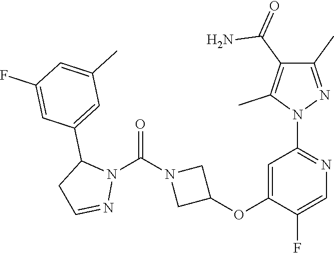 [SMILES: Cc1cc(F)cc(C2CC=NN2C(=O)N2CC(Oc3cc(-n4nc(C)c(C(N)=O)c4C)ncc3F)C2)c1] | 118 |
| 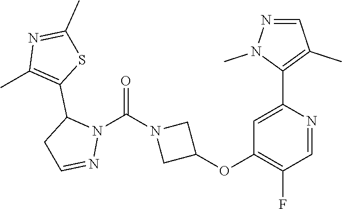 | 119 |
| 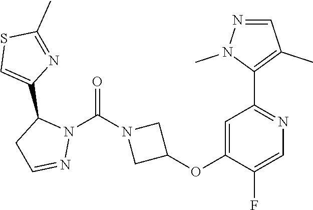 [SMILES: Cc1nc([C@@H]2CC=NN2C(=O)N2CC(Oc3cc(-c4c(C)cnn4C)ncc3F)C2)cs1] | 120 |
| 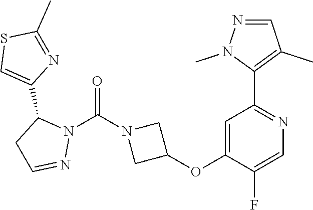 [SMILES: Cc1nc([C@H]2CC=NN2C(=O)N2CC(Oc3cc(-c4c(C)cnn4C)ncc3F)C2)cs1] | 121 |
| 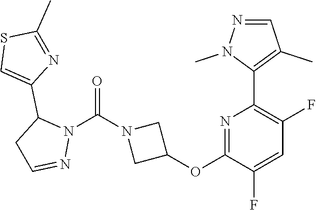 [SMILES: Cc1nc(C2CC=NN2C(=O)N2CC(Oc3nc(-c4c(C)cnn4C)c(F)cc3F)C2)cs1] | 122 |
| 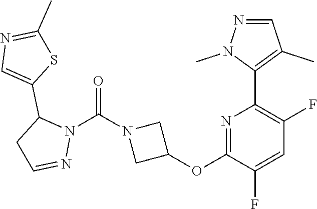 [SMILES: Cc1ncc(C2CC=NN2C(=O)N2CC(Oc3nc(-c4c(C)cnn4C)c(F)cc3F)C2)s1] | 123 |
| 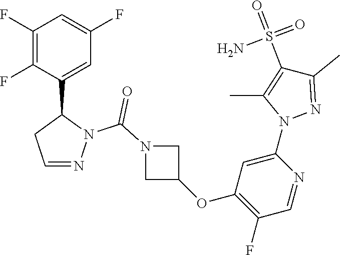 [SMILES: Cc1nn(-c2cc(OC3CN(C(=O)N4N=CC[C@H]4c4cc(F)cc(F)c4F)C3)c(F)cn2)c(C)c1S(N)(=O)=O] | 124 |
| 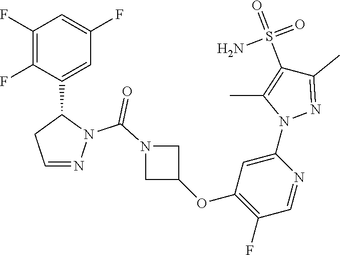 [SMILES: Cc1nn(-c2cc(OC3CN(C(=O)N4N=CC[C@@H]4c4cc(F)cc(F)c4F)C3)c(F)cn2)c(C)c1S(N)(=O)=O] | 125 |
| 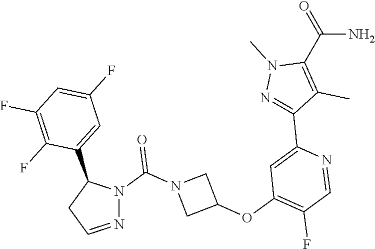 [SMILES: Cc1c(-c2cc(OC3CN(C(=O)N4N=CC[C@H]4c4cc(F)cc(F)c4F)C3)c(F)cn2)nn(C)c1C(N)=O] | 126 |
| 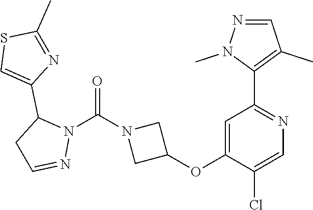 [SMILES: Cc1nc(C2CC=NN2C(=O)N2CC(Oc3cc(-c4c(C)cnn4C)ncc3Cl)C2)cs1] | 127 |
| 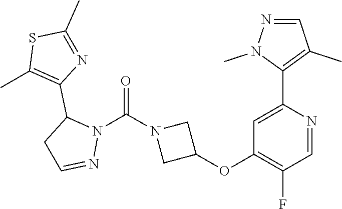 | 128 |
| 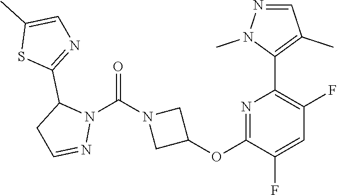 | 129 |
| 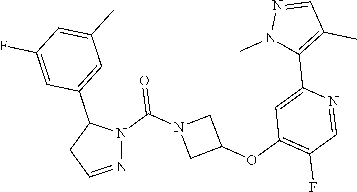 [SMILES: Cc1cc(F)cc(C2CC=NN2C(=O)N2CC(Oc3cc(-c4c(C)cnn4C)ncc3F)C2)c1] | 130 |
| 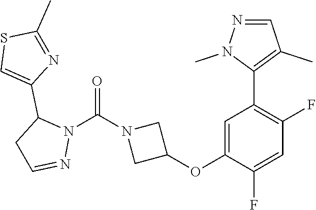 [SMILES: Cc1nc(C2CC=NN2C(=O)N2CC(Oc3cc(-c4c(C)cnn4C)c(F)cc3F)C2)cs1] | 131 |
| 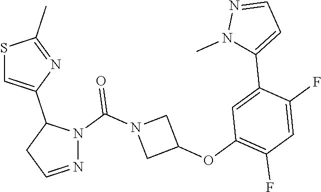 [SMILES: Cc1nc(C2CC=NN2C(=O)N2CC(Oc3cc(-c4ccnn4C)c(F)cc3F)C2)cs1] | 132 |
| 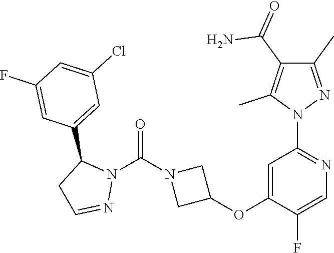 [SMILES: Cc1nn(-c2cc(OC3CN(C(=O)N4N=CC[C@H]4c4cc(F)cc(Cl)c4)C3)c(F)cn2)c(C)c1C(N)=O] | 133 |
| 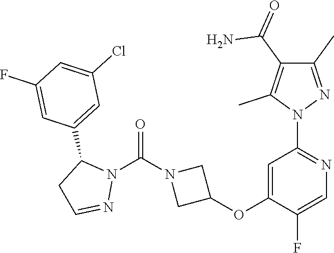 [SMILES: Cc1nn(-c2cc(OC3CN(C(=O)N4N=CC[C@@H]4c4cc(F)cc(Cl)c4)C3)c(F)cn2)c(C)c1C(N)=O] | 134 |
| 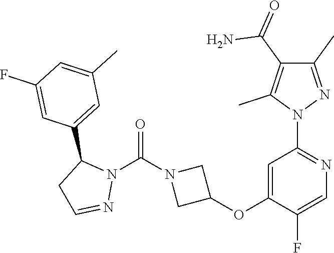 [SMILES: Cc1cc(F)cc([C@@H]2CC=NN2C(=O)N2CC(Oc3cc(-n4nc(C)c(C(N)=O)c4C)ncc3F)C2)c1] | 135 |
| 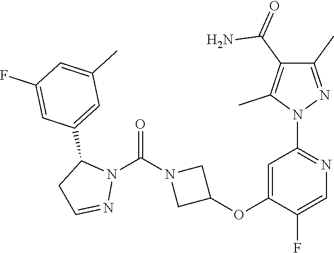 [SMILES: Cc1cc(F)cc([C@H]2CC=NN2C(=O)N2CC(Oc3cc(-n4nc(C)c(C(N)=O)c4C)ncc3F)C2)c1] | 136 |
| 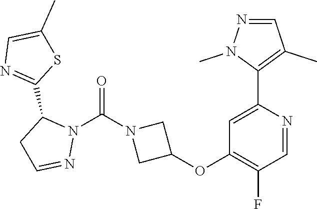 [SMILES: Cc1cnc([C@H]2CC=NN2C(=O)N2CC(Oc3cc(-c4c(C)cnn4C)ncc3F)C2)s1] | 137 |
| 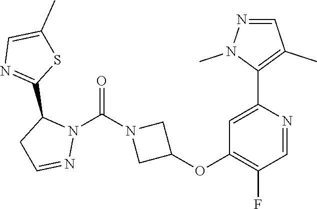 [SMILES: Cc1cnc([C@@H]2CC=NN2C(=O)N2CC(Oc3cc(-c4c(C)cnn4C)ncc3F)C2)s1] | 138 |
| 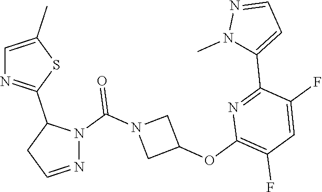 [SMILES: Cc1cnc(C2CC=NN2C(=O)N2CC(Oc3nc(-c4ccnn4C)c(F)cc3F)C2)s1] | 139 |
| 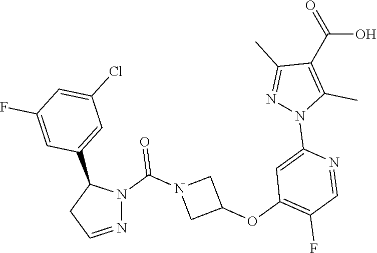 [SMILES: Cc1nn(-c2cc(OC3CN(C(=O)N4N=CC[C@H]4c4cc(F)cc(Cl)c4)C3)c(F)cn2)c(C)c1C(=O)O] | 140 |
| 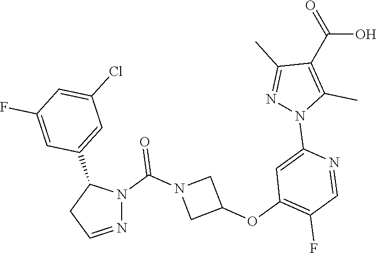 [SMILES: Cc1nn(-c2cc(OC3CN(C(=O)N4N=CC[C@@H]4c4cc(F)cc(Cl)c4)C3)c(F)cn2)c(C)c1C(=O)O] | 141 |
| 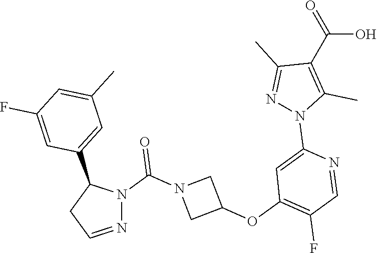 [SMILES: Cc1cc(F)cc([C@@H]2CC=NN2C(=O)N2CC(Oc3cc(-n4nc(C)c(C(=O)O)c4C)ncc3F)C2)c1] | 142 |
| 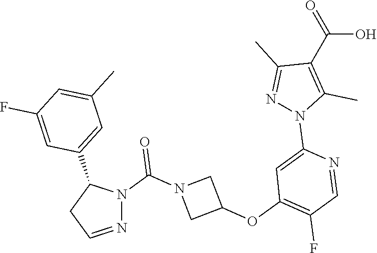 [SMILES: Cc1cc(F)cc([C@H]2CC=NN2C(=O)N2CC(Oc3cc(-n4nc(C)c(C(=O)O)c4C)ncc3F)C2)c1] | 143 |
| 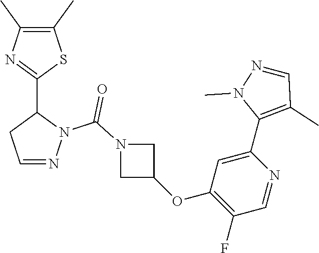 | 144 |
| 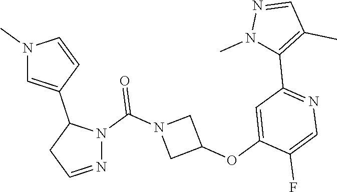 | 145 |
| 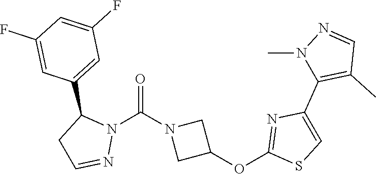 [SMILES: Cc1cnn(C)c1-c1csc(OC2CN(C(=O)N3N=CC[C@H]3c3cc(F)cc(F)c3)C2)n1] | 146 |
| 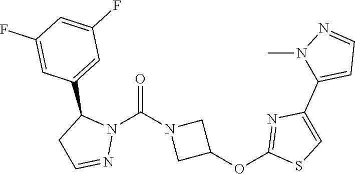 [SMILES: Cn1nccc1-c1csc(OC2CN(C(=O)N3N=CC[C@H]3c3cc(F)cc(F)c3)C2)n1] | 147 |
| 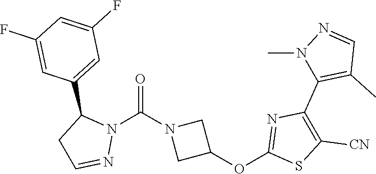 [SMILES: Cc1cnn(C)c1-c1nc(OC2CN(C(=O)N3N=CC[C@H]3c3cc(F)cc(F)c3)C2)sc1C#N] | 148 |
| 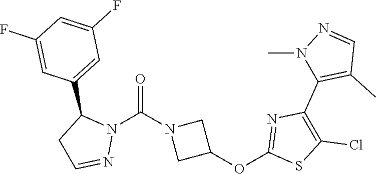 [SMILES: Cc1cnn(C)c1-c1nc(OC2CN(C(=O)N3N=CC[C@H]3c3cc(F)cc(F)c3)C2)sc1Cl] | 149 |
| 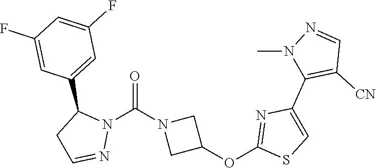 [SMILES: Cn1ncc(C#N)c1-c1csc(OC2CN(C(=O)N3N=CC[C@H]3c3cc(F)cc(F)c3)C2)n1] | 150 |
| 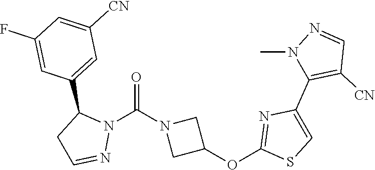 | 151 |
| 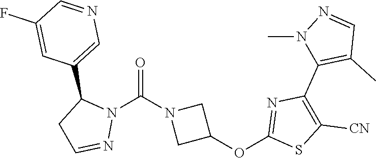 [SMILES: Cc1cnn(C)c1-c1nc(OC2CN(C(=O)N3N=CC[C@H]3c3cncc(F)c3)C2)sc1C#N] | 152 |
| 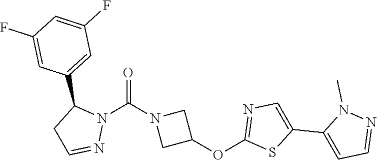 [SMILES: Cn1nccc1-c1cnc(OC2CN(C(=O)N3N=CC[C@H]3c3cc(F)cc(F)c3)C2)s1] | 153 |
| 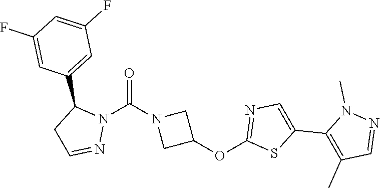 [SMILES: Cc1cnn(C)c1-c1cnc(OC2CN(C(=O)N3N=CC[C@H]3c3cc(F)cc(F)c3)C2)s1] | 154 |
| 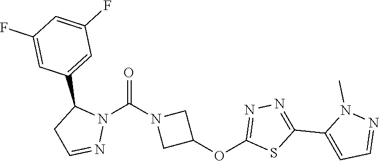 [SMILES: Cn1nccc1-c1nnc(OC2CN(C(=O)N3N=CC[C@H]3c3cc(F)cc(F)c3)C2)s1] | 155 |
| 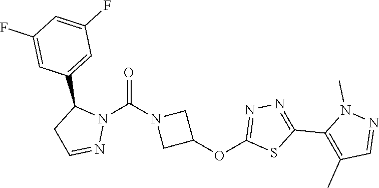 [SMILES: Cc1cnn(C)c1-c1nnc(OC2CN(C(=O)N3N=CC[C@H]3c3cc(F)cc(F)c3)C2)s1] | 156 |
| 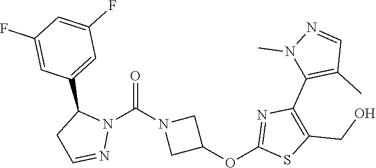 [SMILES: Cc1cnn(C)c1-c1nc(OC2CN(C(=O)N3N=CC[C@H]3c3cc(F)cc(F)c3)C2)sc1CO] | 157 |
| 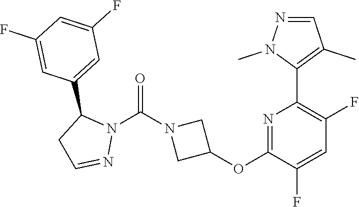 [SMILES: Cc1cnn(C)c1-c1nc(OC2CN(C(=O)N3N=CC[C@H]3c3cc(F)cc(F)c3)C2)c(F)cc1F] | 158 |
| 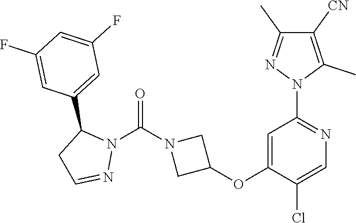 [SMILES: Cc1nn(-c2cc(OC3CN(C(=O)N4N=CC[C@H]4c4cc(F)cc(F)c4)C3)c(Cl)cn2)c(C)c1C#N] | 159 |
| 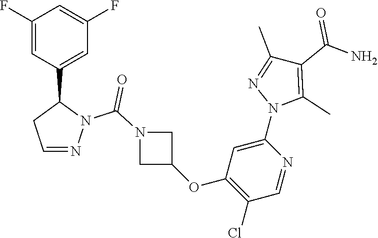 [SMILES: Cc1nn(-c2cc(OC3CN(C(=O)N4N=CC[C@H]4c4cc(F)cc(F)c4)C3)c(Cl)cn2)c(C)c1C(N)=O] | 160 |
| 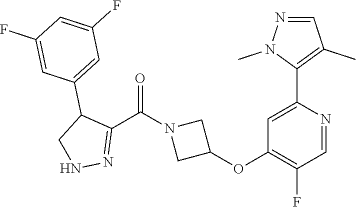 [SMILES: Cc1cnn(C)c1-c1cc(OC2CN(C(=O)C3=NNCC3c3cc(F)cc(F)c3)C2)c(F)cn1] | 161 |
| 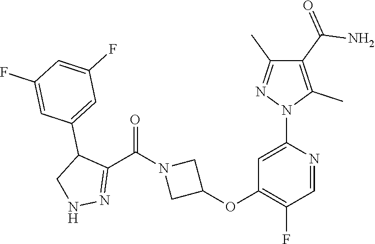 [SMILES: Cc1nn(-c2cc(OC3CN(C(=O)C4=NNCC4c4cc(F)cc(F)c4)C3)c(F)cn2)c(C)c1C(N)=O] | 162 |
| 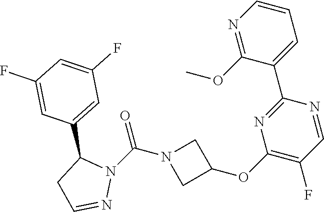 [SMILES: COc1ncccc1-c1ncc(F)c(OC2CN(C(=O)N3N=CC[C@H]3c3cc(F)cc(F)c3)C2)n1] | 163 |
| 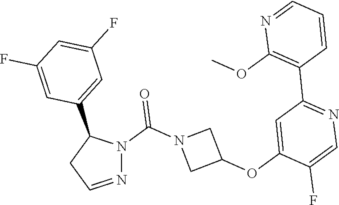 [SMILES: COc1ncccc1-c1cc(OC2CN(C(=O)N3N=CC[C@H]3c3cc(F)cc(F)c3)C2)c(F)cn1] | 164 |
| 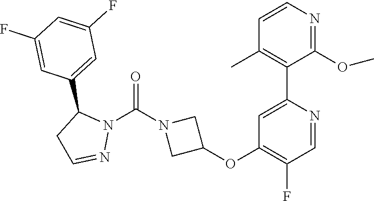 [SMILES: COc1nccc(C)c1-c1cc(OC2CN(C(=O)N3N=CC[C@H]3c3cc(F)cc(F)c3)C2)c(F)cn1] | 165 |
| 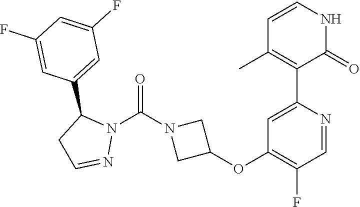 [SMILES: Cc1cc[nH]c(=O)c1-c1cc(OC2CN(C(=O)N3N=CC[C@H]3c3cc(F)cc(F)c3)C2)c(F)cn1] | 166 |
| 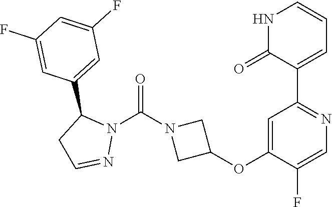 [SMILES: O=C(N1CC(Oc2cc(-c3ccc[nH]c3=O)ncc2F)C1)N1N=CC[C@H]1c1cc(F)cc(F)c1] | 167 |
| 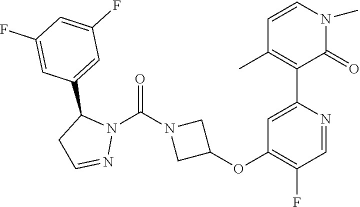 [SMILES: Cc1ccn(C)c(=O)c1-c1cc(OC2CN(C(=O)N3N=CC[C@H]3c3cc(F)cc(F)c3)C2)c(F)cn1] | 168 |
| 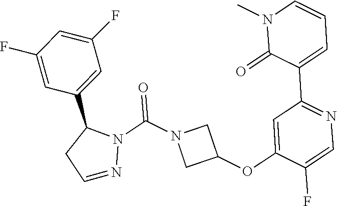 [SMILES: Cn1cccc(-c2cc(OC3CN(C(=O)N4N=CC[C@H]4c4cc(F)cc(F)c4)C3)c(F)cn2)c1=O] | 169 |

Another aspect of the disclosure provides a pharmaceutical composition comprising at least one compound selected from a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a compound selected from a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing can be administered as a separate composition concurrently with, prior to, or subsequent to, a composition comprising an additional active pharmaceutical agent.

As described above, the pharmaceutical compositions disclosed herein comprise a pharmaceutically acceptable carrier. The pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

A compound selected from a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition disclosed herein can be administered orally in solid dosage forms, such as capsules, tablets, troches, dragées, granules and powders, or in liquid dosage forms, such as elixirs, syrups, emulsions, dispersions, and suspensions. The compound, tautomer, hydrate, stereoisomer, or pharmaceutically acceptable salt described herein can also be administered parenterally, in sterile liquid dosage forms, such as dispersions, suspensions or solutions. Other dosages forms that can also be used to administer the compound, tautomer, hydrate, stereoisomer, or pharmaceutically acceptable salt described herein as an ointment, cream, drops, transdermal patch or powder for topical administration, as an ophthalmic solution or suspension formation, e.g., eye drops, for ocular administration, as an aerosol spray or powder composition for inhalation or intranasal administration, or as a cream, ointment, spray or suppository for rectal or vaginal administration.

Gelatin capsules containing the compound and/or the at least one pharmaceutically acceptable salt thereof disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene glycols can be examples of suitable carriers for parenteral solutions. Solutions for parenteral administration may comprise a water soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol, a standard reference text in the art.

For administration by inhalation, the compound, tautomer, hydrate, stereoisomer, or pharmaceutically acceptable salt described herein may be conveniently delivered in the form of an aerosol spray presentation from pressurized packs or nebulisers. The compound, tautomer, hydrate, stereoisomer, or pharmaceutically acceptable salt described herein may also be delivered as powders, which may be formulated, and the powder composition may be inhaled with the aid of an insufflation powder inhaler device. One exemplary delivery system for inhalation can be metered dose inhalation (MDI) aerosol, which may be formulated as a suspension or solution of a compound, tautomer, hydrate, stereoisomer, or pharmaceutically acceptable salt described herein in at least one suitable propellant, selected, for example, from fluorocarbons and hydrocarbons.

For ocular administration, an ophthalmic preparation may be formulated with an appropriate weight percentage of a solution or suspension of the compound, tautomer, hydrate, stereoisomer, or pharmaceutically acceptable salt described herein in an appropriate ophthalmic vehicle, such that the compound, tautomer, hydrate, stereoisomer, or pharmaceutically acceptable salt described herein is maintained in contact with the ocular surface for a sufficient time period to allow the compound to penetrate the corneal and internal regions of the eye.

Useful pharmaceutical dosage-forms for administration of the compound, tautomer, hydrate, stereoisomer, or pharmaceutically acceptable salt described herein include, but are not limited to, hard and soft gelatin capsules, tablets, parenteral injectables, and oral suspensions.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the mimetic is usually a minor component (from about 0.1 to about 50% by weight or preferably from about 1 to about 40% by weight) with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

In some embodiments, unit capsules can be prepared by filling standard two-piece hard gelatin capsules each with, for example, 100 milligrams of the compound, tautomer, hydrate, stereoisomer, or pharmaceutically acceptable salt described herein in powder, 150 milligrams of lactose, 50 milligrams of cellulose, and 6 milligrams magnesium stearate.

In some embodiments, a mixture of the compound, tautomer, hydrate, stereoisomer, or pharmaceutically acceptable salt described herein and a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, tablets can be prepared by conventional procedures so that the dosage unit comprises, for example, 100 milligrams of the compound, stereoisomers thereof, and pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, and pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, tautomer, hydrate, stereoisomer, or pharmaceutically acceptable salt described herein is administered stepwise or in conjunction with at least one other therapeutic agent. When drugs are administered in physical combination, the dosage form and administration route should be selected depending on the compatibility of the combined drugs. Thus, the term coadministration is understood to include the administration of at least two agents concomitantly or sequentially, or alternatively as a fixed dose combination of the at least two active components.

The compound, tautomer, hydrate, stereoisomer, or pharmaceutically acceptable salt disclosed herein can be administered as the sole active ingredient or in combination with at least one second active ingredient.

In some embodiments, the compound, tautomer, hydrate, stereoisomer, or pharmaceutically acceptable salt described herein is incorporated into pharmaceutical compositions or formulations. The compositions may contain pharmaceutically acceptable diluents and/or carriers, e.g., diluents or carriers that are physiologically compatible and substantially free from pathogenic impurities. Suitable excipients or carriers and methods for preparing administrable compositions are known or apparent to those skilled in the art and are described in more detail in such publications as Remington's Pharmaceutical Science, Mack Publishing Co, NJ (1991). The compositions may also be in the form of controlled release or sustained release compositions as known in the art. For many applications, the compound, tautomer, hydrate, stereoisomer, or pharmaceutically acceptable salt described herein is administered for morning/daytime dosing, with off period at night.

The compound, tautomer, hydrate, or stereoisomer described herein may be used per se, or in the form of their pharmaceutically acceptable salts, such as hydrochlorides, hydrobromides, acetates, sulfates, citrates, carbonates, trifluoroacetates and the like. When the compound, tautomer, hydrate, stereoisomer, or pharmaceutically acceptable salt described herein contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When the compound, tautomer, hydrate, or stereoisomer described herein contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al, “Pharmaceutical Salts”, Journal of Pharmaceutical Science, 1977, 66, 1-19).

Neutral forms of the pharmaceutically acceptable salt described herein may be regenerated by contacting the salt with a base or acid, and isolating the parent compound in the conventional manner.

This disclosure provides prodrugs. Prodrugs of the compound, tautomer, hydrate, stereoisomer, or pharmaceutically acceptable salt described herein that readily undergo chemical changes under physiological conditions to provide the compound, tautomer, hydrate, stereoisomer, or pharmaceutically acceptable salt of the present disclosure. Additionally, prodrugs can be converted to the compound, tautomer, hydrate, stereoisomer, or a pharmaceutically acceptable salt of the present disclosure by chemical or biochemical methods in an ex vivo environment. For example, prodrugs can be slowly converted to the compound, tautomer, hydrate, stereoisomer, or pharmaceutically acceptable salt of the present disclosure when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present disclosure which is administered as an ester (the “prodrug”), but then is metabolically hydrolyzed to the carboxylic acid, the active entity.

Certain compound, tautomer, stereoisomer, or pharmaceutically acceptable salt of the disclosure can exist in unsolvated forms as well as solvated forms, including hydrated forms. Certain compound, tautomer, hydrate, stereoisomer, or pharmaceutically acceptable salt of the disclosure may exist in multiple crystalline or amorphous forms.

Certain compound, tautomer, hydrate, or pharmaceutically acceptable salt in this disclosure possesses asymmetric carbon atoms (optical centers) or double bonds; the racemates, enantiomers, diastereomers, geometric isomers and individual isomers are all intended to be encompassed within the scope of the present disclosure.

### III. Methods of Treatment and Uses

In another aspect of this disclosure, a compound, tautomer, hydrate, stereoisomer, or pharmaceutically acceptable salt as described herein, including a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition thereof, is for use in treating a disease or condition selected from an inflammatory disease, an immune disease (e.g., an autoimmune disease), an allergic disease, transplant rejection, a necrotic cell disease, a neurodegenerative disease, a central nervous system (CNS) disease, ischemic brain injury, an ocular disease, an infectious disease, and a malignancy. In some embodiments, the disease or condition is mediated by receptor-interacting protein 1 (RIP1) signaling. In some embodiments, the disease or condition is selected from ulcerative colitis, Crohn's disease, psoriasis, rheumatoid arthritis, amyotrophic lateral sclerosis (ALS), Alzheimer's disease, and a viral infection.

In another aspect, disclosed herein is a compound, tautomer, hydrate, stereoisomer, or pharmaceutically acceptable salt as described herein, including a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition thereof, for use as a medicament.

In another aspect, disclosed herein is use of a compound, tautomer, hydrate, stereoisomer, or pharmaceutically acceptable salt as described herein, including a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition thereof, for the manufacture of a medicament for treating a disease or condition selected from an inflammatory disease, an immune disease (e.g., an autoimmune disease), an allergic disease, transplant rejection, a necrotic cell disease, a neurodegenerative disease, a central nervous system (CNS) disease, ischemic brain injury, an ocular disease, an infectious disease, and a malignancy. In some embodiments, the disease or condition is mediated by RIP1 signaling. In some embodiments, the disease or condition is selected from ulcerative colitis, Crohn's disease, psoriasis, rheumatoid arthritis, amyotrophic lateral sclerosis (ALS), Alzheimer's disease, and a viral infection. In yet another aspect, disclosed herein is a method of treating a disease or condition selected from an inflammatory disease, an immune disease (e.g., an autoimmune disease), an allergic disease, transplant rejection, a necrotic cell disease, a neurodegenerative disease, a central nervous system (CNS) disease, ischemic brain injury, an ocular disease, an infectious disease, and a malignancy in a subject, comprising administering a therapeutically effective amount of a compound, tautomer, a hydrate or stereoisomer of the compound or the tautomer, or pharmaceutically acceptable salt as described herein, including a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition thereof. In some embodiments, the disease or condition is mediated by RIP1 signaling. In some embodiments, the disease or condition is selected from ulcerative colitis, Crohn's disease, psoriasis, rheumatoid arthritis, ALS, Alzheimer's disease, and a viral infection.

In a further aspect of this disclosure, a compound, tautomer, hydrate, stereoisomer, or pharmaceutically acceptable salt as described herein, including a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition thereof, is for use in treating a disease or condition mediated by RIP1 signaling. In some embodiments, the disease or condition is selected from ulcerative colitis, Crohn's disease, psoriasis, rheumatoid arthritis, amyotrophic lateral sclerosis (ALS), Alzheimer's disease, and a viral infection. In another aspect, disclosed herein is use of a compound, tautomer, a hydrate or stereoisomer of the compound or the tautomer, or pharmaceutically acceptable salt as described herein, including a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition thereof, for the manufacture of a medicament for treating a disease or condition mediated by RIP1 signaling. In some embodiments, the disease or condition is selected from ulcerative colitis, Crohn's disease, psoriasis, rheumatoid arthritis, ALS, Alzheimer's disease, and a viral infection. In yet another aspect, disclosed herein is a method of treating a disease or condition mediated by RIP1 signaling in a subject, comprising administering a therapeutically effective amount of a compound, tautomer, a hydrate or stereoisomer of the compound or the tautomer, or pharmaceutically acceptable salt as described herein, including a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition thereof. In some embodiments, the disease or condition is selected from ulcerative colitis, Crohn's disease, psoriasis, rheumatoid arthritis, ALS, Alzheimer's disease, and a viral infection.

In another aspect of this disclosure, a compound, tautomer, a hydrate or stereoisomer of the compound or the tautomer, or pharmaceutically acceptable salt as described herein, including a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition thereof, is for use in mediating, e.g., inhibiting, RIP1 by contacting the RIP1 protein or a fragment thereof (e.g., kinase domain, intermediate domain, and/or death domain) with the compound, tautomer, a hydrate or stereoisomer of the compound or the tautomer, pharmaceutically acceptable salt, or pharmaceutical composition.

In yet another aspect, disclosed herein is a method of inhibiting RIP1, comprising contacting the RIP1 protein or a fragment thereof (e.g., kinase domain, intermediate domain, and/or death domain) with a compound, tautomer, a hydrate or stereoisomer of the compound or the tautomer, or pharmaceutically acceptable salt as described herein to a subject, including a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition thereof.

A compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition thereof may be administered once daily, twice daily, or three times daily, for example, for the treatment of a disease or condition as described above, e.g., a disease or condition selected from an inflammatory disease, an immune disease (e.g., an autoimmune disease), an allergic disease, transplant rejection, a necrotic cell disease, a neurodegenerative disease, CNS disease, ischemic brain injury, an ocular disease, an infectious disease, and a malignancy, including those mediated by RIP1 signaling; a disease or condition selected from ulcerative colitis, Crohn's disease, psoriasis, rheumatoid arthritis, ALS, Alzheimer's disease, and a viral infection, including those mediated by RIP1 signaling; a disease or condition mediated by RIP1 signaling.

In some embodiments, 2 mg to 1500 mg or 5 mg to 1000 mg of a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition thereof are administered once daily, twice daily, or three times daily.

A compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, Compounds 1 to 169, a tautomer thereof, a hydrate or stereoisomer of the compound or the tautomer, or a pharmaceutically acceptable salt of the foregoing, or a pharmaceutical composition thereof may be administered, for example, various manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

The contacting is generally effected by administering to the subject an effective amount of one or more compounds, tautomers, hydrates, stereoisomers, and pharmaceutically acceptable salt disclosed herein. Generally, administration is adjusted to achieve a therapeutic dosage of about 0.1 to 50, preferably 0.5 to 10, more preferably 1 to 10 mg/kg, though optimal dosages are compound specific, and generally empirically determined for each compound.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

The subject compositions may also be coformulated and/or coadministered with a different compound to treat applicable indications, or to treat programmed cell death. In some embodiments, applicable indications include brain injury, neurodegenerative diseases, viral infections, immune tolerance, and cancer, e.g., to promote tumor immunity in pancreatic cancer and melanoma.

### EXAMPLES

In order that the disclosure described herein may be more fully understood, the following examples are disclosed herein. It should be understood that these examples are for illustrative purposes only and are not to be construed as limiting this disclosure in any way.

### Example 1. Synthesis of Exemplary Compounds

The compounds of the disclosure, selected from a compound of Formulae Ia, Ib, IIa, IIb, IIc, IId, IIe, IIf, IIg, IIh, IIIa-1, IIIa-2, IIIb-1, IIIb-2, IIIc-1, IIId-1, IIIe-1, IIIf-1, IIIg-1, IIIg-2, IIIh-1, IVa, IVb, IVc, IVd, IVe, IVf, or IVg, a tautomer thereof, a hydrate or stereoisomer of the compound or the tautomer, or a pharmaceutically acceptable salt of the foregoing, can be made according to standard chemical practices or as illustrated herein, including the following synthetic schemes for Compounds 1 to 169 as representative examples of Formula Ia or Ib.

### Compound Preparation

### (S)-3-fluoro-5-(1-(3-((5-fluoro-2-(1-methyl-1H-pyrazol-5-yl)pyrimidin-4-yl)oxy)azetidine-1-carbonyl)-4,5-dihydro-1H-pyrazol-5-yl)benzonitrile (1)

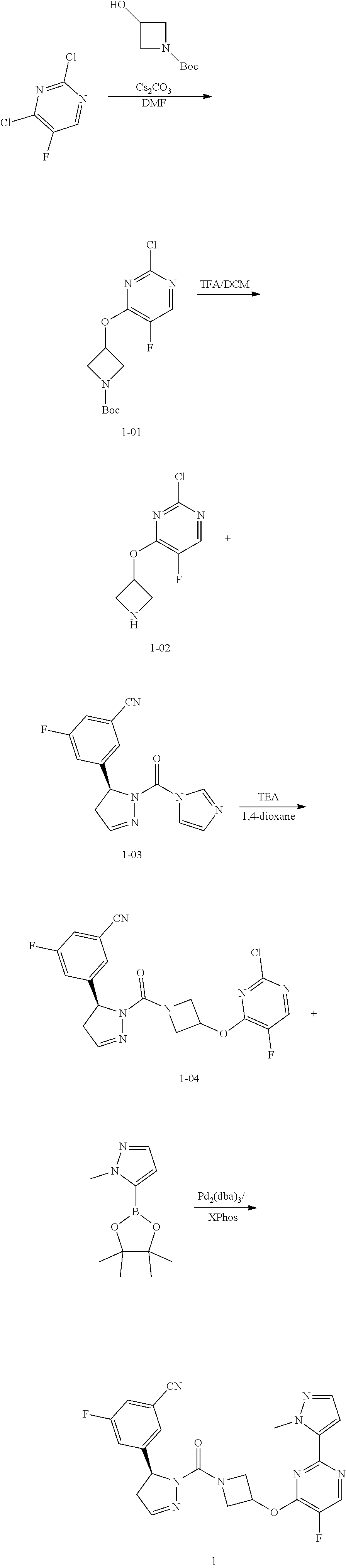

Step 1: 2,4-dichloro-5-fluoropyrimidine (5 g, 29.9 mmol) was added to a solution of Cs₂CO₃ (19.5 g, 59.9 mmol) in DMF (100 mL), and tert-butyl 3-hydroxyazetidine-1-carboxylate (5.7 g, 32.9 mmol) was added. The mixture was stirred at 100° C. for 2 hrs. The reaction mixture was then extracted by EtOAc/H₂O (50 mL/50 mL) 3 times. The organic layer was combined, washed with brine, dried over Na₂SO₄, concentrated, and further purified by silica gel column chromatography (PE/EA=5/1) to give compound 1-01 (3.9 g, 43%) as a colorless oil. Mass (m/z) 304.1 [M+H]⁺.

Step 2: TFA (5 mL) was added to a solution of 1-01 (3.9 g, 12.9 mmol) in DCM (10 mL). The reaction mixture was stirred at room temperature for 0.5 hr. then the solvent was evaporated in vacuo to give crude compound 1-02 (5.2 g). Mass (m/z) 204.1 [M+H]⁺

Step 3: Compound 1-03 (3.3 g, 11.7 mmol) was added to a solution of compound 1-02 and TEA (3.55 g, 35.1 mmol) in 1,4-dioxane (30 mL). The reaction mixture was stirred at room temperature overnight. Then the solvent was evaporated in vacuo. The oil residue was purified by silica gel column chromatography (PE/EA=1/1) to give compound 1-04 (4.35 g, 90%) as a yellow oil. Mass (m/z) 419.1 [M+H]⁺

Step 4: The titled compound 1 was prepared in 87.9% yield according to the procedure outlined for compound 19. Mass (m/z) 465.2 [M+H]⁺; ¹H NMR (400 MHz, Chloroform-d) δ 8.42 (dd, J=2.5, 0.9 Hz, 1H), 7.50 (dd, J=2.1, 0.8 Hz, 1H), 7.34 (q, J=1.3 Hz, 1H), 7.26-7.24 (m, 1H), 7.21 (ddd, J=9.0, 2.7, 1.5 Hz, 1H), 6.94 (dd, J=2.1, 0.9 Hz, 1H), 6.81 (d, J=1.8 Hz, 1H), 5.53 (td, J=6.5, 3.3 Hz, 1H), 5.34-5.30 (m, 1H), 5.30-5.28 (m, 1H), 4.64 (s, 2H), 4.43-4.33 (m, 1H), 4.30-4.28 (m, 3H), 3.40 (ddd, J=18.6, 12.2, 1.6 Hz, 1H), 2.70 (ddd, J=18.6, 6.8, 1.6 Hz, 1H).

### (S)-(3-((5-fluoro-2-(1-methyl-1H-pyrazol-5-yl)pyrimidin-4-yl)oxy)azetidin-1-yl)(5-(5-fluoropyridin-3-yl)-4,5-dihydro-1H-pyrazol-1-yl)methano ne (2)

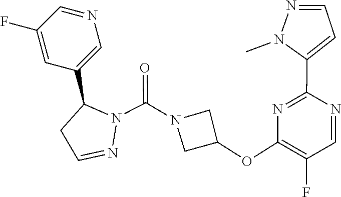 [SMILES: Cn1nccc1-c1ncc(F)c(OC2CN(C(=O)N3N=CC[C@H]3c3cncc(F)c3)C2)n1]

The titled compound 2 was prepared in 56.8% yield according to the procedure outlined for compound 1. Mass (m/z) 441.3 [M+H]⁺; ¹H NMR (400 MHz, Chloroform-d) δ 8.44-8.34 (m, 3H), 7.50 (dd, J=2.0, 1.1 Hz, 1H), 7.40 (d, J=8.6 Hz, 1H), 6.94 (t, J=1.5 Hz, 1H), 6.85 (s, 1H), 5.53 (d, J=4.4 Hz, 1H), 5.41-5.32 (m, 1H), 4.63 (s, 2H), 4.37 (d, J=11.0 Hz, 1H), 4.30 (m, 4H), 3.44 (t, J=14.4 Hz, 1H), 2.78 (d, J=17.8 Hz, 1H).

### (S)-3-(1-(3-((2-(3,5-dimethylisoxazol-4-yl)-5-fluoropyrimidin-4-yl)ox y)azetidine-1-carbonyl)-4,5-dihydro-1H-pyrazol-5-yl)-5-fluorobenzonitrile (3)

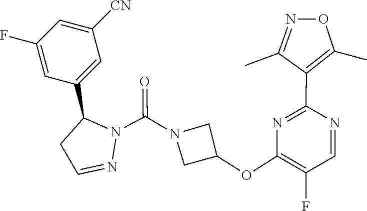 [SMILES: Cc1noc(C)c1-c1ncc(F)c(OC2CN(C(=O)N3N=CC[C@H]3c3cc(F)cc(C#N)c3)C2)n1]

The titled compound 3 was prepared in 52.1% yield according to the procedure outlined for compound 1. ¹H NMR (400 MHz, Chloroform-d) δ 8.40 (dd, J=2.5, 0.5 Hz, 1H), 7.34 (d, J=1.5 Hz, 1H), 7.25 (d, J=4.5 Hz, 1H), 7.23-7.19 (m, 1H), 6.81 (d, J=1.7 Hz, 1H), 5.53 (tt, J=6.6, 4.0 Hz, 1H), 5.35-5.27 (m, 1H), 4.61 (s, 2H), 4.33 (dd, J=32.6, 10.8 Hz, 2H), 3.40 (ddd, J=18.8, 12.3, 1.7 Hz, 1H), 2.77-2.66 (m, 4H), 2.56 (s, 3H); Mass (m/z) 480.3 [M+H]⁺.

### (S)-(3-((2-(3,5-dimethylisoxazol-4-yl)-5-fluoropyrimidin-4-yl)oxy)azetidin-1-yl)(5-(5-fluoropyridin-3-yl)-4,5-dihydro-1H-pyrazol-1-yl)methano ne (4)

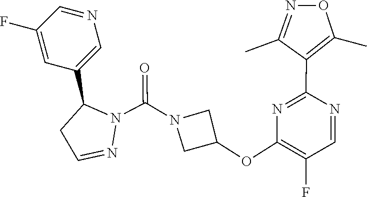 [SMILES: Cc1noc(C)c1-c1ncc(F)c(OC2CN(C(=O)N3N=CC[C@H]3c3cncc(F)c3)C2)n1]

The titled compound 4 was prepared in 53.8% yield according to the procedure outlined for compound 1. ¹H NMR (400 MHz, Chloroform-d) δ 8.39 (dt, J=2.5, 0.8 Hz, 3H), 7.33 (d, J=8.9 Hz, 1H), 6.84 (s, 1H), 5.52 (tt, J=7.1, 4.0 Hz, 1H), 5.37 (dd, J=12.0, 6.3 Hz, 1H), 4.60 (s, 2H), 4.32 (dd, J=34.5, 10.7 Hz, 2H), 3.42 (dd, J=18.5, 12.1 Hz, 1H), 2.80-2.70 (m, 4H), 2.57-2.51 (m, 3H). Mass (m/z) 456.2 [M+H]⁺.

### (S)-3-fluoro-5-(1-(3-((5-fluoro-2-(4-fluoro-1-methyl-1H-pyrazol-5-yl) pyridin-4-yl)oxy)azetidine-1-carbonyl)-4,5-dihydro-1H-pyrazol-5-yl)benzonitrile (5)

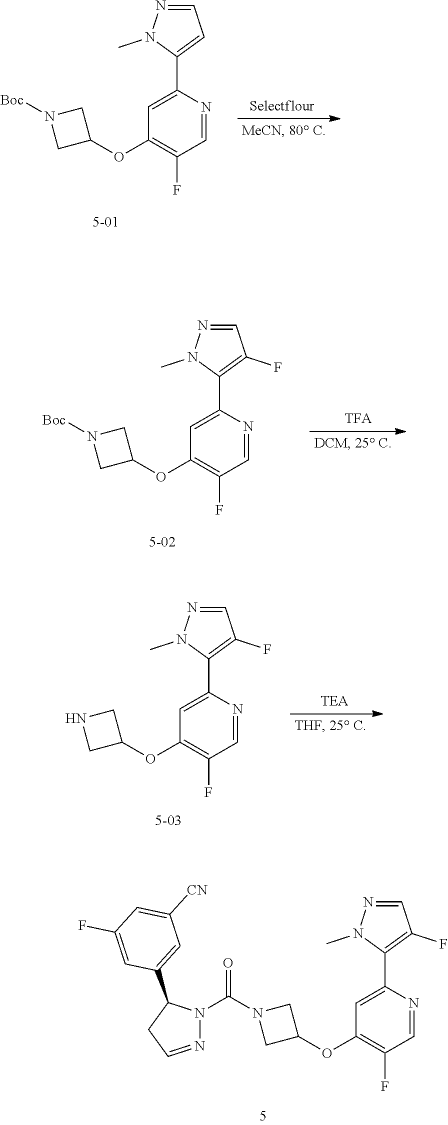 [SMILES: Cn1nccc1-c1cc(OC2CN(C(=O)OC(C)(C)C)C2)c(F)cn1 ; Cn1ncc(F)c1-c1cc(OC2CN(C(=O)OC(C)(C)C)C2)c(F)cn1 ; Cn1ncc(F)c1-c1cc(OC2CNC2)c(F)cn1 ; Cn1ncc(F)c1-c1cc(OC2CN(C(=O)N3N=CC[C@H]3c3cc(F)cc(C#N)c3)C2)c(F)cn1]

Step 1: 5-01 (1.1 g, 3.16 mmol) was dissolved in 15 ml of dry MeCN, Selectfluor (1.18 g, 3.16 mmol) was added to the above solution at 0° C., and the mixture was stirred for 3 h at 80° C. Water was added and extracted with EA, Purification by silica gel chromatography gave the compound 5-02 (320 mg, 27.6%). Mass (m/z) 367.2 [M+H]⁺.

Step 2-3: The title compound 5 was prepared in a yield of 1⁹0.8% from compound 5-02 according to the procedure for compound 1-02. ¹H NMR (300 MHz, Chloroform-d) δ 8.45 (d, J=2.8 Hz, 1H), 7.39 (d, J=4.4 Hz, 1H), 7.34 (t, J=1.5 Hz, 1H), 7.26-7.16 (m, 2H), 6.99 (d, J=6.5 Hz, 1H), 6.83 (s, 1H), 5.31 (dd, J=12.1, 6.7 Hz, 1H), 5.15-5.05 (m, 1H), 4.70-4.55 (s, 2H), 4.33 (dd, J=23.4, 9.5 Hz, 2H), 4.16 (s, 3H), 3.41 (dd, J=18.7, 12.1 Hz, 1H), 2.71 (dd, J=18.5, 6.5 Hz, 1H). Mass (m/z):482.2 [M+H]⁺.

### (S)-3-(1-(3-((2-(4-chloro-1-methyl-1H-pyrazol-5-yl)-5-fluoropyridin-4-yl)oxy)azetidine-1-carbonyl)-4,5-dihydro-1H-pyrazol-5-yl)-5-fluorobenzonitrile (6)

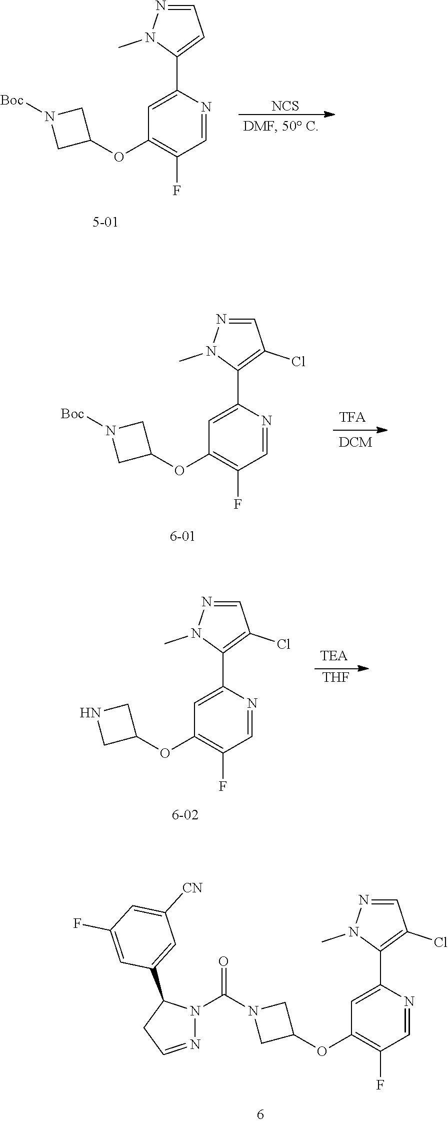 [SMILES: Cn1nccc1-c1cc(OC2CN(C(=O)OC(C)(C)C)C2)c(F)cn1 ; "NCS" ; Cn1ncc(Cl)c1-c1cc(OC2CN(C(=O)OC(C)(C)C)C2)c(F)cn1 ; Cn1ncc(Cl)c1-c1cc(OC2CNC2)c(F)cn1 ; "THF" ; Cn1ncc(Cl)c1-c1cc(OC2CN(C(=O)N3N=CC[C@H]3c3cc(F)cc(C#N)c3)C2)c(F)cn1]

Step 1: 5-01 (600 mg, 1.72 mmol) was dissolved in 10 ml of dry DMF, NCS (230 mg, 1.72 mmol) was added to the above solution at 0° C., and the mixture was stirred for 12 h at 50° C. Water was added and extracted with EA, Purification by silica gel chromatography gave the intermediate 6-01 (360 mg, 54%). Mass (m/z) 383.1 [M+H]⁺.

Step 2-3: The titled compound 6 was prepared in 33.1% yield from 6-01 and 1-03 according to the procedure outlined for compound 1-02. ¹H NMR (300 MHz, DMSO-d₆) δ 8.69 (d, J=3.1 Hz, 1H), 7.74 (ddd, J=8.6, 2.5, 1.3 Hz, 1H), 7.69 (s, 1H), 7.56 (t, J=1.4 Hz, 1H), 7.47-7.39 (m, 1H), 7.29 (d, J=6.8 Hz, 1H), 7.04 (s, 1H), 5.34-5.12 (m, 2H), 4.60-4.45 (m, 2H), 4.14-4.00 (m, J=10.7 Hz, 2H), 3.91 (s, 3H), 3.50-3.36 (m, 1H), 2.77-2.65 (m, 1H). Mass (m/z) 498.1 [M+H]⁺.

### (S)-3-fluoro-5-(1-(3-((5-fluoro-2-(1-methyl-1H-pyrazol-5-yl)pyridin-4-yl)oxy)azetidine-1-carbonyl)-4,5-dihydro-1H-pyrazol-5-yl)benzonitrile (7)

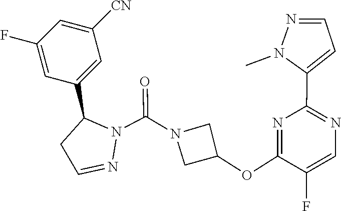 [SMILES: Cn1nccc1-c1ncc(F)c(OC2CN(C(=O)N3N=CC[C@H]3c3cc(F)cc(C#N)c3)C2)n1]

The titled compound 7 was prepared according to the procedure outlined for compound 1. Mass (m/z) 464.3 [M+H]⁺. ¹H NMR (400 MHz, Chloroform-d) δ 8.45-8.42 (m, 1H), 7.52-7.47 (m, 1H), 7.36-7.31 (m, 1H), 7.30-7.26 (m, 1H), 7.23-7.18 (m, 1H), 6.90-6.81 (m, 2H), 6.47 (q, J=2.0 Hz, 1H), 5.39-5.26 (m, 1H), 5.15-5.06 (m, 1H), 4.61 (s, 2H), 4.41-4.25 (m, 2H), 4.21-4.11 (m, 3H), 3.47-3.33 (m, 1H), 2.78-2.65 (m, 1H).

### (S)-5-(4-((1-(5-(3-cyano-5-fluorophenyl)-4,5-dihydro-1H-pyrazole-1-carbonyl)azetidin-3-yl)oxy)-5-fluoropyrimidin-2-yl)-1-methyl-1H-pyrazole-4-carbonitrile (8)

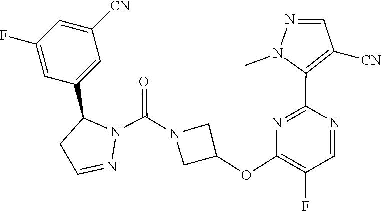 [SMILES: Cn1ncc(C#N)c1-c1ncc(F)c(OC2CN(C(=O)N3N=CC[C@H]3c3cc(F)cc(C#N)c3)C2)n1]

The titled compound 8 was prepared in 21.3% yield as white solid from 1-04 according to the procedure outlined for compound 1. Mass (m/z) 490.1 [M+H]⁺. ¹H NMR (300 MHz, Chloroform-d) δ 8.48 (d, J=2.3 Hz, 1H), 7.84 (s, 1H), 7.33 (d, J=1.5 Hz, 1H), 7.25-7.16 (m, 2H), 6.78 (d, J=1.7 Hz, 11H), 5.76 (tt, J=6.5, 3.7 Hz, 1H), 5.29 (dd, J=12.2, 6.7 Hz, 1H), 4.73 (s, 2H), 4.33 (s, 4H), 4.32-4.22 (m, 2H), 3.38 (dd, J=18.6, 12.3 Hz, 1H), 2.67 (dd, J=18.6, 6.7 Hz, 1H).

### (S)-5-(4-((1-(5-(3-cyano-5-fluorophenyl)-4,5-dihydro-1H-pyrazole-1-carbonyl)azetidin-3-yl)oxy)-5-fluoropyridin-2-yl)-1-methyl-1H-pyrazole-4-carbonitrile (9)

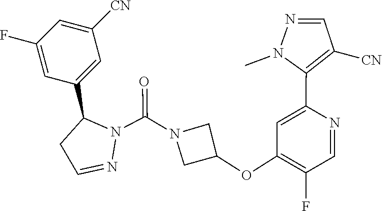 [SMILES: Cn1ncc(C#N)c1-c1cc(OC2CN(C(=O)N3N=CC[C@H]3c3cc(F)cc(C#N)c3)C2)c(F)cn1]

The titled compound 9 was prepared in 26.9% yield as white solid according to the procedure outlined for compound 1. Mass (m/z) 489.1[M+H]⁺. ¹H NMR (300 MHz, Chloroform-d) δ 8.51 (d, J=2.7 Hz, 1H), 7.83 (s, 1H), 7.34 (t, J=1.5 Hz, 1H), 7.30 (d, J=6.4 Hz, 1H), 7.28-7.18 (m, 2H), 6.82 (t, J=1.6 Hz, 1H), 5.30 (dd, J=12.2, 6.7 Hz, 1H), 5.13 (dt, J=6.4, 2.9 Hz, 1H), 4.67 (d, J=9.0 Hz, 2H), 4.33 (t, J=11.7 Hz, 2H), 4.19 (s, 3H), 3.40 (ddd, J=18.6, 12.2, 1.7 Hz, 1H), 2.70 (ddd, J=18.6, 6.8, 1.7 Hz, 1H).

### (S)-5-(4-((1-(5-(3-cyano-5-fluorophenyl)-4,5-dihydro-1H-pyrazole-1-carbonyl)azetidin-3-yl)oxy)-5-fluoropyridin-2-yl)-1-methyl-1H-pyrazole-4-carboxamide (10)

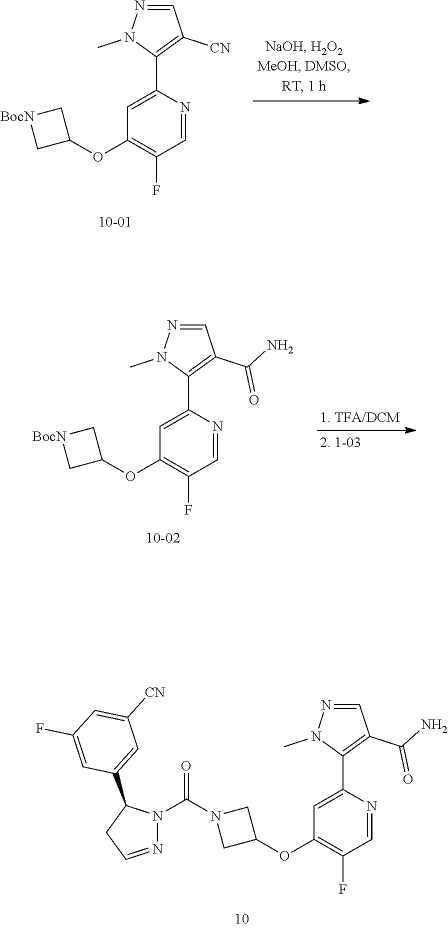 [SMILES: Cn1ncc(C#N)c1-c1cc(OC2CN(C(=O)OC(C)(C)C)C2)c(F)cn1 ; Cn1ncc(C(N)=O)c1-c1cc(OC2CN(C(=O)OC(C)(C)C)C2)c(F)cn1 ; Cn1ncc(C(N)=O)c1-c1cc(OC2CN(C(=O)N3N=CC[C@H]3c3cc(F)cc(C#N)c3)C2)c(F)cn1]

Step 1: To a solution of 10-01 (200 mg, 0.54 mmol) in MeOH (5 mL) and DMSO (5 mL) was added 15% NaOH (3 mL) and 3% H₂O₂ (5 mL). The reaction was stirred at rt for 1 h. The reaction mixture was diluted with water. The aqueous phase was extracted with EA. The combined organic extracts were washed with brine and dried over Na₂SO₄. The solvent was removed under vacuum and the crude product 10-02 was used to next step directly.

Step 2: The titled compound 10 was prepared in 27.10% yield as white solid from Compound 10-02 and 1-03 according to the procedure outlined for compound 1-02. Mass (m/z) 507.1 [M+H]⁺. ¹H NMR (400 MHz, Chloroform-d) δ 8.47 (s, 1H), 7.86 (s, 1H), 7.36 (d, J=1.5 Hz, 1H), 7.25-7.19 (m, 3H), 6.84 (d, J=1.7 Hz, 1H), 5.34 (dd, J=12.2, 6.6 Hz, 1H), 5.13-5.02 (m, 1H), 4.61 (s, 2H), 4.27 (s, 2H), 3.94 (s, 3H), 3.41 (ddd, J=18.7, 12.2, 1.7 Hz, 11H), 2.71 (ddd, J=18.7, 6.7, 1.7 Hz, 11H).

### (S)-3-(1-(3-((2-(1,4-dimethyl-1H-pyrazol-5-yl)-5-fluoropyridin-4-yl)oxy)azetidine-1-carbonyl)-4,5-dihydro-1H-pyrazol-5-yl)-5-fluorobenzonitrile (11)

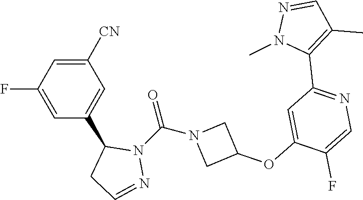 [SMILES: Cc1cnn(C)c1-c1cc(OC2CN(C(=O)N3N=CC[C@H]3c3cc(F)cc(C#N)c3)C2)c(F)cn1]

The titled compound 11 was prepared from 19-01 according to the procedure outlined for compound 1. Mass (m/z) 478.3 [M+H]⁺. ¹H NMR (300 MHz, Chloroform-d) δ 8.48 (d, J=2.9 Hz, 1H), 7.35 (s, 1H), 7.33 (t, J=1.5 Hz, 1H), 7.28-7.26 (m, 1H), 7.25-7.16 (m, 1H), 6.83 (t, J=1.7 Hz, 11H), 6.69 (d, J=6.6 Hz, 11H), 5.30 (dd, J=12.2, 6.7 Hz, 11H), 5.11-5.02 (m, 1H), 4.66-4.52 (m, 2H), 4.43-4.22 (m, 2H), 3.95 (s, 3H), 3.40 (ddd, J=18.7, 12.2, 1.7 Hz, 1H), 2.71 (ddd, J=18.7, 6.8, 1.7 Hz, 1H), 2.11 (s, 3H).

### (S)-3-(1-(3-((2-(1-ethyl-1H-pyrazol-5-yl)-5-fluoropyridin-4-yl)oxy)azetidine-1-carbonyl)-4,5-dihydro-1H-pyrazol-5-yl)-5-fluorobenzonitrile (12)

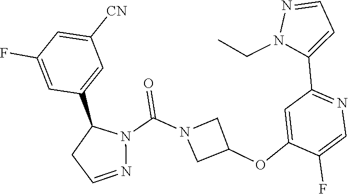 [SMILES: CCn1nccc1-c1cc(OC2CN(C(=O)N3N=CC[C@H]3c3cc(F)cc(C#N)c3)C2)c(F)cn1]

The titled compound 12 was prepared according to the procedure outlined for compound 1. Mass (m/z) 478.3 [M+H]⁺; ¹H NMR (301 MHz, Chloroform-d) δ 8.42 (d, J=2.9 Hz, 1H), 7.50 (d, J=2.0 Hz, 1H), 7.33 (t, J=1.5 Hz, 11H), 7.28-7.26 (m, 11H), 7.25-7.17 (m, 11H), 6.88-6.80 (m, 2H), 6.44 (d, J=2.0 Hz, 1H), 5.30 (dd, J=12.2, 6.7 Hz, 1H), 5.16-5.03 (m, 1H), 4.67-4.53 (m, 4H), 4.41-4.22 (m, 2H), 3.40 (ddd, J=18.7, 12.2, 1.7 Hz, 1H), 2.70 (ddd, J=18.7, 6.7, 1.7 Hz, 1H), 1.41 (t, J=7.2 Hz, 3H).

### (S)-3-fluoro-5-(1-(3-((3-fluoro-6-(1-methyl-1H-imidazol-2-yl)pyridin-2-yl)oxy)azetidine-1-carbonyl)-4,5-dihydro-1H-pyrazol-5-yl)benzonitrile (13)

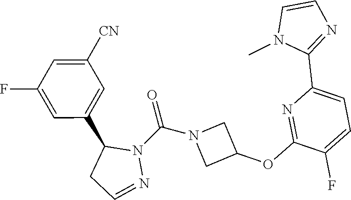 [SMILES: Cn1ccnc1-c1ccc(F)c(OC2CN(C(=O)N3N=CC[C@H]3c3cc(F)cc(C#N)c3)C2)n1]

The titled compound 13 was prepared according to the procedure outlined for compound 1. Mass (m/z) 464.3 [M+H]⁺. ¹H NMR (300 MHz, Chloroform-d) δ 7.74 (dd, J=8.3, 3.2 Hz, 1H), 7.39 (dd, J=9.7, 8.3 Hz, 1H), 7.28 (d, J=1.5 Hz, 1H), 7.23-7.09 (m, 2H), 7.01 (d, J=1.1 Hz, 1H), 6.92-6.86 (m, 1H), 6.74 (d, J=1.7 Hz, 1H), 5.39 (tt, J=6.4, 4.1 Hz, 1H), 5.24 (dd, J=12.2, 6.7 Hz, 1H), 4.50 (d, J=15.3 Hz, 2H), 4.38-4.15 (m, 2H), 3.97 (s, 3H), 3.33 (ddd, J=18.7, 12.2, 1.7 Hz, 1H), 2.62 (ddd, J=18.7, 6.8, 1.7 Hz, 1H).

### (S)-3-fluoro-5-(1-(3-((5-fluoro-2-(1-methyl-1H-1,2,3-triazol-4-yl)pyri din-4-yl)oxy)azetidine-1-carbonyl)-4,5-dihydro-1H-pyrazol-5-yl)benzonitrile (14)

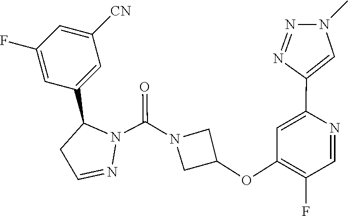 [SMILES: Cn1cc(-c2cc(OC3CN(C(=O)N4N=CC[C@H]4c4cc(F)cc(C#N)c4)C3)c(F)cn2)nn1]

The titled compound 14 was prepared from 17-01 in a yield of 56.6% according to the procedure outlined for compound 17. Mass (m/z) 465.2 [M+H]⁺. ¹H NMR (400 MHz, Chloroform-d) δ 8.41 (d, J=2.9 Hz, 1H), 8.20 (s, 1H), 7.34 (t, J=1.4 Hz, 1H), 7.31-7.26 (m, 2H), 7.21 (dt, J=9.0, 2.0 Hz, 1H), 6.83 (d, J=1.7 Hz, 1H), 5.32 (dd, J=12.2, 6.7 Hz, 1H), 5.18 (s, 1H), 4.66 (s, 2H), 4.45-4.28 (m, 2H), 4.25 (s, 3H), 3.41 (ddd, J=18.7, 12.4, 1.6 Hz, 1H), 2.71 (ddd, J=18.6, 6.7, 1.7 Hz, 1H).

### (S)-3-fluoro-5-(1-(3-((5-fluoro-2-(2-methyl-2H-1,2,3-triazol-4-yl)pyri din-4-yl)oxy)azetidine-1-carbonyl)-4,5-dihydro-1H-pyrazol-5-yl)benzonitrile (15)

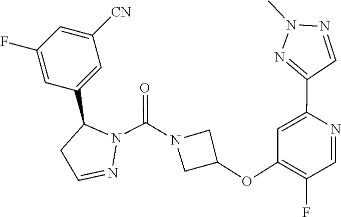 [SMILES: Cn1ncc(-c2cc(OC3CN(C(=O)N4N=CC[C@H]4c4cc(F)cc(C#N)c4)C3)c(F)cn2)n1]

The titled compound 15 was prepared from 17-01 in a yield of 51.8% according to the procedure outlined for compound 17. Mass (m/z) 465.1 [M+H]⁺. ¹H NMR (400 MHz, Chloroform-d) δ 8.47 (d, J=3.3 Hz, 1H), 8.28 (s, 1H), 7.62 (d, J=6.9 Hz, 1H), 7.34 (t, J=1.5 Hz, 1H), 7.28-7.25 (m, 1H), 7.20 (dt, J=9.0, 2.0 Hz, 1H), 6.82 (t, J=1.7 Hz, 1H), 5.31 (dd, J=12.2, 6.6 Hz, 1H), 5.21 (td, J=6.5, 3.2 Hz, 1H), 4.69 (s, 2H), 4.34 (t, J=12.4 Hz, 2H), 4.18 (s, 3H), 3.40 (ddd, J=18.7, 12.3, 1.7 Hz, 1H), 2.71 (ddd, J=18.7, 6.6, 1.7 Hz, 1H).

### S)-3-(1-(3-((2-(4-chloro-1-methyl-1H-pyrazol-5-yl)-5-fluoropyrimidin-4-yl)oxy)azetidine-1-carbonyl)-4,5-dihydro-1H-pyrazol-5-yl)-5-fluorobenzonitrile (16)

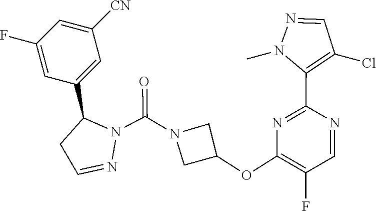 [SMILES: Cn1ncc(Cl)c1-c1ncc(F)c(OC2CN(C(=O)N3N=CC[C@H]3c3cc(F)cc(C#N)c3)C2)n1]

The titled compound 16 was prepared in a 38.9% yield according to the procedure outlined for compound 6. ¹H NMR (400 MHz, Chloroform-d) δ 8.49 (d, J=2.4 Hz, 1H), 7.50 (s, 1H), 7.34 (t, J=1.5 Hz, 1H), 7.26-7.23 (m, 1H), 7.21 (dt, J=9.0, 2.1 Hz, 1H), 6.83-6.78 (m, 1H), 5.59 (tt, J=6.6, 4.0 Hz, 1H), 5.31 (dd, J=12.2, 6.7 Hz, 1H), 4.65 (s, 2H), 4.33 (dd, J=26.2, 9.2 Hz, 2H), 4.18 (s, 3H), 3.39 (ddd, J=18.6, 12.2, 1.7 Hz, 1H), 2.70 (ddd, J=18.6, 6.8, 1.7 Hz, 1H). Mass (m/z) 499.2 [M+H]⁺.

### (S)-3-fluoro-5-(1-(3-((5-fluoro-2-(3-methylisothiazol-4-yl)pyridin-4-yl)oxy)azetidine-1-carbonyl)-4,5-dihydro-1H-pyrazol-5-yl)benzonitrile (17)

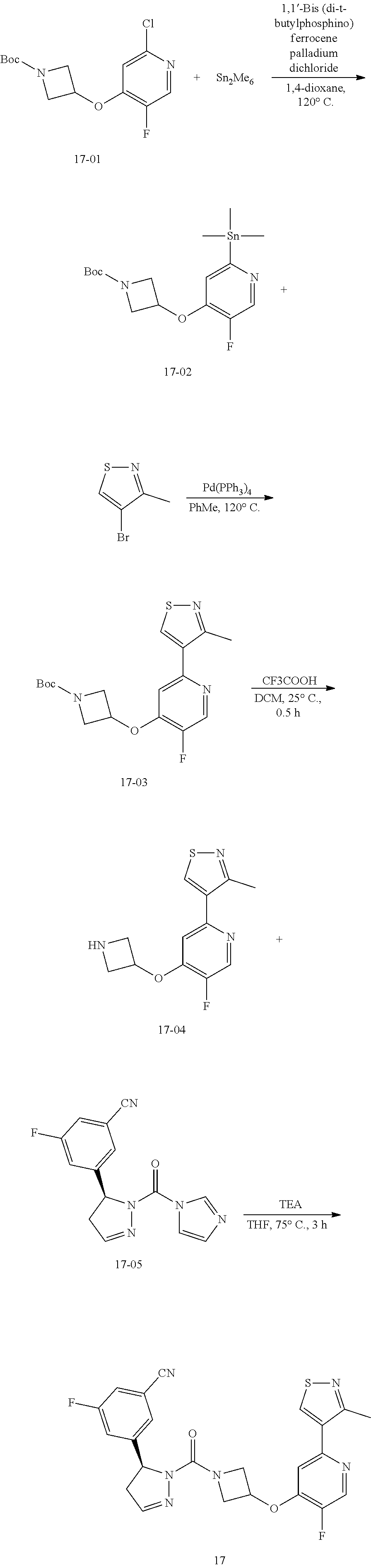

Step 1: compound 17-01 (1.04 g, 3.44 mmol), Sn₂Me₃ (1.7 g, 1.5 mmol), 1,1′-Bis (di-t-butylphosphino)ferrocene palladium dichloride (112 mg, 0.17 mmol) in 1,4-dioxane (15 mL) mixed under N₂ and the whole reaction mixture was stirred at 120° C. for 3 hours. The black suspension was filtered through a plug of Celite and washed with EA (100 mL). Concentrated to give 17-02 (1.47 g, 99.3%) as a brown oil.

Step 2: 17-02 (320 mg, 0.74 mmol), 4-bromo-3-methylisothiazole (132 mg, 0.74 mmol), Pd(PPh₃)₄ (43 mg, 0.037 mmol) in PhMe (3 mL) were mixed under N₂ and the whole reaction mixture was stirred at 120° C. for 15 hours. The mixture was extracted with EA, washed with brine, dried (Na₂SO₄), and concentrated in vacuo. Purification by silica gel chromatography to give 17-03 (180 mg, 66.5%) as a yellow solid.

Step 3: 17-03 (180 mg, 0.49 mmol) was dissolved in 3 mL of DCM, trifluoroacetic acid (560 mg, 4.9 mmol) was added, the mixture was stirred at 25° C. for 0.5 h. Concentrated to give the desired product 17-04, which was used for next step without further purification.

Step 4: compounds 17-04, 17-05 (124 mg, 0.44 mmol) and TEA (2 mL) were dissolved in THE (10 mL) and stirred at 75° C. for 3 h. The mixture was extracted with EA, washed with brine, dried (Na₂SO₄), and concentrated in vacuo. Purification by silica gel chromatography gave the titled compound 17 (85 mg, 40.2%) as a white solid. ¹H NMR (400 MHz, Chloroform-d) δ 8.80 (s, 1H), 8.50 (d, J=3.1 Hz, 1H), 7.33 (d, J=1.5 Hz, 1H), 7.28 (dd, J=2.5, 1.3 Hz, 1H), 7.20 (dt, J=8.9, 1.9 Hz, 1H), 6.88-6.77 (m, 2H), 5.32 (dd, J=12.2, 6.6 Hz, 1H), 5.12 (d, J=3.6 Hz, 1H), 4.62 (s, 2H), 4.35 (dd, J=29.3, 10.4 Hz, 2H), 3.41 (ddd, J=18.7, 12.2, 1.7 Hz, 1H), 2.72 (ddd, J=18.7, 6.6, 1.7 Hz, 1H), 2.64 (s, 3H). Mass (m/z) 481.2 [M+H]⁺.

### (S)-3-fluoro-5-(1-(3-((5-fluoro-2-(4-methylthiazol-5-yl)pyridin-4-yl)oxy)azetidine-1-carbonyl)-4,5-dihydro-1H-pyrazol-5-yl)benzonitrile (18)

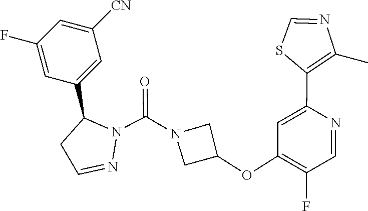 [SMILES: Cc1ncsc1-c1cc(OC2CN(C(=O)N3N=CC[C@H]3c3cc(F)cc(C#N)c3)C2)c(F)cn1]

The titled compound 18 was prepared in 38.9% yield according to the procedure outlined for compound 1. ¹H NMR (400 MHz, Chloroform-d) δ 8.90 (s, 1H), 8.43 (d, J=2.9 Hz, 1H), 7.33 (t, J=1.5 Hz, 1H), 7.27 (dd, J=2.6, 1.3 Hz, 1H), 7.20 (dt, J=9.1, 2.1 Hz, 1H), 6.90 (d, J=6.4 Hz, 1H), 6.85 (d, J=1.7 Hz, 1H), 5.32 (dd, J=12.2, 6.6 Hz, 1H), 5.13 (d, J=3.7 Hz, 1H), 4.62 (s, 2H), 4.35 (d, J=39.3 Hz, 2H), 3.42 (ddd, J=18.6, 12.2, 1.7 Hz, 1H), 2.71 (s, 4H). Mass (m/z) 481.2 [M+H]⁺.

### (S)-3-(1-(3-((2-(3,5-dimethyl-1H-pyrazol-4-yl)-5-fluoropyridin-4-yl)oxy)azetidine-1-carbonyl)-4,5-dihydro-1H-pyrazol-5-yl)-5-fluorobenzonitrile (19)

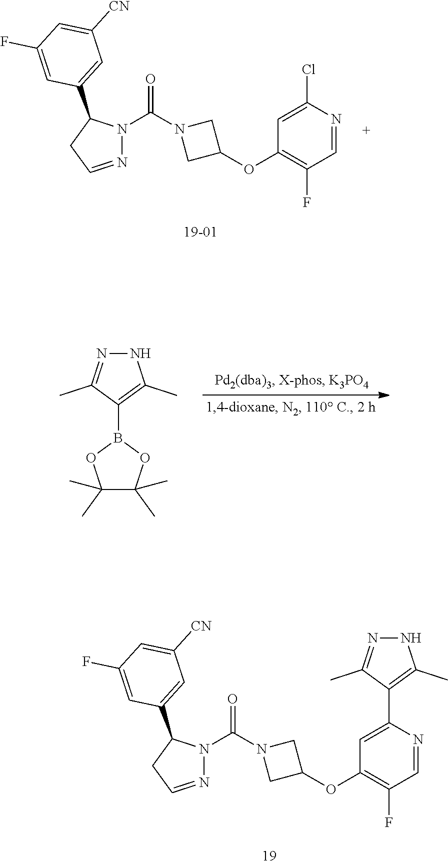

The titled compound 19 was prepared from 19-01 and 3,5-dimethyl-4-(4,4,5,5-tetramethyl-1,3,2-dioxaborolan-2-yl)-1H-pyrazole in 32.4% yield according to the procedure outlined for compound 1. ¹H NMR (400 MHz, Chloroform-d) δ 11.78 (s, 1H), 8.57 (d, J=3.2 Hz, 1H), 7.32 (d, J=1.5 Hz, 1H), 7.24 (dd, J=2.5, 1.3 Hz, 1H), 7.20 (dt, J=8.8, 1.9 Hz, 1H), 6.86 (d, J=1.7 Hz, 1H), 6.70 (d, J=6.4 Hz, 1H), 5.32 (dd, J=12.2, 6.6 Hz, 1H), 5.13 (dd, J=7.6, 3.8 Hz, 1H), 4.63 (s, 2H), 4.36 (d, J=24.6 Hz, 2H), 3.42 (ddd, J=18.7, 12.2, 1.7 Hz, 1H), 2.72 (ddd, J=18.7, 6.6, 1.7 Hz, 1H), 2.48 (s, 6H). Mass (m/z) 478.3 [M+H]⁺.

### (S)-3-fluoro-5-(1-(3-((5-fluoro-2-(1-(2-methoxyethyl)-3,5-dimethyl-1H-pyrazol-4-yl)pyridin-4-yl)oxy)azetidine-1-carbonyl)-4,5-dihydro-1H-pyrazol-5-yl)benzonitrile (20)

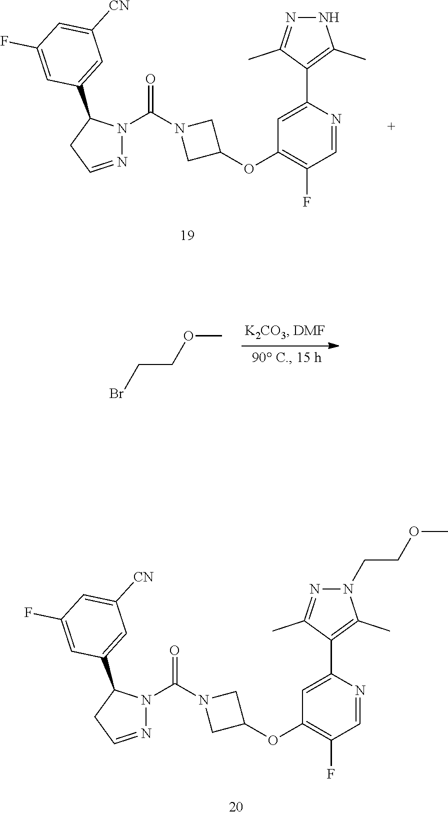

Compound 19 (72 mg, 0.15 mmol), 1-bromo-2-methoxyethane (107 mg, 0.75 mmol), K₂CO₃ (207 mg, 1.50 mmol) were placed in DMF (3 mL). The mixture was stirred 90° C. for overnight. Concentrated. Purification by prep-HPLC gave the titled compound 20 (4.7 mg, 5.9%) as a white solid. ¹H NMR (400 MHz, Chloroform-d) δ 8.73 (s, 1H), 7.32 (d, J=1.5 Hz, 1H), 7.28 (d, J=2.2 Hz, 1H), 7.20 (dd, J=9.1, 2.1 Hz, 1H), 6.86 (d, J=1.7 Hz, 1H), 6.67 (d, J=6.4 Hz, 1H), 5.32 (dd, J=12.2, 6.6 Hz, 1H), 5.14 (s, 1H), 4.63 (s, 2H), 4.45-4.27 (m, 4H), 3.75 (t, J=4.9 Hz, 2H), 3.42 (dd, J=18.7, 12.2 Hz, 1H), 3.31 (s, 3H), 2.73 (dd, J=18.6, 6.5 Hz, 1H), 2.35 (d, J=12.5 Hz, 6H). Mass (m/z) 536.3 [M+H]⁺.

### (S)-2-(4-(4-((1-(5-(3-cyano-5-fluorophenyl)-4,5-dihydro-1H-pyrazole-1-carbonyl)azetidin-3-yl)oxy)-5-fluoropyridin-2-yl)-3,5-dimethyl-1H-pyrazol-1-yl)acetamide (21)

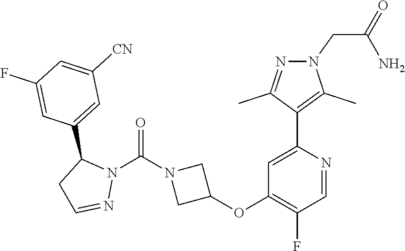 [SMILES: Cc1nn(CC(N)=O)c(C)c1-c1cc(OC2CN(C(=O)N3N=CC[C@H]3c3cc(F)cc(C#N)c3)C2)c(F)cn1]

The titled compound 21 was prepared from 19 in a 8.9% yield according to the procedure outlined for compound 20. ¹H NMR (400 MHz, DMSO-d₆) δ 8.50 (d, J=3.0 Hz, 1H), 7.77-7.73 (m, 1H), 7.57 (t, J=1.5 Hz, 1H), 7.53-7.49 (m, 1H), 7.45 (dt, J=9.5, 2.1 Hz, 1H), 7.24 (s, 1H), 7.06-7.03 (m, 1H), 6.82 (d, J=6.9 Hz, 1H), 5.29-5.25 (m, 1H), 4.68 (s, 2H), 4.53 (s, 2H), 4.22 (t, J=6.6 Hz, 1H), 4.09 (s, 2H), 3.45-3.35 (m, 1H), 3.30 (s, 3H), 2.73-2.65 (m, 1H), 2.30 (s, 3H). Mass (m/z) 535.3 [M+H]⁺.

### (S)-3-(1-(3-((2-(1,4-dimethyl-1H-imidazol-5-yl)-5-fluoropyridin-4-yl)oxy)azetidine-1-carbonyl)-4,5-dihydro-1H-pyrazol-5-yl)-5-fluorobenzonitrile (22)

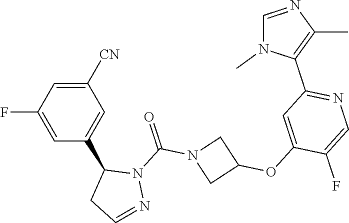 [SMILES: Cc1ncn(C)c1-c1cc(OC2CN(C(=O)N3N=CC[C@H]3c3cc(F)cc(C#N)c3)C2)c(F)cn1]

The titled compound 22 was prepared from 1-01 in a yield of 110% according to the procedure outlined for compound 23. Mass (m/z) 478.3 [M+H]⁺. ¹H NMR (400 MHz, Chloroform-d) δ 8.66-8.56 (m, 1H), 8.52 (d, J=2.7 Hz, 1H), 7.32 (s, 1H), 7.25-7.22 (m, 1H), 7.22-7.17 (m, 1H), 6.85 (s, 1H), 6.81-6.75 (m, 1H), 5.40-5.29 (m, 1H), 5.15-5.06 (m, 1H), 4.72-4.54 (m, 2H), 4.41-4.26 (m, 2H), 3.93 (s, 3H), 3.48-3.34 (m, 1H), 2.76-2.66 (m, 1H), 2.47 (s, 3H).

### (S)-3-(1-(3-((2-(1,4-dimethyl-1H-imidazol-2-yl)-5-fluoropyridin-4-yl)oxy)azetidine-1-carbonyl)-4,5-dihydro-1H-pyrazol-5-yl)-5-fluorobenzonitrile (23)

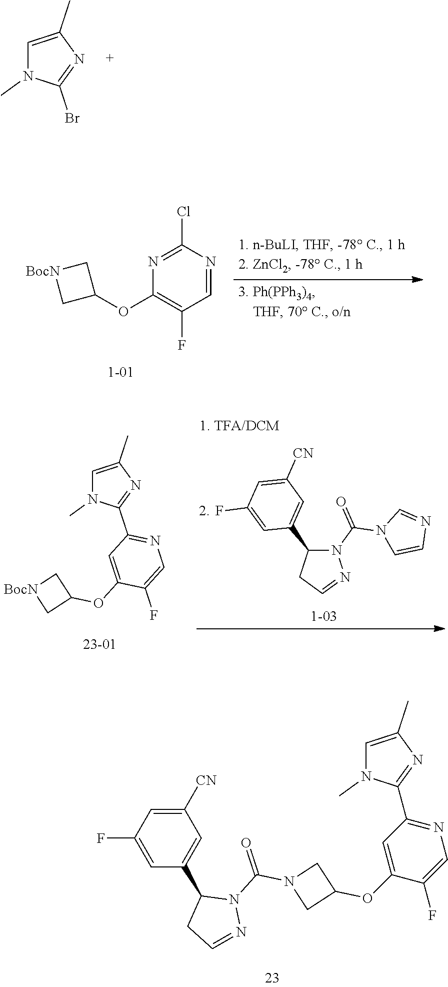

Step 1: To a solution of 2-bromo-1,4-dimethyl-1H-imidazole (87 mg, 0.5 mmol) in THF (5 mL) was added n-BuLi (1.6 M, 0.38 mL, 0.6 mmol) under Ar at −78° C. The reaction was stirred at −78° C. for 1 h. Then ZnCl₂ (1M in THF, 0.6 mL, 0.6 mmol) was added to the reaction and stirred at −78° C. for another 0.5 h. The reaction mixture was allowed to warm to rt over 1 h at which time 1-01 (152 mg, 0.5 mmol) and Pd(PPh₃)₄ (115 mg, 0.1 mmol) in THF was added. The reaction mixture was stirred at 70° C. for 16 h. The reaction mixture was cooled to rt and diluted with water. The aqueous phase was extracted with EA. The combined organic extracts were washed with brine and dried over Na₂SO₄. The solvent was removed under vacuum and the crude product 23-01 was used to next step directly.

Step 2: To a solution of 23-01 (101 mg, 0.28 mmol) in DCM (5 mL) was added TFA (2 mL). The reaction was stirred at rt for 1 h. The solvent was removed under vacuum. To the resulting residue in THF (5 mL) was added TEA (3 mL) and 1-03 (76 mg, 0.28 mmol). The reaction mixture was stirred at rt for 1 h. The reaction was cooled to rt and concentrated. The crude product was purified by Pre-TLC to give required product 23 (27 mg, 20.2%) as a white solid. Mass (m/z) 478.3 [M+H]⁺. ¹H NMR (400 MHz, Chloroform-d) δ 8.45 (d, J=2.4 Hz, 1H), 7.93-7.86 (m, 1H), 7.34 (s, 1H), 7.21 (t, J=8.6 Hz, 2H), 6.86 (s, 1H), 6.82-6.79 (m, 1H), 5.40-5.29 (m, 1H), 5.28-5.20 (m, 1H), 4.84-4.62 (m, 2H), 4.37-4.19 (m, 2H), 4.16 (s, 3H), 3.38 (dd, J=18.5, 12.1 Hz, 1H), 2.67 (dd, J=18.8, 6.1 Hz, 1H), 2.44 (s, 3H).

### (S)-(3-((2-(1,4-dimethyl-1H-pyrazol-5-yl)-5-fluoropyrimidin-4-yl)oxy)azetidin-1-yl)(5-(5-fluoropyridin-3-yl)-4,5-dihydro-1H-pyrazol-1-yl)methanone (24)

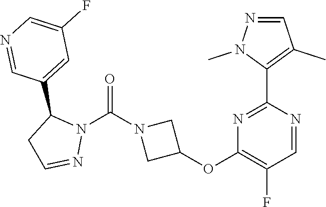 [SMILES: Cc1cnn(C)c1-c1ncc(F)c(OC2CN(C(=O)N3N=CC[C@H]3c3cncc(F)c3)C2)n1]

The titled compound 24 was prepared in 56.9% yield according to the procedure outlined for compound 1. ¹H NMR (400 MHz, Chloroform-d) δ 8.45 (d, J=2.4 Hz, 11H), 8.41-8.35 (m, 2H), 7.35 (d, J=0.7 Hz, 11H), 7.29-7.26 (m, 1H), 6.83 (t, J=1.7 Hz, 1H), 5.53 (tt, J=6.5, 4.0 Hz, 1H), 5.36 (dd, J=12.2, 6.7 Hz, 1H), 4.63 (d, J=16.3 Hz, 2H), 4.32 (dd, J=31.9, 8.7 Hz, 2H), 4.18 (s, 3H), 3.41 (ddd, J=18.6, 12.3, 1.7 Hz, 1H), 2.76 (ddd, J=18.6, 6.7, 1.7 Hz, 1H), 2.32 (s, 3H). Mass (m/z) 455.2 [M+H]⁺.

### (S)-3-(1-(3-((2-(1,4-dimethyl-1H-pyrazol-5-yl)-5-fluoropyrimidin-4-yl)oxy)azetidine-1-carbonyl)-4,5-dihydro-1H-pyrazol-5-yl)-5-fluorobenzonitrile (25)

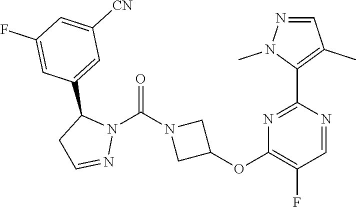 [SMILES: Cc1cnn(C)c1-c1ncc(F)c(OC2CN(C(=O)N3N=CC[C@H]3c3cc(F)cc(C#N)c3)C2)n1]

The titled compound 25 was prepared in a 46.8% yield according to the procedure outlined for compound 1. ¹H NMR (400 MHz, Chloroform-d) δ 8.44 (d, J=2.4 Hz, 1H), 7.33 (d, J=1.8 Hz, 2H), 7.26-7.15 (m, 2H), 6.82-6.78 (m, 1H), 5.53 (tt, J=6.5, 3.9 Hz, 1H), 5.29 (dd, J=12.2, 6.8 Hz, 1H), 4.60 (d, J=8.6 Hz, 2H), 4.41-4.24 (m, 2H), 4.16 (s, 3H), 3.38 (ddd, J=18.6, 12.2, 1.7 Hz, 1H), 2.69 (ddd, J=18.7, 6.8, 1.7 Hz, 1H), 2.30 (s, 3H). Mass (m/z) 479.3 [M+H]⁺.

### (S)-5-(5-fluoro-4-((1-(5-(5-fluoropyridin-3-yl)-4,5-dihydro-1H-pyrazole-1-carbonyl)azetidin-3-yl)oxy)pyridin-2-yl)-1-methyl-1H-pyrazole-4-carbonitrile (26)

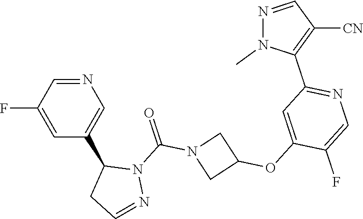 [SMILES: Cn1ncc(C#N)c1-c1cc(OC2CN(C(=O)N3N=CC[C@H]3c3cncc(F)c3)C2)c(F)cn1]

The titled compound 26 was prepared according to the procedure outlined for compound 9. Mass (m/z) 465.3 [M+H]⁺. ¹H NMR (400 MHz, Chloroform-d) δ 8.51 (d, J=2.7 Hz, 1H), 8.41-8.33 (m, 2H), 7.82 (s, 1H), 7.32-7.27 (m, 2H), 6.84-6.82 (m, 1H), 5.36 (dd, J=12.2, 6.6 Hz, 1H), 5.16-5.08 (m, 1H), 4.73-4.59 (m, 2H), 4.39-4.25 (m, 2H), 4.19 (s, 3H), 3.41 (ddd, J=18.6, 12.3, 1.7 Hz, 1H), 2.75 (ddd, J=18.5, 6.6, 1.7 Hz, 1H).

### (S)-(3-((5-fluoro-2-(1-methyl-1H-pyrazol-5-yl)pyridin-4-yl)oxy)azetidin-1-yl)(5-(5-fluoropyridin-3-yl)-4,5-dihydro-1H-pyrazol-1-yl)methanone (27)

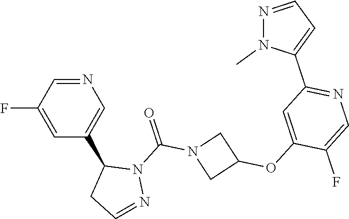 [SMILES: Cn1nccc1-c1cc(OC2CN(C(=O)N3N=CC[C@H]3c3cncc(F)c3)C2)c(F)cn1]

The titled compound 27 was prepared according to the procedure outlined for compound 7. Mass (m/z) 440.2 [M+H]⁺. ¹H NMR (400 MHz, Methanol-d₄) δ 8.46 (d, J=3.1 Hz, 1H), 8.41 (s, 1H), 8.37 (s, 1H), 7.62-7.56 (m, 1H), 7.49 (d, J=2.1 Hz, 1H), 7.19 (d, J=6.7 Hz, 1H), 6.98 (t, J=1.7 Hz, 11H), 6.69 (d, J=2.1 Hz, 11H), 5.38 (dd, J=12.1, 6.7 Hz, 11H), 5.30-5.22 (m, 1H), 4.64 (s, 2H), 4.30-4.15 (m, 2H), 3.48 (ddd, J=18.8, 12.2, 1.7 Hz, 1H), 2.79 (ddd, J=18.8, 6.7, 1.8 Hz, 1H).

### (S)-(3-((2-(1-ethyl-1H-pyrazol-5-yl)-5-fluoropyridin-4-yl)oxy)azetidin-1-yl)(5-(5-fluoropyridin-3-yl)-4,5-dihydro-1H-pyrazol-1-yl)methanone (28)

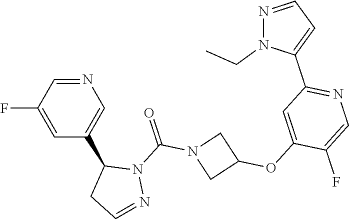 [SMILES: CCn1nccc1-c1cc(OC2CN(C(=O)N3N=CC[C@H]3c3cncc(F)c3)C2)c(F)cn1]

The titled compound 28 was prepared according to the procedure outlined for compound 12. Mass (m/z) 454.3 [M+H]⁺. ¹H NMR (400 MHz, Methanol-d₄) δ 8.46 (d, J=3.0 Hz, 1H), 8.40 (d, J=2.7 Hz, 1H), 8.35 (s, 1H), 7.60-7.54 (m, 1H), 7.51 (d, J=2.1 Hz, 1H), 7.18 (d, J=6.7 Hz, 1H), 7.00-6.98 (m, 1H), 6.68 (d, J=2.1 Hz, 1H), 5.38 (dd, J=12.2, 6.7 Hz, 1H), 5.31-5.23 (m, 1H), 4.70-4.61 (m, 2H), 4.56 (q, J=7.2 Hz, 2H), 4.29-4.15 (m, 2H), 3.48 (ddd, J=18.8, 12.1, 1.7 Hz, 1H), 2.79 (ddd, J=18.7, 6.7, 1.8 Hz, 1H), 1.35 (t, J=7.1 Hz, 3H).

### (S)-(3-((2-(1,3-dimethyl-1H-pyrazol-5-yl)-5-fluoropyridin-4-yl)oxy)azetidin-1-yl)(5-(5-fluoropyridin-3-yl)-4,5-dihydro-1H-pyrazol-1-yl)methan one (29)

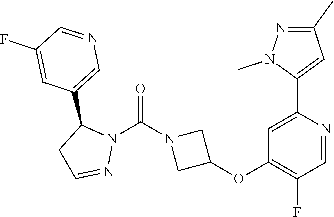 [SMILES: Cc1cc(-c2cc(OC3CN(C(=O)N4N=CC[C@H]4c4cncc(F)c4)C3)c(F)cn2)n(C)n1]

The titled compound 29 was prepared according to the procedure outlined for compound 1. Mass (m/z) 454.3 [M+H]⁺. ¹H NMR (400 MHz, Methanol-d₄) δ 8.43 (d, J=3.0 Hz, 1H), 8.41-8.38 (m, 1H), 8.35 (s, 1H), 7.61-7.55 (m, 1H), 7.14 (d, J=6.7 Hz, 1H), 6.98-6.96 (m, 1H), 6.49 (s, 1H), 5.40-5.33 (m, 1H), 5.27-5.21 (m, 1H), 4.62 (s, 2H), 4.28-4.12 (m, 2H), 4.01 (s, 3H), 3.47 (ddd, J=18.8, 12.2, 1.7 Hz, 1H), 2.78 (ddd, J=18.8, 6.7, 1.7 Hz, 1H), 2.24 (s, 3H).

### (S)-(3-((2-(1,4-dimethyl-1H-pyrazol-5-yl)-5-fluoropyridin-4-yl)oxy)azetidin-1-yl)(5-(5-fluoropyridin-3-yl)-4,5-dihydro-1H-pyrazol-1-yl)methan one 30)

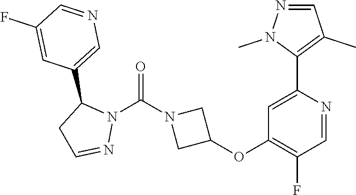 [SMILES: Cc1cnn(C)c1-c1cc(OC2CN(C(=O)N3N=CC[C@H]3c3cncc(F)c3)C2)c(F)cn1]

The titled compound 30 was prepared according to the procedure outlined for compound 1. Mass (m/z) 454.3 [M+H]⁺. ¹H NMR (400 MHz, Methanol-d₄) δ 8.43 (d, J=3.0 Hz, 11H), 8.41-8.38 (m, 11H), 8.35 (s, 11H), 7.60-7.55 (m, 1H), 7.14 (d, J=6.7 Hz, 1H), 6.98-6.96 (m, 1H), 6.49 (s, 1H), 5.40-5.33 (m, 1H), 5.27-5.21 (m, 1H), 4.62 (s, 2H), 4.28-4.13 (m, 2H), 4.01 (s, 3H), 3.47 (ddd, J=18.8, 12.2, 1.7 Hz, 1H), 2.78 (ddd, J=18.8, 6.7, 1.7 Hz, 1H), 2.24 (s, 3H).

### (S)-3-(1-(3-((2-(3,5-dimethylisothiazol-4-yl)-5-fluoropyridin-4-yl)oxy)azetidine-1-carbonyl)-4,5-dihydro-1H-pyrazol-5-yl)-5-fluorobenzonitrile (31)

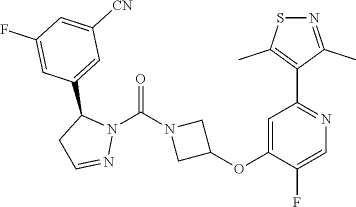 [SMILES: Cc1nsc(C)c1-c1cc(OC2CN(C(=O)N3N=CC[C@H]3c3cc(F)cc(C#N)c3)C2)c(F)cn1]

The titled compound 31 was prepared in a 4.1% yield according to the procedure outlined for compound 1. Mass (m/z) 495.2 [M+H]⁺. ¹H NMR (400 MHz, Chloroform-d) δ 8.55 (d, J=3.1 Hz, 1H), 7.33 (s, 1H), 7.28 (s, 1H), 7.20 (dd, J=8.9, 2.1 Hz, 1H), 6.84 (d, J=1.6 Hz, 1H), 6.62 (d, J=6.5 Hz, 1H), 5.31 (dd, J=12.2, 6.6 Hz, 1H), 5.08 (d, J=3.8 Hz, 1H), 4.60 (s, 2H), 4.35 (d, J=27.1 Hz, 2H), 3.41 (dd, J=18.6, 12.3 Hz, 1H), 2.78-2.66 (m, 1H), 2.47 (s, 3H), 2.41 (s, 3H).

### (S)-(3-((2-(4-chloro-1-methyl-1H-pyrazol-5-yl)-5-fluoropyrimidin-4-yl)oxy)azetidin-1-yl)(5-(5-fluoropyridin-3-yl)-4,5-dihydro-1H-pyrazol-1-yl)methanone (32)

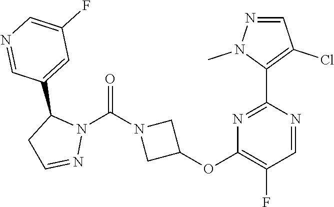 [SMILES: Cn1ncc(Cl)c1-c1ncc(F)c(OC2CN(C(=O)N3N=CC[C@H]3c3cncc(F)c3)C2)n1]

The titled compound 32 was prepared in a 9.7% yield according to the procedure outlined for compound 6. ¹H NMR (400 MHz, Chloroform-d) δ 8.48 (d, J=2.4 Hz, 1H), 8.40-8.34 (m, 2H), 7.50 (s, 1H), 7.29 (t, J=2.3 Hz, 11H), 6.82 (d, J=1.8 Hz, 11H), 5.58 (tt, J=6.6, 4.0 Hz, 11H), 5.36 (dd, J=12.3, 6.7 Hz, 1H), 4.66 (d, J=17.8 Hz, 2H), 4.32 (dd, J=26.4, 10.8 Hz, 2H), 4.18 (s, 3H), 3.41 (ddd, J=18.6, 12.3, 1.7 Hz, 1H), 2.75 (ddd, J=18.7, 6.7, 1.7 Hz, 1H). Mass (m/z) 475.2 [M+H]⁺.

### (S)-5-(1-(3-((2-(1,4-dimethyl-1H-pyrazol-5-yl)-5-fluoropyrimidin-4-yl)oxy)azetidine-1-carbonyl)-4,5-dihydro-1H-pyrazol-5-yl)nicotinonitrile (33)

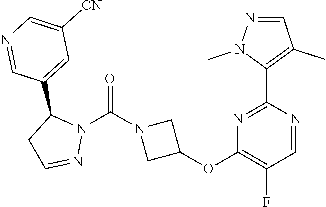 [SMILES: Cc1cnn(C)c1-c1ncc(F)c(OC2CN(C(=O)N3N=CC[C@H]3c3cncc(C#N)c3)C2)n1]

The titled compound 33 was prepared in a 67.3% yield according to the procedure outlined for compound 1. ¹H NMR (400 MHz, Chloroform-d) δ 8.76 (dd, J=21.8, 2.1 Hz, 2H), 8.45 (d, J=2.4 Hz, 1H), 7.82 (t, J=2.1 Hz, 1H), 7.34 (s, 1H), 6.89-6.82 (m, 1H), 5.53 (tt, J=6.6, 4.0 Hz, 1H), 5.36 (dd, J=12.3, 7.0 Hz, 1H), 4.61 (s, 2H), 4.42-4.24 (m, 2H), 4.17 (s, 3H), 3.44 (ddd, J=18.6, 12.3, 1.7 Hz, 1H), 2.74 (ddd, J=18.6, 7.0, 1.7 Hz, 1H), 2.31 (s, 3H). Mass (m/z) 462.3 [M+H]⁺.

### (S)-5-(5-fluoro-6-((1-(5-(5-fluoropyridin-3-yl)-4,5-dihydro-1H-pyrazole-1-carbonyl)azetidin-3-yl)oxy)pyridin-2-yl)-1-methyl-1H-pyrazole-4-carbonitrile (34)

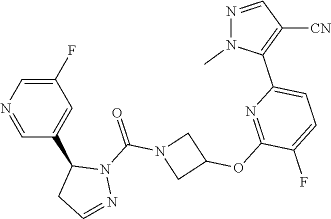 [SMILES: Cn1ncc(C#N)c1-c1ccc(F)c(OC2CN(C(=O)N3N=CC[C@H]3c3cncc(F)c3)C2)n1]

The title compound 34 was prepared in a yield of 40% as a white solid according to the procedure outlined for compound 10. Mass (m/z) 465.3 [M+H]⁺. ¹H NMR (400 MHz, Chloroform-d) δ 8.47-8.36 (m, 2H), 7.82 (s, 1H), 7.60-7.52 (m, 1H), 7.48 (dd, J=8.1, 2.9 Hz, 1H), 7.38 (d, J=8.9 Hz, 1H), 6.85-6.82 (m, 1H), 5.50-5.43 (m, 1H), 5.38 (dd, J=12.2, 6.7 Hz, 1H), 4.68-4.53 (m, 2H), 4.42-4.23 (m, 2H), 4.10 (s, 3H), 3.43 (dd, J=18.6, 12.2 Hz, 1H), 2.76 (dd, J=18.5, 6.6 Hz, 1H).

### (S)-3-(1-(3-((2-(3-amino-1-methyl-1H-pyrazol-5-yl)-5-fluoropyridin-4-yl)oxy)azetidine-1-carbonyl)-4,5-dihydro-1H-pyrazol-5-yl)-5-fluorobenzonitrile (35)

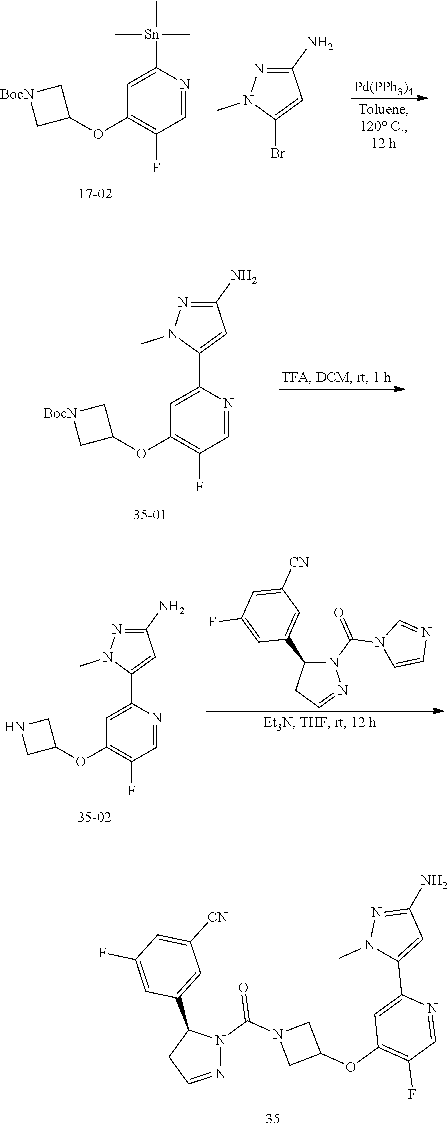

The title compound 35 was prepared in a yield of 38% as a white solid from 17-02 according to the procedure outlined for compound 17. Mass (m/z) 479.3 [M+H]⁺. ¹H NMR (400 MHz, Chloroform-d) δ 8.46 (d, J=2.7 Hz, 1H), 7.29-7.23 (m, 1H), 7.24 (d, J=3.4 Hz, OH), 7.22-7.17 (m, 1H), 6.88 (d, J=6.3 Hz, 1H), 6.86-6.83 (m, 1H), 5.83 (s, 1H), 5.39-5.27 (m, 1H), 5.16-5.06 (m, 1H), 4.71-4.54 (m, 2H), 4.43-4.25 (m, 2H), 4.04 (s, 3H), 3.41 (dd, J=18.7, 12.2 Hz, 1H), 2.71 (dd, J=19.0, 6.3 Hz, 1H).

### (S)-3-fluoro-5-(1-(3-((5-fluoro-2-(1-methyl-1H-imidazol-2-yl)pyridin-4-yl)oxy)azetidine-1-carbonyl)-4,5-dihydro-1H-pyrazol-5-yl)benzonitrile (36)

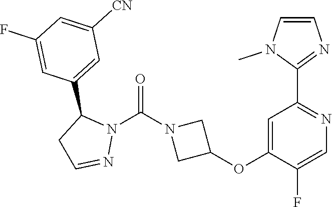 [SMILES: Cn1ccnc1-c1cc(OC2CN(C(=O)N3N=CC[C@H]3c3cc(F)cc(C#N)c3)C2)c(F)cn1]

The title compound 36 was prepared according to the procedure outlined for compound 13. ¹H NMR (400 MHz, Chloroform-d) δ 8.34 (d, J=2.6 Hz, 1H), 7.66 (s, 1H), 7.33 (d, J=1.4 Hz, 1H), 7.27-7.24 (m, 1H), 7.20 (dt, J=9.0, 2.0 Hz, 1H), 7.12 (s, 1H), 6.98 (s, 1H), 6.79 (d, J=1.7 Hz, 1H), 5.30 (dd, J=12.2, 6.7 Hz, 1H), 5.23-5.15 (m, 1H), 4.66 (s, 2H), 4.29 (s, 2H), 4.09 (s, 3H), 3.39 (ddd, J=18.6, 12.3, 1.7 Hz, 1H), 2.69 (ddd, J=18.6, 6.7, 1.7 Hz, 1H).

### (S)-(3-((5-fluoro-2-(1-methyl-1H-imidazol-2-yl)pyridin-4-yl)oxy)azetidin-1-yl)(5-(5-fluoropyridin-3-yl)-4,5-dihydro-1H-pyrazol-1-yl)methanone (37)

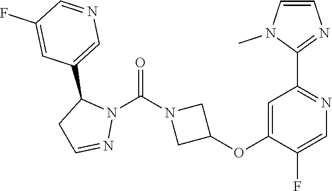 [SMILES: Cn1ccnc1-c1cc(OC2CN(C(=O)N3N=CC[C@H]3c3cncc(F)c3)C2)c(F)cn1]

The title compound 37 was prepared according to the procedure outlined for compound 13. ¹H NMR (400 MHz, Chloroform-d) δ 8.42-8.30 (m, 3H), 7.62 (d, J=6.8 Hz, 1H), 7.27 (t, J=2.3 Hz, 1H), 7.25 (d, J=2.3 Hz, 1H), 7.11 (d, J=1.2 Hz, 1H), 6.97 (d, J=1.1 Hz, 1H), 6.85-6.77 (m, 11H), 5.35 (dd, J=12.2, 6.6 Hz, 11H), 5.16 (td, J=6.4, 3.3 Hz, 11H), 4.65 (s, 2H), 4.29 (t, J=12.2 Hz, 2H), 4.09 (s, 3H), 3.40 (ddd, J=18.6, 12.2, 1.7 Hz, 1H), 2.74 (ddd, J=18.6, 6.7, 1.7 Hz, 1H).

### (S)-5-(1-(3-((5-fluoro-2-(1-methyl-1H-imidazol-2-yl)pyridin-4-yl)oxy)azetidine-1-carbonyl)-4,5-dihydro-1H-pyrazol-5-yl)nicotinonitrile (38)

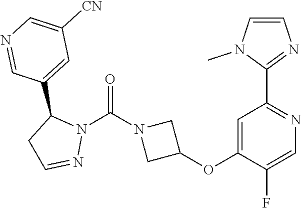 [SMILES: Cn1ccnc1-c1cc(OC2CN(C(=O)N3N=CC[C@H]3c3cncc(C#N)c3)C2)c(F)cn1]

The title compound 38 was prepared according to the procedure outlined for compound 13. ¹H NMR (400 MHz, Chloroform-d) δ 8.79 (d, J=1.9 Hz, 11H), 8.73 (d, J=2.2 Hz, 11H), 8.33 (d, J=2.6 Hz, 11H), 7.82 (t, J=2.1 Hz, 1H), 7.61 (d, J=6.7 Hz, 1H), 7.10 (s, 1H), 6.97 (d, J=1.1 Hz, 1H), 6.83 (d, J=1.7 Hz, 1H), 5.36 (dd, J=12.3, 6.9 Hz, 1H), 5.17 (dt, J=6.5, 2.8 Hz, 1H), 4.65 (s, 2H), 4.29 (s, 2H), 4.08 (s, 3H), 3.43 (ddd, J=18.6, 12.3, 1.7 Hz, 1H), 2.73 (ddd, J=18.6, 6.9, 1.7 Hz, 1H).

### (S)-3-(1-(3-((2-(2,5-dimethyl-1H-pyrrol-1-yl)-5-fluoropyridin-4-yl)ox y)azetidine-1-carbonyl)-4,5-dihydro-1H-pyrazol-5-yl)-5-fluorobenzonitrile (39)

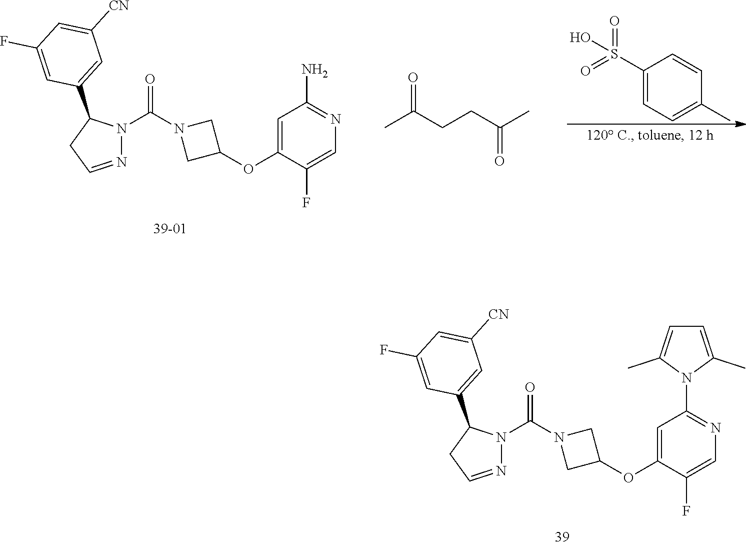

Compound 39-01 (100 mg, 0.25 mmol) was dissolved in 3 mL of toluene, hexane-2,5-dione (115 mg, 1 mmol) and 4-methylbenzenesulfonic acid (20 mg, 0.025 mmol) and the mixture was stirred at 120° C. for 12 h. Concentrated and purification by prep-HPLC gave the titled compound 39 (12 mg, 10.2%) as a white solid. ¹H NMR (400 MHz, Chloroform-d) δ 8.35 (d, J=2.7 Hz, 1H), 7.33 (t, J=1.5 Hz, 1H), 7.29-7.24 (m, 1H), 7.22-7.19 (m, 1H), 6.83 (t, J=1.7 Hz, 1H), 6.52 (d, J=6.0 Hz, 1H), 5.89 (s, 2H), 5.30 (dd, J=12.2, 6.7 Hz, 1H), 5.02 (td, J=6.5, 3.3 Hz, 1H), 4.67-4.52 (m, 2H), 4.40-4.24 (m, 2H), 3.41 (ddd, J=18.7, 12.3, 1.7 Hz, 1H), 2.71 (ddd, J=18.6, 6.7, 1.7 Hz, 1H), 2.11 (s, 6H). Mass (m/z) 477.2 [M+H]⁺.

### (S)-5-(6-((1-(5-(3-cyano-5-fluorophenyl)-4,5-dihydro-1H-pyrazole-1-carbonyl)azetidin-3-yl)oxy)-5-fluoropyridin-2-yl)-1-methyl-1H-pyrazole-4-carbonitrile (40)

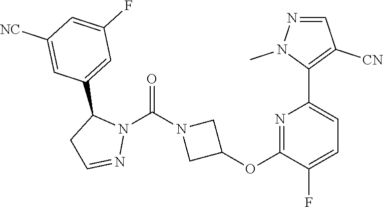 [SMILES: Cn1ncc(C#N)c1-c1ccc(F)c(OC2CN(C(=O)N3N=CC[C@H]3c3cc(F)cc(C#N)c3)C2)n1]

The title compound 40 was prepared in a yield of 57% as a white solid according to the procedure outlined for compound 8. Mass (m/z) 489.3 [M+H]⁺. ¹H NMR (400 MHz, Chloroform-d) δ 7.82 (s, 1H), 7.60-7.53 (m, 1H), 7.48 (dd, J=8.1, 2.9 Hz, 1H), 7.36-7.32 (m, 1H), 7.25-7.18 (m, 2H), 6.84-6.79 (m, 1H), 5.50-5.42 (m, 1H), 5.31 (dd, J=12.2, 6.8 Hz, 1H), 4.67-4.54 (m, 2H), 4.43-4.24 (m, 2H), 4.11 (s, 3H), 3.39 (ddd, J=18.6, 12.2, 1.7 Hz, 1H), 2.69 (ddd, J=18.6, 6.8, 1.7 Hz, 1H).

### (S)-5-(1-(3-((6-(4-cyano-1-methyl-1H-pyrazol-5-yl)-3-fluoropyridin-2-yl)oxy)azetidine-1-carbonyl)-4,5-dihydro-1H-pyrazol-5-yl)nicotinonitrile (41)

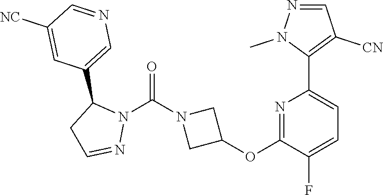 [SMILES: Cn1ncc(C#N)c1-c1ccc(F)c(OC2CN(C(=O)N3N=CC[C@H]3c3cncc(C#N)c3)C2)n1]

The title compound 41 was prepared in a yield of 50% as a white solid according to the procedure outlined for compound 8. Mass (m/z) 489.3 [M+H]⁺. ¹H NMR (400 MHz, Chloroform-d) δ 8.80 (s, 1H), 8.74 (s, 1H), 7.84 (t, J=2.1 Hz, 11H), 7.82 (s, 11H), 7.61-7.52 (m, 11H), 7.48 (dd, J=8.1, 2.9 Hz, 1H), 6.89-6.83 (m, 1H), 5.50-5.43 (m, 1H), 5.38 (dd, J=12.3, 6.9 Hz, 1H), 4.69-4.54 (m, 2H), 4.44-4.24 (m, 2H), 4.10 (s, 3H), 3.45 (ddd, J=18.7, 12.3, 1.7 Hz, 1H), 2.75 (ddd, J=18.6, 6.9, 1.7 Hz, 1H).

### (S)-5-(1-(3-((2-(1,4-dimethyl-1H-pyrazol-5-yl)-5-fluoropyridin-4-yl)oxy)azetidine-1-carbonyl)-4,5-dihydro-1H-pyrazol-5-yl)nicotinonitrile (42)

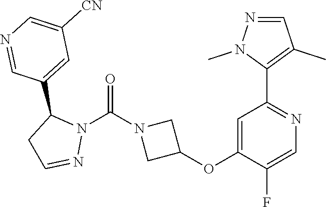 [SMILES: Cc1cnn(C)c1-c1cc(OC2CN(C(=O)N3N=CC[C@H]3c3cncc(C#N)c3)C2)c(F)cn1]

The title compound 42 was prepared in a yield of 23% as a white solid according to the procedure outlined for compound 11. Mass (m/z) 461.3 [M+H]⁺. ¹H NMR (400 MHz, Chloroform-d) δ 8.80 (d, J=1.9 Hz, 1H), 8.74 (d, J=2.3 Hz, 1H), 8.50 (d, J=2.9 Hz, 1H), 7.82 (t, J=2.1 Hz, 1H), 7.36 (s, 1H), 6.90-6.85 (m, 1H), 6.70 (d, J=6.6 Hz, 1H), 5.37 (dd, J=12.2, 6.9 Hz, 1H), 5.11-5.03 (m, 1H), 4.66-4.53 (m, 2H), 4.43-4.23 (m, 2H), 3.96 (s, 3H), 3.46 (ddd, J=18.7, 12.3, 1.8 Hz, 1H), 2.76 (ddd, J=18.7, 7.0, 1.7 Hz, 1H), 2.12 (s, 3H).

### (S)-(3-((2-(4-chloro-1-methyl-1H-pyrazol-5-yl)-5-fluoropyridin-4-yl)oxy)azetidin-1-yl)(5-(5-fluoropyridin-3-yl)-4,5-dihydro-1H-pyrazol-1-yl) methanone (43)

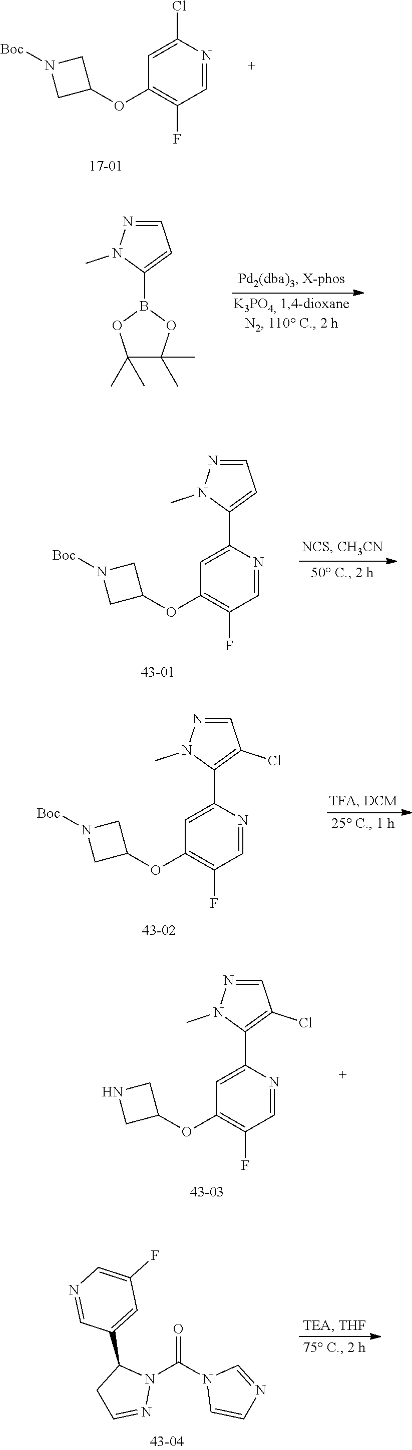

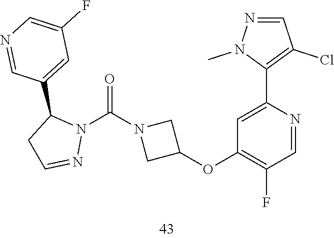 [SMILES: Cn1ncc(Cl)c1-c1cc(OC2CN(C(=O)N3N=CC[C@H]3c3cncc(F)c3)C2)c(F)cn1]

Step 1: 17-01 (1515 mg, 5.0 mmol), 1-methyl-5-(4,4,5,5-tetramethyl-1,3,2-dioxaborolan-2-yl)-1H-pyrazole (1248 mg, 6.0 mmol), Pd₂(dba)₃ (458 mg, 0.5 mmol) and X-phos (477 mg, 1.0 mmol) were added to a solution of K₃PO₄ (5N, 5 mL, 25 mmol) in 1,4-dioxane (25 mL) under N₂ and the whole reaction mixture was stirred at 110° C. for 2 hours. After the mixture was concentrated and further purification by silica gel chromatography to give 43-01 (1650 mg, 94.8%) as a yellow oil. Mass (m/z) 349.2 [M+H]⁺.

Step 2: 43-01 (275 mg, 0.8 mmol) and NCS (118 mg, 0.88 mmol) in CH₃CN (10 mL) were mixed under N₂ and the whole reaction mixture was stirred at 50° C. for 2 hours. After the mixture was concentrated and further purification by silica gel chromatography to give 43-02 (300 mg, 98.0%) as a clear oil. Mass (m/z) 383.2 [M+H]⁺.

Step 3: 43-02 (150 mg, 0.39 mmol) was dissolved in 3 mL of DCM, trifluoroacetic acid (445 mg, 3.9 mmol) was added, the mixture was stirred at 25° C. for 1 hour. Concentrated and gave the desired product 43-03, which was used for next step without further purification.

Step 4: 43-03, 43-04 (91 mg, 0.35 mmol) and TEA (2 mL) were dissolved in THE (5 mL) and stirred at 75° C. for 2 h. The mixture was extracted with EA, washed with brine, dried (Na₂SO₄), and concentrated in vacuo. Purification by silica gel chromatography to give the titled compound 43 (64 mg, 38.8%) as a white solid. ¹H NMR (400 MHz, Chloroform-d) δ 8.49 (d, J=2.8 Hz, 1H), 8.41-8.35 (m, 2H), 7.49 (s, 1H), 7.30-7.27 (m, 11H), 7.11 (d, J=6.6 Hz, 11H), 6.84 (t, J=1.6 Hz, 11H), 5.36 (dd, J=12.2, 6.7 Hz, 1H), 5.08 (ddd, J=10.3, 6.5, 4.0 Hz, 1H), 4.61 (s, 2H), 4.33 (dd, J=32.5, 10.3 Hz, 2H), 4.06 (s, 3H), 3.42 (ddd, J=18.6, 12.2, 1.7 Hz, 1H), 2.77 (ddd, J=18.7, 6.7, 1.7 Hz, 1H). Mass (m/z) 474.2 [M+H]⁺.

### ((S)-5-(1-(3-((2-(4-chloro-1-methyl-1H-pyrazol-5-yl)-5-fluoropyridin-4-yl)oxy)azetidine-1-carbonyl)-4,5-dihydro-1H-pyrazol-5-yl)nicotinonitrile (44)

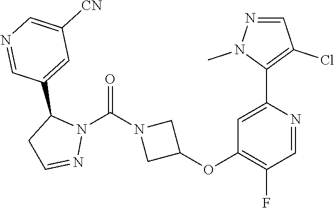 [SMILES: Cn1ncc(Cl)c1-c1cc(OC2CN(C(=O)N3N=CC[C@H]3c3cncc(C#N)c3)C2)c(F)cn1]

The titled compound 44 was prepared in a 37.5% yield according to the procedure outlined for compound 43. ¹H NMR (400 MHz, Chloroform-d) δ 8.77 (dd, J=23.7, 2.1 Hz, 2H), 8.49 (d, J=2.8 Hz, 1H), 7.82 (t, J=2.1 Hz, 1H), 7.49 (s, 1H), 7.12 (d, J=6.6 Hz, 1H), 6.90-6.85 (m, 11H), 5.37 (dd, J=12.2, 6.9 Hz, 11H), 5.09 (td, J=6.5, 3.3 Hz, 11H), 4.62 (s, 2H), 4.35 (d, J=31.5 Hz, 2H), 4.06 (s, 3H), 3.45 (ddd, J=18.6, 12.3, 1.7 Hz, 1H), 2.76 (ddd, J=18.6, 7.0, 1.7 Hz, 1H). Mass (m/z) 481.3 [M+H]⁺.

### (S)-(3-((5-fluoro-2-(4-fluoro-1-methyl-1H-pyrazol-5-yl)pyridin-4-yl)oxy)azetidin-1-yl)(5-(5-fluoropyridin-3-yl)-4,5-dihydro-1H-pyrazol-1-yl) methanone (45)

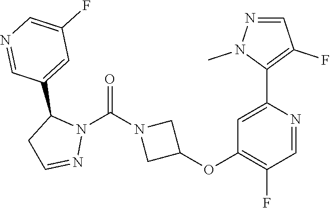 [SMILES: Cn1ncc(F)c1-c1cc(OC2CN(C(=O)N3N=CC[C@H]3c3cncc(F)c3)C2)c(F)cn1]

The titled compound 45 was prepared in a 22.7% yield according to the procedure outlined for compound 5. ¹H NMR (400 MHz, Chloroform-d) δ 8.44 (d, J=2.8 Hz, 11H), 8.41-8.35 (m, 2H), 7.38 (d, J=4.4 Hz, 11H), 7.28 (t, J=2.3 Hz, 11H), 6.98 (d, J=6.6 Hz, 11H), 6.84 (d, J=1.7 Hz, 11H), 5.36 (dd, J=12.2, 6.7 Hz, 1H), 5.08 (td, J=6.5, 3.3 Hz, 1H), 4.60 (s, 2H), 4.31 (dd, J=30.4, 10.7 Hz, 2H), 4.18-4.12 (m, 3H), 3.42 (ddd, J=18.6, 12.2, 1.7 Hz, 1H), 2.76 (ddd, J=18.6, 6.7, 1.7 Hz, 1H). Mass (m/z) 458.2 [M+H]⁺.

### (S)-5-(1-(3-((5-fluoro-2-(4-fluoro-1-methyl-1H-pyrazol-5-yl)pyridin-4-yl)oxy)azetidine-1-carbonyl)-4,5-dihydro-1H-pyrazol-5-yl)nicotinonitrile (46)

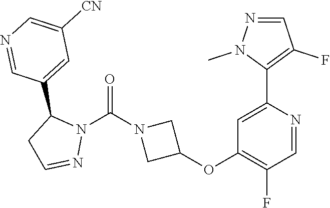 [SMILES: Cn1ncc(F)c1-c1cc(OC2CN(C(=O)N3N=CC[C@H]3c3cncc(C#N)c3)C2)c(F)cn1]

The titled compound 46 was prepared in a 8.6% yield according to the procedure outlined for compound 5. ¹H NMR (301 MHz, Chloroform-d) δ 8.78 (d, J=18.2 Hz, 2H), 8.46 (d, J=2.8 Hz, 1H), 7.86 (s, 1H), 7.40 (d, J=4.4 Hz, 1H), 6.99 (d, J=6.5 Hz, 1H), 6.88 (s, 1H), 5.44-5.36 (m, 1H), 5.11 (s, 1H), 4.62 (s, 2H), 4.41-4.29 (m, 2H), 4.16 (s, 3H), 3.47 (d, J=5.9 Hz, 1H), 2.79-2.70 (m, 1H). Mass (m/z) 465.2 [M+H]⁺.

### (S)-3-fluoro-5-(1-(3-((5-fluoro-2-(1,3,5-trimethyl-1H-pyrazol-4-yl)pyridin-4-yl)oxy)azetidine-1-carbonyl)-4,5-dihydro-1H-pyrazol-5-yl)benzonitrile (47)

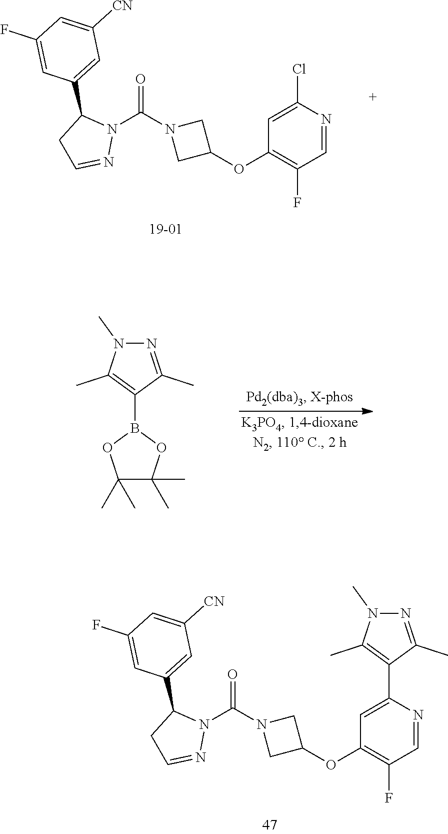

Compound 19-01 (104 mg, 0.25 mmol), 1,3,5-trimethyl-4-(4,4,5,5-tetramethyl-1,3,2-dioxaborolan-2-yl)-1H-pyrazole (88 mg, 0.375 mmol), Pd₂(dba)₃ (23 mg, 0.025 mmol), X-phos (24 mg, 0.05 mmol) were added to a solution of K₃PO₄ (5N, 0.25 mL, 1.25 mmol) in 1,4-dioxane (2.5 mL) under N₂ and the whole reaction mixture was stirred at 110° C. for 2 hours. After the mixture was concentrated and further purification by prep-HPLC to give 47 (50 mg, 40.7%) as a white solid. ¹H NMR (400 MHz, Chloroform-d) δ 8.40 (d, J=3.0 Hz, 1H), 7.33 (d, J=1.6 Hz, 1H), 7.25 (dd, J=2.5, 1.4 Hz, 1H), 7.20 (dt, J=9.1, 2.0 Hz, 1H), 6.82 (d, J=1.7 Hz, 1H), 6.58 (d, J=6.6 Hz, 1H), 5.30 (dd, J=12.2, 6.8 Hz, 1H), 5.05 (dq, J=6.4, 3.9, 3.2 Hz, 1H), 4.58 (s, 2H), 4.33 (dd, J=32.3, 10.5 Hz, 2H), 3.76 (s, 3H), 3.40 (ddd, J=18.7, 12.2, 1.7 Hz, 1H), 2.71 (ddd, J=18.6, 6.8, 1.7 Hz, 1H), 2.35 (d, J=23.0 Hz, 6H). Mass (m/z) 492.3 [M+H]⁺.

### (S)-3-(1-(3-((2-(4-(ethylsulfonyl)-3,5-dimethyl-1H-pyrazol-1-yl)-5-fluoropyridin-4-yl)oxy)azetidine-1-carbonyl)-4,5-dihydro-1H-pyrazol-5-yl)-5-fluorobenzonitrile (48)

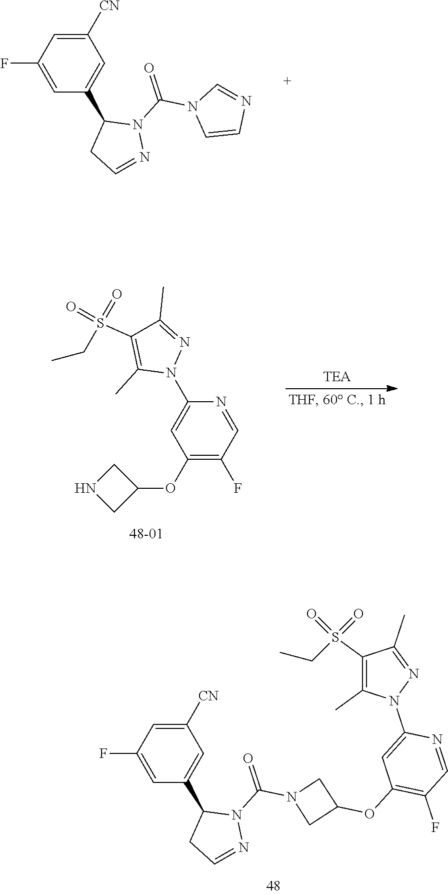 [SMILES: N#Cc1cc(F)cc([C@@H]2CC=NN2C(=O)n2ccnc2)c1 ; CCS(=O)(=O)c1c(C)nn(-c2cc(OC3CNC3)c(F)cn2)c1C ; CCS(=O)(=O)c1c(C)nn(-c2cc(OC3CN(C(=O)N4N=CC[C@H]4c4cc(F)cc(C#N)c4)C3)c(F)cn2)c1C]

The titled compound 48 was prepared from 48-01 in a yield of 3.1% according to the procedure outlined for compound 1-02. Mass (m/z) 570.5 [M+H]⁺. ¹H NMR (300 MHz, DMSO-d₆) δ 8.50 (d, J=2.8 Hz, 1H), 7.77-7.71 (m, 1H), 7.56 (t, J=1.6 Hz, 1H), 7.44 (dt, J=9.6, 2.0 Hz, 1H), 7.29 (d, J=6.0 Hz, 1H), 7.03 (d, J=1.6 Hz, 1H), 5.31-5.25 (m, 2H), 4.56-4.51 (m, 2H), 4.10-4.06 (m, 2H), 3.47-3.33 (m, 1H), 3.24 (q, J=7.2 Hz, 2H), 2.78-2.67 (m, 1H), 2.65 (s, 3H), 2.39 (s, 3H), 1.17 (t, J=7.2 Hz, 3H).

### (S)-3-(1-(3-((2-(3-amino-1,4-dimethyl-1H-pyrazol-5-yl)-5-fluoropyridin-4-yl)oxy)azetidine-1-carbonyl)-4,5-dihydro-1H-pyrazol-5-yl)-5-fluoro benzonitrile (49)

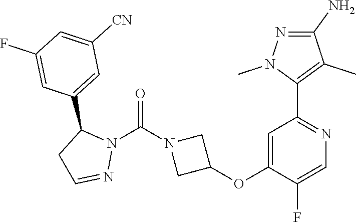 [SMILES: Cc1c(N)nn(C)c1-c1cc(OC2CN(C(=O)N3N=CC[C@H]3c3cc(F)cc(C#N)c3)C2)c(F)cn1]

The titled compound 49 was prepared in a 56.9% yield according to the procedure outlined for compound 35. ¹H NMR (400 MHz, Chloroform-d) δ 8.53 (d, J=2.8 Hz, 1H), 7.42 (s, 2H), 7.32 (t, J=1.4 Hz, 1H), 7.26-7.15 (m, 2H), 6.87-6.82 (m, 1H), 6.76 (d, J=6.4 Hz, 1H), 5.38-5.23 (m, 1H), 5.10 (tt, J=6.5, 3.9 Hz, 1H), 4.61 (s, 2H), 4.48-4.25 (m, 2H), 3.81 (s, 3H), 3.41 (ddd, J=18.7, 12.2, 1.7 Hz, 1H), 2.72 (ddd, J=18.7, 6.7, 1.7 Hz, 1H), 1.96 (s, 3H). Mass (m/z) 493.3 [M+H]⁺.

### (S)-3-(1-(3-((2-(3-chloro-1,4-dimethyl-1H-pyrazol-5-yl)-5-fluoropyridin-4-yl)oxy)azetidine-1-carbonyl)-4,5-dihydro-1H-pyrazol-5-yl)-5-fluoro benzonitrile

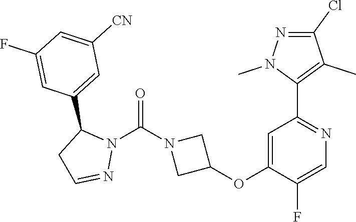 [SMILES: Cc1c(Cl)nn(C)c1-c1cc(OC2CN(C(=O)N3N=CC[C@H]3c3cc(F)cc(C#N)c3)C2)c(F)cn1]

The titled compound 50 was prepared in an 86.1% yield according to the procedure outlined for compound 6. ¹H NMR (400 MHz, Chloroform-d) δ 8.50 (d, J=2.9 Hz, 1H), 7.33 (t, J=1.5 Hz, 1H), 7.26 (s, 1H), 7.20 (dt,J=9.0, 2.0 Hz, 1H), 6.84 (t, J=1.6 Hz, 1H), 6.70 (d, J=6.4 Hz, 1H), 5.31 (dd, J=12.2, 6.6 Hz, 1H), 5.08 (tt, J=6.5, 3.9 Hz, 1H), 4.60 (s, 2H), 4.34 (d, J=29.5 Hz, 2H), 3.90 (s, 3H), 3.41 (ddd, J=18.6, 12.3, 1.7 Hz, 1H), 2.72 (ddd, J=18.7, 6.7, 1.7 Hz, 1H), 2.06 (s, 3H). Mass (m/z) 512.3 [M+H]⁺.

### (S)-5-(4-((1-(5-(3-cyano-5-fluorophenyl)-4,5-dihydro-1H-pyrazole-1-carbonyl)azetidin-3-yl)oxy)-5-fluoropyridin-2-yl)-1,4-dimethyl-1H-pyrazole-3-carbonitrile (51)

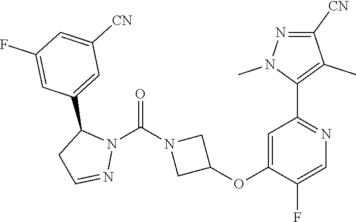 [SMILES: Cc1c(C#N)nn(C)c1-c1cc(OC2CN(C(=O)N3N=CC[C@H]3c3cc(F)cc(C#N)c3)C2)c(F)cn1]

The titled compound 51 was prepared in 28.6% yield according to the procedure outlined for compound 8. ¹H NMR (301 MHz, Chloroform-d) δ 8.52 (d, J=2.9 Hz, 1H), 7.40-7.14 (m, 3H), 6.84 (s, 1H), 6.73 (d, J=6.4 Hz, 1H), 5.31 (dd, J=12.3, 6.6 Hz, 1H), 5.15-5.05 (m, 1H), 4.70-4.52 (m, 2H), 4.43-4.23 (m, 2H), 4.00 (s, 3H), 3.42 (dd, J=18.7, 12.2 Hz, 1H), 2.72 (dd, J=18.8, 6.8 Hz, 1H), 2.23 (s, 3H). Mass (m/z) 503.3 [M+H]⁺.

### (S)-3-(1-(3-((2-(1-ethyl-3,5-dimethyl-1H-pyrazol-4-yl)-5-fluoropyridin-4-yl)oxy)azetidine-1-carbonyl)-4,5-dihydro-1H-pyrazol-5-yl)-5-fluorobenzonitrile (52)

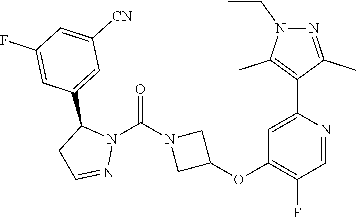 [SMILES: CCn1nc(C)c(-c2cc(OC3CN(C(=O)N4N=CC[C@H]4c4cc(F)cc(C#N)c4)C3)c(F)cn2)c1C]

The titled compound 52 was prepared in a 48.5% yield according to the procedure outlined for compound 1. ¹H NMR (400 MHz, Chloroform-d) δ 8.71 (d, J=3.5 Hz, 1H), 7.32 (t, J=1.4 Hz, 1H), 7.27 (d, J=2.3 Hz, 1H), 7.20 (dt, J=9.0, 1.9 Hz, 1H), 6.85 (d, J=1.8 Hz, 1H), 6.66 (d, J=6.4 Hz, 1H), 5.36-5.27 (m, 1H), 5.14 (s, 1H), 4.63 (s, 2H), 4.37 (d, J=29.6 Hz, 2H), 4.18 (q, J=7.2 Hz, 2H), 3.42 (ddd, J=18.8, 12.1, 1.5 Hz, 1H), 2.73 (ddd, J=18.7, 6.6, 1.6 Hz, 1H), 2.35 (d, J=7.9 Hz, 6H), 1.44 (t, J=7.1 Hz, 3H). Mass (m/z) 506.3 [M+H]⁺.

### (S)-3-(1-(3-((6-(1,4-dimethyl-1H-pyrazol-5-yl)-3-fluoropyridin-2-yl)oxy)azetidine-1-carbonyl)-4,5-dihydro-1H-pyrazol-5-yl)-5-fluorobenzonitrile (53)

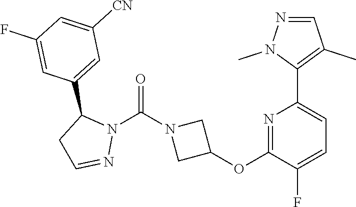 [SMILES: Cc1cnn(C)c1-c1ccc(F)c(OC2CN(C(=O)N3N=CC[C@H]3c3cc(F)cc(C#N)c3)C2)n1]

The title compound 53 was prepared in a yield of 54% as a white solid according to the procedure outlined for compound 11. Mass (m/z): 478.2 [M+H]⁺. ¹H NMR (400 MHz, Chloroform-d) δ 7.55-7.48 (m, 1H), 7.46 (s, 1H), 7.36-7.32 (m, 1H), 7.25-7.18 (m, 2H), 7.05 (dd, J=8.0, 2.8 Hz, 1H), 6.83-6.79 (m, 1H), 5.46-5.37 (m, 1H), 5.30 (dd, J=12.2, 6.8 Hz, 1H), 4.68-4.47 (m, 2H), 4.40-4.22 (m, 2H), 4.04 (s, 3H), 3.39 (ddd, J=18.6, 12.3, 1.7 Hz, 1H), 2.69 (ddd, J=18.6, 6.8, 1.7 Hz, 1H), 2.15 (s, 3H).

### (S)-(3-((6-(1,4-dimethyl-1H-pyrazol-5-yl)-3-fluoropyridin-2-yl)oxy)azetidin-1-yl)(5-(5-fluoropyridin-3-yl)-4,5-dihydro-1H-pyrazol-1-yl)methan one (54)

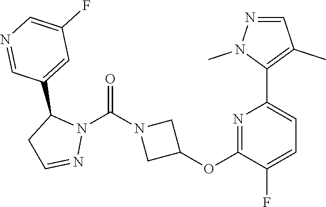 [SMILES: Cc1cnn(C)c1-c1ccc(F)c(OC2CN(C(=O)N3N=CC[C@H]3c3cncc(F)c3)C2)n1]

The title compound 54 was prepared in a yield of 55% as a white solid according to the procedure outlined for compound 11. Mass (m/z) 478.2 [M+H]⁺. ¹H NMR (301 MHz, Chloroform-d) δ 8.43-8.34 (m, 2H), 7.55-7.44 (m, 1H), 7.38 (s, 1H), 7.36-7.30 (m, 1H), 7.01 (dd, J=8.1, 2.9 Hz, 1H), 6.84-6.80 (m, 1H), 5.51-5.27 (m, 2H), 4.65-4.47 (m, 2H), 4.41-4.20 (mz, 1H), 3.97 (s, 3H), 3.41 (ddd, J=18.6, 12.2, 1.7 Hz, 1H), 2.75 (ddd, J=18.6, 6.8, 1.7 Hz, 1H), 2.13 (s, 3H).

### Preparation of (S)-5-(1-(3-((6-(1,4-dimethyl-1H-pyrazol-5-yl)-3-fluoropyridin-2-yl)oxy)azetidine-1-carbonyl)-4,5-dihydro-1H-pyrazol-5-yl)nicotinonitrile (55)

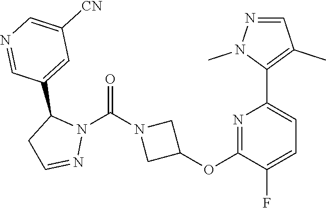 [SMILES: Cc1cnn(C)c1-c1ccc(F)c(OC2CN(C(=O)N3N=CC[C@H]3c3cncc(C#N)c3)C2)n1]

The title compound 55 was prepared in a yield of 52% as a white solid according to the procedure outlined for compound 11. Mass (m/z) 461.2 [M+H]⁺. ¹H NMR (400 MHz, Chloroform-d) δ 8.79 (s, 1H), 8.74 (s, 1H), 7.82 (t, J=2.1 Hz, 1H), 7.53-7.46 (m, 1H), 7.42 (s, 1H), 7.03 (dd, J=8.1, 2.8 Hz, 1H), 6.86-6.83 (m, 1H), 5.49-5.30 (m, 2H), 4.68-4.50 (m, 2H), 4.40-4.20 (m, 2H), 4.01 (s, 3H), 3.54-3.34 (m, 1H), 2.74 (ddd, J=18.7, 7.0, 1.6 Hz, 1H), 2.14 (s, 3H).

### (S)-5-(6-((1-(5-(3-cyano-5-fluorophenyl)-4,5-dihydro-1H-pyrazole-1-carbonyl)azetidin-3-yl)oxy)-5-fluoropyridin-2-yl)-1,4-dimethyl-1H-pyrazole-3-carbonitrile (56)

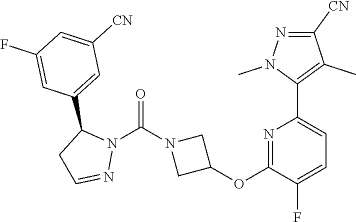 [SMILES: Cc1c(C#N)nn(C)c1-c1ccc(F)c(OC2CN(C(=O)N3N=CC[C@H]3c3cc(F)cc(C#N)c3)C2)n1]

The title compound 56 was prepared in a yield of 55% as a white solid according to the procedure outlined for compound 8. Mass (m/z) 496.3 [M+H]⁺. ¹H NMR (400 MHz, Chloroform-d) δ 7.56-7.49 (m, 1H), 7.35-7.32 (m, 1H), 7.25-7.17 (m, 2H), 7.03 (dd, J=8.0, 2.8 Hz, 1H), 6.83-6.80 (m, 1H), 5.45-5.36 (m, 1H), 5.30 (dd, J=12.2, 6.7 Hz, 1H), 4.68-4.48 (m, 2H), 4.42-4.21 (m, 2H), 3.99 (s, 3H), 3.40 (ddd, J=18.5, 12.2, 1.7 Hz, 1H), 2.70 (ddd, J=18.6, 6.7, 1.7 Hz, 1H), 2.23 (s, 3H).

### (S)-3-(1-(3-((2-(1-(cyclopropylmethyl)-3,5-dimethyl-1H-pyrazol-4-yl)-5-fluoropyridin-4-yl)oxy)azetidine-1-carbonyl)-4,5-dihydro-1H-pyrazol-5-yl)-5-fluorobenzonitrile (57)

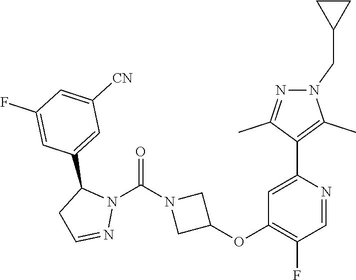 [SMILES: Cc1nn(CC2CC2)c(C)c1-c1cc(OC2CN(C(=O)N3N=CC[C@H]3c3cc(F)cc(C#N)c3)C2)c(F)cn1]

The titled compound 57 was prepared in a 37.6% yield according to the procedure outlined for compound 20. ¹H NMR (301 MHz, Chloroform-d) δ 8.61 (d, J=3.4 Hz, 1H), 7.33 (s, 1H), 7.20 (d, J=8.6 Hz, 2H), 6.84 (s, 1H), 6.63 (d, J=6.5 Hz, 1H), 5.31 (dd, J=12.3, 6.6 Hz, 1H), 5.11 (s, 1H), 4.61 (s, 2H), 4.44-4.27 (m, 2H), 3.99 (d, J=7.1 Hz, 2H), 3.42 (dd, J=18.6, 12.3 Hz, 1H), 2.72 (dd, J=18.7, 6.7 Hz, 1H), 2.49 (s, 3H), 2.37 (d, J=12.0 Hz, 3H), 1.25 (s, 1H), 0.60 (t, J=6.7 Hz, 2H), 0.42 (t, J=5.0 Hz, 2H). Mass (m/z) 532.3 [M+H]⁺.

### (S)-5-(4-((1-(5-(3-cyano-5-fluorophenyl)-4,5-dihydro-1H-pyrazole-1-carbonyl)azetidin-3-yl)oxy)-5-fluoropyridin-2-yl)-1,4-dimethyl-1H-pyrazole-3-carboxamide (58)

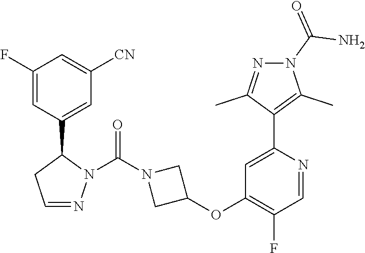 [SMILES: Cc1nn(C(N)=O)c(C)c1-c1cc(OC2CN(C(=O)N3N=CC[C@H]3c3cc(F)cc(C#N)c3)C2)c(F)cn1]

The titled compound 58 was prepared in a 46.2% yield according to the procedure outlined for compound 10. ¹H NMR (301 MHz, Chloroform-d) δ 8.52 (d, J=2.9 Hz, 1H), 7.33 (s, 1H), 7.27-7.15 (m, 2H), 6.95 (s, 1H), 6.84 (t, J=1.8 Hz, 1H), 6.72 (d, J=6.5 Hz, 1H), 6.25 (s, 1H), 5.31 (dd, J=12.2, 6.6 Hz, 1H), 5.14-5.04 (m, 1H), 4.70-4.52 (m, 2H), 4.42-4.24 (m, 2H), 3.95 (s, 3H), 3.41 (ddd, J=18.7, 12.2, 1.7 Hz, 1H), 2.71 (ddd, J=18.7, 6.7, 1.7 Hz, 1H), 2.35 (s, 3H). Mass (m/z) 521.3 [M+H]⁺.

### (S)-5-(6-((1-(5-(3-cyano-5-fluorophenyl)-4,5-dihydro-1H-pyrazole-1-carbonyl)azetidin-3-yl)oxy)-5-fluoropyridin-2-yl)-1,4-dimethyl-1H-pyrazole-3-carboxamide (59)

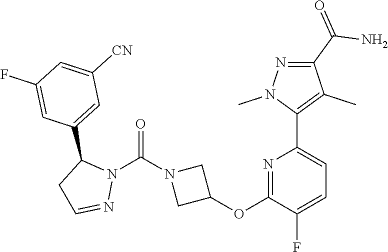 [SMILES: Cc1c(C(N)=O)nn(C)c1-c1ccc(F)c(OC2CN(C(=O)N3N=CC[C@H]3c3cc(F)cc(C#N)c3)C2)n1]

The title compound 59 was prepared in a yield of 55% as a white solid according to the procedure for 10. Mass (m/z) 521.3 [M+H]⁺. ¹H NMR (400 MHz, Chloroform-d) δ 7.51 (dd, J=9.5, 8.1 Hz, 1H), 7.35-7.32 (m, 1H), 7.25-7.18 (m, 2H), 7.03 (dd, J=8.1, 2.9 Hz, 1H), 6.90 (s, 1H), 6.83-6.80 (m, 1H), 5.93 (s, 1H), 5.46-5.37 (m, 1H), 5.31 (dd, J=12.2, 6.7 Hz, 1H), 4.67-4.48 (m, 2H), 4.42-4.20 (m, 2H), 3.93 (s, 3H), 3.39 (ddd, J=18.6, 12.2, 1.7 Hz, 1H), 2.69 (ddd, J=18.7, 6.8, 1.8 Hz, 1H), 2.35 (s, 3H).

### (S)-3-(1-(3-(5-(1,4-dimethyl-1H-pyrazol-5-yl)-2-fluorophenoxy)azetidine-1-carbonyl)-4,5-dihydro-1H-pyrazol-5-yl)-5-fluorobenzonitrile (60)

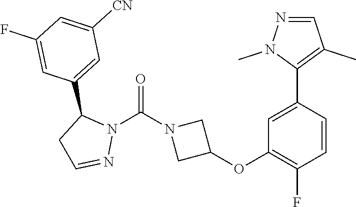 [SMILES: Cc1cnn(C)c1-c1ccc(F)c(OC2CN(C(=O)N3N=CC[C@H]3c3cc(F)cc(C#N)c3)C2)c1]

The titled compound 60 was prepared in a 47.2% yield according to the procedure outlined for compound 11. ¹H NMR (400 MHz, Chloroform-d) δ 7.38-7.32 (m, 2H), 7.26-7.23 (m, 1H), 7.22-7.18 (m, 2H), 6.89 (ddd, J=8.3, 4.3, 2.0 Hz, 1H), 6.84-6.79 (m, 1H), 6.62 (dd, J=7.8, 2.1 Hz, 1H), 5.30 (dd, J=12.2, 6.8 Hz, 1H), 4.98 (tt, J=6.4, 4.1 Hz, 1H), 4.54 (s, 2H), 4.30 (t, J=17.2 Hz, 2H), 3.74 (s, 3H), 3.39 (ddd, J=18.7, 12.3, 1.7 Hz, 1H), 2.75-2.64 (m, 1H), 1.98 (d, J=0.5 Hz, 3H). Mass (m/z) 477.3 [M+H]⁺.

### (S)-(3-((2-(3-amino-1,4-dimethyl-1H-pyrazol-5-yl)-5-fluoropyridin-4-yl)oxy)azetidin-1-yl)(5-(5-fluoropyridin-3-yl)-4,5-dihydro-1H-pyrazol-1-yl)methanone (61)

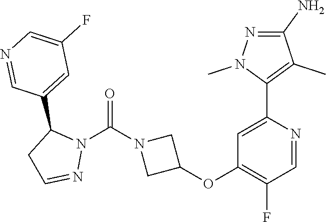 [SMILES: Cc1c(N)nn(C)c1-c1cc(OC2CN(C(=O)N3N=CC[C@H]3c3cncc(F)c3)C2)c(F)cn1]

The titled compound 61 was prepared in 51.2% yield according to the procedure outlined for compound 35. ¹H NMR (400 MHz, Chloroform-d) δ 8.54 (d, J=2.8 Hz, 1H), 8.43 (s, 2H), 7.44 (d, J=8.4 Hz, 1H), 6.87 (d, J=1.7 Hz, 1H), 6.76 (d, J=6.3 Hz, 1H), 5.39 (dd, J=12.2, 6.7 Hz, 1H), 5.10 (s, 1H), 4.60 (s, 2H), 4.34 (d, J=28.1 Hz, 2H), 3.81 (s, 3H), 3.53-3.41 (m, 1H), 2.84-2.75 (m, 1H), 1.96 (s, 3H). Mass (m/z) 469.2 [M+H]⁺.

### (S)-5-(1-(3-((2-(3-amino-1,4-dimethyl-1H-pyrazol-5-yl)-5-fluoropyridin-4-yl)oxy)azetidine-1-carbonyl)-4,5-dihydro-1H-pyrazol-5-yl)nicotinonitrile (62)

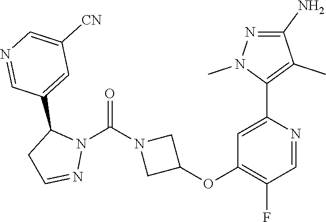 [SMILES: Cc1c(N)nn(C)c1-c1cc(OC2CN(C(=O)N3N=CC[C@H]3c3cncc(C#N)c3)C2)c(F)cn1]

The titled compound 62 was prepared in 42.10% yield according to the procedure outlined for compound 11. ¹H NMR (400 MHz, Chloroform-d) δ 8.77 (d, J=24.9 Hz, 2H), 8.54 (d, J=2.9 Hz, 11H), 7.84 (t, J=2.0 Hz, 11H), 6.89 (d, J=1.7 Hz, 1H), 6.77 (d, J=6.3 Hz, 1H), 5.38 (dd, J=12.2, 6.8 Hz, 1H), 5.11 (s, 1H), 4.61 (s, 2H), 4.45-4.22 (m, 2H), 3.82 (s, 3H), 3.53-3.41 (m, 1H), 2.83-2.72 (m, 1H), 1.96 (s, 3H). Mass (m/z) 476.3 [M+H]⁺.

### (S)-1-(5-fluoro-4-((1-(5-(5-fluoropyridin-3-yl)-4,5-dihydro-1H-pyrazole-1-carbonyl)azetidin-3-yl)oxy)pyridin-2-yl)-3,5-dimethyl-1H-pyrazole-4-carboxamide (63)

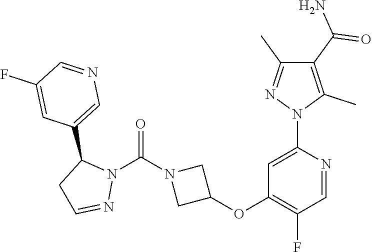 [SMILES: Cc1nn(-c2cc(OC3CN(C(=O)N4N=CC[C@H]4c4cncc(F)c4)C3)c(F)cn2)c(C)c1C(N)=O]

The titled compound 63 was prepared in 39.7% yield according to the procedure outlined for compound 10. ¹H NMR (400 MHz, Chloroform-d) δ 8.46 (s, 2H), 8.24 (d, J=2.4 Hz, 1H), 7.54 (d, J=8.5 Hz, 1H), 7.19 (s, 1H), 6.89 (s, 1H), 5.42 (dd, J=12.1, 6.3 Hz, 1H), 5.24-5.11 (m, 1H), 4.73-4.56 (s, 2H), 4.36 (d, J=30.7 Hz, 2H), 3.48 (dd, J=18.4, 12.1 Hz, 1H), 2.84 (s, 3H), 2.78 (d, J=5.8 Hz, 1H), 2.50 (s, 3H). Mass (m/z) 497.3 [M+H]⁺.

### (S)-(3-((5-fluoro-2-(4-methylthiazol-5-yl)pyridin-4-yl)oxy)azetidin-1-yl)(5-(5-fluoropyridin-3-yl)-4,5-dihydro-1H-pyrazol-1-yl)methanone (64)

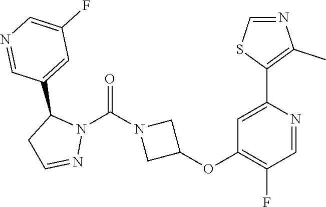 [SMILES: Cc1ncsc1-c1cc(OC2CN(C(=O)N3N=CC[C@H]3c3cncc(F)c3)C2)c(F)cn1]

The titled compound 64 was prepared in 27.6% yield according to the procedure outlined for compound 1. ¹H NMR (400 MHz, Chloroform-d) δ 8.78 (s, 1H), 8.41 (d, J=2.9 Hz, 3H), 7.38-7.32 (m, 1H), 6.91-6.83 (m, 2H), 5.37 (dd, J=12.2, 6.5 Hz, 1H), 5.11 (q, J=5.3 Hz, 1H), 4.60 (s, 2H), 4.33 (dd, J=33.8, 10.4 Hz, 2H), 3.48-3.38 (m, 1H), 2.84-2.73 (m, 1H), 2.69 (s, 3H). Mass (m/z) 457.2 [M+H]⁺.

### (S)-5-(5-fluoro-4-((1-(5-(5-fluoropyridin-3-yl)-4,5-dihydro-1H-pyrazole-1-carbonyl)azetidin-3-yl)oxy)pyridin-2-yl)-1,4-dimethyl-1H-pyrazole-3-carbonitrile (65)

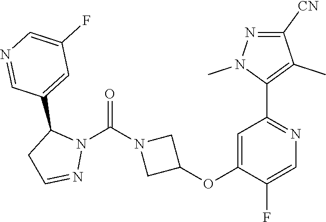 [SMILES: Cc1c(C#N)nn(C)c1-c1cc(OC2CN(C(=O)N3N=CC[C@H]3c3cncc(F)c3)C2)c(F)cn1]

The title compound 65 was prepared in a yield of 58% as a white solid according to the procedure outlined for compound 51. Mass (m/z) 479.3 [M+H]⁺. ¹H NMR (400 MHz, Chloroform-d) δ 8.51 (d, J=2.9 Hz, 1H), 8.38 (d, J=2.7 Hz, 1H), 8.37-8.34 (m, 1H), 7.31-7.23 (m, 1H), 6.87-6.81 (m, 1H), 6.72 (d, J=6.4 Hz, 1H), 5.35 (dd, J=12.2, 6.6 Hz, 1H), 5.11-5.03 (m, 1H), 4.69-4.50 (m, 2H), 4.42-4.23 (m, 2H), 4.00 (s, 3H), 3.43 (ddd, J=18.7, 12.3, 1.7 Hz, 1H), 2.77 (ddd, J=18.7, 6.7, 1.7 Hz, 1H), 2.23 (s, 3H).

### (S)-1-(4-((1-(5-(3-cyano-5-fluorophenyl)-4,5-dihydro-1H-pyrazole-1-carbonyl)azetidin-3-yl)oxy)-5-fluoropyridin-2-yl)-2,5-dimethyl-1H-imidazole-4-carbonitrile (66)

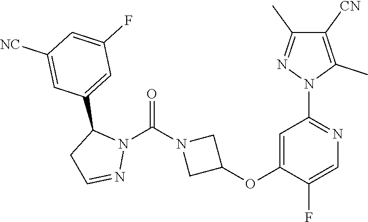 [SMILES: Cc1nn(-c2cc(OC3CN(C(=O)N4N=CC[C@H]4c4cc(F)cc(C#N)c4)C3)c(F)cn2)c(C)c1C#N]

The titled compound 66 was prepared in a yield of 31.5% according to the procedure outlined for compound 8. Mass (m/z) 503.3 [M+H]⁺. ¹H NMR (300 MHz, Chloroform-d) δ 8.37 (t, J=14.6 Hz, 1H), 7.33 (s, 1H), 7.23 (dd, J=16.1, 7.6 Hz, 2H), 6.85 (s, 1H), 6.72 (d, J=5.7 Hz, 1H), 5.28 (dt, J=26.7, 13.4 Hz, 1H), 5.12 (s, 1H), 4.63 (d, J=4.9 Hz, 2H), 4.34 (d, J=9.8 Hz, 2H), 3.42 (dd, J=18.6, 12.3 Hz, 1H), 2.73 (dd, J=18.7, 6.6 Hz, 1H), 2.26 (d, J=6.7 Hz, 6H).

### (S)-(3-((6-(3-amino-1,4-dimethyl-1H-pyrazol-5-yl)-3-fluoropyridin-2-yl)oxy)azetidin-1-yl)(5-(5-fluoropyridin-3-yl)-4,5-dihydro-1H-pyrazol-1-yl)methanone (67)

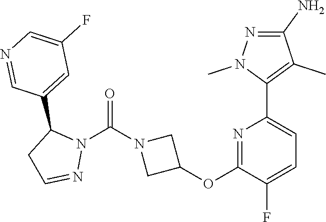 [SMILES: Cc1c(N)nn(C)c1-c1ccc(F)c(OC2CN(C(=O)N3N=CC[C@H]3c3cncc(F)c3)C2)n1]

The title compound 67 was prepared in a yield of 53% as a white solid according to the procedure for 35. Mass (m/z) 469.3 [M+H]⁺. ¹H NMR (400 MHz, Chloroform-d) δ 8.41-8.33 (m, 2H), 7.47 (dd, J=9.6, 8.1 Hz, 1H), 7.30-7.24 (m, 1H), 6.98 (dd, J=8.1, 2.9 Hz, 1H), 6.83-6.79 (m, 1H), 5.44-5.38 (m, 1H), 5.35 (dd, J=12.1, 6.7 Hz, 1H), 4.68-4.45 (m, 2H), 4.37-4.21 (m, 2H), 3.77 (s, 3H), 3.40 (ddd, J=18.6, 12.2, 1.7 Hz, 1H), 2.74 (ddd, J=18.6, 6.8, 1.7 Hz, 1H), 2.62-2.40 (m, 2H), 1.96 (s, 3H).

### (S)-1-(5-fluoro-4-((1-(5-(5-fluoropyridin-3-yl)-4,5-dihydro-1H-pyrazole-1-carbonyl)azetidin-3-yl)oxy)pyridin-2-yl)-2,5-dimethyl-1H-imidazole-4-carbonitrile (68)

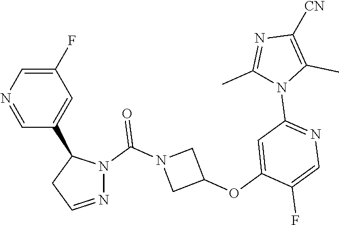 [SMILES: Cc1nc(C#N)c(C)n1-c1cc(OC2CN(C(=O)N3N=CC[C@H]3c3cncc(F)c3)C2)c(F)cn1]

The titled compound 68 was prepared in a yield of 24.1% from 5 according to the procedure outlined for compound 8. Mass (m/z) 459.2 [M+H]⁺. ¹H NMR (400 MHz, Chloroform-d) δ 8.43 (d, J=2.6 Hz, 1H), 7.32 (d, J=8.6 Hz, 1H), 7.28 (s, 2H), 6.89 (s, 1H), 6.63 (d, J=5.8 Hz, 1H), 5.39 (dd, J=12.2, 6.6 Hz, 1H), 5.15-5.07 (m, 1H), 4.70-4.54 (m, 2H), 4.43-4.25 (m, 2H), 3.46 (ddd, J=18.6, 12.2, 1.5 Hz, 1H), 2.81 (ddd, J=18.7, 6.6, 1.6 Hz, 1H), 2.29 (d, J=12.7 Hz, 6H).

### (S)-1-(5-fluoro-4-((1-(5-(5-fluoropyridin-3-yl)-4,5-dihydro-1H-pyrazole-1-carbonyl)azetidin-3-yl)oxy)pyridin-2-yl)-2,5-dimethyl-1H-imidazole-4-carboxamide (69)

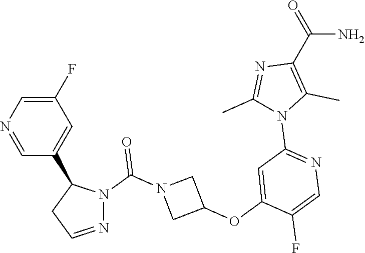 [SMILES: Cc1nc(C(N)=O)c(C)n1-c1cc(OC2CN(C(=O)N3N=CC[C@H]3c3cncc(F)c3)C2)c(F)cn1]

The titled compound 69 was prepared in a yield of 42% according to the procedure outlined for compound 10. Mass (m/z) 497.5 [M+H]⁺; ¹H NMR (400 MHz, CDCl₃) δ 8.56-8.26 (m, 3H), 6.99 (s, 1H), 6.86 (t, J=1.7 Hz, 1H), 6.56 (d, J=5.8 Hz, 1H), 5.42-5.26 (m, 2H), 5.07 (tt, J=6.5, 3.8 Hz, 1H), 4.58 (d, J=21.4 Hz, 2H), 4.31 (t, J=14.9 Hz, 2H), 3.43 (ddd, J=18.7, 12.3, 1.7 Hz, 1H), 2.84-2.69 (m, 1H), 2.42 (s, 3H), 2.24 (s, 3H).

### (S)-1-(4-((1-(5-(3-cyano-5-fluorophenyl)-4,5-dihydro-1H-pyrazole-1-carbonyl)azetidin-3-yl)oxy)-5-fluoropyridin-2-yl)-3,5-dimethyl-1H-pyrazole-4-carboxamide (70)

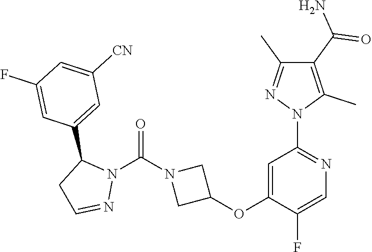 [SMILES: Cc1nn(-c2cc(OC3CN(C(=O)N4N=CC[C@H]4c4cc(F)cc(C#N)c4)C3)c(F)cn2)c(C)c1C(N)=O]

The titled compound 70 was prepared in a yield of 19.2% according to the procedure outlined for compound 10. ¹H NMR (400 MHz, Chloroform-d) δ 8.22 (d, J=2.4 Hz, 1H), 7.34 (t, J=1.5 Hz, 1H), 7.28-7.24 (m, 1H), 7.20 (dt, J=9.1, 1.9 Hz, 1H), 7.17 (d, J=6.1 Hz, 1H), 6.84-6.81 (m, 1H), 5.31 (dd, J=12.2, 6.7 Hz, 1H), 5.16 (td, J=6.5, 3.4 Hz, 1H), 4.68-4.55 (s, 2H), 4.41-4.24 (m, 2H), 3.41 (ddd, J=18.6, 12.2, 1.7 Hz, 1H), 2.82 (s, 3H), 2.71 (ddd, J=18.7, 6.7, 1.7 Hz, 1H), 2.48 (s, 3H). Mass (m/z) 521.3 [M+H]⁺.

### (S)-1-(5-fluoro-4-((1-(5-phenyl-4,5-dihydro-1H-pyrazole-1-carbonyl) azetidin-3-yl)oxy)pyridin-2-yl)-3,5-dimethyl-1H-pyrazole-4-carboxamide (71)

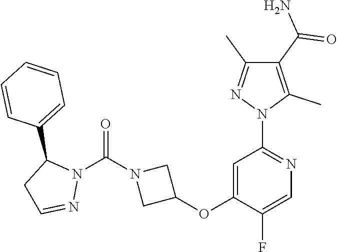 [SMILES: Cc1nn(-c2cc(OC3CN(C(=O)N4N=CC[C@H]4c4ccccc4)C3)c(F)cn2)c(C)c1C(N)=O]

The titled compound 70 was prepared in a yield of 21.3% according to the procedure outlined for compound 10. ¹H NMR (400 MHz, Chloroform-d) δ 8.20 (d, J=2.4 Hz, 1H), 7.37-7.31 (m, 2H), 7.28 (t, J=1.4 Hz, 1H), 7.24-7.21 (m, 2H), 7.15 (d, J=6.1 Hz, 1H), 6.82-6.78 (m, 1H), 5.63 (s, 2H), 5.32 (dd, J=12.2, 6.3 Hz, 1H), 5.12 (td, J=6.6, 3.3 Hz, 1H), 4.68-4.51 (m, 2H), 4.30 (ddd, J=32.7, 10.5, 3.9 Hz, 2H), 3.36 (ddd, J=18.6, 12.2, 1.7 Hz, 1H), 2.81 (s, 3H), 2.76 (ddd, J=18.6, 6.3, 1.8 Hz, 1H), 2.48 (s, 3H). Mass (m/z) 478.3 [M+H]⁺.

### (S)-1-(5-fluoro-4-((1-(5-(5-fluoropyridin-3-yl)-4,5-dihydro-1H-pyrazole-1-carbonyl)azetidin-3-yl)oxy)pyridin-2-yl)-3,5-dimethyl-1H-pyrazole-4-carboxamide (72)

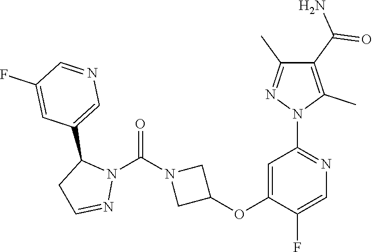 [SMILES: Cc1nn(-c2cc(OC3CN(C(=O)N4N=CC[C@H]4c4cncc(F)c4)C3)c(F)cn2)c(C)c1C(N)=O]

The titled compound 72 was prepared in a yield of 49.3% according to the procedure outlined for compound 10. ¹H NMR (400 MHz, Chloroform-d) δ 8.45-8.39 (m, 2H), 8.22 (d, J=2.4 Hz, 1H), 7.43 (d, J=8.7 Hz, 1H), 7.16 (d, J=6.1 Hz, 1H), 6.86 (d, J=1.5 Hz, 1H), 5.39 (dd, J=12.2, 6.6 Hz, 1H), 5.19-5.13 (m, 1H), 4.70-4.56 (m, 2H), 4.41-4.25 (m, 2H), 3.50-3.41 (m, 1H), 2.81 (s, 3H), 2.81-2.73 (m, 1H), 2.48 (s, 3H). Mass (m/z) 497.3 [M+H]⁺.

### (S)-1-(5-fluoro-4-((1-(5-(3-fluorophenyl)-4,5-dihydro-1H-pyrazole-1-carbonyl)azetidin-3-yl)oxy)pyridin-2-yl)-3,5-dimethyl-1H-pyrazole-4-carb oxamide (73)

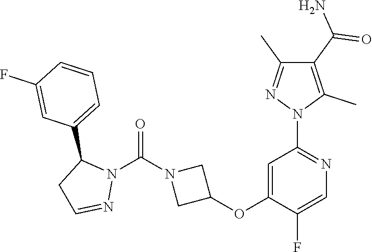 [SMILES: Cc1nn(-c2cc(OC3CN(C(=O)N4N=CC[C@H]4c4cccc(F)c4)C3)c(F)cn2)c(C)c1C(N)=O]

The titled compound 73 was prepared in a yield of 32.3% according to the procedure outlined for compound 10. ¹H NMR (400 MHz, Chloroform-d) δ 8.22 (d, J=2.5 Hz, 1H), 7.33-7.27 (m, 1H), 7.15 (d, J=6.1 Hz, 1H), 7.01 (dd, J=7.8, 1.5 Hz, 1H), 6.99-6.89 (m, 2H), 6.81 (t, J=1.7 Hz, 1H), 5.32 (dd, J=12.2, 6.3 Hz, 1H), 5.14 (ddd, J=10.2, 6.4, 3.9 Hz, 1H), 4.69-4.56 (m, 2H), 4.32 (dd, J=32.8, 9.9 Hz, 2H), 3.37 (ddd, J=18.7, 12.2, 1.7 Hz, 1H), 3.20-3.10 (m, 2H), 2.81 (s, 3H), 2.74 (ddd, J=18.7, 6.3, 1.8 Hz, 1H), 2.49 (s, 3H). Mass (m/z) 496.3 [M+H]⁺.

### (S)-1-(5-fluoro-4-((1-(5-phenyl-4,5-dihydro-1H-pyrazole-1-carbonyl) azetidin-3-yl)oxy)pyridin-2-yl)-N-(2-hydroxyethyl)-3,5-dimethyl-1H-pyrazole-4-carboxamide (74)

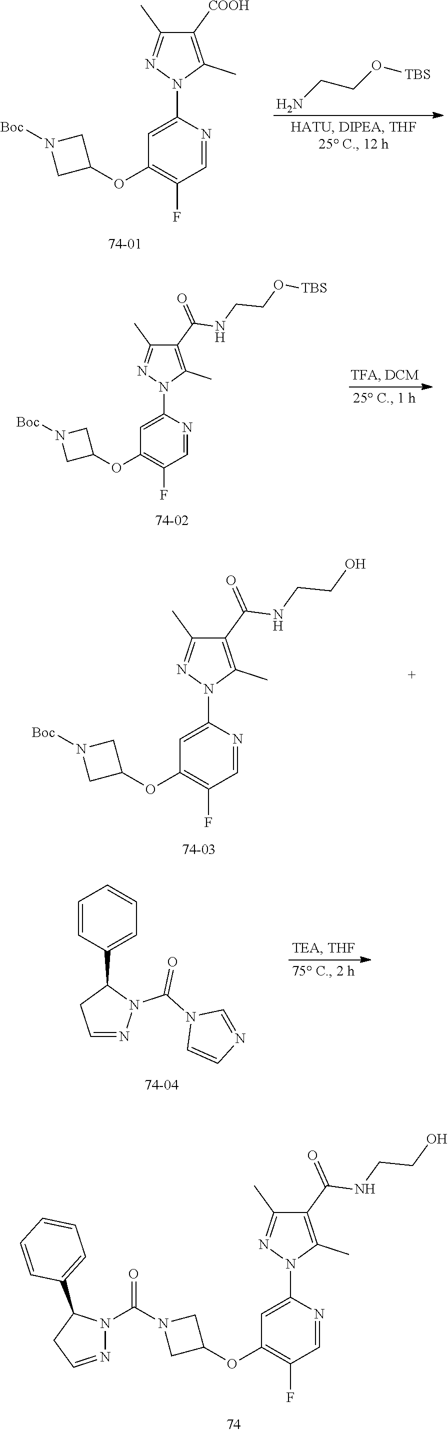

Step 1: 74-01 (150 mg, 0.37 mmol), 2-((tert-butyldimethylsilyl)oxy)ethan-1-amine (98 mg, 0.555 mmol), HATU (211 mg, 0.555 mmol), DIPEA (144 mg, 1.11 mmol) in THE (5 mL) were mixed and the whole reaction mixture was stirred at 25° C. for 12 hours. After the mixture was concentrated and further purification by silica gel chromatography to give 74-02 (192 mg, 90.0%) as a yellow solid. Mass (m/z) 564.4 [M+H]⁺.

Step 2: 74-02 (192 mg, 0.34 mmol) was dissolved in 3 mL of DCM, trifluoroacetic acid (388 mg, 3.4 mmol) was added, the mixture was stirred at 25° C. for 1 hour. Concentrated to give the desired product 74-03, which was used for next step without further purification.

Step 3: 74-03 (60 mg, 0.172 mmol), 74-04 (37.2 mg, 0.155 mmol) and TEA (1 mL) were dissolved in THE (2 mL) and stirred at 75° C. for 2 h. The mixture was extracted with EA, washed with brine, dried (Na₂SO₄), and concentrated in vacuo. Purification by prep-HPLC to give the titled compound 74 (15 mg, 18.6%) as a white solid. ¹H NMR (400 MHz, Chloroform-d) δ 8.19 (d, J=2.4 Hz, 1H), 7.33 (dd, J=8.2, 6.7 Hz, 2H), 7.27 (t, J=1.4 Hz, 2H), 7.22 (dd, J=8.2, 1.4 Hz, 2H), 7.14 (d, J=6.1 Hz, 11H), 6.80 (t, J=1.7 Hz, 1H), 6.11 (s, 1H), 5.31 (dd, J=12.2, 6.2 Hz, 1H), 5.12 (dd, J=7.0, 3.4 Hz, 1H), 4.60 (dt, J=23.1, 8.6 Hz, 2H), 4.37-4.22 (m, 2H), 3.83 (dd, J=5.5, 4.3 Hz, 2H), 3.62 (q, J=5.2 Hz, 2H), 3.36 (ddd, J=18.6, 12.1, 1.7 Hz, 1H), 2.82-2.71 (m, 4H), 2.45 (s, 3H). Mass (m/z) 522.3 [M+H]⁺.

### (S)-1-(5-fluoro-4-((1-(5-(3-fluorophenyl)-4,5-dihydro-1H-pyrazole-1-carbonyl)azetidin-3-yl)oxy)pyridin-2-yl)-N-(2-hydroxyethyl)-3,5-dimethyl-1H-pyrazole-4-carboxamide (75)

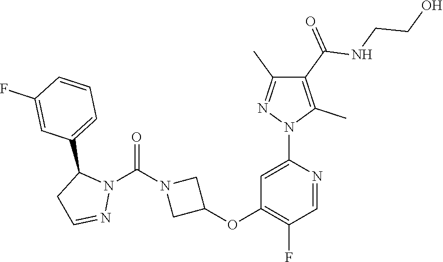 [SMILES: Cc1nn(-c2cc(OC3CN(C(=O)N4N=CC[C@H]4c4cccc(F)c4)C3)c(F)cn2)c(C)c1C(=O)NCCO]

The titled compound 75 was prepared in a 25.1% yield according to the procedure outlined for compound 74. ¹H NMR (400 MHz, Chloroform-d) δ 8.20 (d, J=2.4 Hz, 1H), 7.34-7.28 (m, 1H), 7.27 (s, 1H), 7.15 (d, J=6.1 Hz, 1H), 7.02 (dt, J=7.7, 1.3 Hz, 1H), 6.99-6.89 (m, 2H), 6.80 (t, J=1.7 Hz, 1H), 6.12 (t, J=5.5 Hz, 1H), 5.30 (dd, J=12.2, 6.3 Hz, 1H), 5.13 (td, J=6.4, 3.2 Hz, 1H), 4.61 (dt, J=17.2, 8.7 Hz, 2H), 4.39-4.23 (m, 2H), 3.83 (dd, J=5.5, 4.3 Hz, 2H), 3.61 (td, J=5.6, 4.3 Hz, 2H), 3.36 (ddd, J=18.6, 12.2, 1.7 Hz, 1H), 2.82-2.68 (m, 4H), 2.45 (s, 3H). Mass (m/z) 540.3 [M+H]⁺.

### (S)-1-(5-fluoro-4-((1-(5-phenyl-4,5-dihydro-1H-pyrazole-1-carbonyl) azetidin-3-yl)oxy)pyridin-2-yl)-3,5-dimethyl-N-(oxetan-3-yl)-1H-pyrazole-4-carboxamide (76)

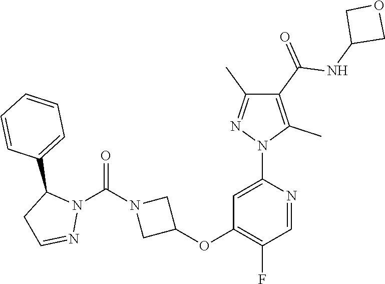 [SMILES: Cc1nn(-c2cc(OC3CN(C(=O)N4N=CC[C@H]4c4ccccc4)C3)c(F)cn2)c(C)c1C(=O)NC1COC1]

The titled compound 76 was prepared in a 64.1% yield according to the procedure outlined for compound 74. ¹H NMR (400 MHz, Chloroform-d) δ 8.21 (d, J=2.4 Hz, 1H), 7.34 (t, J=7.4 Hz, 2H), 7.27 (s, 1H), 7.26-7.19 (m, 2H), 7.16 (d, J=6.1 Hz, 1H), 6.80 (s, 1H), 5.32 (dd, J=12.1, 6.2 Hz, 1H), 5.13 (s, 1H), 4.63 (s, 3H), 4.51 (s, 2H), 4.43 (s, 1H), 4.35 (s, 1H), 4.27 (s, 1H), 4.08 (s, 1H), 3.69 (d, J=11.6 Hz, 1H), 3.36 (dd, J=18.8, 13.2 Hz, 1H), 2.89 (s, 3H), 2.82-2.66 (m, 1H), 2.53 (s, 3H). Mass (m/z) 534.4 [M+H]⁺.

### (S)-1-(5-fluoro-4-((1-(5-(3-fluorophenyl)-4,5-dihydro-1H-pyrazole-1-carbonyl)azetidin-3-yl)oxy)pyridin-2-yl)-3,5-dimethyl-N-(oxetan-3-yl)-1H-pyrazole-4-carboxamide (77)

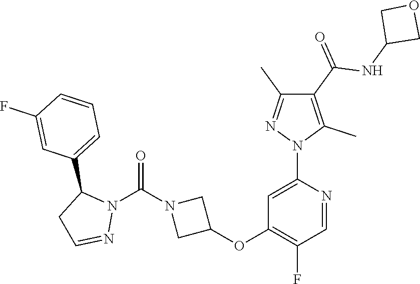 [SMILES: Cc1nn(-c2cc(OC3CN(C(=O)N4N=CC[C@H]4c4cccc(F)c4)C3)c(F)cn2)c(C)c1C(=O)NC1COC1]

The titled compound 77 was prepared in a 67.9% yield according to the procedure outlined for compound 74. ¹H NMR (400 MHz, Chloroform-d) δ 8.20 (d, J=2.4 Hz, 1H), 7.34-7.28 (m, 1H), 7.27 (s, 1H), 7.17 (d, J=6.2 Hz, 1H), 7.06-6.88 (m, 3H), 6.82-6.77 (m, 1H), 5.31 (dd, J=12.2, 6.4 Hz, 1H), 5.14 (td, J=6.3, 3.2 Hz, 1H), 4.63 (dd, J=18.1, 9.6 Hz, 2H), 4.48-4.39 (m, 2H), 4.35 (d, J=9.4 Hz, 1H), 4.26 (d, J=13.3 Hz, 2H), 3.92 (dd, J=11.3, 3.3 Hz, 1H), 3.64 (dd, J=11.2, 3.9 Hz, 1H), 3.36 (ddd, J=18.6, 12.1, 1.7 Hz, 1H), 2.84 (s, 3H), 2.73 (ddd, J=18.6, 6.4, 1.8 Hz, 1H), 2.47 (s, 3H). Mass (m/z) 552.3 [M+H]⁺.

### S)-1-(5-fluoro-4-((1-(5-phenyl-4,5-dihydro-1H-pyrazole-1-carbonyl)azetidin-3-yl)oxy)pyridin-2-yl)-N,3,5-trimethyl-1H-pyrazole-4-carboxamide (78)

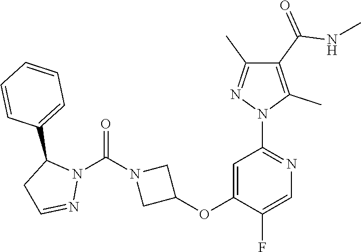 [SMILES: CNC(=O)c1c(C)nn(-c2cc(OC3CN(C(=O)N4N=CC[C@H]4c4ccccc4)C3)c(F)cn2)c1C]

The titled compound 78 was prepared in a 22.9% yield according to the procedure outlined for compound 74. ¹H NMR (400 MHz, DMSO-d₆) δ 8.44 (d, J=2.7 Hz, 1H), 7.74 (d, J=4.6 Hz, 1H), 7.37-7.29 (m, 2H), 7.28-7.22 (m, 2H), 7.20-7.16 (m, 2H), 7.05-7.00 (m, 1H), 5.33 (dd, J=6.4, 3.2 Hz, 1H), 5.22 (dd, J=12.1, 6.0 Hz, 1H), 4.58-4.43 (m, 2H), 4.12-3.98 (m, 2H), 3.41 (ddd, J=18.6, 12.1, 1.6 Hz, 1H), 2.75 (d, J=4.5 Hz, 3H), 2.62 (dd, J=6.1, 1.8 Hz, 1H), 2.57 (s, 3H), 2.31 (s, 3H). Mass (m/z) 492.3 [M+H]⁺.

### (S)-1-(5-fluoro-4-((1-(5-(3-fluorophenyl)-4,5-dihydro-1H-pyrazole-1-carbonyl)azetidin-3-yl)oxy)pyridin-2-yl)-N,3,5-trimethyl-1H-pyrazole-4-carboxamide (79)

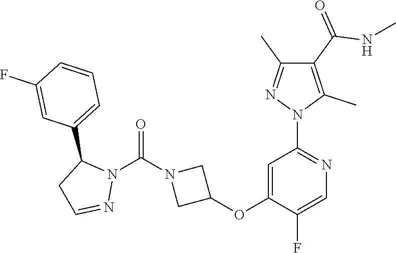 [SMILES: CNC(=O)c1c(C)nn(-c2cc(OC3CN(C(=O)N4N=CC[C@H]4c4cccc(F)c4)C3)c(F)cn2)c1C]

The titled compound 79 was prepared in 40.2% yield according to the procedure outlined for compound 74. ¹H NMR (400 MHz, Chloroform-d) δ 8.19 (d, J=2.4 Hz, 1H), 7.36-7.27 (m, 1H), 7.15 (d, J=6.1 Hz, 1H), 7.06-6.87 (m, 3H), 6.80 (t, J=1.7 Hz, 1H), 5.59 (d, J=5.6 Hz, 1H), 5.31 (dd, J=12.2, 6.4 Hz, 1H), 5.13 (td, J=6.4, 3.2 Hz, 1H), 4.62 (dt, J=17.7, 8.9 Hz, 2H), 4.38-4.22 (m, 2H), 3.36 (ddd, J=18.6, 12.2, 1.7 Hz, 1H), 3.00 (d, J=4.8 Hz, 3H), 2.76 (s, 3H), 2.76-2.68 (m, 1H), 2.44 (s, 3H). Mass (m/z) 510.4 [M+H]⁺.

### (S)-1-(4-((1-(5-(3-cyano-5-fluorophenyl)-4,5-dihydro-1H-pyrazole-1-carbonyl)azetidin-3-yl)oxy)-5-fluoropyridin-2-yl)-N,3,5-trimethyl-1H-pyrazole-4-carboxamide (80)

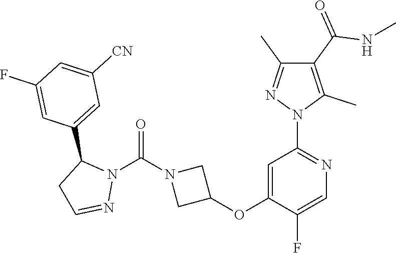 [SMILES: CNC(=O)c1c(C)nn(-c2cc(OC3CN(C(=O)N4N=CC[C@H]4c4cc(F)cc(C#N)c4)C3)c(F)cn2)c1C]

The titled compound 80 was prepared in 40.2% yield according to the procedure outlined for compound 74. ¹H NMR (400 MHz, Chloroform-d) δ 8.20 (d, J=2.4 Hz, 1H), 7.34 (t, J=1.5 Hz, 1H), 7.29-7.24 (m, 1H), 7.20 (dt, J=9.0, 2.1 Hz, 1H), 7.16 (d, J=6.1 Hz, 1H), 6.84-6.81 (m, 1H), 5.65-5.54 (m, 1H), 5.36-5.27 (m, 1H), 5.16 (td, J=6.5, 3.4 Hz, 1H), 4.63 (s, 2H), 4.43-4.24 (m, 2H), 3.41 (ddd, J=18.7, 12.2, 1.7 Hz, 1H), 3.00 (d, J=4.8 Hz, 3H), 2.76 (s, 3H), 2.76-2.68 (m, 1H), 2.44 (s, 3H). Mass (m/z) 535.4 [M+H]⁺.

### (S)-1-(4-((1-(5-(5-cyanopyridin-3-yl)-4,5-dihydro-1H-pyrazole-1-carbonyl)azetidin-3-yl)oxy)-5-fluoropyridin-2-yl)-N,3,5-trimethyl-1H-pyrazole-4-carboxamide (81)

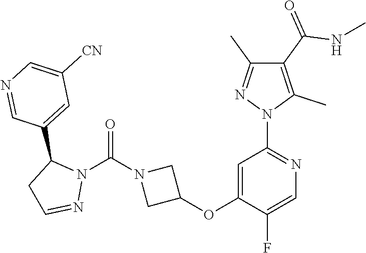 [SMILES: CNC(=O)c1c(C)nn(-c2cc(OC3CN(C(=O)N4N=CC[C@H]4c4cncc(C#N)c4)C3)c(F)cn2)c1C]

The titled compound 81 was prepared in a 15.8% yield according to the procedure outlined for compound 74. ¹H NMR (400 MHz, Chloroform-d) δ 8.80 (d, J=2.0 Hz, 1H), 8.74 (d, J=2.2 Hz, 1H), 8.20 (d, J=2.4 Hz, 11H), 7.82 (t, J=2.1 Hz, 11H), 7.16 (d, J=6.1 Hz, 11H), 6.89-6.83 (m, 1H), 5.59 (s, 1H), 5.37 (dd, J=12.2, 6.9 Hz, 1H), 5.15 (td, J=6.4, 3.2 Hz, 1H), 4.69-4.53 (m, 2H), 4.40-4.23 (m, 2H), 3.45 (ddd, J=18.6, 12.2, 1.7 Hz, 1H), 3.00 (d, J=4.9 Hz, 3H), 2.81-2.70 (m, 1H), 2.74 (s, 3H), 2.44 (s, 3H). Mass (m/z) 518.4 [M+H]⁺.

### (S)-1-(5-fluoro-4-((1-(5-(3-fluorophenyl)-4,5-dihydro-1H-pyrazole-1-carbonyl)azetidin-3-yl)oxy)pyridin-2-yl)-3,5-dimethyl-1H-pyrazole-4-carb oxylic acid (82)

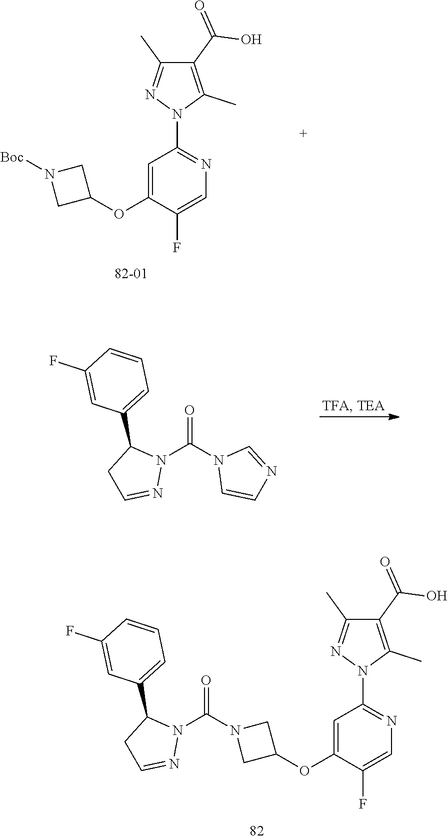 [SMILES: Cc1nn(-c2cc(OC3CN(C(=O)OC(C)(C)C)C3)c(F)cn2)c(C)c1C(=O)O ; O=C(N1N=CC[C@H]1c1cccc(F)c1)n1ccnc1 ; Cc1nn(-c2cc(OC3CN(C(=O)N4N=CC[C@H]4c4cccc(F)c4)C3)c(F)cn2)c(C)c1C(=O)O]

The titled compound 82 was prepared in a 35.7% yield from 82-01 according to the procedure outlined for compound 1-02. ¹H NMR (400 MHz, Chloroform-d) δ 8.23 (d, J=2.4 Hz, 1H), 7.35-7.28 (m, 1H), 7.18 (d, J=6.1 Hz, 1H), 7.02 (dt, J=7.6, 1.3 Hz, 1H), 6.99-6.86 (m, 2H), 6.80 (t, J=1.7 Hz, 1H), 5.31 (dd, J=12.2, 6.4 Hz, 1H), 5.15 (td, J=6.4, 3.3 Hz, 1H), 4.63 (dt, J=17.6, 8.5 Hz, 2H), 4.32 (dd, J=31.8, 10.9 Hz, 2H), 3.36 (ddd, J=18.6, 12.1, 1.7 Hz, 1H), 2.88 (s, 3H), 2.80-2.70 (m, 1H), 2.51 (s, 3H). Mass (m/z) 497.3 [M+H]⁺.

### (S)-1-(4-((1-(5-(3-cyano-5-fluorophenyl)-4,5-dihydro-1H-pyrazole-1-carbonyl)azetidin-3-yl)oxy)-5-fluoropyridin-2-yl)-3,5-dimethyl-1H-pyrazole-4-carboxylic acid (83)

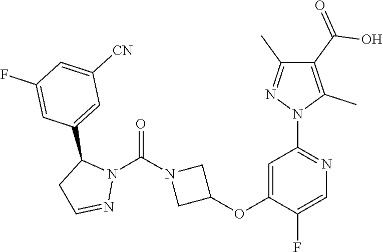 [SMILES: Cc1nn(-c2cc(OC3CN(C(=O)N4N=CC[C@H]4c4cc(F)cc(C#N)c4)C3)c(F)cn2)c(C)c1C(=O)O]

The titled compound 83 was prepared in a 28.5% yield from 82-01 according to the procedure outlined for compound 1-02. ¹H NMR (400 MHz, Chloroform-d) δ 8.24 (d, J=2.4 Hz, 1H), 7.34 (t, J=1.5 Hz, 1H), 7.29-7.25 (m, 1H), 7.23-7.17 (m, 2H), 6.85-6.80 (m, 1H), 5.32 (dd, J=12.2, 6.7 Hz, 1H), 5.17 (tt, J=6.6, 3.9 Hz, 1H), 4.72-4.57 (m, 2H), 4.43-4.24 (m, 2H), 3.41 (ddd, J=18.7, 12.2, 1.7 Hz, 1H), 2.88 (s, 3H), 2.71 (ddd, J=18.7, 6.7, 1.8 Hz, 1H), 2.51 (s, 3H). Mass (m/z) 522.2 [M+H]⁺.

### (S)-1-(5-fluoro-4-((1-(5-phenyl-4,5-dihydro-1H-pyrazole-1-carbonyl) azetidin-3-yl)oxy)pyridin-2-yl)-3,5-dimethyl-1H-pyrazole-4-carboxylic acid (84)

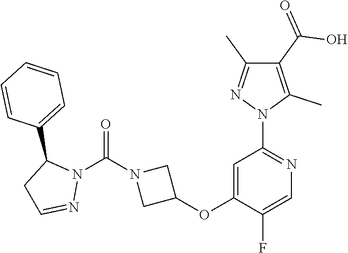 [SMILES: Cc1nn(-c2cc(OC3CN(C(=O)N4N=CC[C@H]4c4ccccc4)C3)c(F)cn2)c(C)c1C(=O)O]

The titled compound 84 was prepared in 29.6% yield from 82-01 according to the procedure outlined for compound 1-02. ¹H NMR (400 MHz, Chloroform-d) δ 8.22 (d, J=2.4 Hz, 1H), 7.36-7.31 (m, 2H), 7.29-7.20 (m, 3H), 7.17 (d, J=6.1 Hz, 1H), 6.80 (t, J=1.7 Hz, 1H), 5.32 (dd, J=12.1, 6.3 Hz, 11H), 5.13 (td, J=6.4, 3.2 Hz, 11H), 4.61 (dt, J=24.3, 8.2 Hz, 2H), 4.31 (ddd, J=31.9, 10.6, 3.9 Hz, 2H), 3.36 (ddd, J=18.6, 12.2, 1.7 Hz, 1H), 2.87 (s, 3H), 2.76 (ddd, J=18.6, 6.3, 1.8 Hz, 1H), 2.51 (s, 3H). Mass (m/z) 479.3 [M+H]⁺.

### (S)-1-(4-((1-(5-(3-cyano-5-fluorophenyl)-4,5-dihydro-1H-pyrazole-1-carbonyl)azetidin-3-yl)oxy)-5-fluoropyridin-2-yl)-3,5-dimethyl-N-(oxetan-3-yl)-1H-pyrazole-4-carboxamide (85)

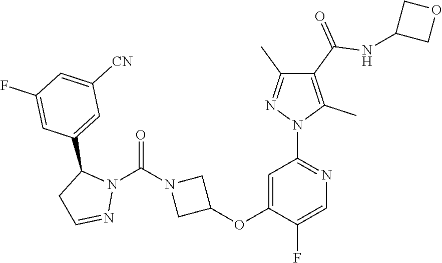 [SMILES: Cc1nn(-c2cc(OC3CN(C(=O)N4N=CC[C@H]4c4cc(F)cc(C#N)c4)C3)c(F)cn2)c(C)c1C(=O)NC1COC1]

The titled compound 85 was prepared in a 45.8% yield according to the procedure outlined for compound 74. ¹H NMR (400 MHz, Chloroform-d) δ 8.21 (d, J=2.4 Hz, 1H), 7.34 (t, J=1.5 Hz, 1H), 7.30-7.24 (m, 1H), 7.23-7.19 (m, 1H), 7.17 (d, J=6.1 Hz, 1H), 6.84-6.82 (m, 11H), 6.09 (d, J=7.4 Hz, 11H), 5.31 (dd, J=12.3, 6.7 Hz, 11H), 5.24 (q, J=6.7 Hz, 1H), 5.16 (td, J=6.5, 3.3 Hz, 1H), 5.03 (t, J=7.1 Hz, 2H), 4.69-4.52 (m, 4H), 4.34 (d, J=30.8 Hz, 2H), 3.41 (ddd, J=18.6, 12.3, 1.7 Hz, 1H), 2.79 (s, 3H), 2.71 (ddd, J=18.7, 6.7, 1.7 Hz, 1H), 2.47 (s, 3H). Mass (m/z) 577.4 [M+H]⁺.

### (S)-1-(4-((1-(5-(3-cyano-5-fluorophenyl)-4,5-dihydro-1H-pyrazole-1-carbonyl)azetidin-3-yl)oxy)-5-fluoropyridin-2-yl)-N-cyclopropyl-3,5-dimethyl-1H-pyrazole-4-carboxamide (86)

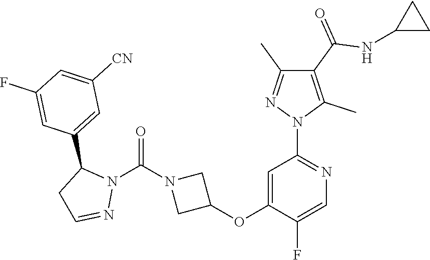 [SMILES: Cc1nn(-c2cc(OC3CN(C(=O)N4N=CC[C@H]4c4cc(F)cc(C#N)c4)C3)c(F)cn2)c(C)c1C(=O)NC1CC1]

The titled compound 86 was prepared in 25.8% yield according to the procedure outlined for compound 74. ¹H NMR (400 MHz, Chloroform-d) δ 8.20 (d, J=2.5 Hz, 1H), 7.34 (t, J=1.5 Hz, 1H), 7.30-7.24 (m, 1H), 7.23-7.19 (m, 1H), 7.15 (d, J=6.1 Hz, 1H), 6.85-6.79 (m, 1H), 5.73 (s, 1H), 5.31 (dd, J=12.2, 6.7 Hz, 1H), 5.15 (td, J=6.4, 3.3 Hz, 1H), 4.70-4.54 (m, 2H), 4.33 (d, J=31.4 Hz, 2H), 3.40 (ddd, J=18.7, 12.2, 1.7 Hz, 1H), 2.87 (dd, J=7.2, 3.9 Hz, 1H), 2.75 (s, 3H), 2.74-2.64 (m, 1H), 2.41 (s, 3H), 0.88 (q, J=6.3 Hz, 2H), 0.63-0.57 (m, 2H). Mass (m/z) 577.4 [M+H]⁺.

### (S)-1-(4-((1-(5-(3-cyano-5-fluorophenyl)-4,5-dihydro-1H-pyrazole-1-carbonyl)azetidin-3-yl)oxy)-5-fluoropyridin-2-yl)-N-ethyl-3,5-dimethyl-1H-pyrazole-4-carboxamide (87)

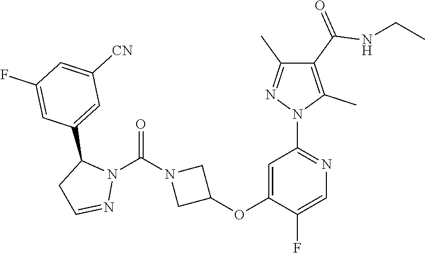 [SMILES: CCNC(=O)c1c(C)nn(-c2cc(OC3CN(C(=O)N4N=CC[C@H]4c4cc(F)cc(C#N)c4)C3)c(F)cn2)c1C]

The titled compound 87 was prepared in 26.9% yield according to the procedure outlined for compound 74. ¹H NMR (400 MHz, Chloroform-d) δ 8.20 (d, J=2.4 Hz, 1H), 7.34 (t, J=1.5 Hz, 1H), 7.28-7.24 (m, 1H), 7.20 (dt, J=9.2, 2.0 Hz, 11H), 7.16 (d, J=6.1 Hz, 1H), 6.82 (t, J=1.7 Hz, 1H), 5.56 (s, 1H), 5.31 (dd, J=12.2, 6.7 Hz, 1H), 5.15 (td, J=6.5, 3.4 Hz, 1H), 4.63 (s, 2H), 4.33 (d, J=29.5 Hz, 2H), 3.55-3.36 (m, 3H), 2.76 (s, 3H), 2.70 (ddd, J=18.6, 6.7, 1.7 Hz, 1H), 2.43 (s, 3H), 1.25 (t, J=7.3 Hz, 3H). Mass (m/z) 549.4 [M+H]⁺.

### (3-((2-(1,4-dimethyl-1H-pyrazol-5-yl)-5-fluoropyridin-4-yl)oxy)azetidin-1-yl)(5-(2-methylthiazol-5-yl)-4,5-dihydro-1H-pyrazol-1-yl)methanone (88)

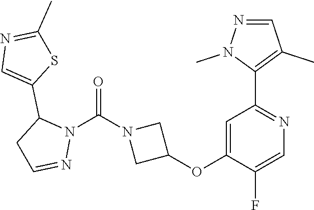 [SMILES: Cc1ncc(C2CC=NN2C(=O)N2CC(Oc3cc(-c4c(C)cnn4C)ncc3F)C2)s1]

The title compound 88 was prepared in a yield of 31% as an orange solid according to the procedure for 11. Mass (m/z) 456.2 [M+H]⁺. ¹H NMR (400 MHz, DMSO-d₆) δ 8.64 (d, J=3.0 Hz, 1H), 7.56-7.54 (m, 1H), 7.36-7.32 (m, 1H), 7.13-7.05 (m, 2H), 5.50 (dd, J=11.7, 5.9 Hz, 1H), 5.30-5.23 (m, 1H), 4.59-4.43 (m, 2H), 4.14-3.96 (m, 2H), 3.85 (s, 3H), 3.37 (ddd, J=18.6, 11.8, 1.7 Hz, 1H), 2.87 (ddd, J=18.6, 5.9, 1.8 Hz, 1H), 2.60 (s, 3H), 2.06 (s, 3H).

### (S)-3-(1-(3-((2-(3,5-dimethylisoxazol-4-yl)-5-fluoropyridin-4-yl)oxy) azetidine-1-carbonyl)-4,5-dihydro-1H-pyrazol-5-yl)-5-fluorobenzonitrile (89)

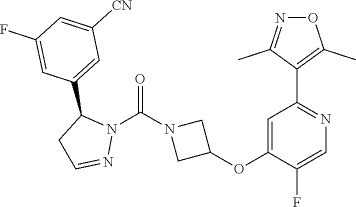 [SMILES: Cc1noc(C)c1-c1cc(OC2CN(C(=O)N3N=CC[C@H]3c3cc(F)cc(C#N)c3)C2)c(F)cn1]

The title compound 89 was prepared in a yield of 18.6% as an orange solid according to the procedure for 11. Mass (m/z) 479.4 [M+H]⁺. ¹H NMR (400 MHz, DMSO-d₆) δ 8.54 (d, J=3.0 Hz, 1H), 7.72 (ddd, J=8.6, 2.4, 1.6 Hz, 1H), 7.53 (t, J=1.4 Hz, 1H), 7.41 (ddd, J=9.7, 2.6, 1.5 Hz, 1H), 7.03-6.97 (m, 2H), 5.29-5.20 (m, 2H), 4.52 (s, 2H), 4.06 (s, 2H), 3.37 (ddd, J=18.8, 12.2, 1.8 Hz, 1H), 2.67 (ddd, J=18.4, 7.2, 1.6 Hz, 1H), 2.50 (s, 3H), 2.31 (s, 3H).

### (3-((2-(1,4-dimethyl-1H-pyrazol-5-yl)-5-fluoropyridin-4-yl)oxy)azetidin-1-yl)(5-(2-methyloxazol-4-yl)-4,5-dihydro-1H-pyrazol-1-yl)methanone (90)

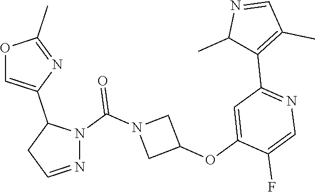 [SMILES: CC1=C(c2cc(OC3CN(C(=O)N4N=CCC4c4coc(C)n4)C3)c(F)cn2)C(C)N=C1]

The title compound 90 was prepared in a yield of 2.4% as an orange solid according to the procedure for 11. Mass (m/z) 440.4 [M+H]⁺. ¹H NMR (400 MHz, Chloroform-d) δ 8.48 (d, J=2.8 Hz, 1H), 7.55 (s, 1H), 7.36 (s, 1H), 6.84 (t, J=1.6 Hz, 1H), 6.68 (d, J=6.4 Hz, 1H), 5.31 (dd, J=10.4, 8.4 Hz, 1H), 5.02 (td, J=6.4, 3.3 Hz, 1H), 4.60-4.49 (m, 2H), 4.37-4.22 (m, 2H), 3.95 (s, 3H), 2.43 (s, 3H), 2.11 (s, 3H).

### (3-((2-(1,4-dimethyl-1H-pyrazol-5-yl)-5-fluoropyridin-4-yl)oxy)azetidin-1-yl)(5-(thiazol-5-yl)-4,5-dihydro-1H-pyrazol-1-yl)methanone (91)

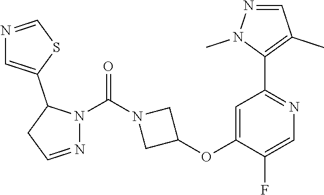 [SMILES: Cc1cnn(C)c1-c1cc(OC2CN(C(=O)N3N=CCC3c3cncs3)C2)c(F)cn1]

The title compound 91 was prepared in a yield of 24% as a white solid according to the procedure for 11. Mass (m/z) 442.2 [M+H]⁺. ¹H NMR (400 MHz, DMSO-d₆) δ 8.99-8.96 (m, 1H), 8.64 (d, J=3.1 Hz, 1H), 7.83-7.80 (m, 1H), 7.34 (s, 1H), 7.14-7.06 (m, 2H), 5.60 (dd, J=11.8, 5.8 Hz, 1H), 5.32-5.23 (m, 1H), 4.59-4.43 (m, 2H), 4.17-3.96 (m, 2H), 3.85 (s, 3H), 3.41 (ddd, J=18.8, 11.8, 1.7 Hz, 1H), 2.88 (ddd, J=18.7, 6.0, 1.8 Hz, 1H), 2.06 (s, 3H).

### (3-((2-(1,4-dimethyl-1H-pyrazol-5-yl)-5-fluoropyridin-4-yl)oxy)azetidin-1-yl)(5-(thiazol-2-yl)-4,5-dihydro-1H-pyrazol-1-yl)methanone (92)

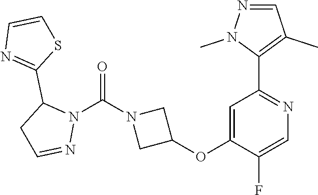 [SMILES: Cc1cnn(C)c1-c1cc(OC2CN(C(=O)N3N=CCC3c3nccs3)C2)c(F)cn1]

The title compound 92 was prepared in a yield of 60% as a white solid according to the procedure for 11. Mass (m/z) 442.3 [M+H]⁺. ¹H NMR (400 MHz, DMSO-d₆) δ 8.64 (d, J=3.1 Hz, 1H), 7.73 (d, J=3.3 Hz, 1H), 7.66 (d, J=3.3 Hz, 1H), 7.34 (s, 1H), 7.14-7.08 (m, 2H), 5.59 (dd, J=12.0, 5.9 Hz, 1H), 5.34-5.24 (m, 1H), 4.62-4.48 (m, 2H), 4.17-3.99 (m, 2H), 3.85 (s, 3H), 3.40 (ddd, J=18.6, 12.0, 1.7 Hz, 1H), 3.12 (ddd, J=18.6, 6.0, 1.8 Hz, 1H), 2.07 (s, 3H).

### (5-(3-chloropyridin-2-yl)-4,5-dihydro-1H-pyrazol-1-yl)(3-((2-(1,4-di methyl-1H-pyrazol-5-yl)-5-fluoropyridin-4-yl)oxy)azetidin-1-yl)methanone (93)

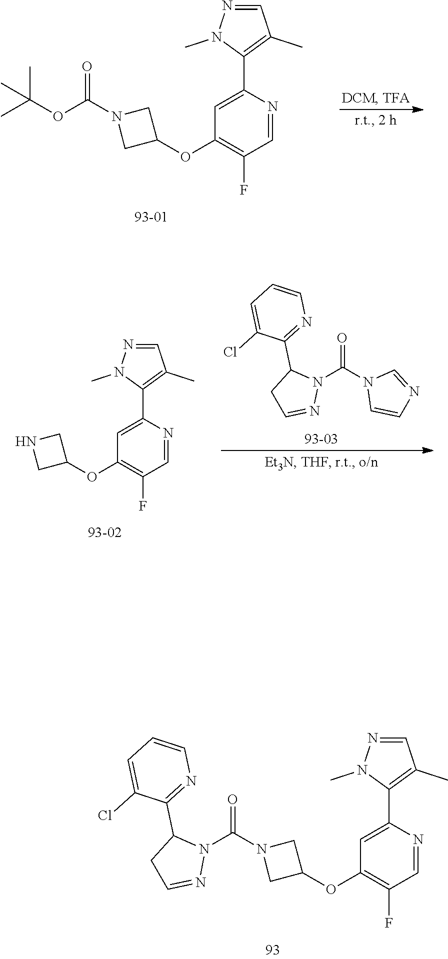 [SMILES: Cc1cnn(C)c1-c1cc(OC2CN(C(=O)OC(C)(C)C)C2)c(F)cn1 ; Cc1cnn(C)c1-c1cc(OC2CNC2)c(F)cn1 ; O=C(N1N=CCC1c1ncccc1Cl)n1ccnc1 ; Cc1cnn(C)c1-c1cc(OC2CN(C(=O)N3N=CCC3c3ncccc3Cl)C2)c(F)cn1]

Step 1: TFA (1 mL) was added to a solution of 93-01 (160 mg, 0.43 mmol) in DCM (3 mL). the reaction mixture was stirred at room temperature for 0.5 hr. Then the solvent was evaporated in vacuo to give titled compound 93-02 as a colorless oil. Mass (m/z) 263.1 [M+H]⁺.

Step 2: 93-03 was added to a solution of 93-02 (100 mg, 0.36 mmol) and TEA (110 mg, 1.09 mmol) in THE (2 mL). The reaction mixture was stirred at room temperature overnight. Then the solvent was evaporated in vacuo. The oil residue was purified by prep-HPLC to give 20 mg of 93 as a yellow oil (7%). Mass (m/z) 470.1 [M+H]⁺. ¹H NMR (400 MHz, CDCl₃) δ 8.50-8.38 (m, 2H), 7.65 (dd, J=8.1, 1.5 Hz, 1H), 7.33 (s, 1H), 7.14 (dd, J=8.1, 4.7 Hz, 11H), 6.79 (t, J=1.6 Hz, 11H), 6.67 (d, J=6.6 Hz, 1H), 5.83 (dd, J=12.1, 6.4 Hz, 1H), 5.07-4.95 (m, 1H), 4.55 (dd, J=19.6, 9.3 Hz, 2H), 4.31 (d, J=9.7 Hz, 2H), 3.93 (s, 3H), 3.32 (tt, J=18.1, 3.8 Hz, 1H), 2.90-2.76 (m, 1H), 2.08 (s, 3H).

### (3-((2-(1,4-dimethyl-1H-pyrazol-5-yl)-5-fluoropyridin-4-yl)oxy)azetidin-1-yl)(5-(5-methylthiazol-2-yl)-4,5-dihydro-1H-pyrazol-1-yl)methanone (94)

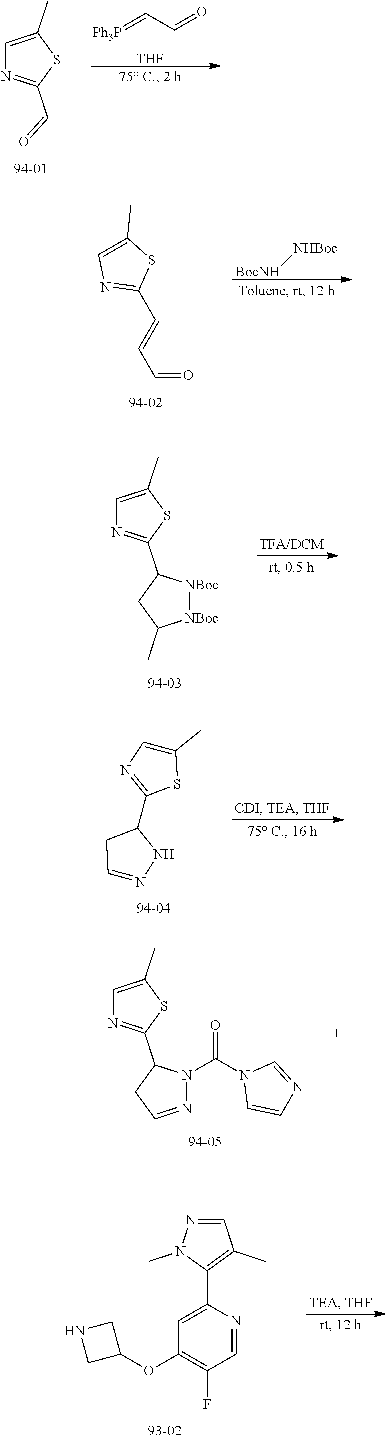

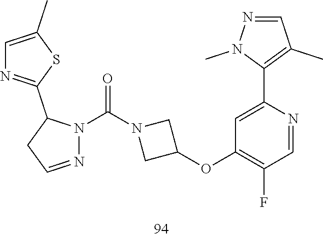 [SMILES: Cc1cnc(C2CC=NN2C(=O)N2CC(Oc3cc(-c4c(C)cnn4C)ncc3F)C2)s1]

Step 1: A mixture of 2-(triphenyl-15-phosphaneylidene)acetaldehyde (2.87 g, 9.44 mmol) and 94-01 (1 g, 7.86 mmol) in THE (15 mL) was stirred at 75° C. for 2 h. The reaction mixture was cooled to room temperature and concentrated under reduce pressure. The residue was purified by silica gel column chromatography, eluted with (PE:EtOAc) (1:1) to afford compound 94-02 (1.2 g, 99.6%) as a brown yellow oil. Mass (m/z) 154.2 [M+H]⁺.

Step 2: To a stirred solution of 94-02 (1 g, 6.54 mmol) and di-tert-butyl hydrazine-1,2-dicarboxylate (2.3 g, 9.80 mmol) in toluene (20 mL) was added (S)-2-(diphenyl((trimethylsilyl)oxy)methyl)pyrrolidine (489 mg, 1.50 mmol) at room temperature. The resulting mixture was stirred for additional 12 h at room temperature. The resulting mixture was concentrated under reduce pressure. The residue was purified by silica gel column chromatography, eluted with (PE:EtOAc) (1:1) to afford compound 94-03 (2.2 g, 87.4%) as a brown yellow solid. Mass (m/z) 386.4 [M+H]⁺.

Step 3: To a stirred solution of compound 94-03 (2.2 g, 5.71 mmol) in DCM (8 mL) was added TFA (5 mL) at room temperature. The resulting mixture was stirred for additional 0.5 h at room temperature. The resulting mixture was concentrated under reduce pressure to give compound 94-04 (0.95 g, 99.5%) as a yellow oil. Mass (m/z) 168.2 [M+H]⁺.

Step 4: To a stirred solution of 94-04 (0.95 g, 5.68 mmol) and CDI (4.60 g, 28.40 mmol) in THE (20 mL) was added TEA (1.72 g, 17.06 mmol) at room temperature under N₂ atmosphere. The resulting mixture was stirred for additional 16 h at 75° C. The mixture was allowed to cool down to room temperature. The resulting mixture was concentrated under reduce pressure.

The residue was purified by silica gel column chromatography, eluted with (PE:EtOAc) (1:2) to afford 94-05 (0.8 g, 53.8%) as a brown yellow oil. Mass (m/z) 262.3 [M+H]⁺.

Step 5: To a stirred solution of 94-05 (100 mg, 0.38 mmol) and 93-02 (120 mg, 0.46 mmol) in THE (5 mL) was added TEA (3 mL) at room temperature. The resulting mixture was stirred for additional 12 h at room temperature. The resulting mixture was concentrated under reduce pressure. The resulting mixture was extracted with EtOAc (3×10 mL). The combined organic layers were washed with brine (10 mL), dried over anhydrous Na₂SO₄, after filtration, the filtrate was concentrated under reduce pressure. The residue was purified by silica gel column chromatography, eluted with (PE:EtOAc) (1:1) to afford the titled compound 94 (54.5 mg, 31.3%) as an off-white solid. Mass (m/z) 456.5 [M+H]⁺. ¹H NMR (400 MHz, DMSO-d₆) δ 8.61 (d, J=3.2 Hz, 1H), 7.34 (q, J=1.2 Hz, 1H), 7.31 (s, 1H), 7.09-7.02 (m, 2H), 5.45 (dd, J=12.0, 5.6 Hz, 1H), 5.26 (tt, J=6.6, 3.7 Hz, 1H), 4.53 (d, J=15.2 Hz, 2H), 4.04 (dd, J=31.0, 9.6 Hz, 2H), 3.82 (s, 3H), 3.37-3.27 (m, 1H), 3.09 (ddd, J=18.4, 6.0, 1.6 Hz, 1H), 2.36 (s, 3H), 2.04 (s, 3H).

### (3-((2-(1,4-dimethyl-1H-pyrazol-5-yl)-5-fluoropyridin-4-yl)oxy)azetidin-1-yl)(5-(thiazol-4-yl)-4,5-dihydro-1H-pyrazol-1-yl)methanone (95)

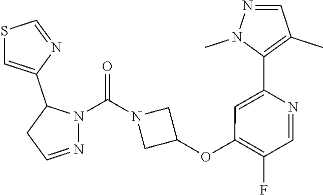 [SMILES: Cc1cnn(C)c1-c1cc(OC2CN(C(=O)N3N=CCC3c3cscn3)C2)c(F)cn1]

The titled compound 95 was prepared in a yield of 10.3% from compound 93-02 according to the procedure outlined for compound 94. Mass (m/z) 442.4 [M+H]⁺. ¹H NMR (400 MHz, Chloroform-d) δ 8.79 (d, J=1.6 Hz, 1H), 8.50 (d, J=2.8 Hz, 1H), 7.40 (s, 1H), 7.35 (d, J=2.0 Hz, 1H), 6.88 (s, 1H), 6.69 (d, J=6.4 Hz, 1H), 5.56 (dd, J=11.6, 6.0 Hz, 1H), 5.06-5.00 (m, 1H), 4.64-4.52 (m, 2H), 4.40-4.25 (m, 2H), 3.96 (s, 3H), 3.36-3.24 (m, 1H), 3.23-3.12 (m, 1H), 2.11 (s, 3H).

### (3-((2-(1,4-dimethyl-1H-pyrazol-5-yl)-5-fluoropyridin-4-yl)oxy)azetidin-1-yl)(5-(2-methylthiazol-4-yl)-4,5-dihydro-1H-pyrazol-1-yl)methanone (96)

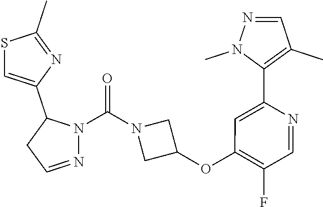 [SMILES: Cc1nc(C2CC=NN2C(=O)N2CC(Oc3cc(-c4c(C)cnn4C)ncc3F)C2)cs1]

The titled compound 96 was prepared in a yield of 27.7% from compound 93-02 according to the procedure outlined for compound 94. ¹H NMR (400 MHz, Chloroform-d) δ 8.48 (d, J=2.9 Hz, 1H), 7.36 (s, 1H), 7.06 (s, 1H), 6.84 (s, 1H), 6.68 (d, J=6.6 Hz, 1H), 5.44 (dd, J=11.7, 6.6 Hz, 1H), 5.03 (s, 1H), 4.57 (d, J=21.3 Hz, 2H), 4.31 (dd, J=28.3, 10.3 Hz, 2H), 3.95 (s, 3H), 3.29-3.08 (m, 2H), 2.68 (s, 3H), 2.11 (s, 3H). Mass (m/z) 456.3 [M+H]⁺.

### (3-((2-(1,4-dimethyl-1H-pyrazol-5-yl)-5-fluoropyridin-4-yl)oxy)azetidin-1-yl)(5-(4-methylthiazol-2-yl)-4,5-dihydro-1H-pyrazol-1-yl)methanone (97)

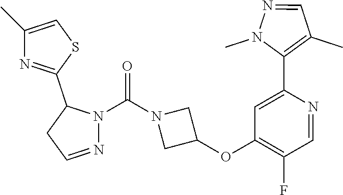

The titled compound 97 was prepared in a yield of 13.4% from compound 93-02 according to the procedure outlined for compound 94. ¹H NMR (400 MHz, Methanol-d₄) δ 8.42 (d, J=3.2 Hz, 1H), 7.27 (s, 1H), 7.02-6.93 (m, 2H), 6.89 (t, J=1.7 Hz, 1H), 5.50 (dd, J=12.1, 6.1 Hz, 1H), 5.17 (td, J=6.3, 3.1 Hz, 1H), 4.53 (s, 2H), 4.24-4.09 (m, 2H), 3.77 (s, 3H), 3.34 (ddd, J=18.6, 12.1, 1.7 Hz, 1H), 2.95 (ddd, J=18.7, 6.2, 1.8 Hz, 1H), 2.29 (d, J=1.0 Hz, 3H), 2.01 (s, 3H). Mass (m/z) 456.2 [M+H]⁺.

### (R)-1-(5-fluoro-4-((1-(5-(3-fluorophenyl)-4,5-dihydro-1H-pyrazole-1-carbonyl)azetidin-3-yl)oxy)pyridin-2-yl)-3,5-dimethyl-1H-pyrazole-4-carb oxamide (98)

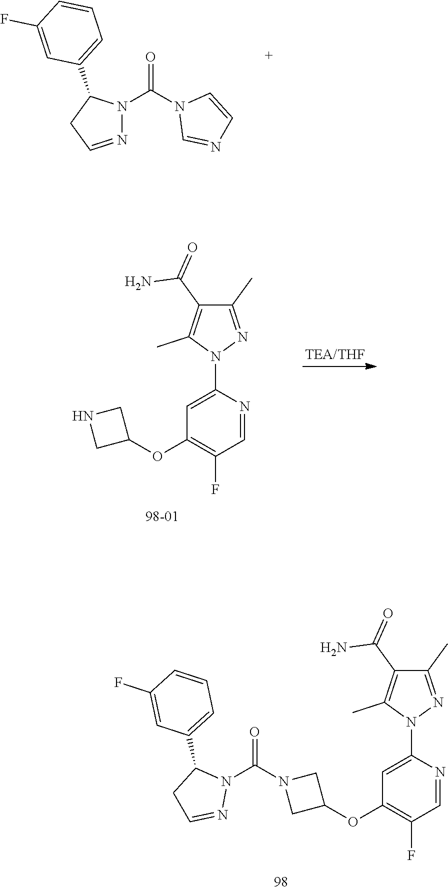 [SMILES: O=C(N1N=CC[C@@H]1c1cccc(F)c1)n1ccnc1 ; Cc1nn(-c2cc(OC3CNC3)c(F)cn2)c(C)c1C(N)=O ; Cc1nn(-c2cc(OC3CN(C(=O)N4N=CC[C@@H]4c4cccc(F)c4)C3)c(F)cn2)c(C)c1C(N)=O]

The titled compound 98 was prepared in a yield of 19.2% from compound 98-01 according to the procedure outlined for compound 1-02. ¹H NMR (400 MHz, DMSO-d₆) δ 8.44 (d, J=2.7 Hz, 1H), 7.37 (td, J=8.0, 6.1 Hz, 1H), 7.24 (d, J=6.2 Hz, 3H), 7.10-6.97 (m, 4H), 5.33 (tt, J=6.6, 3.6 Hz, 1H), 5.24 (dd, J=12.1, 6.3 Hz, 1H), 4.51 (s, 2H), 4.08 (d, J=12.8 Hz, 2H), 3.40 (ddd, J=18.7, 12.1, 1.7 Hz, 1H), 2.65 (dd, J=6.3, 1.8 Hz, 1H), 2.60 (s, 3H), 2.33 (s, 3H). Mass (m/z) 496.2 [M+H]⁺.

### (3-((2-(1,4-dimethyl-1H-pyrazol-5-yl)-5-fluoropyridin-4-yl)oxy)azetidin-1-yl)(5-(pyridin-3-yl)-4,5-dihydro-1H-pyrazol-1-yl)methanone (99)

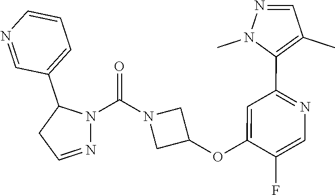 [SMILES: Cc1cnn(C)c1-c1cc(OC2CN(C(=O)N3N=CCC3c3cccnc3)C2)c(F)cn1]

The title compound 99 was prepared in a yield of 69% as a white solid according to the procedure outlined for compound 11. Mass (m/z) 436.3 [M+H]⁺. ¹H NMR (400 MHz, Chloroform-d) δ 8.55-8.51 (m, 2H), 8.49 (d, J=2.9 Hz, 1H), 7.57 (dt, J=7.9, 2.0 Hz, 1H), 7.36-7.34 (m, 1H), 7.31-7.27 (m, 1H), 6.84 (t, J=1.7 Hz, 1H), 6.69 (d, J=6.6 Hz, 1H), 5.34 (dd, J=12.2, 6.6 Hz, 1H), 5.09-4.99 (m, 1H), 4.65-4.50 (m, 2H), 4.40-4.22 (m, 2H), 3.95 (s, 3H), 3.41 (ddd, J=18.7, 12.2, 1.7 Hz, 1H), 2.78 (ddd, J=18.6, 6.6, 1.8 Hz, 1H), 2.12-2.09 (m, 3H).

### (3-((2-(1,4-dimethyl-1H-pyrazol-5-yl)-5-fluoropyridin-4-yl)oxy)azetidin-1-yl)(5-(pyridin-2-yl)-4,5-dihydro-1H-pyrazol-1-yl)methanone (100)

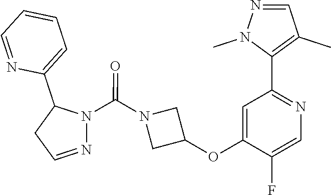 [SMILES: Cc1cnn(C)c1-c1cc(OC2CN(C(=O)N3N=CCC3c3ccccn3)C2)c(F)cn1]

The title compound 100 was prepared in a yield of 62% as an off white solid according to the procedure outlined for compound 11. Mass (m/z) 436.3 [M+H]⁺. ¹H NMR (400 MHz, Chloroform-d) δ 8.59 (ddd, J=4.9, 1.8, 0.9 Hz, 1H), 8.48 (d, J=2.9 Hz, 1H), 7.67 (td, J=7.7, 1.8 Hz, 1H), 7.39-7.33 (m, 2H), 7.20 (ddd, J=7.6, 4.9, 1.2 Hz, 1H), 6.84 (t, J=1.7 Hz, 1H), 6.68 (d, J=6.6 Hz, 1H), 5.41 (dd, J=12.0, 6.6 Hz, 1H), 5.07-4.99 (m, 1H), 4.64-4.51 (m, 2H), 4.39-4.25 (m, 2H), 3.95 (s, 3H), 3.32 (ddd, J=18.4, 12.0, 1.7 Hz, 1H), 3.13 (ddd, J=18.4, 6.6, 1.7 Hz, 1H), 2.11-2.08 (m, 3H).

### (3-((2-(1,4-dimethyl-1H-pyrazol-5-yl)-5-fluoropyridin-4-yl)oxy)azetidin-1-yl)(5-(5-methylpyridin-3-yl)-4,5-dihydro-1H-pyrazol-1-yl)methanone (101)

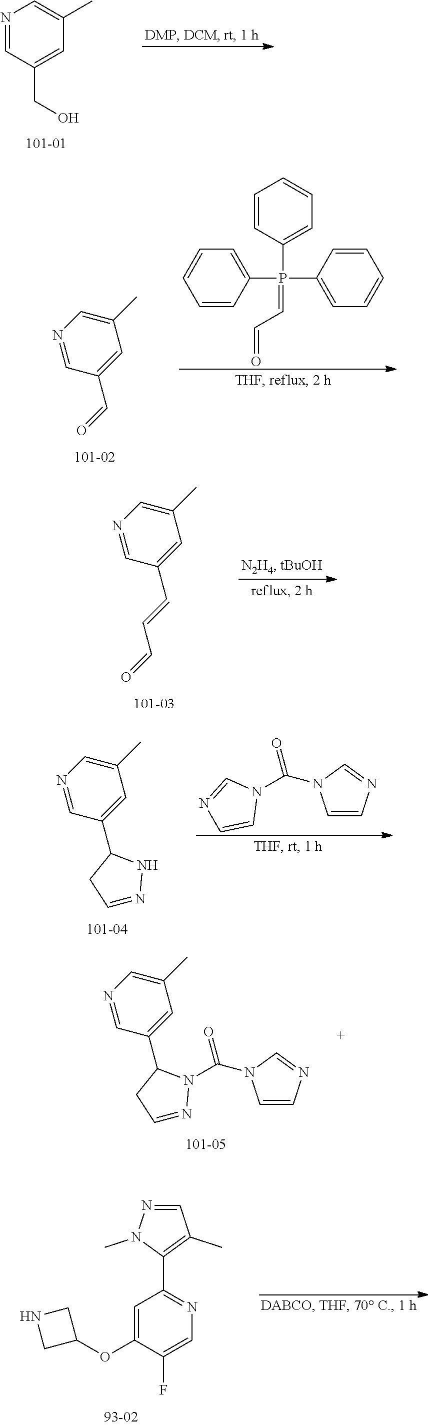

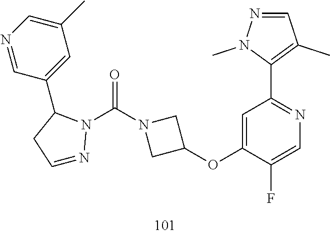 [SMILES: Cc1cncc(C2CC=NN2C(=O)N2CC(Oc3cc(-c4c(C)cnn4C)ncc3F)C2)c1]

The titled compound 101 was prepared in a yield of 20% from compound 101-01 according to the procedure outlined for compound 94. Mass (m/z) 450.3 [M+H]⁺. H NMR (400 MHz, Chloroform-d) δ 8.48 (d, J=3.0 Hz, 1H), 8.37-8.30 (m, 2H), 7.39-7.36 (m, 1H), 7.36-7.33 (d, J=0.6 Hz, 1H), 6.83 (t, J=1.7 Hz, 1H), 6.69 (d, J=6.6 Hz, 1H), 5.29 (dd, J=12.2, 6.6 Hz, 1H), 5.08-5.00 (m, 1H), 4.64-4.50 (m, 2H), 4.41-4.23 (m, 2H), 3.95 (s, 3H), 3.39 (ddd, J=18.6, 12.2, 1.7 Hz, 1H), 2.75 (ddd, J=18.6, 6.6, 1.8 Hz, 1H), 2.34 (s, 3H), 2.10 (s, 3H).

### (S)-1-(5-fluoro-4-((1-(5-(2,3,5-trifluorophenyl)-4,5-dihydro-1H-pyrazole-1-carbonyl)azetidin-3-yl)oxy)pyridin-2-yl)-3,5-dimethyl-1H-pyrazole-4-carboxamide (102)

### (R)-1-(5-fluoro-4-((1-(5-(2,3,5-trifluorophenyl)-4,5-dihydro-1H-pyrazole-1-carbonyl)azetidin-3-yl)oxy)pyridin-2-yl)-3,5-dimethyl-1H-pyrazole-4-carboxamide (103)

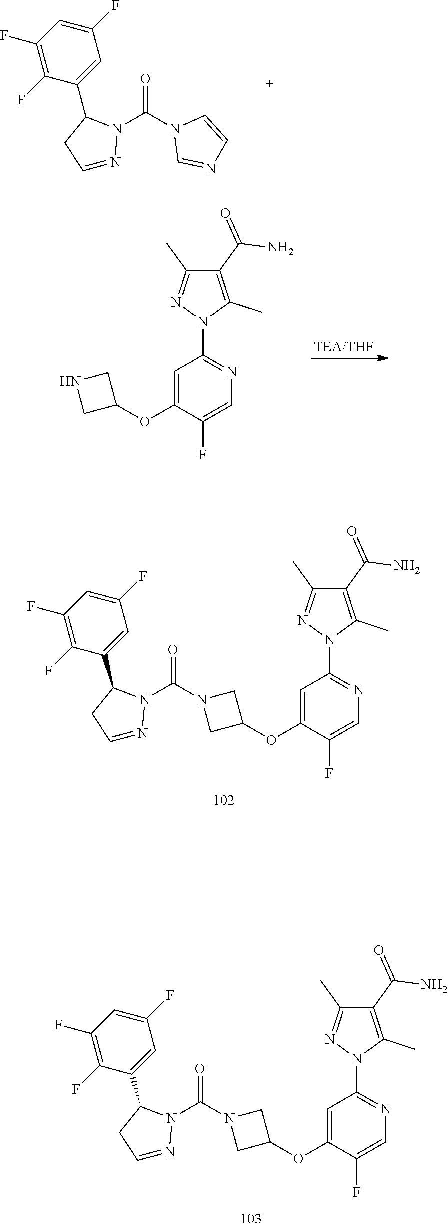

The titled compound 102 was prepared in a yield of 16.2% according to the procedure outlined for compound 98. ¹H NMR (400 MHz, DMSO-d₆) δ 8.44 (d, J=2.7 Hz, 1H), 7.44 (dddd, J=10.7, 8.7, 6.1, 3.1 Hz, 1H), 7.23 (d, J=6.3 Hz, 3H), 7.07 (d, J=1.8 Hz, 1H), 6.95-6.86 (m, 1H), 5.39 (dd, J=12.4, 6.9 Hz, 1H), 5.32 (tt, J=6.6, 3.6 Hz, 1H), 4.52 (s, 2H), 4.08 (s, 2H), 3.44 (ddd, J=18.7, 12.3, 1.7 Hz, 2H), 2.78-2.71 (m, 1H), 2.60 (s, 3H), 2.33 (s, 3H).

Mass (m/z) 532.1 [M+H]⁺.

The titled compound 103 was prepared in a yield of 15.8% according to the procedure outlined for compound 98. ¹H NMR (400 MHz, DMSO-d₆) δ 8.44 (d, J=2.7 Hz, 1H), 7.44 (qd, J=9.0, 3.2 Hz, 1H), 7.23 (d, J=6.2 Hz, 3H), 7.07 (d, J=1.7 Hz, 1H), 6.91 (q, J=3.8, 3.1 Hz, 1H), 5.45-5.37 (m, 1H), 5.32 (tt, J=6.5, 3.6 Hz, 1H), 4.52 (s, 2H), 4.08 (s, 2H), 3.43 (ddd, J=18.7, 12.4, 1.7 Hz, 1H), 2.81-2.69 (m, 1H), 2.60 (s, 3H), 2.33 (s, 4H). Mass (m/z) 532.1 [M+H]⁺.

### (S)-1-(5-fluoro-4-((1-(5-(2,3,5-trifluorophenyl)-4,5-dihydro-1H-pyrazole-1-carbonyl)azetidin-3-yl)oxy)pyridin-2-yl)-3,5-dimethyl-1H-pyrazole-4-carboxylic acid (104)

### (R)-1-(5-fluoro-4-((1-(5-(2,3,5-trifluorophenyl)-4,5-dihydro-1H-pyrazole-1-carbonyl)azetidin-3-yl)oxy)pyridin-2-yl)-3,5-dimethyl-1H-pyrazole-4-carboxylic acid (105)

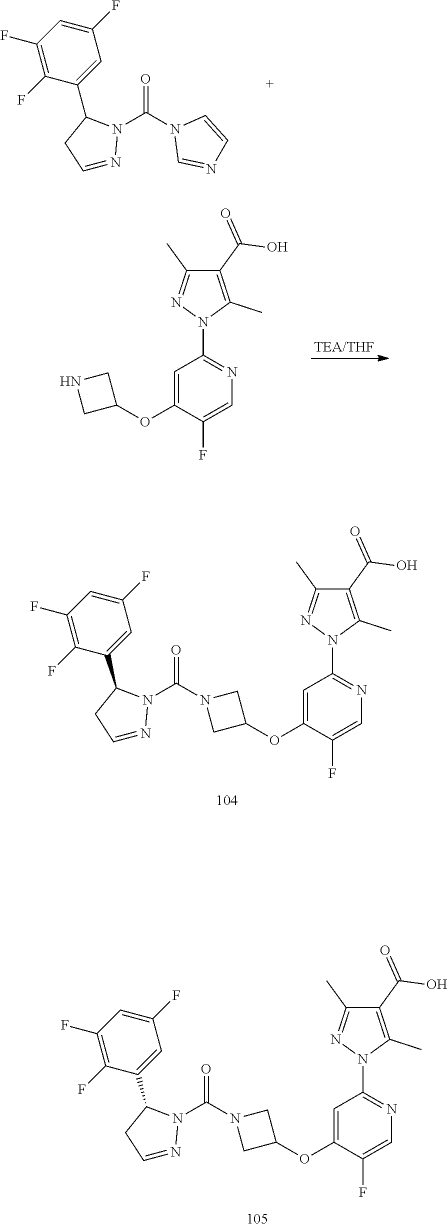

The titled compound 104 was prepared in a yield of 20.6% according to the procedure outlined for compound 98. ¹H NMR (400 MHz, DMSO-d₆) δ 12.45 (s, 1H), 8.47 (d, J=2.6 Hz, 1H), 7.44 (tdd, J=9.5, 6.0, 3.1 Hz, 1H), 7.27 (d, J=6.3 Hz, 1H), 7.07 (d, J=1.6 Hz, 1H), 6.91 (dd, J=7.9, 4.0 Hz, 11H), 5.39 (dd, J=12.4, 6.9 Hz, 11H), 5.32 (tt, J=6.5, 3.6 Hz, 11H), 4.52 (s, 2H), 4.08 (s, 2H), 3.43 (ddd, J=18.7, 12.4, 1.7 Hz, 1H), 2.79-2.72 (m, 1H), 2.70 (s, 3H), 2.39 (s, 3H). Mass (m/z) 533.1 [M+H]⁺.

The titled compound 105 was prepared in a yield of 22.1% according to the procedure outlined for compound 98. ¹H NMR (400 MHz, DMSO-d₆) δ 12.45 (s, 1H), 8.47 (d, J=2.6 Hz, 1H), 7.44 (tdd, J=9.5, 6.0, 3.1 Hz, 1H), 7.27 (d, J=6.3 Hz, 1H), 7.07 (d, J=1.6 Hz, 1H), 6.91 (dd, J=7.9, 4.0 Hz, 11H), 5.39 (dd, J=12.4, 6.9 Hz, 11H), 5.32 (tt, J=6.5, 3.6 Hz, 11H), 4.52 (s, 2H), 4.08 (s, 2H), 3.43 (ddd, J=18.7, 12.4, 1.7 Hz, 1H), 2.79-2.72 (m, 1H), 2.70 (s, 3H), 2.39 (s, 3H). Mass (m/z) 533.1 [M+H]⁺.

### (3-((6-(1,4-dimethyl-1H-pyrazol-5-yl)-3-fluoropyridin-2-yl)oxy)azetidin-1-yl)(5-(2-methylthiazol-5-yl)-4,5-dihydro-1H-pyrazol-1-yl)methanone (106)

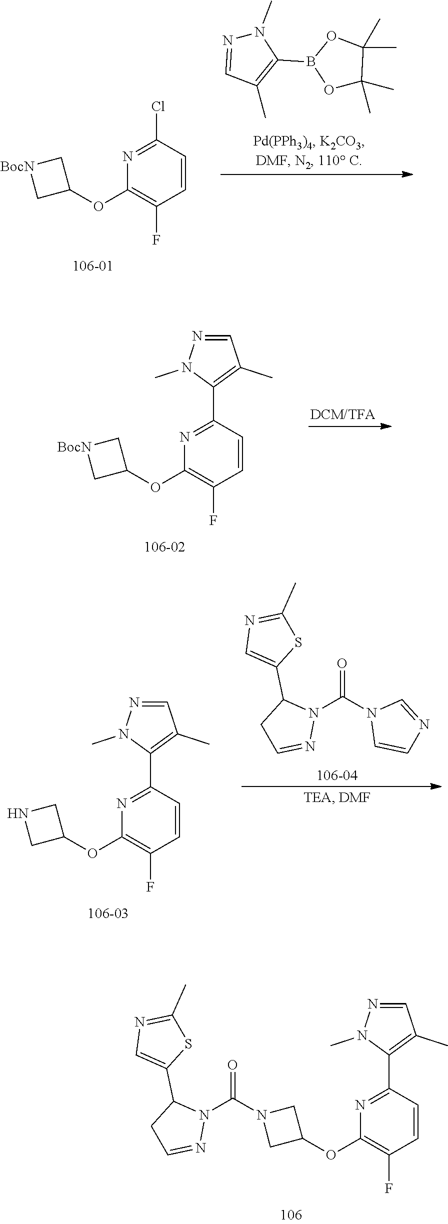 [SMILES: CC(C)(C)OC(=O)N1CC(Oc2nc(Cl)ccc2F)C1 ; Cc1cnn(C)c1B1OC(C)(C)C(C)(C)O1 ; Cc1cnn(C)c1-c1ccc(F)c(OC2CN(C(=O)OC(C)(C)C)C2)n1 ; Cc1cnn(C)c1-c1ccc(F)c(OC2CNC2)n1 ; Cc1ncc(C2CC=NN2C(=O)n2ccnc2)s1 ; Cc1ncc(C2CC=NN2C(=O)N2CC(Oc3nc(-c4c(C)cnn4C)ccc3F)C2)s1]

Step 1: The mixture of 106-01 (1 g, 3.30 mmol), 1,4-dimethyl-5-(4,4,5,5-tetramethyl-1,3,2-dioxaborolan-2-yl)-1H-pyrazole (1.1 g, 4.95 mmol), tetrakis(triphenylphosphine)palladium (173 mg, 0.17 mmol) and Potassium carbonate (911 mg, 6.6 mmol) in 20 mL of DMF were degassed and purged in nitrogen, the mixture was stirred at 110° C. for 16 hrs. After being cooled, to the mixture was added 40 mL of water and extracted with ethyl acetate (50 mL×2). The combined organic layers were successively washed with brine (30 mL) and water (30 mL), dried over sodium sulfate, filtered, and concentrated in vacuo. The residue was purified by column chromatography (Petroleum ether:Ethyl acetate=10:1 to 3:1) to afford the product (1.1 g, yield=92%) as white solid. Mass (m/z) 363 [M+H]⁺.

Step 2: The solution of 106-02 (100 mg, 0.28 mmol) in dioxane (1N HCl) was stirred at rt for 1 h. The reaction mixture was concentrated in vacuo to afford the crude product (80 mg) as brown oil without further purification to use next step. Mass (m/z) 263 [M+H]⁺.

Step 3: The mixture of 106-03 (80 mg, 0.31 mol), 106-04 (79 mg, 0.31 mmol) and TEA (63 mg, 0.62 mmol) in DMF (2 mL) was stirred at rt for 6 hrs. To the mixture was added 4 mL of water and extracted with ethyl acetate (8 mL×2). The combined organic layers were successively washed with brine (5 mL) and water (5 mL), dried over sodium sulfate, filtered, and concentrated in vacuo. The residue was purified by column chromatography (methanol:dichloromethane=1:100 to 3:100) to afford the product 106 (50 mg, yield 36%) as white solid. Mass (m/z) 456 [M+H]⁺. ¹H NMR (400 MHz, MeOH-d₄) δ ppm 7.65 (dd, J=10.0, 8.1 Hz, 1H), 7.48 (s, 1H), 7.32 (s, 1H), 7.15 (dd, J=8.1, 2.9 Hz, 1H), 6.96 (t, J=1.6 Hz, 1H), 5.61-5.52 (m, 1H), 5.48-5.39 (m, 1H), 4.65-4.51 (m, 2H), 4.28-4.12 (m, 1H), 3.89 (s, 3H), 3.44-3.33 (m, 1H), 2.91-2.82 (m, 1H), 2.62 (s, 3H), 2.09 (s, 3H).

### (3-((2-(3-amino-1,4-dimethyl-1H-pyrazol-5-yl)-5-fluoropyridin-4-yl)oxy)azetidin-1-yl)(5-(2-methylthiazol-5-yl)-4,5-dihydro-1H-pyrazol-1-yl) methanone (107)

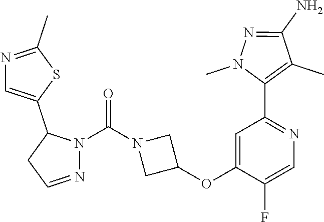 [SMILES: Cc1ncc(C2CC=NN2C(=O)N2CC(Oc3cc(-c4c(C)c(N)nn4C)ncc3F)C2)s1]

The title compound 107 was prepared in a yield of 34% as a light yellow solid according to the procedure outlined for compound 49. Mass (m/z) 471.3 [M+H]⁺. ¹H NMR (400 MHz, Chloroform-d) δ 8.55 (d, J=2.8 Hz, 1H), 7.74 (s, 1H), 6.91 (s, 1H), 6.77 (d, J=6.3 Hz, 1H), 6.13-5.78 (m, 2H), 5.56 (dd, J=11.8, 5.9 Hz, 1H), 5.16-5.04 (m, 1H), 4.70-4.52 (m, 2H), 4.44-4.24 (m, 2H), 3.82 (s, 3H), 3.46-3.33 (m, 1H), 3.05-2.95 (m, 1H), 2.80 (s, 3H), 1.96 (s, 3H).

### (S)-(3-((2-(1,4-dimethyl-1H-pyrazol-5-yl)-5-fluoropyridin-4-yl)oxy)azetidin-1-yl)(5-(2-methylthiazol-5-yl)-4,5-dihydro-1H-pyrazol-1-yl)methanone (108)

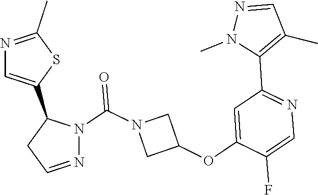 [SMILES: Cc1ncc([C@@H]2CC=NN2C(=O)N2CC(Oc3cc(-c4c(C)cnn4C)ncc3F)C2)s1]

The title compound 108 was prepared according to the procedure outlined for compound 49. ¹H NMR (400 MHz, DMSO-d₆) δ 8.64 (d, J=3.0 Hz, 1H), 7.51 (s, 1H), 7.34 (s, 1H), 7.09 (dd, J=4.2, 2.5 Hz, 2H), 5.50 (dd, J=11.7, 5.8 Hz, 1H), 5.27 (tt, J=6.6, 3.7 Hz, 1H), 4.51 (s, 2H), 4.04 (dd, J=32.3, 10.5 Hz, 2H), 3.85 (s, 3H), 3.43-3.36 (m, 1H), 2.86 (ddd, J=18.6, 5.9, 1.8 Hz, 1H), 2.59 (s, 3H), 2.07 (s, 3H).

### (3-((2-(1,4-dimethyl-1H-pyrazol-5-yl)-5-fluoropyrimidin-4-yl)oxy)azetidin-1-yl)(5-(2-methylthiazol-5-yl)-4,5-dihydro-1H-pyrazol-1-yl)methan one (109)

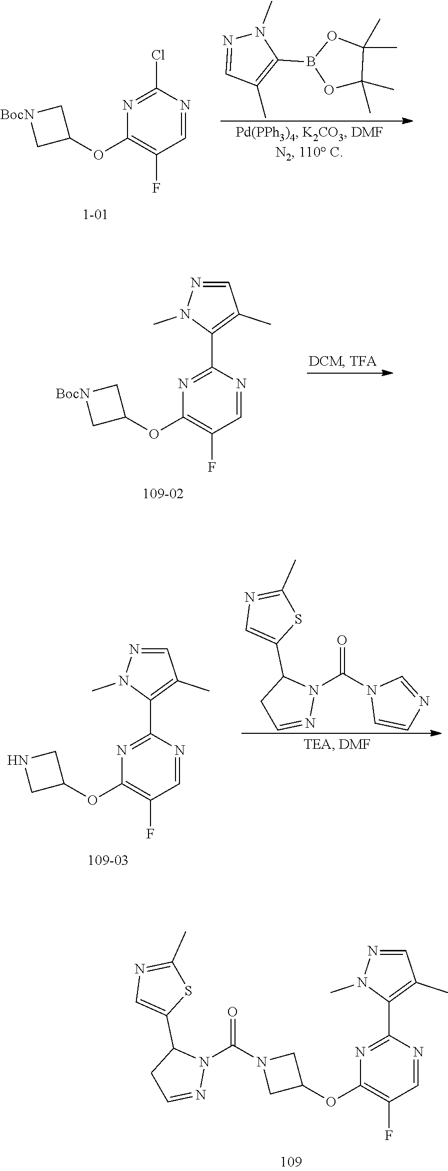 [SMILES: CC(C)(C)OC(=O)N1CC(Oc2nc(Cl)ncc2F)C1 ; Cc1cnn(C)c1B1OC(C)(C)C(C)(C)O1 ; Cc1cnn(C)c1-c1ncc(F)c(OC2CN(C(=O)OC(C)(C)C)C2)n1 ; Cc1cnn(C)c1-c1ncc(F)c(OC2CNC2)n1 ; Cc1ncc(C2CC=NN2C(=O)n2ccnc2)s1 ; Cc1ncc(C2CC=NN2C(=O)N2CC(Oc3nc(-c4c(C)cnn4C)ncc3F)C2)s1]

The titled compound 109 was prepared in 21.3% yield as white solid from 1-01 according to the procedure outlined for compound 106. Mass (m/z) 456.6 [M+H]⁺. ¹H NMR (400 MHz, CDCl₃) δ 8.45 (d, J=2.4 Hz, 1H), 7.53 (s, 1H), 7.35 (s, 1H), 6.84 (s, 1H), 5.63-5.48 (m, 2H), 4.69-4.52 (m, 2H), 4.36 (d, J=6.9 Hz, 1H), 4.25 (dd, J=10.5, 3.4 Hz, 1H), 4.18 (s, 3H), 3.34 (dd, J=18.4, 11.8 Hz, 1H), 2.91 (dd, J=18.9, 5.8 Hz, 1H), 2.68 (s, 3H), 2.31 (s, 3H).

### 3-(5-fluoro-4-((1-(5-(2,3,5-trifluorophenyl)-4,5-dihydro-1H-pyrazole-1-carbonyl)azetidin-3-yl)oxy)pyridin-2-yl)-1,4-dimethyl-1H-pyrazole-5-carboxamide (110)

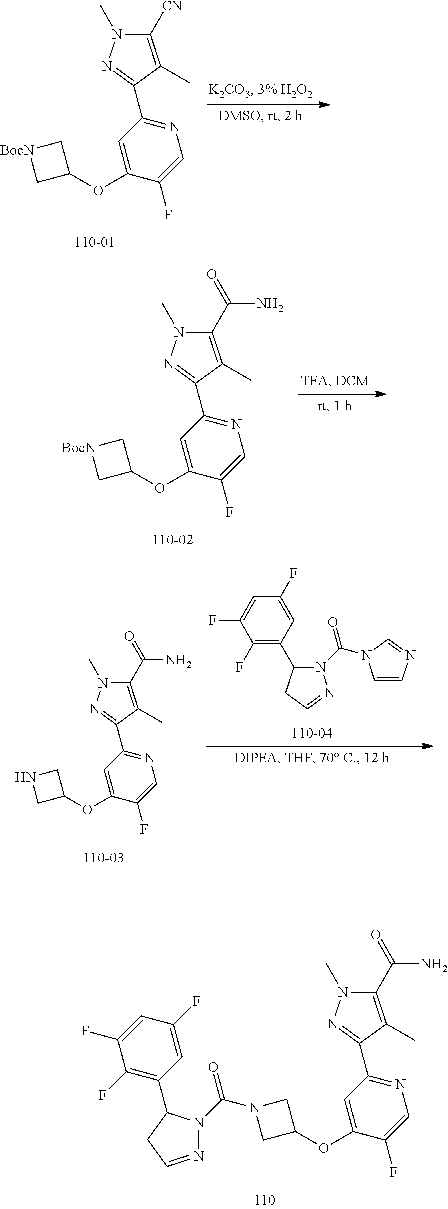 [SMILES: Cc1c(-c2cc(OC3CN(C(=O)OC(C)(C)C)C3)c(F)cn2)nn(C)c1C#N ; Cc1c(-c2cc(OC3CN(C(=O)OC(C)(C)C)C3)c(F)cn2)nn(C)c1C(N)=O ; Cc1c(-c2cc(OC3CNC3)c(F)cn2)nn(C)c1C(N)=O ; O=C(N1N=CCC1c1cc(F)cc(F)c1F)n1ccnc1 ; Cc1c(-c2cc(OC3CN(C(=O)N4N=CCC4c4cc(F)cc(F)c4F)C3)c(F)cn2)nn(C)c1C(N)=O]

Step 1: To a solution of 110-01 (400 mg, 1.03 mmol) in DMSO (40 mL) was added K₂CO₃ (713 mg, 5.16 mmol) and 3% H₂O₂ (4 mL). The reaction mixture was stirred at rt for 2 h. The crude was purified by column chromatography on silica gel to give compound 110-02 (227 mg, 54%) as a white solid. Mass (m/z) 406.1 [M+H]⁺.

Step 2: To a solution of 110-02 (60 mg, 148.0 umol) in DCM (3 mL) was added TFA (3 mL). The reaction mixture was stirred at rt for 1 h. The solvent was concentrated under vacuum. The crude compound 110-03 was used to next step directly Mass (m/z) 306.1 [M+H]⁺.

Step 3: To a solution of 110-03 (50 mg, 169.9 umol) in THE (4 mL) was added 3-(4-(azetidin-3-yloxy)-5-fluoropyridin-2-yl)-1,4-dimethyl-1H-pyrazole-5-carboxamide (52 mg, 169.9 umol) and DIPEA (110 mg, 849.7 umol). The reaction mixture was stirred at 70° C. for 12 h. The crude was purified by Pre-HPLC to give compound 110 (16 mg, 18%) as a white solid. Mass (m/z): 532.1 [M+H]⁺.

### (S)-3-(5-fluoro-4-((1-(5-(3-fluorophenyl)-4,5-dihydro-1H-pyrazole-1-carbonyl)azetidin-3-yl)oxy)pyridin-2-yl)-1,4-dimethyl-1H-pyrazole-5-carb oxamide (111)

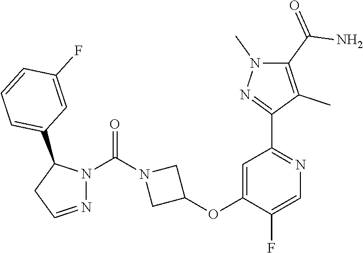 [SMILES: Cc1c(-c2cc(OC3CN(C(=O)N4N=CC[C@H]4c4cccc(F)c4)C3)c(F)cn2)nn(C)c1C(N)=O]

The title compound 111 was prepared in a yield of 22% according to the procedure outlined for compound 98. Mass (m/z) 496.1 [M+H]⁺.

### 1-(5-fluoro-4-((1-(5-(2,3,5-trifluorophenyl)-4,5-dihydro-1H-pyrazole-1-carbonyl)azetidin-3-yl)oxy)pyridin-2-yl)-3,5-dimethyl-1H-pyrazole-4-sulfonamide (112)

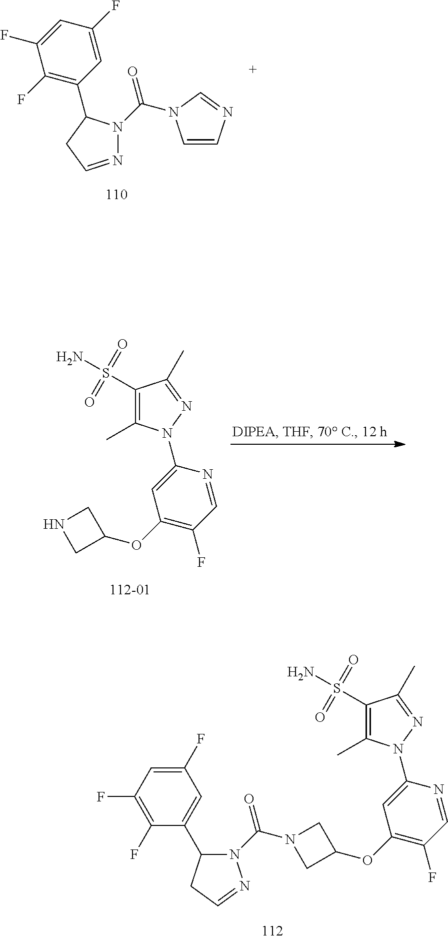 [SMILES: O=C(N1N=CCC1c1cc(F)cc(F)c1F)n1ccnc1 ; Cc1nn(-c2cc(OC3CNC3)c(F)cn2)c(C)c1S(N)(=O)=O ; Cc1nn(-c2cc(OC3CN(C(=O)N4N=CCC4c4cc(F)cc(F)c4F)C3)c(F)cn2)c(C)c1S(N)(=O)=O]

The titled compound 112 was prepared from compound 112-01 in a yield of 24.0% according to the procedure outlined for compound 110. Mass (m/z) 568.5 [M+H]⁺. ¹H NMR (400 MHz, Chloroform-d) δ 8.22 (d, J=2.4 Hz, 1H), 7.14 (d, J=6.0 Hz, 1H), 6.89-6.78 (m, 2H), 6.70 (ddt, J=7.6, 5.2, 2.0 Hz, 1H), 5.51 (dd, J=12.4, 6.8 Hz, 1H), 5.14 (dd, J=6.4, 3.6 Hz, 1H), 4.84 (brs, 2H), 4.69-4.57 (m, 2H), 4.39-4.25 (m, 2H), 3.40 (ddd, J=18.4, 12.4, 1.2 Hz, 1H), 2.82 (s, 3H), 2.73 (dd, J=18.4, 6.4 Hz, 1H), 2.48 (s, 3H).

### (S)-1-(5-fluoro-4-((1-(5-phenyl-4,5-dihydro-1H-pyrazole-1-carbonyl) azetidin-3-yl)oxy)pyridin-2-yl)-3,5-dimethyl-1H-pyrazole-4-sulfonamide (113)

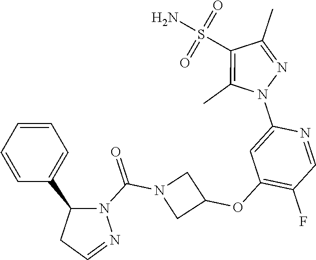 [SMILES: Cc1nn(-c2cc(OC3CN(C(=O)N4N=CC[C@H]4c4ccccc4)C3)c(F)cn2)c(C)c1S(N)(=O)=O]

The titled compound 113 was prepared from compound 112-01 in a yield of 18.5% according to the procedure outlined for compound 110. Mass (m/z) 514.4 [M+H]⁺. ¹H NMR (400 MHz, Chloroform-d) δ 8.21 (d, J=2.4 Hz, 1H), 7.36-7.30 (m, 2H), 7.28-7.24 (m, 1H), 7.25-7.20 (m, 2H), 7.13 (d, J=6.0 Hz, 1H), 6.81-6.78 (m, 1H), 5.31 (dd, J=12.0, 6.4 Hz, 1H), 5.12 (td, J=6.4, 3.2 Hz, 1H), 4.83 (s, 2H), 4.69-4.52 (m, 2H), 4.37-4.20 (m, 2H), 3.36 (ddd, J=18.6, 12.0, 1.6 Hz, 1H), 2.81 (s, 3H), 2.76 (ddd, J=18.4, 6.4, 1.6 Hz, 1H), 2.48 (s, 3H).

### (S)-1-(5-fluoro-4-((1-(5-(3-fluorophenyl)-4,5-dihydro-1H-pyrazole-1-carbonyl)azetidin-3-yl)oxy)pyridin-2-yl)-3,5-dimethyl-1H-pyrazole-4-sulfonamide (114)

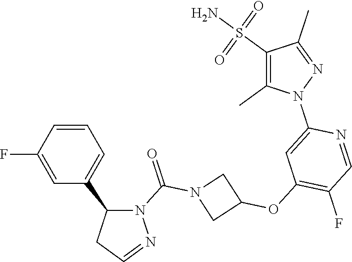 [SMILES: Cc1nn(-c2cc(OC3CN(C(=O)N4N=CC[C@H]4c4cccc(F)c4)C3)c(F)cn2)c(C)c1S(N)(=O)=O]

The titled compound 114 was prepared from compound 112-01 in a yield of 17.0% according to the procedure outlined for compound 110. Mass (m/z) 532.3 [M+H]⁺. ¹H NMR (400 MHz, Chloroform-d) δ 8.22 (d, J=2.4 Hz, 1H), 7.30 (td, J=8.0, 5.6 Hz, 1H), 7.14 (d, J=6.0 Hz, 1H), 7.02 (dt, J=7.6, 1.2 Hz, 1H), 6.98-6.89 (m, 2H), 6.80 (t, J=1.6 Hz, 1H), 5.31 (dd, J=12.0, 6.4 Hz, 1H), 5.13 (td, J=6.4, 3.2 Hz, 1H), 4.81 (brs, 2H), 4.68-4.55 (m, 2H), 4.37-4.21 (m, 2H), 3.36 (ddd, J=18.4, 12.0, 1.6 Hz, 1H), 2.82 (s, 3H), 2.73 (ddd, J=18.4, 6.4, 1.6 Hz, 1H), 2.48 (s, 3H).

### (3-((2-(3-amino-1,4-dimethyl-1H-pyrazol-5-yl)-5-fluoropyridin-4-yl)oxy)azetidin-1-yl)(5-(2,3,5-trifluorophenyl)-4,5-dihydro-1H-pyrazol-1-yl) methanone (115)

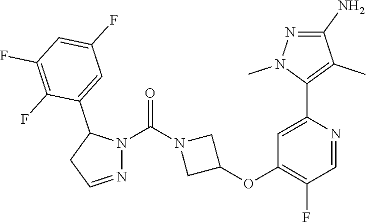 [SMILES: Cc1c(N)nn(C)c1-c1cc(OC2CN(C(=O)N3N=CCC3c3cc(F)cc(F)c3F)C2)c(F)cn1]

The titled compound 115 was prepared in 5.0% yield according to the procedure outlined for compound 107. ¹H NMR (301 MHz, Chloroform-d) δ 8.48 (d, J=3.0 Hz, 1H), 6.84 (d, J=5.4 Hz, 2H), 6.73-6.62 (m, 2H), 5.50 (dd, J=12.3, 6.7 Hz, 1H), 5.06 (tt, J=6.6, 3.9 Hz, 1H), 4.59 (q, J=9.8, 9.3 Hz, 2H), 4.40-4.24 (m, 2H), 3.76 (s, 3H), 3.49-3.35 (m, 1H), 2.74 (dd, J=18.7, 6.7 Hz, 1H), 1.96 (s, 3H). Mass (m/z) 504.3 [M+H]⁺.

### (3-((2-(1,4-dimethyl-1H-pyrazol-5-yl)-5-fluoropyridin-4-yl)oxy)azetidin-1-yl)(5-(2,3,5-trifluorophenyl)-4,5-dihydro-1H-pyrazol-1-yl)methanone (116)

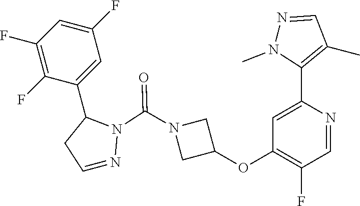 [SMILES: Cc1cnn(C)c1-c1cc(OC2CN(C(=O)N3N=CCC3c3cc(F)cc(F)c3F)C2)c(F)cn1]

The titled compound 116 was prepared in 16.0% yield according to the procedure outlined for compound 11. ¹H NMR (301 MHz, Chloroform-d) δ 8.49 (d, J=2.9 Hz, 1H), 7.35 (s, 1H), 6.83 (s, 2H), 6.69 (d, J=6.6 Hz, 2H), 5.50 (dd, J=12.4, 6.7 Hz, 1H), 5.06 (tt, J=6.5, 3.6 Hz, 1H), 4.59 (q, J=9.1, 8.6 Hz, 2H), 4.43-4.24 (m, 2H), 3.95 (s, 3H), 3.40 (dd, J=18.6, 12.3 Hz, 1H), 2.73 (dd, J=18.6, 6.7 Hz, 1H), 2.11 (s, 3H). Mass (m/z) 489.3 [M+H]⁺.

### 1-(4-((1-(5-(3-chloro-5-fluorophenyl)-4,5-dihydro-1H-pyrazole-1-carbonyl)azetidin-3-yl)oxy)-5-fluoropyridin-2-yl)-3,5-dimethyl-1H-pyrazole-4-carboxamide (117)

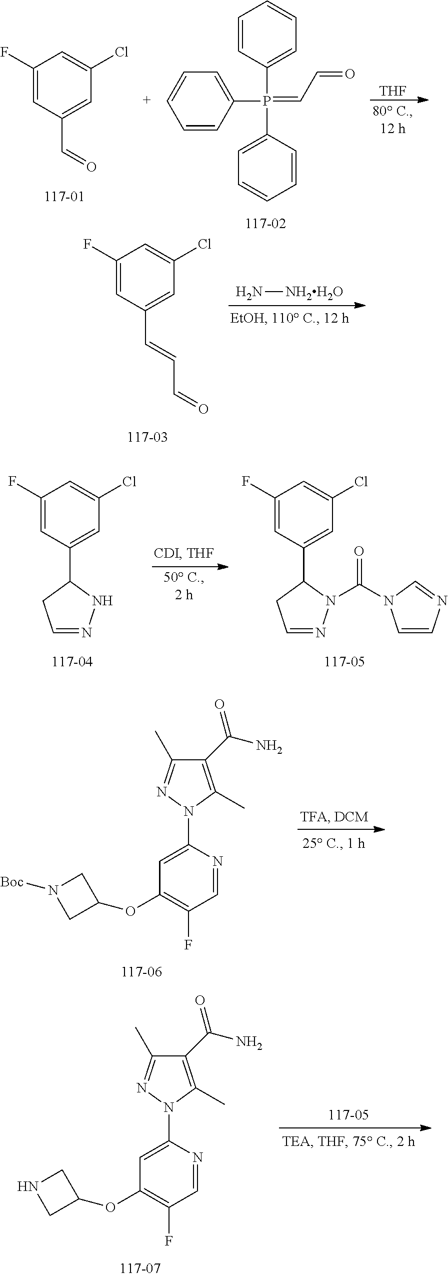

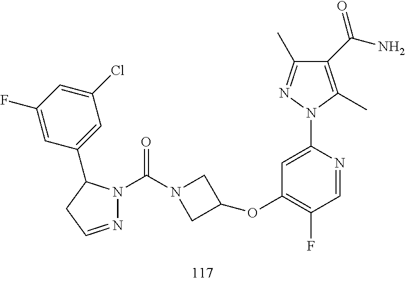 [SMILES: Cc1nn(-c2cc(OC3CN(C(=O)N4N=CCC4c4cc(F)cc(Cl)c4)C3)c(F)cn2)c(C)c1C(N)=O]

Step 1: 117-01 (5.55 g, 35 mmol), 117-02 (10.64 g, 35 mmol) was dissolved in 40 mL of THF, the mixture was stirred at 80° C. for 12 hours. After the mixture was concentrated and further purification by silica gel chromatography to give 117-03 (6.0 g, 92.8%) as a yellow solid.

Step 2: 117-03 (1.0 g, 32.5 mmol), Hydrazine (20 mL, 325 mmol) were dissolved in EtOH (40 mL). The whole reaction mixture was stirred at 110° C. for 12 hours. The mixture was extracted with EA, washed with brine, dried (Na₂SO₄), and concentrated in vacuo. Purification by silica gel chromatography to give 117-04 (6.0 g, 92.9%, crude) as a yellow oil.

Step 3: 117-04 (6.0 g, 30 mmol), CDI (5.9 g, 36 mmol) was dissolved in 40 mL of THF, the mixture was stirred at 50° C. for 2 hours. After the mixture was concentrated and further purification by silica gel chromatography gave 117-05 (4.0 g, 32.9%) as a yellow solid.

Step 4-5: The titled compound 117 was prepared in 19.9% yield according to the procedure outlined for compound 106. ¹H NMR (400 MHz, Chloroform-d) δ 8.20 (d, J=2.5 Hz, 1H), 7.15 (d, J=6.1 Hz, 1H), 7.03-6.93 (m, 2H), 6.87-6.75 (m, 2H), 5.62 (s, 2H), 5.24 (dd, J=12.2, 6.5 Hz, 1H), 5.14 (tt, J=6.4, 4.0 Hz, 1H), 4.61 (d, J=11.4 Hz, 2H), 4.40-4.21 (m, 2H), 3.35 (ddd, J=18.6, 12.2, 1.7 Hz, 1H), 2.80 (s, 3H), 2.69 (ddd, J=18.6, 6.5, 1.8 Hz, 1H), 2.46 (s, 3H). Mass (m/z) 530.2 [M+H]⁺.

### 1-(5-fluoro-4-((1-(5-(3-fluoro-5-methylphenyl)-4,5-dihydro-1H-pyrazole-1-carbonyl)azetidin-3-yl)oxy)pyridin-2-yl)-3,5-dimethyl-1H-pyrazole-4-carboxamide (118)

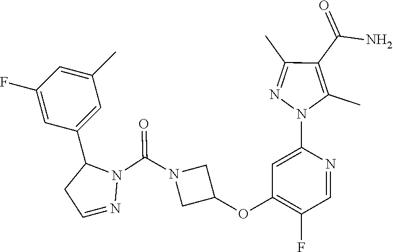 [SMILES: Cc1cc(F)cc(C2CC=NN2C(=O)N2CC(Oc3cc(-n4nc(C)c(C(N)=O)c4C)ncc3F)C2)c1]

The titled compound 118 was prepared in 36.0% yield according to the procedure outlined for compound 117. ¹H NMR (400 MHz, Chloroform-d) δ 8.20 (d, J=2.5 Hz, 1H), 7.15 (d, J=6.1 Hz, 1H), 6.83-6.67 (m, 4H), 5.59 (s, 2H), 5.25 (dd, J=12.2, 6.4 Hz, 1H), 5.14 (tt, J=6.4, 3.9 Hz, 1H), 4.61 (dt, J=16.8, 8.7 Hz, 2H), 4.31 (ddd, J=33.2, 10.7, 3.9 Hz, 2H), 3.34 (ddd, J=18.6, 12.2, 1.7 Hz, 1H), 2.81 (s, 3H), 2.74-2.65 (m, 1H), 2.48 (s, 3H), 2.32 (d, J=0.7 Hz, 3H). Mass (m/z) 510.2 [M+H]⁺.

### (3-((2-(1,4-dimethyl-1H-pyrazol-5-yl)-5-fluoropyridin-4-yl)oxy)azetidin-1-yl)(5-(2,4-dimethylthiazol-5-yl)-4,5-dihydro-1H-pyrazol-1-yl)methanone (119)

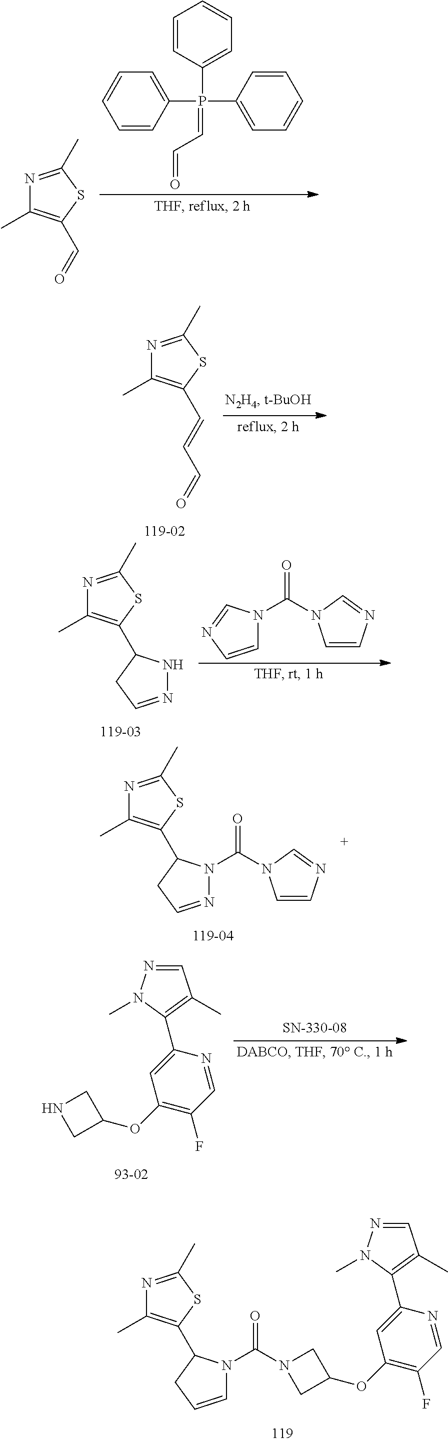

The title compound 119 was prepared in a yield of 38% as a white solid from 119-01 according to the procedure for 117. Mass (m/z) 246.2 [M+H]⁺. ¹H NMR (400 MHz, Chloroform-d) δ 8.48 (d, J=2.9 Hz, 1H), 7.35 (s, 1H), 6.83 (t, J=1.7 Hz, 1H), 6.68 (d, J=6.5 Hz, 1H), 5.52 (dd, J=12.0, 6.3 Hz, 1H), 5.07-4.99 (m, 1H), 4.64-4.55 (m, 1H), 4.54-4.46 (m, 1H), 4.38-4.30 (m, 1H), 4.27-4.19 (m, 1H), 3.95 (s, 3H), 3.34 (ddd, J=18.6, 12.0, 1.7 Hz, 1H), 2.77 (ddd, J=18.6, 6.3, 1.7 Hz, 1H), 2.60 (s, 3H), 2.41 (s, 3H), 2.10 (s, 3H).

### (S)-(3-((2-(1,4-dimethyl-1H-pyrazol-5-yl)-5-fluoropyridin-4-yl)oxy)azetidin-1-yl)(5-(2-methylthiazol-4-yl)-4,5-dihydro-1H-pyrazol-1-yl)methanone (120)

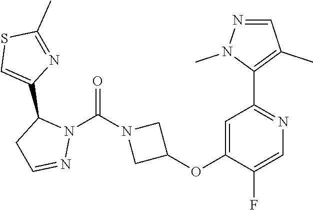 [SMILES: Cc1nc([C@@H]2CC=NN2C(=O)N2CC(Oc3cc(-c4c(C)cnn4C)ncc3F)C2)cs1]

The title compound 120 was prepared as a white solid according to the procedure for 123. ¹H NMR (400 MHz, Chloroform-d) δ 8.48 (d, J=2.9 Hz, 1H), 7.40-7.31 (m, 1H), 7.07 (d, J=0.6 Hz, 1H), 6.85 (d, J=1.7 Hz, 1H), 6.69 (d, J=6.6 Hz, 1H), 5.45 (dd, J=11.7, 6.7 Hz, 1H), 5.03 (td, J=6.4, 3.2 Hz, 1H), 4.66-4.46 (m, 2H), 4.42-4.27 (m, 2H), 3.96 (s, 3H), 3.26 (ddd, J=18.4, 11.7, 1.7 Hz, 1H), 3.15 (ddd, J=18.4, 6.7, 1.7 Hz, 1H), 2.68 (s, 3H), 2.11 (s, 3H).

### (R)-(3-((2-(1,4-dimethyl-1H-pyrazol-5-yl)-5-fluoropyridin-4-yl)oxy)azetidin-1-yl)(5-(2-methylthiazol-4-yl)-4,5-dihydro-1H-pyrazol-1-yl)methanone (121)

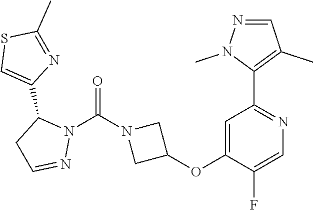 [SMILES: Cc1nc([C@H]2CC=NN2C(=O)N2CC(Oc3cc(-c4c(C)cnn4C)ncc3F)C2)cs1]

The title compound 121 was prepared as a white solid according to the procedure for 123. ¹H NMR (400 MHz, Chloroform-d) δ 8.47 (d, J=2.9 Hz, 1H), 7.35 (d, J=0.7 Hz, 11H), 7.05 (d, J=0.5 Hz, 11H), 6.83 (t, J=1.7 Hz, 11H), 6.68 (d, J=6.6 Hz, 11H), 5.43 (dd, J=11.8, 6.7 Hz, 11H), 5.02 (ddd, J=10.4, 6.4, 3.9 Hz, 1H), 4.68-4.49 (m, 2H), 4.30 (ddd, J=28.9, 10.6, 3.8 Hz, 2H), 3.94 (s, 3H), 3.24 (ddd, J=18.4, 11.8, 1.7 Hz, 1H), 3.20-3.10 (m, 1H), 2.67 (s, 3H), 2.10 (s, 3H).

### (3-((6-(1,4-dimethyl-1H-pyrazol-5-yl)-3,5-difluoropyridin-2-yl)oxy)azetidin-1-yl)(5-(2-methylthiazol-4-yl)-4,5-dihydro-11H-pyrazol-1-yl)methanone (122)

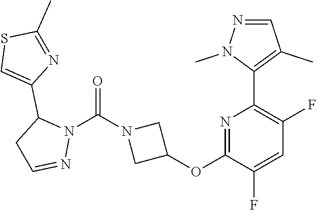 [SMILES: Cc1nc(C2CC=NN2C(=O)N2CC(Oc3nc(-c4c(C)cnn4C)c(F)cc3F)C2)cs1]

The title compound 122 was prepared in a yield of 27% as a white solid according to the procedure for 123. Mass (m/z) 474.1 [M+H]⁺. ¹H NMR (400 MHz, Chloroform-d) δ 7.36 (d, J=17.0 Hz, 2H), 7.05 (s, 1H), 6.81 (s, 1H), 5.51-5.41 (m, 1H), 5.34 (s, 1H), 4.53 (d, J=24.4 Hz, 2H), 4.26 (d, J=25.5 Hz, 2H), 3.81 (s, 3H), 3.24 (dd, J=18.3, 11.9 Hz, 1H), 3.15-3.04 (m, 1H), 2.67 (s, 3H).

### (3-((6-(1,4-dimethyl-1H-pyrazol-5-yl)-3,5-difluoropyridin-2-yl)oxy)azetidin-1-yl)(5-(2-methylthiazol-5-yl)-4,5-dihydro-1H-pyrazol-1-yl)methanone (123)

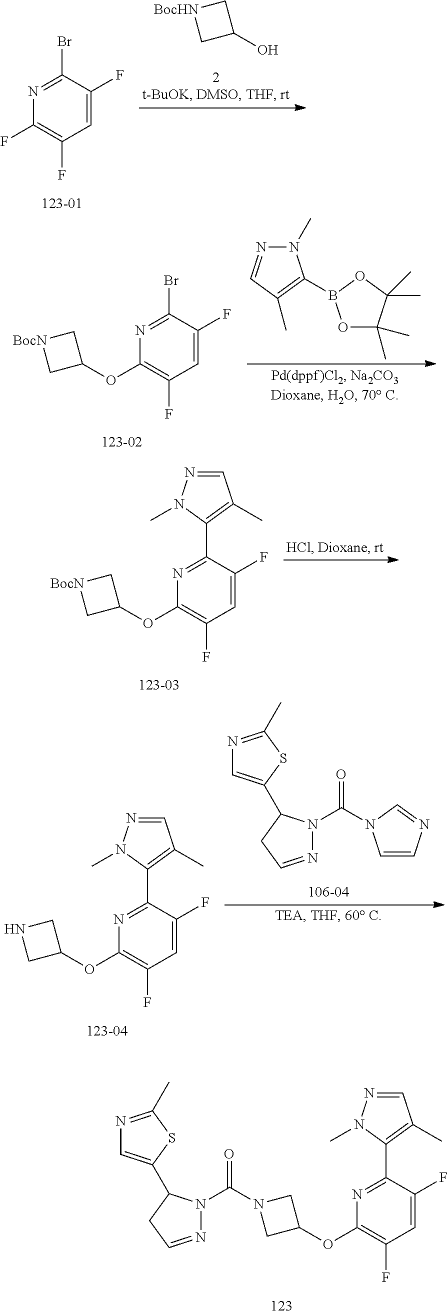

Step 1: To a solution 123-01 (1 g, 4.74 mmol) and t-BuOK (796 mg, 7.11 mmol) in DCM (10 mL), added tert-butyl 3-hydroxyazetidine-1-carboxylate (0.984 g, 5.69 mmol) in DMSO (10 mL) when stirred at rt. after stirred the reaction mixture at rt for 16 hrs, added DCM (50 mL) and washed with water (50×3 mL), collected organic phase, dried with anhydrous Na₂SO₄, filtered, and concentrated under reduced pressure, the crude product was purified by chromatography on silica gel, eluting (PE/EA=4/1) get 123-02 (700 mg, 40.6%) as white solid. Mass (m/z) 365.9 [M+H]⁺.

Step 2: To a solution of 123-02 (650 mg, 1.78 mmol), 1,4-dimethyl-5-(4,4,5,5-tetramethyl-1,3,2-dioxaborolan-2-yl)-1H-pyrazole (593 mg, 2.63 mmol), Pd(dppf)Cl₂ (130 mg, 0.178 mmol), Na₂CO₃ (374 mg, 3.56 mmol) in dioxane (15 mL), put the reaction at N₂ atmosphere, after the reaction stirred at 70° C. for overnight, added DCM (30 mL) and washed with water (30×2 mL), collected organic phase, dried with anhydrous Na₂SO₄, filtered, and concentrated under reduced pressure, the crude product was purified by chromatography on silica gel, eluting (PE/EA=4/1) to get 123-03 (550 mg, 81.3%) as a white solid. Mass (m/z) 380.8 [M+H]⁺.

Step 3: To a solution of 123-03 (250 mg, 0.66 mmol) in HCl/dioxane (4N,10 mL), stirred the reaction at rt for 2 hrs., removed the solvent at reduced pressure. The crude product was washed with tert-Butyl methyl ether (5 mL×2), obtained 123-04 (170 mg, 91.2%) as white solid. Mass (m/z) 280.8 [M+H]⁺.

Step 4: To a solution of 123-04 (170 mg, 0.61 mmol), 106-04 (159 mg, 0.61 mmol) and TEA (93 mg, 0.92 mmol) in THE (10 mL), stirred the reaction at 60° C. overnight. Removed the solvent at reduced pressure. The crude was purified by chromatography on silica gel, eluting (DCM/MeOH=30/1) get 123 (80 mg, 27.7%) as a white solid. Mass (m/z) 473.9 [M+H]⁺. ¹H NMR (400 MHz, DMSO-d₆) δ 10.10-10.09 (m, 1H), 7.50 (s, 1H), 7.37 (s, 1H), 7.08 (t, J=1.6 Hz, 1H), 5.54-5.43 (m, 1H), 5.40-5.32 (m, 1H), 4.55-4.24 (m, 2H), 4.19-3.96 (m, 2H), 3.75 (s, 3H), 2.93-2.79 (m, 1H), 2.58 (s, 3H), 2.46-2.28 (m, 1H), 1.95 (d, J=1.6 Hz, 3H).

### (S)-1-(5-fluoro-4-((1-(5-(2,3,5-trifluorophenyl)-4,5-dihydro-1H-pyrazole-1-carbonyl)azetidin-3-yl)oxy)pyridin-2-yl)-3,5-dimethyl-1H-pyrazole-4-sulfonamide (124)

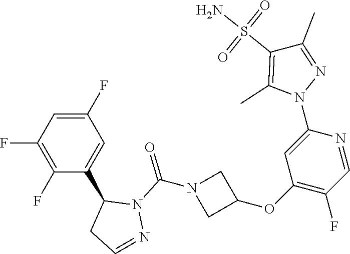 [SMILES: Cc1nn(-c2cc(OC3CN(C(=O)N4N=CC[C@H]4c4cc(F)cc(F)c4F)C3)c(F)cn2)c(C)c1S(N)(=O)=O]

The titled compound 124 was prepared in a yield of 20.0% according to the procedure outlined for compound 113. Mass (m/z) 568.3 [M+H]⁺. ¹H NMR (400 MHz, Chloroform-d) δ 8.23 (d, J=2.4 Hz, 1H), 7.14 (d, J=6.0 Hz, 1H), 6.89-6.78 (m, 2H), 6.74-6.63 (m, 1H), 5.51 (dd, J=12.4, 6.7 Hz, 1H), 5.19-5.10 (m, 1H), 4.82 (brs, 2H), 4.69-4.57 (s, 2H), 4.41-4.26 (m, 1H), 3.40 (dd, J=18.4, 12.4 Hz, 1H), 2.82 (s, 3H), 2.73 (dd, J=18.4, 6.8 Hz, 1H), 2.49 (s, 3H).

### (R)-1-(5-fluoro-4-((1-(5-(2,3,5-trifluorophenyl)-4,5-dihydro-1H-pyrazole-1-carbonyl)azetidin-3-yl)oxy)pyridin-2-yl)-3,5-dimethyl-1H-pyrazole-4-sulfonamide (125)

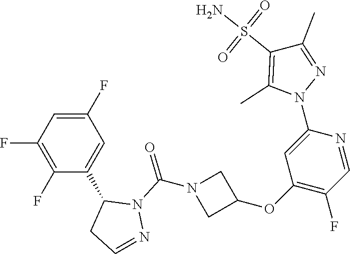 [SMILES: Cc1nn(-c2cc(OC3CN(C(=O)N4N=CC[C@@H]4c4cc(F)cc(F)c4F)C3)c(F)cn2)c(C)c1S(N)(=O)=O]

The titled compound 125 was prepared in a yield of 21.0% according to the procedure outlined for compound 113. Mass (m/z) 568.4 [M+H]⁺. ¹H NMR (400 MHz, Chloroform-d) δ 8.22 (d, J=2.4 Hz, 11H), 7.14 (d, J=6.0 Hz, 1H), 6.87-6.79 (m, 2H), 6.70 (dp, J=7.8, 2.6 Hz, 1H), 5.51 (dd, J=12.4, 6.8 Hz, 1H), 5.15 (tt, J=6.4, 4.0 Hz, 1H), 4.86 (brs, 2H), 4.69-4.57 (m, 2H), 4.40-4.25 (m, 2H), 3.40 (ddd, J=18.4, 12.4, 1.6 Hz, 1H), 2.82 (s, 3H), 2.79-2.67 (m, 1H), 2.48 (s, 3H).

### 3-(5-fluoro-4-((1-(5-(2,3,5-trifluorophenyl)-4,5-dihydro-1H-pyrazole-1-carbonyl)azetidin-3-yl)oxy)pyridin-2-yl)-1,4-dimethyl-1H-pyrazole-5-carboxamide (126)

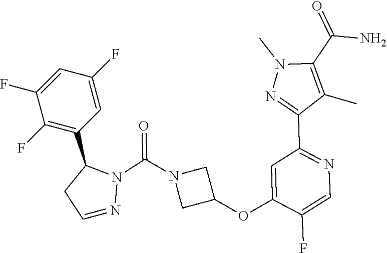 [SMILES: Cc1c(-c2cc(OC3CN(C(=O)N4N=CC[C@H]4c4cc(F)cc(F)c4F)C3)c(F)cn2)nn(C)c1C(N)=O]

The titled compound 126 was prepared according to the procedure outlined for compound 110. Mass (m/z) 532.2 [M+H]⁺.

### (3-((5-chloro-2-(1,4-dimethyl-1H-pyrazol-5-yl)pyridin-4-yl)oxy)azetidin-1-yl)(5-(2-methylthiazol-4-yl)-4,5-dihydro-1H-pyrazol-1-yl)methanone (127)

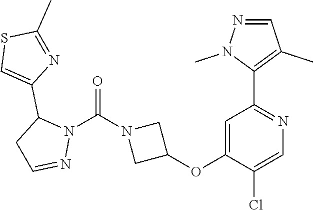 [SMILES: Cc1nc(C2CC=NN2C(=O)N2CC(Oc3cc(-c4c(C)cnn4C)ncc3Cl)C2)cs1]

The titled compound 127 was prepared in a yield of 23% according to the procedure outlined for compound 11. ¹H NMR (400 MHz, Chloroform-d) δ 7.65 (d, J=2.0 Hz, 1H), 7.39 (d, J=1.9 Hz, OH), 7.35-7.27 (m, 3H), 7.25-7.19 (m, 2H), 7.13 (ddd, J=14.7, 8.1, 1.2 Hz, 1H), 6.98 (dd, J=7.6, 1.2 Hz, 1H), 6.90 (dd, J=7.6, 1.2 Hz, OH), 6.42-6.37 (m, 1H), 6.17 (s, 1H), 5.97-5.93 (m, 1H), 4.63 (s, 1H), 4.24 (s, 5H), 4.05 (d, J=2.6 Hz, 2H), 3.71 (d, J=6.2 Hz, 3H).

### (3-((2-(1,4-dimethyl-1H-pyrazol-5-yl)-5-fluoropyridin-4-yl)oxy)azetidin-1-yl)(5-(2,5-dimethylthiazol-4-yl)-4,5-dihydro-1H-pyrazol-1-yl)methanone (128)

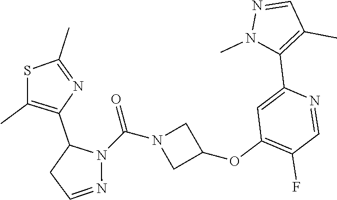

The title compound 128 was prepared in a yield of 67% as a white solid according to the procedure outlined for compound 94. Mass (m/z) 470.3 [M+H]⁺. ¹H NMR (400 MHz, Chloroform-d) δ 8.47 (d, J=2.9 Hz, 1H), 7.36-7.34 (m, 1H), 6.87-6.85 (m, 1H), 6.66 (d, J=6.5 Hz, 1H), 5.31 (dd, J=11.6, 7.5 Hz, 1H), 5.04-4.96 (m, 1H), 4.62-4.46 (m, 2H), 4.38-4.30 (m, 1H), 4.28-4.21 (m, 1H), 3.94 (s, 3H), 3.24-3.04 (m, 2H), 2.58 (s, 3H), 2.48 (s, 3H), 2.09 (s, 3H).

### (3-((6-(1,4-dimethyl-1H-pyrazol-5-yl)-3,5-difluoropyridin-2-yl)oxy)azetidin-1-yl)(5-(5-methylthiazol-2-yl)-4,5-dihydro-1H-pyrazol-1-yl)methanone (129)

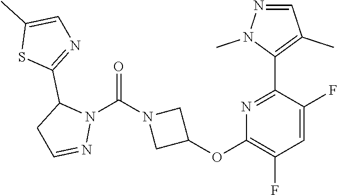

The titled compound 129 was prepared in 2.1% yield according to the procedure outlined for compound 123. ¹H NMR (400 MHz, Chloroform-d) δ 7.40-7.32 (m, 3H), 6.83 (t, J=1.7 Hz, 1H), 5.60 (dd, J=11.8, 6.2 Hz, 1H), 5.36 (tt, J=6.6, 4.2 Hz, 1H), 4.65-4.48 (m, 2H), 4.36-4.19 (m, 2H), 3.81 (s, 3H), 3.40 (ddd, J=18.5, 6.2, 1.8 Hz, 1H), 3.30-3.20 (m, 1H), 2.42 (d, J=1.2 Hz, 3H), 2.01 (d, J=1.8 Hz, 3H). Mass (m/z) 474.3 [M+H]⁺.

### (3-((2-(1,4-dimethyl-1H-pyrazol-5-yl)-5-fluoropyridin-4-yl)oxy)azetidin-1-yl)(5-(3-fluoro-5-methylphenyl)-4,5-dihydro-1H-pyrazol-1-yl)methanone (130)

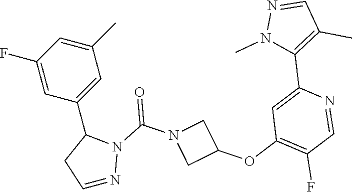 [SMILES: Cc1cc(F)cc(C2CC=NN2C(=O)N2CC(Oc3cc(-c4c(C)cnn4C)ncc3F)C2)c1]

The title compound 130 was prepared in a yield of 48% as a white solid according to the procedure outlined for compound 11. Mass (m/z) 467.3 [M+H]⁺. ¹H NMR (400 MHz, Chloroform-d) δ 8.49 (d, J=2.9 Hz, 1H), 7.37-7.36 (m, 1H), 6.82 (s, 1H), 6.80 (t, J=1.7 Hz, 1H), 6.78-6.74 (m, 1H), 6.73-6.68 (m, 2H), 5.24 (dd, J=12.2, 6.4 Hz, 1H), 5.09-5.02 (m, 1H), 4.64-4.51 (m, 2H), 4.40-4.24 (m, 2H), 3.97 (s, 3H), 3.34 (ddd, J=18.6, 12.2, 1.7 Hz, 1H), 2.72 (ddd, J=18.6, 6.4, 1.7 Hz, 1H), 2.34-2.31 (m, 3H), 2.12 (s, 3H).

### (3-(5-(1,4-dimethyl-1H-pyrazol-5-yl)-2,4-difluorophenoxy)azetidin-1-yl)(5-(2-methylthiazol-4-yl)-4,5-dihydro-1H-pyrazol-1-yl)methanone (131)

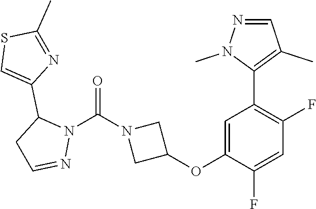 [SMILES: Cc1nc(C2CC=NN2C(=O)N2CC(Oc3cc(-c4c(C)cnn4C)c(F)cc3F)C2)cs1]

The title compound 131 was prepared in a yield of 11% as a white solid according to the procedure outlined for compound 123. Mass (m/z) 473.3 [M+H]⁺. ¹H NMR (400 MHz, Chloroform-d) δ 7.52-7.40 (m, 1H), 7.18-6.98 (m, 2H), 6.92-6.80 (m, 1H), 6.65-6.52 (m, 1H), 5.45 (dd, J=12.2, 7.0 Hz, 1H), 5.01-4.86 (m, 1H), 4.65-4.41 (m, 2H), 4.40-4.19 (m, 2H), 3.73 (s, 3H), 3.46-3.05 (m, 2H), 2.70 (s, 3H), 1.96 (s, 3H).

### (3-(2,4-difluoro-5-(1-methyl-1H-pyrazol-5-yl)phenoxy)azetidin-1-yl)(5-(2-methylthiazol-4-yl)-4,5-dihydro-1H-pyrazol-1-yl)methanone (132)

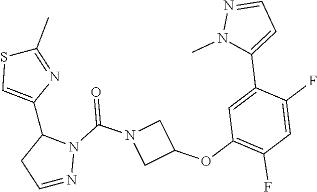 [SMILES: Cc1nc(C2CC=NN2C(=O)N2CC(Oc3cc(-c4ccnn4C)c(F)cc3F)C2)cs1]

The title compound 132 was prepared in a yield of 25% as a white solid according to the procedure outlined for compound 123. Mass (m/z) 459.3 [M+H]⁺. ¹H NMR (400 MHz, Chloroform-d) δ 7.58 (d, J=2.0 Hz, 11H), 7.09 (s, 11H), 7.02 (dd, J=10.6, 9.1 Hz, 11H), 6.84 (t, J=1.5 Hz, 11H), 6.65 (dd, J=8.9, 6.7 Hz, 1H), 6.31 (d, J=2.0 Hz, 1H), 5.47 (dd, J=11.9, 6.7 Hz, 1H), 4.99-4.88 (m, OH), 4.59-4.42 (m, 2H), 4.35-4.18 (m, 2H), 3.81 (d, J=1.6 Hz, 3H), 3.27 (ddd, J=18.4, 11.9, 1.7 Hz, 1H), 3.11 (ddd, J=18.4, 6.7, 1.7 Hz, 1H), 2.72 (s, 3H).

### (S)-1-(4-((1-(5-(3-chloro-5-fluorophenyl)-4,5-dihydro-1H-pyrazole-1-carbonyl)azetidin-3-yl)oxy)-5-fluoropyridin-2-yl)-3,5-dimethyl-1H-pyrazole-4-carboxamide (133)

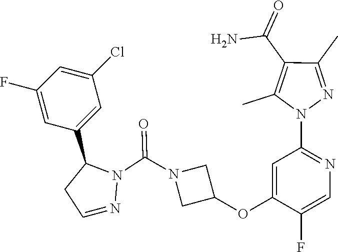 [SMILES: Cc1nn(-c2cc(OC3CN(C(=O)N4N=CC[C@H]4c4cc(F)cc(Cl)c4)C3)c(F)cn2)c(C)c1C(N)=O]

The title compound 133 was prepared as a white solid according to the procedure outlined for compound 126. ¹H NMR (400 MHz, Chloroform-d) δ 8.22 (d, J=2.5 Hz, 1H), 7.18 (d, J=6.1 Hz, 1H), 7.06-6.93 (m, 2H), 6.92-6.76 (m, 2H), 5.75 (s, 2H), 5.26 (dd, J=12.1, 6.5 Hz, 1H), 5.16 (td, J=6.4, 3.3 Hz, 1H), 4.63 (s, 2H), 4.32 (dd, J=32.6, 10.2 Hz, 2H), 3.36 (ddd, J=18.7, 12.2, 1.7 Hz, 1H), 2.82 (s, 3H), 2.71 (ddd, J=18.7, 6.5, 1.7 Hz, 1H), 2.49 (s, 3H).

### (R)-1-(4-((1-(5-(3-chloro-5-fluorophenyl)-4,5-dihydro-1H-pyrazole-1-carbonyl)azetidin-3-yl)oxy)-5-fluoropyridin-2-yl)-3,5-dimethyl-1H-pyrazole-4-carboxamide (134)

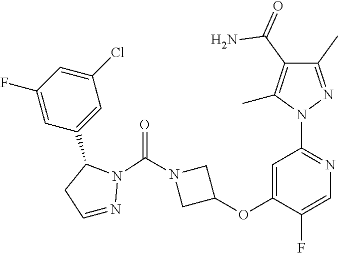 [SMILES: Cc1nn(-c2cc(OC3CN(C(=O)N4N=CC[C@@H]4c4cc(F)cc(Cl)c4)C3)c(F)cn2)c(C)c1C(N)=O]

The title compound 134 was prepared as a white solid according to the procedure outlined for compound 126. ¹H NMR (400 MHz, Chloroform-d) δ 8.22 (d, J=2.4 Hz, 1H), 7.17 (d, J=6.1 Hz, 1H), 7.05-6.96 (m, 2H), 6.90-6.79 (m, 2H), 5.69 (s, 2H), 5.33-5.24 (m, 1H), 5.16 (s, 1H), 4.63 (s, 2H), 4.32 (dd, J=32.8, 10.7 Hz, 2H), 3.43-3.32 (m, 1H), 2.82 (s, 3H), 2.71 (ddd, J=18.7, 6.5, 1.7 Hz, 1H), 2.49 (s, 3H).

### (S)-1-(5-fluoro-4-((1-(5-(3-fluoro-5-methylphenyl)-4,5-dihydro-1H-pyrazole-1-carbonyl)azetidin-3-yl)oxy)pyridin-2-yl)-3,5-dimethyl-1H-pyrazole-4-carboxamide (135)

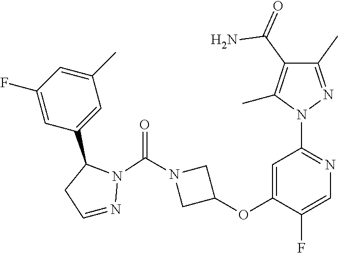 [SMILES: Cc1cc(F)cc([C@@H]2CC=NN2C(=O)N2CC(Oc3cc(-n4nc(C)c(C(N)=O)c4C)ncc3F)C2)c1]

The title compound 135 was prepared as a white solid according to the procedure outlined for compound 126. ¹H NMR (400 MHz, Chloroform-d) δ 8.21 (d, J=2.4 Hz, 1H), 7.16 (d, J=6.1 Hz, 1H), 6.89-6.67 (m, 4H), 5.66 (s, 2H), 5.25 (dd, J=12.2, 6.3 Hz, 1H), 5.14 (q, J=3.2, 2.7 Hz, 1H), 4.63 (dd, J=15.9, 9.8 Hz, 2H), 4.31 (dd, J=33.3, 10.3 Hz, 2H), 3.34 (ddd, J=18.6, 12.2, 1.7 Hz, 1H), 2.81 (s, 3H), 2.71 (ddd, J=18.5, 6.4, 1.7 Hz, 1H), 2.48 (s, 3H), 2.33 (s, 3H).

### (R)-1-(5-fluoro-4-((1-(5-(3-fluoro-5-methylphenyl)-4,5-dihydro-1H-pyrazole-1-carbonyl)azetidin-3-yl)oxy)pyridin-2-yl)-3,5-dimethyl-1H-pyrazole-4-carboxamide (136)

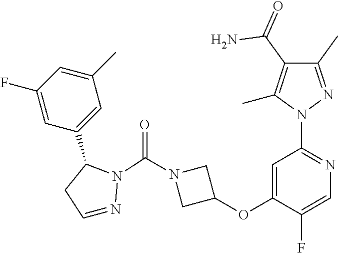 [SMILES: Cc1cc(F)cc([C@H]2CC=NN2C(=O)N2CC(Oc3cc(-n4nc(C)c(C(N)=O)c4C)ncc3F)C2)c1]

The title compound 136 was prepared as a white solid according to the procedure outlined for compound 126. ¹H NMR (400 MHz, Chloroform-d) δ 8.21 (d, J=2.4 Hz, 1H), 7.15 (d, J=6.1 Hz, 1H), 6.80 (dd, J=9.5, 7.8 Hz, 2H), 6.76-6.68 (m, 2H), 5.60 (s, 2H), 5.25 (dd, J=12.1, 6.4 Hz, 1H), 5.14 (td, J=6.4, 3.2 Hz, 1H), 4.63 (dd, J=17.5, 10.4 Hz, 2H), 4.31 (dd, J=33.0, 9.7 Hz, 2H), 3.34 (ddd, J=18.6, 12.2, 1.7 Hz, 1H), 2.81 (s, 3H), 2.71 (ddd, J=18.6, 6.4, 1.7 Hz, 1H), 2.48 (s, 3H), 2.33 (s, 3H).

### (R)-(3-((2-(1,4-dimethyl-1H-pyrazol-5-yl)-5-fluoropyridin-4-yl)oxy)azetidin-1-yl)(5-(5-methylthiazol-2-yl)-4,5-dihydro-1H-pyrazol-1-yl)methanone (137)

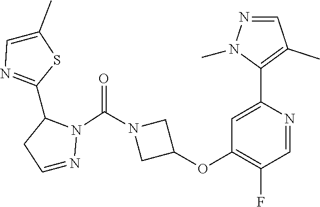 [SMILES: Cc1cnc(C2CC=NN2C(=O)N2CC(Oc3cc(-c4c(C)cnn4C)ncc3F)C2)s1]

The title compound 137 was prepared as a white solid according to the procedure outlined for compound 11. ¹H NMR (400 MHz, Chloroform-d) δ 8.49 (d, J=3.1 Hz, 1H), 7.38 (t, J=5.3 Hz, 2H), 6.86 (s, 1H), 6.70 (d, J=6.4 Hz, 1H), 5.61 (dd, J=13.2, 5.2 Hz, 1H), 5.05 (s, 1H), 4.61 (dd, J=18.9, 10.9 Hz, 2H), 4.34 (dd, J=35.9, 10.6 Hz, 2H), 3.98 (d, J=3.3 Hz, 3H), 3.30 (dd, J=19.5, 11.5 Hz, 1H), 2.44 (s, 3H), 2.13 (s, 3H), 1.66 (s, 1H).

### (S)-(3-((2-(1,4-dimethyl-1H-pyrazol-5-yl)-5-fluoropyridin-4-yl)oxy)azetidin-1-yl)(5-(5-methylthiazol-2-yl)-4,5-dihydro-1H-pyrazol-1-yl)methanone (138)

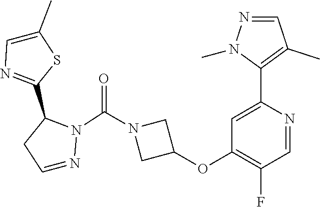 [SMILES: Cc1cnc([C@@H]2CC=NN2C(=O)N2CC(Oc3cc(-c4c(C)cnn4C)ncc3F)C2)s1]

The title compound 138 was prepared as a white solid according to the procedure outlined for compound 11. ¹H NMR (400 MHz, Chloroform-d) δ 8.51 (s, 1H), 7.41 (d, J=14.1 Hz, 2H), 6.87 (s, 1H), 6.72 (d, J=6.4 Hz, 1H), 5.74-5.61 (m, 1H), 5.06 (s, 1H), 4.61 (s, 2H), 4.35 (dd, J=34.7, 10.5 Hz, 2H), 4.02 (s, 3H), 3.36 (d, J=12.0 Hz, 1H), 2.45 (s, 3H), 2.13 (s, 3H), 1.53 (s, 1H).

### (3-((3,5-difluoro-6-(1-methyl-1H-pyrazol-5-yl)pyridin-2-yl)oxy)azetidin-1-yl)(5-(5-methylthiazol-2-yl)-4,5-dihydro-1H-pyrazol-1-yl)methanone (139)

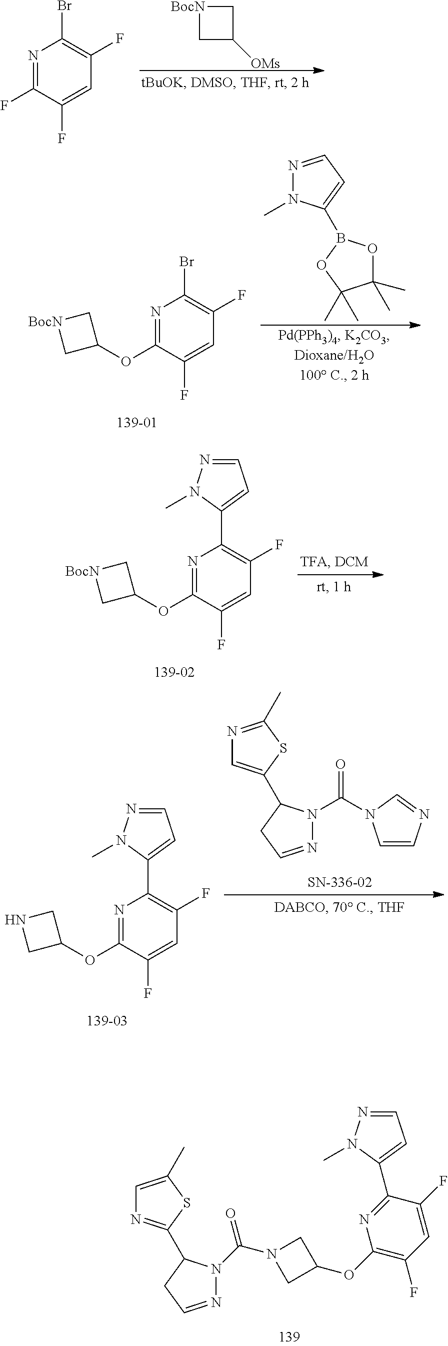

The title compound 139 was prepared in a yield of 7% as a white solid according to the procedure outlined for compound 123. Mass (m/z) 460.2 [M+H]⁺. ¹H NMR (400 MHz, Chloroform-d) δ 7.54 (d, J=2.1 Hz, 1H), 7.41-7.32 (m, 2H), 6.85-6.82 (m, 1H), 6.69-6.65 (m, 1H), 5.60 (dd, J=11.9, 6.2 Hz, 1H), 5.45-5.36 (m, 1H), 4.67-4.47 (m, 2H), 4.39-4.19 (m, 2H), 4.12 (s, 3H), 3.40 (ddd, J=18.5, 6.2, 1.8 Hz, 1H), 3.27 (ddd, J=18.5, 11.8, 1.6 Hz, 1H), 2.45-2.41 (m, 3H).

### (S)-1-(4-((1-(5-(3-chloro-5-fluorophenyl)-4,5-dihydro-1H-pyrazole-1-carbonyl)azetidin-3-yl)oxy)-5-fluoropyridin-2-yl)-3,5-dimethyl-1H-pyrazole-4-carboxylic acid (140)

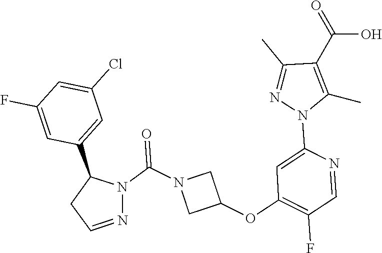 [SMILES: Cc1nn(-c2cc(OC3CN(C(=O)N4N=CC[C@H]4c4cc(F)cc(Cl)c4)C3)c(F)cn2)c(C)c1C(=O)O]

The title compound 140 was prepared according to the procedure outlined for compound 82. ¹H NMR (400 MHz, Chloroform-d) δ 8.24 (d, J=2.3 Hz, 1H), 7.18 (d, J=6.1 Hz, 1H), 7.04-6.95 (m, 2H), 6.84 (dt, J=9.1, 2.0 Hz, 11H), 6.80 (t, J=1.7 Hz, 1H), 5.27 (dd, J=12.2, 6.5 Hz, 1H), 5.21-5.11 (m, 1H), 4.64 (d, J=8.6 Hz, 2H), 4.33 (dd, J=31.3, 10.3 Hz, 2H), 3.36 (ddd, J=18.6, 12.2, 1.7 Hz, 1H), 2.88 (s, 3H), 2.71 (ddd, J=18.7, 6.5, 1.8 Hz, 1H), 2.51 (s, 3H).

### (R)-1-(4-((1-(5-(3-chloro-5-fluorophenyl)-4,5-dihydro-1H-pyrazole-1-carbonyl)azetidin-3-yl)oxy)-5-fluoropyridin-2-yl)-3,5-dimethyl-1H-pyrazole-4-carboxylic acid (141)

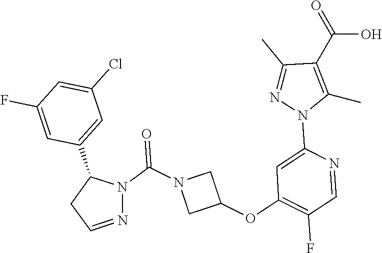 [SMILES: Cc1nn(-c2cc(OC3CN(C(=O)N4N=CC[C@@H]4c4cc(F)cc(Cl)c4)C3)c(F)cn2)c(C)c1C(=O)O]

The title compound 141 was prepared according to the procedure outlined for compound 82. ¹H NMR (400 MHz, Chloroform-d) δ 8.23 (d, J=2.4 Hz, 1H), 7.18 (d, J=6.1 Hz, 1H), 7.05-6.97 (m, 2H), 6.84 (dt, J=9.1, 1.9 Hz, 1H), 6.80 (t, J=1.7 Hz, 1H), 5.27 (dd, J=12.2, 6.5 Hz, 1H), 5.16 (td, J=6.4, 3.2 Hz, 1H), 4.64 (s, 2H), 4.33 (dd, J=31.7, 10.2 Hz, 2H), 3.36 (ddd, J=18.6, 12.2, 1.7 Hz, 1H), 2.88 (s, 3H), 2.71 (ddd, J=18.6, 6.5, 1.7 Hz, 1H), 2.51 (s, 3H).

### (S)-1-(5-fluoro-4-((1-(5-(3-fluoro-5-methylphenyl)-4,5-dihydro-1H-pyrazole-1-carbonyl)azetidin-3-yl)oxy)pyridin-2-yl)-3,5-dimethyl-1H-pyrazole-4-carboxylic acid (142)

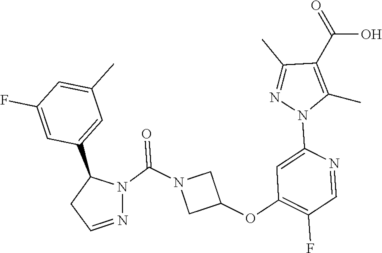 [SMILES: Cc1cc(F)cc([C@@H]2CC=NN2C(=O)N2CC(Oc3cc(-n4nc(C)c(C(=O)O)c4C)ncc3F)C2)c1]

The title compound 142 was prepared according to the procedure outlined for compound 82.

¹H NMR (400 MHz, Chloroform-d) δ 8.23 (s, 1H), 7.18 (d, J=5.9 Hz, 11H), 6.81 (d, J=9.2 Hz, 2H), 6.73 (dd, J=22.6, 9.4 Hz, 2H), 5.26 (dd, J=12.7, 6.3 Hz, 1H), 5.15 (s, 1H), 4.73-4.54 (m, 2H), 4.32 (dd, J=31.3, 10.5 Hz, 2H), 3.34 (dd, J=18.7, 12.2 Hz, 1H), 2.88 (d, J=2.3 Hz, 3H), 2.71 (dd, J=18.8, 6.4 Hz, 1H), 2.51 (s, 3H), 2.33 (s, 3H)

### (R) 1-(5-fluoro-4-((1-(5-(3-fluoro-5-methylphenyl)-4,5-dihydro-1H-pyrazole-1-carbonyl)azetidin-3-yl)oxy)pyridin-2-yl)-3,5-dimethyl-1H-pyrazole-4-carboxylic acid (143)

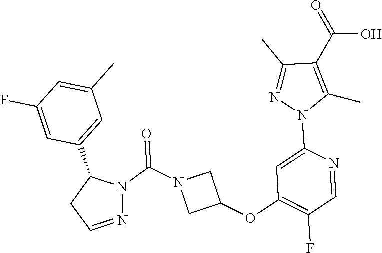 [SMILES: Cc1cc(F)cc([C@H]2CC=NN2C(=O)N2CC(Oc3cc(-n4nc(C)c(C(=O)O)c4C)ncc3F)C2)c1]

The title compound 143 was prepared according to the procedure outlined for compound 82.

¹H NMR (400 MHz, Chloroform-d) δ 8.23 (s, 1H), 7.18 (d, J=6.0 Hz, 1H), 6.80 (d, J=11.5 Hz, 2H), 6.73 (dd, J=21.5, 9.4 Hz, 2H), 5.26 (dd, J=12.7, 6.3 Hz, 1H), 5.15 (d, J=7.3 Hz, 1H), 4.61 (d, J=17.7 Hz, 2H), 4.32 (dd, J=31.5, 10.6 Hz, 2H), 3.34 (dd, J=18.7, 12.2 Hz, 1H), 2.88 (d, J=2.2 Hz, 3H), 2.71 (dd, J=18.5, 6.4 Hz, 1H) 2.51 (s, 3H), 2.33 (s, 3H).

### (3-((2-(1,4-dimethyl-1H-pyrazol-5-yl)-5-fluoropyridin-4-yl)oxy)azetidin-1-yl)(5-(4,5-dimethylthiazol-2-yl)-4,5-dihydro-1H-pyrazol-1-yl)methanone (144)

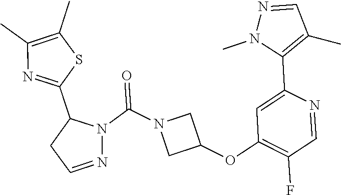

The titled compound 144 was prepared in a yield of 27.4% according to the procedure outlined for compound 11. Mass (m/z) 470.4 [M+H]⁺. ¹H NMR (400 MHz, Chloroform-d) δ 8.48 (d, J=2.8 Hz, 1H), 7.38-7.33 (m, 1H), 6.83 (t, J=1.6 Hz, 1H), 6.69 (d, J=6.4 Hz, 1H), 5.56 (dd, J=11.6, 6.8 Hz, 1H), 5.04 (tt, J=6.4, 4.0 Hz, 1H), 4.68-4.51 (m, 2H), 4.42-4.26 (m, 2H), 3.95 (s, 3H), 3.40-3.21 (m, 2H), 2.29 (dd, J=8.8, 0.8 Hz, 6H), 2.11 (s, 3H).

### (3-((2-(1,4-dimethyl-1H-pyrazol-5-yl)-5-fluoropyridin-4-yl)oxy)azetidin-1-yl)(5-(1-methyl-1H-pyrrol-3-yl)-4,5-dihydro-1H-pyrazol-1-yl)methanone (145)

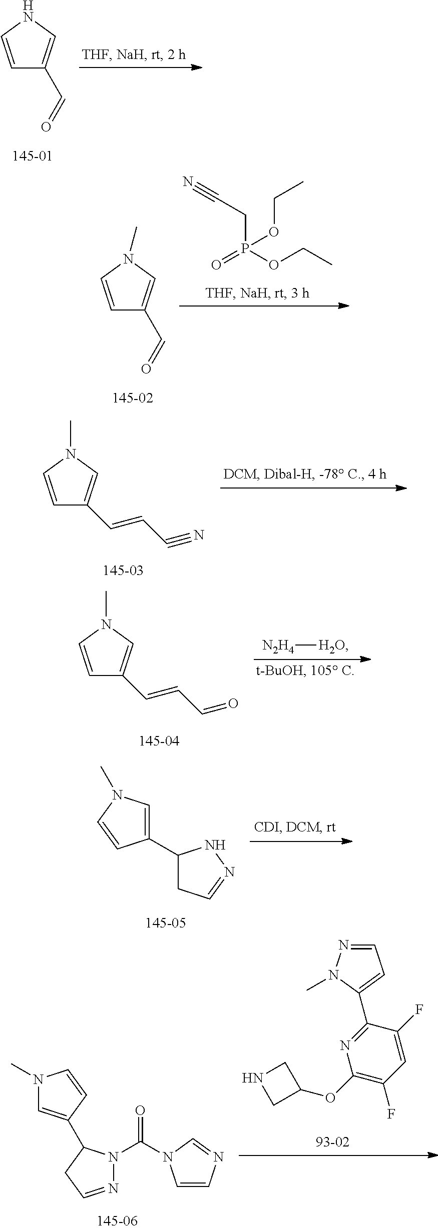

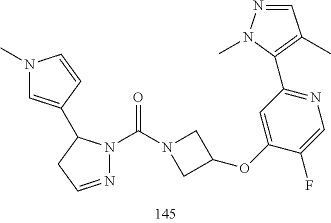

Step 1: Add a solution of 145-01 (7.5 g, 7.9 mmol, 1.0 equivalent) in THF (50 mL) into a suspension of NaH (2.9 g, 12 mmol, 1.1 equivalents) in THF (8 mL). After 15 minutes, add CH₃I (6 mL, 13.5 g 1.1 equivalents) into the solution. Quench the reaction with saturated NH₄Cl after 4 hours. Separate the aqueous and organic layers. Extract the aqueous layer with ether (2×150 mL), washed (water then brine), dried (MgSO₄, filtered and concentrated. Purification by chromatography (10% EtOAc-hexanes). Target product 145-02 was obtained as a white solid. (5.5 g, 80%). Mass (m/z) 110.0 [M+H]⁺.

Step 2: To 8.1 g (46 mmol) of diethylcyanomethylphosphonate in 5 mL of dry THE was added 1.87 g (78 mmol) of sodium hydride (60% dispersion in mineral oil). The mixture was stirred for 5 min., followed by addition of 5 g (46 mmol) of compound 145-02 in dry THE. The reaction was heated to reflux for 30 min, cooled to RT, quenched with saturated aqueous NH₄Cl, extracted with ether (2×150 mL), washed (water then brine), dried (MgSO₄, filtered and concentrated. Purification by chromatography (10% EtOAc-hexanes). Target product 145-03 was obtained as a white solid (3.7 g, 75%). Mass (m/z) 133.0 [M+H]⁺.

Step 3: To 3.5 g (27 mmol) of 145-03 in 25 mL of CH₂Cl₂ at −78° C. was added 22.5 mL (22.5 mmol) of DIBAL (1 M in toluene). The reaction mixture was stirred for 10 min and quenched with 10 mL of a saturated aqueous solution of Rochelle salt. The product was extracted with ether (2×20 mL), washed (water then brine), dried (MgSO₄, filtered, concentrated and purified by chromatography (CH₂Cl₂/MeOH=20/1). Target product 145-04 was obtained as a white solid (1.2 g, 30.1%). Mass (m/z) 136.2 [M+H]⁺.

Step 4: To a solution of 145-04 (1200 mg, 9 mmol), Hydrazine hydrate (8 mL) in t-BuOH was stirred under nitrogen at 105° C. for 3 h. The mixture was concentrated under 50° C. to get crude product 145-05 (1.5 g) as a yellow solid. Mass (m/z) 150.2 [M+H]⁺.

Step 5: To a solution of CDI (5.5 g, 33.6 mmol) in DCM stirred under nitrogen at 0° C. was added a solution of 145-05 (1.25 g, 8.4 mmol) in DCM dropwise. The reaction mixture was stirred at 0° C. for 1 h. The mixture was extracted with ether (2×150 mL), washed (water then brine), dried (MgSO₄, filtered and concentrated. Purification by chromatography (CH₂Cl₂/MeOH=20/1). Target product 145-06 was obtained as white solid (520 mg, 24.10%). Mass (m/z) 244.2 [M+H]⁺.

Step 6: To a solution of 145-06 (60 mg, 0.25 mmol), 93-02 (78 mg, 0.29 mmol) and DIEA (160 mg, 1.2 mmol) in DCM was stirred under nitrogen at 45° C. for 48 hrs. The reaction was concentrated under vacuum. The residue was purified by perp-HPLC to afford the desired product 145 as a white solid (45 mg, 40.2%). Mass (m/z) 438.2 [M+H]⁺. ¹H NMR (400 MHz, CDCl₃) δ 8.49 (d, J=2.9 Hz, 1H), 7.41 (s, 1H), 6.81 (t, J=1.6 Hz, 1H), 6.70 (d, J=6.5 Hz, 1H), 6.63 (t, J=2.0 Hz, 1H), 6.51 (t, J=2.5 Hz, 1H), 6.04-5.97 (m, 1H), 5.32 (dd, J=11.7, 5.8 Hz, 1H), 5.02 (s, 1H), 4.65-4.55 (m, 1H), 4.53-4.45 (m, 1H), 4.40-4.30 (m, 1H), 4.24 (dd, J=10.3, 3.7 Hz, 1H), 4.00 (s, 3H), 3.59 (s, 3H), 3.22 (ddd, J=18.4, 11.7, 1.6 Hz, 1H), 2.87 (ddd, J=18.3, 5.8, 1.8 Hz, 1H), 2.12 (s, 3H).

### (S)-(5-(3,5-difluorophenyl)-4,5-dihydro-1H-pyrazol-1-yl)(3-((4-(1,4-dimethyl-1H-pyrazol-5-yl)thiazol-2-yl)oxy)azetidin-1-yl)methanone (146)

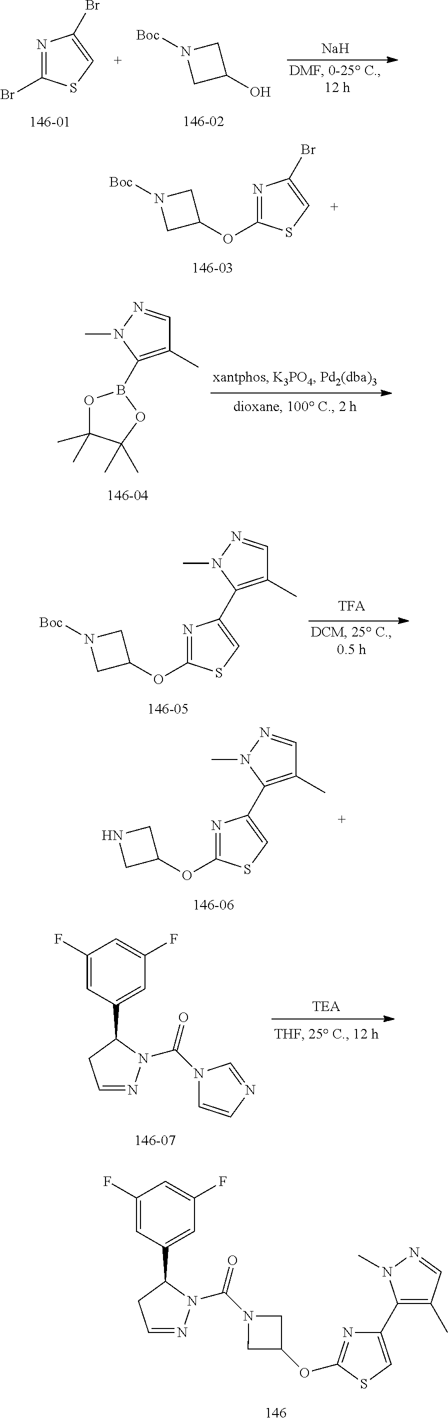

Step 1: 146-02 (713 mg, 4.12 mmol) was dissolved in 10 mL of dry DMF, NaH (148 mg, 6.18 mmol) was added to the above solution at 0° C., the mixture was stirred for 30 min. Then 146-01 (1 g, 4.12 mmol) was added to the above solution, the mixture was stirred for 12 h. The mixture was added water and extracted with EA, washed with brine, dried (Na₂SO₄), and concentrated in vacuo. Purification by silica gel chromatography to give the titled compound 146-03 (620 mg, 45.2%) as a white solid. Mass (m/z) 335.1 [M+H]⁺.

Step 2: 146-03 (500 mg, 1.5 mmol), 146-04 (331 mg, 1.5 mmol), x-phos (71.4 mg, 0.15 mmol), Pd₂(dba)₃ (137.2 mg, 0.15 mmol), K₃PO₄ (5N, 5 mL) were placed in dioxane (10 mL). The mixture was stirred 100° C. for 12 h under N₂. The mixture added water and extracted with DCM, washed with brine, dried (Na₂SO₄), and concentrated in vacuo. Purification by silica gel chromatography gave the titled compound 146-05 (310 mg, 59.3%) as a white solid. Mass (m/z) 351.2 [M+H]⁺.

Step 3: 146-05 (100 mg, 0.29 mmol) were dissolved in DCM (2 mL), TFA (1 mL) was added to the above solution, the mixture was stirred for 30 min. Concentrated to give the desired product 146-06, which was used for next step without further purification. Mass (m/z) 251.2 [M+H]+, Step 4: 146-06 (120 mg, crude), 146-07 (80 mg, 0.29 mmol) were placed in THE (10 mL), TFA (1 mL) was added to the above solution. The mixture was stirred 12 h. The mixture added water and extracted with EA, washed with brine, dried (Na₂SO₄), and concentrated in vacuo. Purification by silica gel chromatography gave the titled compound 146 (17 mg, 12.8%) as a white solid. Mass (m/z) 459.1 [M+H]⁺. ¹H NMR (400 MHz, Chloroform-d) δ 7.36 (s, 1H), 6.80-6.73 (m, 4H), 6.69 (tt, J=8.9, 2.3 Hz, 1H), 5.43 (ddd, J=10.5, 6.7, 4.1 Hz, 1H), 5.27 (dd, J=12.1, 6.5 Hz, 1H), 4.64-4.48 (m, 2H), 4.30 (dd, J=25.9, 10.3 Hz, 2H), 4.03 (s, 3H), 3.35 (ddd, J=18.5, 12.2, 1.7 Hz, 1H), 2.70 (ddd, J=18.7, 6.5, 1.7 Hz, 1H), 2.16 (s, 3H).

### (S)-(5-(3,5-difluorophenyl)-4,5-dihydro-1H-pyrazol-1-yl)(3-((4-(1-methyl-1H-pyrazol-5-yl)thiazol-2-yl)oxy)azetidin-1-yl)methanone (147)

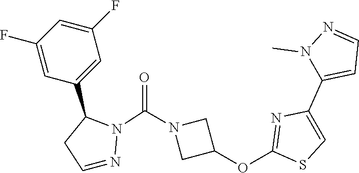 [SMILES: Cn1nccc1-c1csc(OC2CN(C(=O)N3N=CC[C@H]3c3cc(F)cc(F)c3)C2)n1]

The titled compound 147 was prepared in a 7% yield according to the procedure outlined for compound 146. ¹H NMR (400 MHz, Chloroform-d) δ 7.48 (s, 1H), 6.86 (s, 1H), 6.78 (q, J=1.8 Hz, 1H), 6.75 (dt, J=6.5, 2.1 Hz, 2H), 6.69 (tt, J=8.8, 2.3 Hz, 1H), 6.49 (s, 1H), 5.50-5.40 (m, 1H), 5.27 (dd, J=12.2, 6.5 Hz, 1H), 4.65-4.51 (m, 2H), 4.39-4.22 (m, 2H), 4.11 (s, 3H), 3.35 (ddd, J=18.6, 12.2, 1.7 Hz, 1H), 2.70 (ddd, J=18.6, 6.5, 1.7 Hz, 1H). Mass (m/z) 445.2 [M+H]⁺.

### (S)-5-(2-((1-(5-(3,5-difluorophenyl)-4,5-dihydro-1H-pyrazole-1-carbonyl)azetidin-3-yl)oxy)thiazol-4-yl)-1,4-dimethyl-1H-pyrazole-3-carbonitrile (148)

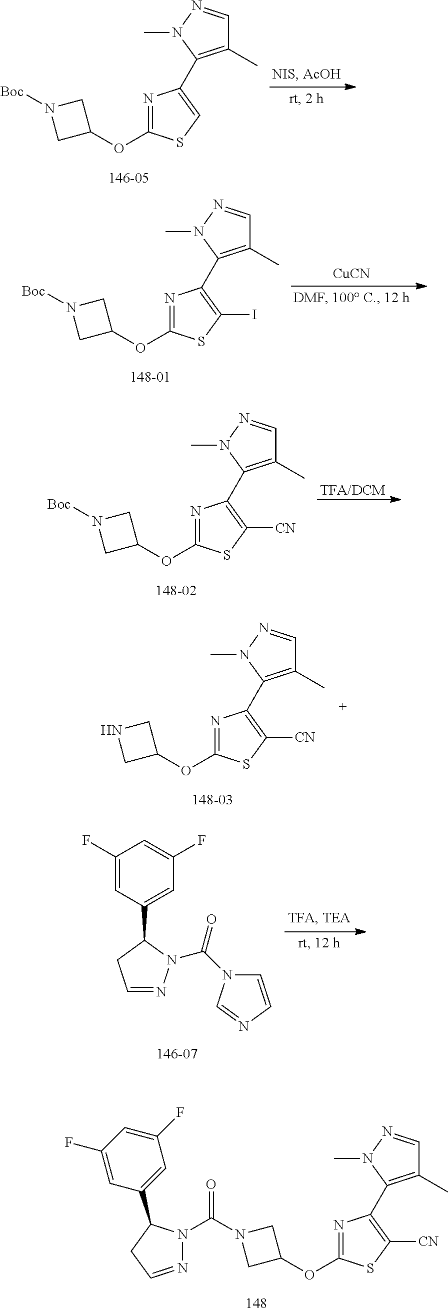

Step 1: 146-05 (700 mg, 2 mmol) was dissolved in 10 ml of dry AcOH, NIS (450 mg, 2 mmol) was added to the above solution at 25° C., the mixture was stirred for 2 h. The mixture was added water and extracted with EA, washed with brine, dried (Na₂SO₄), and concentrated in vacuo. Purification by silica gel chromatography gave the titled compound 148-01 (620 mg, 63%) as a white solid. MS (m/z) 477.2 [M+H]⁺

Step 2: 148-01 (476 mg, 1 mmol), CuCN (180 mg, 2 mmol) were placed in DMF (5 mL). The mixture was stirred 150° C. for 12 h under N₂. The mixture added water and extracted with DCM, washed with brine, dried (Na₂SO₄), and concentrated in vacuo. Purification by silica gel chromatography gave the titled compound 148-02 (210 mg, 56%) as a white solid. MS (m/z) 376.2 [M+H]⁺.

Step 3-4: The titled compound 148 was prepared in 23.4% yield from compound 148-02 according to the procedure outlined for compound 146. ¹H NMR (400 MHz, Chloroform-d) δ 7.41 (d, J=0.7 Hz, 1H), 6.83-6.81 (m, 1H), 6.79-6.75 (m, 2H), 6.72 (tt, J=8.9, 2.3 Hz, 1H), 5.51 (dq, J=6.5, 3.8, 3.3 Hz, 2H), 5.29 (dd, J=12.2, 6.5 Hz, 2H), 4.61 (dd, J=20.5, 11.5 Hz, 3H), 4.33 (dd, J=25.6, 11.0 Hz, 3H), 3.91 (s, 3H), 3.43-3.34 (m, 2H), 2.78-2.70 (m, 1H), 2.19 (s, 3H). Mass (m/z) 484.2 [M+H]⁺.

### (S)-(3-((5-chloro-4-(1,4-dimethyl-1H-pyrazol-5-yl)thiazol-2-yl)oxy)azetidin-1-yl)(5-(3,5-difluorophenyl)-4,5-dihydro-1H-pyrazol-1-yl)methanone (149)

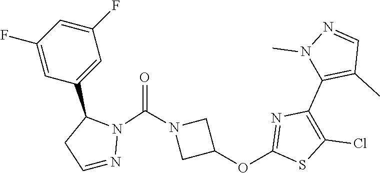 [SMILES: Cc1cnn(C)c1-c1nc(OC2CN(C(=O)N3N=CC[C@H]3c3cc(F)cc(F)c3)C2)sc1Cl]

The titled compound 149 was prepared in a 29% yield according to the procedure outlined for compound 146. Mass (m/z) 493.1 [M+H]⁺. ¹H NMR (400 MHz, Chloroform-d) δ 7.30 (s, 1H), 6.75-6.57 (m, 4H), 5.41 (dq, J=6.5, 3.9 Hz, 1H), 5.21 (dd, J=12.2, 6.4 Hz, 1H), 4.62-4.45 (m, 2H), 4.31-4.11 (m, 2H), 3.71 (s, 3H), 3.29 (ddd, J=18.6, 12.2, 1.6 Hz, 1H), 2.64 (ddd, J=18.6, 6.5, 1.7 Hz, 1H), 1.94 (s, 3H).

### (S)-5-(2-((1-(5-(3,5-difluorophenyl)-4,5-dihydro-1H-pyrazole-1-carbonyl)azetidin-3-yl)oxy)thiazol-4-yl)-1-methyl-1H-pyrazole-4-carbonitrile (150)

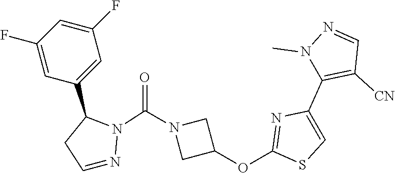 [SMILES: Cn1ncc(C#N)c1-c1csc(OC2CN(C(=O)N3N=CC[C@H]3c3cc(F)cc(F)c3)C2)n1]

The titled compound 150 was prepared in a 30.2% yield according to the procedure outlined for compound 156. ¹H NMR (400 MHz, Chloroform-d) δ 7.79 (s, 1H), 7.46 (s, 1H), 6.84-6.66 (m, 4H), 5.48 (ddd, J=6.6, 4.1, 2.5 Hz, 1H), 5.30 (dd, J=12.2, 6.5 Hz, 1H), 4.69-4.54 (m, 2H), 4.34 (ddd, J=32.5, 11.0, 3.9 Hz, 2H), 4.16 (s, 3H), 3.38 (ddd, J=18.6, 12.2, 1.7 Hz, 1H), 2.73 (ddd, J=18.6, 6.5, 1.7 Hz, 1H). Mass (m/z) 470.2 [M+H]⁺.

### (S)-(5-(3,5-difluorophenyl)-4,5-dihydro-1H-pyrazol-1-yl)(3-((5-(1,4-dimethyl-1H-pyrazol-5-yl)thiazol-2-yl)oxy)azetidin-1-yl)methanone (151)

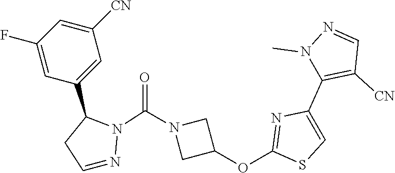 [SMILES: Cn1ncc(C#N)c1-c1csc(OC2CN(C(=O)N3N=CC[C@H]3c3cc(F)cc(C#N)c3)C2)n1]

The titled compound 151 was prepared in 34.6% yield according to the procedure outlined for compound 156. ¹H NMR (400 MHz, Chloroform-d) δ 7.79 (s, 1H), 7.46 (s, 1H), 6.84-6.66 (m, 4H), 5.48 (ddd, J=6.6, 4.1, 2.5 Hz, 1H), 5.30 (dd, J=12.2, 6.5 Hz, 1H), 4.69-4.54 (m, 2H), 4.34 (ddd, J=32.5, 11.0, 3.9 Hz, 2H), 4.16 (s, 3H), 3.38 (ddd, J=18.6, 12.2, 1.7 Hz, 1H), 2.73 (ddd, J=18.6, 6.5, 1.7 Hz, 1H). Mass (m/z) 477.2 [M+H]⁺.

### (S)-4-(1,4-dimethyl-1H-pyrazol-5-yl)-2-((1-(5-(5-fluoropyridin-3-yl)-4,5-dihydro-1H-pyrazole-1-carbonyl)azetidin-3-yl)oxy)thiazole-5-carbonitrile (152)

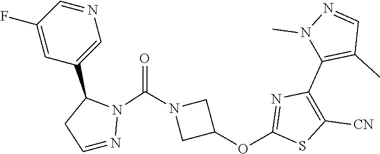 [SMILES: Cc1cnn(C)c1-c1nc(OC2CN(C(=O)N3N=CC[C@H]3c3cncc(F)c3)C2)sc1C#N]

The titled compound 152 was prepared in a 36.26% yield according to the procedure outlined for compound 156. ¹H NMR (400 MHz, Chloroform-d) δ 8.49 (s, 2H), 7.64 (d, J=8.0 Hz, 1H), 7.44 (s, 1H), 6.90 (s, 1H), 5.52 (dd, J=7.0, 3.2 Hz, 1H), 5.42 (dd, J=12.1, 6.6 Hz, 1H), 4.70-4.52 (m, 2H), 4.43-4.26 (m, 2H), 3.93 (s, 3H), 3.50 (dd, J=18.5, 12.1 Hz, 1H), 2.87-2.78 (m, 1H). Mass (m/z) 467.2 [M+H]⁺.

### (S)-(5-(3,5-difluorophenyl)-4,5-dihydro-1H-pyrazol-1-yl)(3-((5-(1-methyl-1H-pyrazol-5-yl)thiazol-2-yl)oxy)azetidin-1-yl)methanone (153)

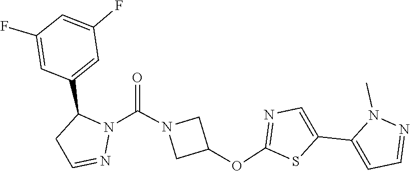 [SMILES: Cn1nccc1-c1cnc(OC2CN(C(=O)N3N=CC[C@H]3c3cc(F)cc(F)c3)C2)s1]

The titled compound 153 was prepared in a 11.4% yield according to the procedure outlined for compound 146. ¹H NMR (300 MHz, Chloroform-d) δ 7.48 (d, J=2.0 Hz, 1H), 7.13 (s, 1H), 6.83-6.62 (m, 4H), 6.33 (d, J=2.0 Hz, 1H), 5.44 (tt, J=6.6, 3.9 Hz, 1H), 5.27 (dd, J=12.2, 6.4 Hz, 1H), 4.58 (q, J=12.3, 10.6 Hz, 2H), 4.39-4.21 (m, 2H), 3.93 (s, 3H), 3.35 (ddd, J=18.6, 12.1, 1.7 Hz, 1H), 2.70 (ddd, J=18.6, 6.5, 1.7 Hz, 1H). Mass (m/z) 445.2 [M+H]⁺.

### (S)-(5-(3,5-difluorophenyl)-4,5-dihydro-1H-pyrazol-1-yl)(3-((5-(1,4-dimethyl-1H-pyrazol-5-yl)thiazol-2-yl)oxy)azetidin-1-yl)methanone (154)

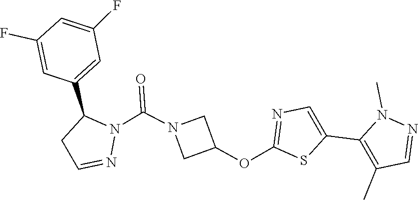 [SMILES: Cc1cnn(C)c1-c1cnc(OC2CN(C(=O)N3N=CC[C@H]3c3cc(F)cc(F)c3)C2)s1]

The titled compound 154 was prepared in a 19.4% yield according to the procedure outlined for compound 146. ¹H NMR (400 MHz, Chloroform-d) δ 7.42 (s, 1H), 7.08 (s, 1H), 6.87-6.69 (m, 4H), 5.51-5.44 (m, 1H), 5.35-5.26 (m, 1H), 4.70-4.54 (m, 2H), 4.35 (d, J=24.2 Hz, 2H), 3.87 (s, 3H), 3.43-3.35 (m, 1H), 2.73 (dd, J=18.5, 5.8 Hz, 1H), 2.08 (s, 3H). Mass (m/z) 459.2 [M+H]⁺.

### (S)-3-fluoro-5-(1-(3-((4-(1-methyl-1H-pyrazol-5-yl)thiazol-2-yl)oxy) azetidine-1-carbonyl)-4,5-dihydro-1H-pyrazol-5-yl)benzonitrile (155)

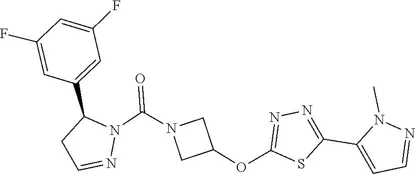 [SMILES: Cn1nccc1-c1nnc(OC2CN(C(=O)N3N=CC[C@H]3c3cc(F)cc(F)c3)C2)s1]

The titled compound 155 was prepared in a 22.6% yield according to the procedure outlined for compound 156. ¹H NMR (400 MHz, Chloroform-d) δ 7.54 (s, 1H), 6.85-6.65 (m, 4H), 6.56 (s, 1H), 5.56 (s, 1H), 5.28 (dd, J=12.3, 6.3 Hz, 1H), 4.74-4.55 (m, 2H), 4.34 (dd, J=22.6, 11.3 Hz, 2H), 4.27 (d, J=2.4 Hz, 3H), 3.36 (dd, J=18.6, 12.1 Hz, 1H), 2.71 (dd, J=18.7, 6.2 Hz, 1H). Mass (m/z) 466.2 [M+H]⁺.

### (S)-3-(1-(3-((4-(1,4-dimethyl-1H-pyrazol-5-yl)thiazol-2-yl)oxy)azetidine-1-carbonyl)-4,5-dihydro-1H-pyrazol-5-yl)-5-fluorobenzonitrile (156)

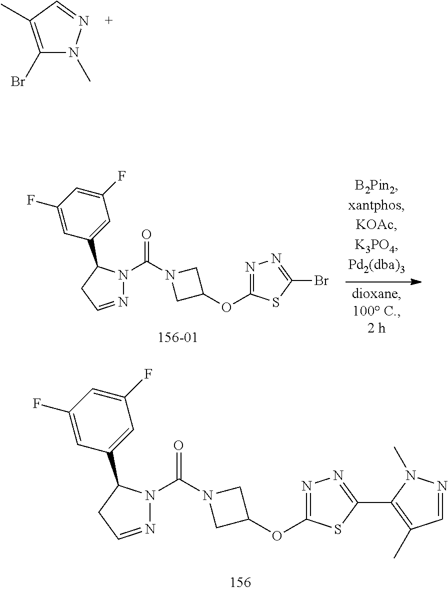

5-bromo-1,4-dimethyl-1H-pyrazole (175 mg, 1 mmol), x-phos (71.4 mg, 0.15 mmol), Pd₂(dba)₃ (137.2 mg, 0.15 mmol), KOAc (571 mg, 3 mmol), B₂Pin₂ (476 mg, 2 mmol) were placed in dioxane (10 mL). The mixture was stirred at 100° C. for 1 h under N₂. 156-01 (444 mg, 1 mmol) and K₃PO₄ (5N, 1 mL) was added after stirring at 100° C. for 1 h under N₂. The reaction was quenched with water and extracted with DCM, washed with brine, dried over Na₂SO₄, and concentrated in vacuo to give crude product. The crude product was purified by silica gel chromatography to give the titled compound 156 (50 mg, 11%) as a white solid. Mass (m/z) 460.2 [M+H]⁺. ¹H NMR (400 MHz, Chloroform-d) δ 7.41 (s, 1H), 6.83-6.66 (m, 4H), 5.57 (s, 1H), 5.28 (dd, J=12.3, 6.5 Hz, 1H), 4.75-4.56 (m, 2H), 4.34 (dd, J=22.5, 10.9 Hz, 2H), 4.20 (s, 3H), 3.36 (dd, J=18.8, 12.0 Hz, 1H), 2.71 (dd, J=18.6, 6.2 Hz, 1H), 2.22 (s, 3H).

### (S)-(5-(3,5-difluorophenyl)-4,5-dihydro-11H-pyrazol-1-yl)(3-((4-(1,4-dimethyl-1H-pyrazol-5-yl)-5-(hydroxymethyl)thiazol-2-yl)oxy)azetidin-1-yl)methanone (157)

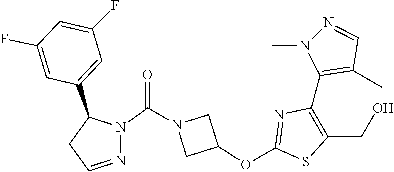 [SMILES: Cc1cnn(C)c1-c1nc(OC2CN(C(=O)N3N=CC[C@H]3c3cc(F)cc(F)c3)C2)sc1CO]

The titled compound 157 was prepared according to the procedure outlined for compound 146. ¹H NMR (400 MHz, Chloroform-d) δ 7.36 (s, 1H), 6.81 (t, J=1.7 Hz, 1H), 6.79-6.75 (m, 2H), 6.71 (tt, J=8.7, 2.3 Hz, 1H), 5.42 (td, J=6.6, 3.4 Hz, 1H), 5.29 (dd, J=12.2, 6.5 Hz, 1H), 4.62 (d, J=5.5 Hz, 3H), 4.55 (d, J=15.0 Hz, 2H), 4.30 (dd, J=22.3, 10.8 Hz, 2H), 3.78 (s, 3H), 3.37 (ddd, J=18.6, 12.2, 1.7 Hz, 1H), 2.72 (ddd, J=18.6, 6.5, 1.7 Hz, 1H), 1.99 (s, 3H).

### (5-(3,5-difluorophenyl)-4,5-dihydro-1H-pyrazol-1-yl)(3-((6-(1,4-dimethyl-1H-pyrazol-5-yl)-3,5-difluoropyridin-2-yl)oxy)azetidin-1-yl)methanone (158)

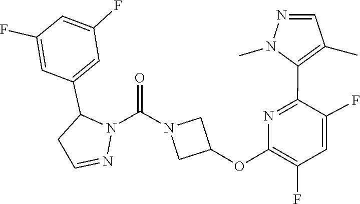 [SMILES: Cc1cnn(C)c1-c1nc(OC2CN(C(=O)N3N=CCC3c3cc(F)cc(F)c3)C2)c(F)cc1F]

The titled compound 158 was prepared in a 45.5% yield according to the procedure outlined for compound 123. ¹H NMR (400 MHz, Chloroform-d) δ 7.42-7.33 (m, 2H), 6.82-6.62 (m, 4H), 5.37 (tt, J=6.6, 4.2 Hz, 1H), 5.26 (dd, J=12.2, 6.5 Hz, 1H), 4.57 (t, J=16.6 Hz, 2H), 4.34-4.18 (m, 2H), 3.82 (d, J=0.6 Hz, 3H), 3.34 (ddd, J=18.6, 12.2, 1.7 Hz, 1H), 2.69 (ddd, J=18.6, 6.6, 1.8 Hz, 1H), 2.01 (d, J=1.8 Hz, 3H). Mass (m/z) 489.3 [M+H]⁺.

### (S)-1-(5-chloro-4-((1-(5-(3,5-difluorophenyl)-4,5-dihydro-1H-pyrazole-1-carbonyl)azetidin-3-yl)oxy)pyridin-2-yl)-3,5-dimethyl-1H-pyrazole-4-carbonitrile (159)

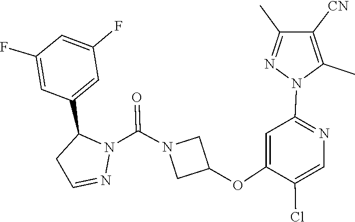 [SMILES: Cc1nn(-c2cc(OC3CN(C(=O)N4N=CC[C@H]4c4cc(F)cc(F)c4)C3)c(Cl)cn2)c(C)c1C#N]

The titled compound 159 was prepared in a 25% yield according to the procedure outlined for compound 1. Mass (m/z) 512.2 [M+H]⁺. ¹H NMR (400 MHz, Chloroform-d) δ 8.29 (s, 1H), 7.17 (s, 1H), 6.73 (dd, J=12.1, 6.3 Hz, 4H), 5.34-5.25 (m, 1H), 5.15 (s, 1H), 4.65 (s, 2H), 4.32 (d, J=34.8 Hz, 2H), 3.36 (dd, J=18.5, 12.2 Hz, 1H), 2.78 (s, 3H), 2.70 (d, J=24.8 Hz, 1H), 2.39 (s, 3H).

### (S)-1-(5-chloro-4-((1-(5-(3,5-difluorophenyl)-4,5-dihydro-1H-pyrazole-1-carbonyl)azetidin-3-yl)oxy)pyridin-2-yl)-3,5-dimethyl-1H-pyrazole-4-carboxamide (160)

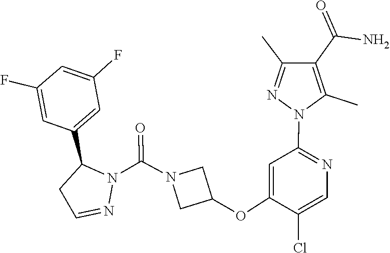 [SMILES: Cc1nn(-c2cc(OC3CN(C(=O)N4N=CC[C@H]4c4cc(F)cc(F)c4)C3)c(Cl)cn2)c(C)c1C(N)=O]

The titled compound 160 was prepared in 22% yield according to the procedure outlined for compound 63. Mass (m/z) 530.1 (M+H⁺). ¹H NMR (300 MHz, Chloroform-d) δ 8.32 (s, 1H), 7.11 (s, 1H), 6.89-6.54 (m, 4H), 5.59 (s, 2H), 5.34-5.25 (m, 1H), 5.23-5.13 (m, 1H), 4.64 (d, J=8.6 Hz, 2H), 4.33 (dd, J=27.3, 10.3 Hz, 2H), 3.36 (ddd, J=18.5, 12.2, 1.7 Hz, 1H), 2.84 (s, 3H), 2.71 (ddd, J=18.6, 6.5, 1.7 Hz, 1H), 2.48 (s, 3H).

### (4-(3,5-difluorophenyl)-4,5-dihydro-1H-pyrazol-3-yl)(3-((2-(1,4-dimethyl-1H-pyrazol-5-yl)-5-fluoropyridin-4-yl)oxy)azetidin-1-yl)methanone (161)

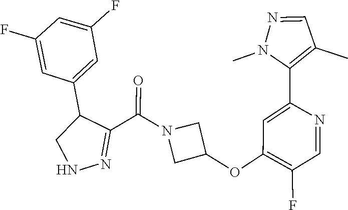 [SMILES: Cc1cnn(C)c1-c1cc(OC2CN(C(=O)C3=NNCC3c3cc(F)cc(F)c3)C2)c(F)cn1]

The titled compound 161 was prepared as white solid in a yield of 7.4% according to the procedure outlined for compound 162. Mass (m/z) 471.3 [M+H]⁺. ¹H NMR (400 MHz, Chloroform-d) δ 8.50 (s, 1H), 7.37 (s, 1H), 6.85-6.74 (m, 2H), 6.72-6.64 (m, 2H), 5.35 (t, J=4.8 Hz, 1H), 5.12-5.05 (m, 1H), 4.65-4.55 (m, 2H), 4.50-4.40 (m, 2H), 4.23-4.15 (m, 1H), 3.94 (s, 3H), 3.66-3.60 (m, 1H), 2.10 (s, 3H).

### 1-(4-((1-(4-(3,5-difluorophenyl)-4,5-dihydro-1H-pyrazole-3-carbonyl)azetidin-3-yl)oxy)-5-fluoropyridin-2-yl)-3,5-dimethyl-1H-pyrazole-4-carb oxamide (162)

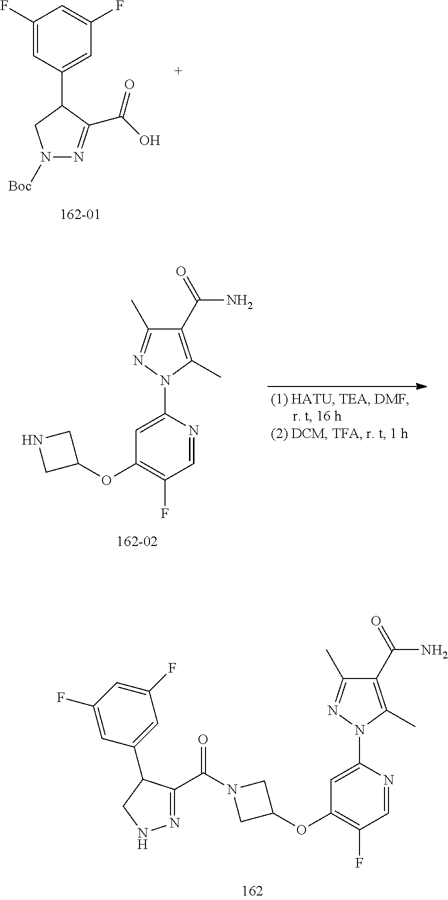 [SMILES: CC(C)(C)OC(=O)N1CC(c2cc(F)cc(F)c2)C(C(=O)O)=N1 ; Cc1nn(-c2cc(OC3CNC3)c(F)cn2)c(C)c1C(N)=O ; Cc1nn(-c2cc(OC3CN(C(=O)C4=NNCC4c4cc(F)cc(F)c4)C3)c(F)cn2)c(C)c1C(N)=O]

Step 1: Compound 162-01 (70 mg, 0.173 mmol) was dissolved in 2 mL DCM. 2 mL DCM/TFA (1/1) was added slowly to the solution at 0° C. Let it stir at r.t for 1 h. The solvent was evaporated to dryness and used for next step without further purification. MS (m/z): 406.4 [M+H]⁺.

Step 2: A mixture of compound 162-02, 162-01 (58 mg, 0.178 mmol), HATU (98 mg, 0.258 mmol) and TEA (0.5 mL) in 2 mL DMF were stirred at r.t for 16 h. The solvent was evaporated to dryness and purified by column chromatography (DCM/MeOH=9/1) to give tert-butyl 3-(3-((2-(4-carbamoyl-3,5-dimethyl-1H-pyrazol-1-yl)-5-fluoropyridin-4-yl)oxy)azetidine-1-carbonyl)-4-(3,5-difluorophenyl)-4,5-dihydro-1H-pyrazole-1-carboxylate (55 mg, 51.9%) as brown oil. Mass (m/z) 614.3 [M+H]⁺. The above oil (55 mg, 0.09 mmol) was dissolved in 2 mL DCM, 2 mL DCM/TFA (1/1) was added slowly to the solution at 0° C. The reaction mixture was stirred at r.t for 1 h. The solvent was evaporated to dryness and purified by prep-TLC to give compound 162 (1 mg, 1%) as a white solid. Mass (m/z) 514.4 [M+H]⁺. ¹H NMR (400 MHz, Chloroform-d) δ 8.23 (s, 1H), 6.88-6.60 (m, 4H), 5.59 (brs, 2H), 5.35 (t, J=4.8 Hz, 2H), 4.67-4.56 (m, 2H), 4.52-4.42 (m, 2H), 4.23-4.15 (m, 1H), 3.68-3.62 (m, 1H), 2.82 (s, 3H), 2.47 (s, 3H).

### (S)-(5-(3,5-difluorophenyl)-4,5-dihydro-1H-pyrazol-1-yl)(3-((5-fluoro-2-(2-methoxypyridin-3-yl)pyrimidin-4-yl)oxy)azetidin-1-yl)methanone (163)

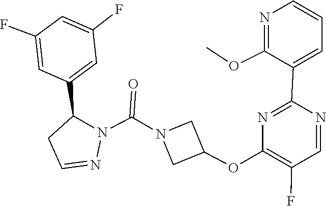 [SMILES: COc1ncccc1-c1ncc(F)c(OC2CN(C(=O)N3N=CC[C@H]3c3cc(F)cc(F)c3)C2)n1]

The titled compound 163 was prepared in a 59.3% yield according to the procedure outlined for compound 165. ¹H NMR (400 MHz, Chloroform-d) δ 8.48 (d, J=2.5 Hz, 1H), 8.27 (dd, J=4.9, 2.0 Hz, 1H), 8.09 (dd, J=7.4, 2.0 Hz, 1H), 7.00 (dd, J=7.4, 4.9 Hz, 1H), 6.85-6.73 (m, 3H), 6.72-6.65 (m, 1H), 5.52-5.45 (m, 1H), 5.32-5.20 (m, 1H), 4.71-4.54 (m, 2H), 4.35 (dd, J=28.3, 10.2 Hz, 2H), 4.04 (s, 3H), 3.36 (ddd, J=18.6, 12.2, 1.7 Hz, 1H), 2.70 (ddd, J=18.6, 6.5, 1.7 Hz, 1H). Mass (m/z) 485.3 [M+H]⁺.

### (S)-(5-(3,5-difluorophenyl)-4,5-dihydro-1H-pyrazol-1-yl)(3-((5-fluoro-2′-methoxy-[2,3′-bipyridin]-4-yl)oxy)azetidin-1-yl)methanone (164)

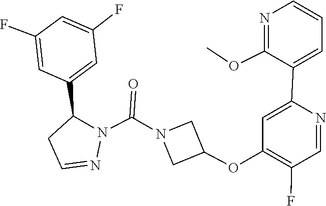 [SMILES: COc1ncccc1-c1cc(OC2CN(C(=O)N3N=CC[C@H]3c3cc(F)cc(F)c3)C2)c(F)cn1]

The titled compound 164 was prepared in 16.9% yield according to the procedure outlined for compound 165. ¹H NMR (400 MHz, Chloroform-d) δ 8.54 (d, J=3.1 Hz, 1H), 8.25 (dd, J=5.0, 1.8 Hz, 1H), 8.17 (dd, J=7.5, 1.9 Hz, 1H), 7.39 (d, J=6.9 Hz, 1H), 7.06 (dd, J=7.5, 4.9 Hz, 1H), 6.82-6.70 (m, 1H), 6.79-6.57 (m, 3H), 5.34-5.22 (m, 1H), 5.12 (d, J=7.5 Hz, 1H), 4.69-4.56 (m, 2H), 4.37 (dd, J=34.4, 10.6 Hz, 2H), 4.04 (s, 3H), 3.44-3.31 (m, 1H), 2.72 (ddd, J=18.8, 6.4, 1.7 Hz, 1H). Mass (m/z) 484.2 [M+H]⁺.

### (S)-(5-(3,5-difluorophenyl)-4,5-dihydro-1H-pyrazol-1-yl)(3-((5-fluoro-2′-methoxy-4′-methyl-[2,3′-bipyridin]-4-yl)oxy)azetidin-1-yl)methanone (165)

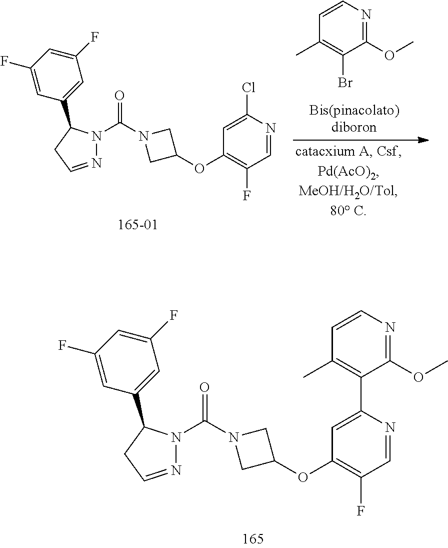

Compound 165-01 (300 mg, 0.732 mmol), 3-bromo-2-methoxy-4-methylpyridine (164 mg, 0.804 mmol), catacxium A (52.5 mg, 0.146 mmol), bis(pinacolato)diboron (278.8 mg, 1.097 mmol), CsF (578 mg, 3.8 mmol) and Pd(AcO)₂ (16.4 mg, 0.073 mmol) were mixed in 15 mL MeOH/H₂O (9/1). Let it stir at 60° C. for 3 h. Then another part of catacxium A (36 mg, 0.1 mmol) and Pd(AcO)₂ (11 mg, 0.05 mmol) in 2 mL toluene were added. Let it stir at 80° C. for 16 h. The solvent was evaporated to dryness and purified by prep-TLC (DCM/MeOH=15/1) to give compound 165 (6 mg, 1.6%) as a white solid. Mass (m/z) 498.4 [M+H]⁺. ¹H NMR (400 MHz, Chloroform-d) δ 8.44 (d, J=3.1 Hz, 1H), 7.12 (t, J=6.4 Hz, 1H), 6.89 (d, J=6.9 Hz, 1H), 6.81-6.63 (m, 4H), 6.16 (dd, J=11.6, 6.9 Hz, 1H), 5.27 (dd, J=12.2, 6.4 Hz, 1H), 5.18-5.05 (m, 1H), 4.70-4.49 (m, 2H), 4.37-4.21 (m, 2H), 3.54 (s, 3H), 3.35 (ddd, J=18.6, 12.2, 1.7 Hz, 1H), 2.69 (ddd, J=18.5, 6.4, 1.7 Hz, 1H), 2.52 (s, 3H).

### (S)-4-((1-(5-(3,5-difluorophenyl)-4,5-dihydro-11H-pyrazole-1-carbonyl)azetidin-3-yl)oxy)-5-fluoro-4′-methyl-[2,3′-bipyridin]-2′(1′H)-one (166)

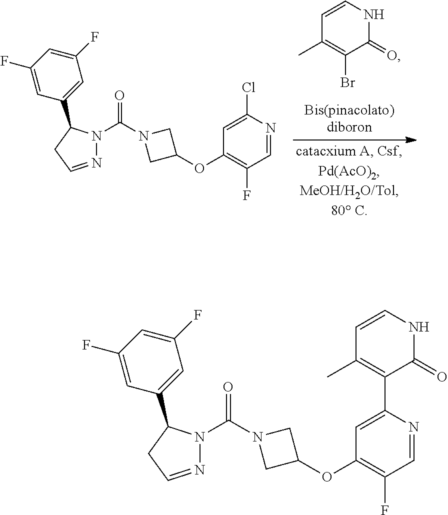

The titled compound 166 was prepared as white solid in a yield of 3% according to the procedure outlined for compound 165. Mass (m/z) 484.3 [M+H]⁺. ¹H NMR (400 MHz, Chloroform-d) δ 12.13 (brs, 1H), 8.43 (s, 1H), 6.86 (s, 1H), 6.79-6.63 (m, 4H), 6.21 (d, J=6.3 Hz, 1H), 5.27 (dd, J=12.2, 6.4 Hz, 1H), 5.13-5.02 (m, 1H), 4.63-4.48 (m, 2H), 4.40-4.20 (m, 2H), 3.35 (ddd, J=18.3, 11.8, 1.8 Hz, 1H), 2.70 (ddd, J=18.3, 9.9, 1.6 Hz, 1H), 2.14 (s, 3H).

### (S)-4-((1-(5-(3,5-difluorophenyl)-4,5-dihydro-1H-pyrazole-1-carbonyl)azetidin-3-yl)oxy)-5-fluoro-[2,3′-bipyridin]-2′(1′H)-one (167)

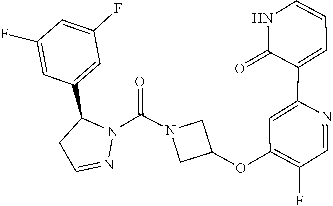 [SMILES: O=C(N1CC(Oc2cc(-c3ccc[nH]c3=O)ncc2F)C1)N1N=CC[C@H]1c1cc(F)cc(F)c1]

The titled compound 167 was prepared in a 11.2% yield according to the procedure outlined for compound 1. ¹H NMR (400 MHz, DMSO-d₆) δ 12.09 (s, 1H), 8.57 (d, J=2.9 Hz, 1H), 8.52-8.35 (m, 2H), 7.60 (d, J=6.3 Hz, 1H), 7.16 (tt, J=9.3, 2.4 Hz, 1H), 7.08 (d, J=1.8 Hz, 1H), 7.02-6.88 (m, 2H), 6.45 (t, J=6.7 Hz, 1H), 5.29 (dd, J=12.2, 6.6 Hz, 1H), 5.24 (q, J=3.4, 2.6 Hz, 1H), 4.57 (s, 2H), 4.11 (d, J=22.8 Hz, 2H), 3.44 (ddd, J=18.8, 12.2, 1.8 Hz, 1H), 2.74-2.65 (m, 1H). Mass (m/z) 470.2 [M+H]⁺.

### (S)-4-((1-(5-(3,5-difluorophenyl)-4,5-dihydro-1H-pyrazole-1-carbonyl)azetidin-3-yl)oxy)-5-fluoro-1′,4′-dimethyl-[2,3′-bipyridin]-2′(1′H)-one (168)

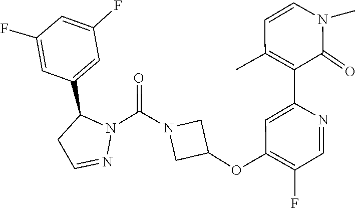 [SMILES: Cc1ccn(C)c(=O)c1-c1cc(OC2CN(C(=O)N3N=CC[C@H]3c3cc(F)cc(F)c3)C2)c(F)cn1]

The titled compound 168 was prepared as white solid in a yield of 3.9% according to the procedure outlined for compound 165. Mass (m/z) 498.3 [M+H]⁺. ¹H NMR (400 MHz, Chloroform-d) δ 8.78 (s, 1H), 8.41 (d, J=3.0 Hz, 1H), 6.86 (d, J=6.9 Hz, 1H), 6.79-6.62 (m, 4H), 6.17-6.12 (m, 1H), 5.26 (dd, J=12.1, 6.4 Hz, 1H), 5.12-5.05 (m, 1H), 4.66-4.46 (m, 2H), 4.38-4.22 (m, 2H), 3.54 (s, 3H), 3.35 (ddd, J=18.3, 11.8, 1.8 Hz, 1H), 2.68 (ddd, J=18.5, 6.4, 1.8 Hz, 1H), 2.52 (s, 3H).

### 4-((1-(5-(3,5-difluorophenyl)-4,5-dihydro-1H-pyrazole-1-carbonyl)azetidin-3-yl)oxy)-5-fluoro-1′-methyl-[2,3′-bipyridin]-2′(1′H)-one (169)

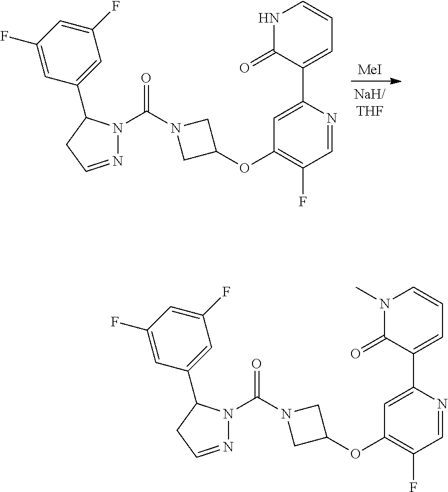 [SMILES: "MeI" ; O=C(N1CC(Oc2cc(-c3ccc[nH]c3=O)ncc2F)C1)N1N=CCC1c1cc(F)cc(F)c1 ; Cn1cccc(-c2cc(OC3CN(C(=O)N4N=CCC4c4cc(F)cc(F)c4)C3)c(F)cn2)c1=O]

The titled compound 169 was prepared in 93.2% yield according to the procedure outlined for compound 20. ¹H NMR (300 MHz, Chloroform-d) δ 8.49-8.41 (m, 1H), 8.38 (d, J=2.8 Hz, 1H), 8.31 (d, J=7.3 Hz, 1H), 7.43 (dd, J=6.5, 2.1 Hz, 1H), 6.89-6.43 (m, 4H), 6.37 (t, J=6.9 Hz, 1H), 5.27 (dd, J=12.2, 6.4 Hz, 1H), 5.23-5.13 (m, 1H), 4.76-4.50 (m, 2H), 4.41-4.16 (m, 2H), 3.65 (s, 3H), 3.34 (ddd, J=18.5, 12.2, 1.7 Hz, 1H), 2.68 (ddd, J=18.5, 6.5, 1.8 Hz, 1H). Mass (m/z) 484.3 [M+H]⁺.

### Example 2. Biological Assays

### Protocol for HT29 Cells 0FBS Assay In Vitro

### 1. Materials

Cell line: HT-29 (ATCC® HTB-38™)
Culture medium: McCOY's 5A, Gibco, Cat No. 16600-082
FBS, Gibco, Cat No. 10099-141C
Trypsin: Gibco, Cat No. 25200-056
DMSO: Sigma, Cat No. 67-68-5, 1L
Assay plate: Corning #3903
Compound dilution plate: Corning #3357
Inducers: TNFα, GenScript, Cat No. Z01001-50,
SmacM, Cat. No., HY-15989, MedChemExpress (MCE)
Z_VAD FMK, TargetMol, T6013
Cell Titer-Glo® Luminescent Cell Viability Assay Kit: Promega, Cat No. G7573
EnVision: PerkinElmer, 2105-0010

### 2. Cell Seeding of HT-29 Cells

1) HT-29 cells were checked every day to make sure they are healthy and growing as expected. They were split sub-culturing when were approximately 80% confluent.

2) First pre-warm the culture medium of McCOY's 5A medium (Gibco, Cat No. 16600-082) with 10% FBS (Gibco, Cat No. 10099-141C) in 37° C. water bath for at least 30 min.

3) Growing cells to desired level of confluency 80% in a T75 flask, aspirate the medium, and wash with warm PBS two times.

4) Add 2-3 ml fresh warm trypsin (Gibco, Cat No. 25200-056) solution. Transfer the flask to a 37° C. incubator.

5) After 5 minutes, tap the side of the flask, and examine the flask under a microscope for lifting. If necessary, return the cells to the incubator for an additional 5-10 minutes, with occasional tapping, until lifting is complete.

6) Quickly neutralize the trypsin reaction by adding 6-9 ml cell culture medium, then transfer the cells to sterile 15 ml conical tubes. Pellet the cells by centrifugation at 300×g for 7 minutes, then decant the supernatant.

7) Resuspend the cells in fresh cell culture medium. Do cell counting with hemocytometer.

8) Seed 100 μl of 5,000 cells into each well of the sterile 96-well cell culture plate (Corning 3903) and culture overnight at 37° C. with 5% CO2.

### 3. Compounds Titration and Treatment of HT-29 Cells

1) All batches of compounds (CPDs0 (e.g., compound 1-169) were dissolved in DMSO (Dimethyl sulfoxide) as 20 mM stock solution.

2) Take 3 ul 20 mM stock solution of CPDs to 27 ul DMSO and mix well, continue the titration ratio of 1:3 (20 ul CPDs+40 ul DMSO), e.g., to give solutions of CPDS at 6.6 mM, 2.2 mM, etc., till the 10 points end.

3) Take out assay plates with HT-29 cells from incubator, remove all culture medium then wash 1 time with 1×PBS, and change fresh McCOY's 5A medium of FBS free with a cocktail of TNF-α (10 ng/ml), the SMAC mimetic (6 uM) and zVAD-FMK (10 μM) to stimulate the HT-29 cells to increase pRIPK1 levels and necroptosis.

4) Add 0.5 μL of the diluted compound to the corresponding 96-well assay plates.

5) Incubate the assay plates for 20 hours at 37° C. with 5% CO2.

4. Performing Cell Viability Detection of HT-29 Cells after Treated with Compounds

1) The CellTiter-Glo® Luminescent Cell Viability Assay was employed to detect the ATP levels of viable HT-29 cells.

2) Equilibrate the CellTiter-Glo® buffer and the lyophilized substrate to room temperature prior to use.

3) Resuspend the CellTiter-Glo® substrate with CellTiter-Glo® buffer, mix by gently vortexing to obtain a homogeneous solution.

4) Pipette 20 μl the enzyme/substrate mixture by multi-channel pipette into the 96-well assay plates from step 5) under the Compounds titration and treatment stage.

5) Place the plates on an orbital shaker and mix the contents for 3 minutes to induce cell lysis.

6) Then allow the plates to incubate at room temperature for 10 minutes to stabilize luminescent signal.

7) Read and record luminescence signal with EnVision.

8) The geometric mean EC₅₀ of the compounds were calculated from 10 points response dose with duplicate. RIP1 inhibitory activity of compounds 1-169 is summarized in Table 2. In Table 2, activity is provided as follows: +++=0.1 nM≤EC50<100 nM; ++=100 nM≤EC5 1000 nM; ++=1000 nM≤EC5<10000 nM.

TABLE 2
EC50 Values of Compounds 1 to 169

| # | EC₅₀ | # | EC₅₀ | # | EC₅₀ | # | EC₅₀ |
|---|---|---|---|---|---|---|---|
| 1 | +++ | 2 | +++ | 3 | +++ | 4 | +++ |
| 5 | +++ | 6 | +++ | 7 | +++ | 8 | +++ |
| 9 | +++ | 10 | ++ | 11 | +++ | 12 | +++ |
| 13 | +++ | 14 | ++ | 15 | ++ | 16 | +++ |
| 17 | +++ | 18 | +++ | 19 | +++ | 20 | +++ |
| 21 | +++ | 22 | +++ | 23 | +++ | 24 | +++ |
| 25 | +++ | 26 | +++ | 27 | +++ | 28 | +++ |
| 29 | +++ | 30 | +++ | 31 | +++ | 32 | +++ |
| 33 | +++ | 34 | +++ | 35 | +++ | 36 | +++ |
| 37 | +++ | 38 | ++ | 39 | +++ | 40 | +++ |
| 41 | +++ | 42 | +++ | 43 | +++ | 44 | +++ |
| 45 | +++ | 46 | +++ | 47 | +++ | 48 | +++ |
| 49 | +++ | 50 | +++ | 51 | +++ | 52 | +++ |
| 53 | +++ | 54 | +++ | 55 | +++ | 56 | +++ |
| 57 | +++ | 58 | +++ | 59 | +++ | 60 | +++ |
| 61 | +++ | 62 | +++ | 63 | ++ | 64 | +++ |
| 65 | +++ | 66 | +++ | 67 | +++ | 68 | ++ |
| 69 | ++ | 70 | +++ | 71 | +++ | 72 | ++ |
| 73 | +++ | 74 | +++ | 75 | +++ | 76 | +++ |
| 77 | +++ | 78 | +++ | 79 | +++ | 80 | +++ |
| 81 | ++ | 82 | +++ | 83 | +++ | 84 | +++ |
| 85 | +++ | 86 | +++ | 87 | +++ | 88 | +++ |
| 89 | +++ | 90 | +++ | 91 | +++ | 92 | +++ |
| 93 | +++ | 94 | +++ | 95 | +++ | 96 | +++ |
| 97 | ++ | 98 | + | 99 | +++ | 100 | +++ |
| 101 | +++ | 102 | +++ | 103 | + | 104 | +++ |
| 105 | + | 106 | +++ | 107 | +++ | 108 | +++ |
| 109 | +++ | 110 | +++ | 111 | +++ | 112 | +++ |
| 113 | +++ | 114 | +++ | 115 | +++ | 116 | +++ |
| 117 | +++ | 118 | +++ | 119 | ++ | 120 | +++ |
| 121 | + | 122 | +++ | 123 | +++ | 124 | +++ |
| 125 | ++ | 126 | +++ | 127 | + | 128 | ++ |
| 129 | +++ | 130 | +++ | 131 | +++ | 132 | +++ |
| 133 | +++ | 134 | ++ | 135 | +++ | 136 | ++ |
| 137 | + | 138 | +++ | 139 | +++ | 140 | +++ |
| 141 | ++ | 142 | +++ | 143 | ++ | 144 | ++ |
| 145 | +++ | 146 | +++ | 147 | +++ | 148 | +++ |
| 149 | +++ | 150 | +++ | 151 | +++ | 152 | +++ |
| 153 | +++ | 154 | +++ | 155 | ++ | 156 | +++ |
| 157 | + | 158 | +++ | 159 | +++ | 160 | +++ |
| 161 | +++ | 162 | +++ | 163 | +++ | 164 | +++ |
| 165 | ++ | 166 | ++ | 167 | +++ | 168 | + |
| 169 | ++ | | | | | | |

All publications, including but not limited to disclosures and disclosure applications, cited in this specification are herein incorporated by reference as though fully set forth. If certain content of a publication cited herein contradicts or is inconsistent with the present disclosure, the present disclosure controls.

One skilled in the art will readily recognize from the disclosure and claims that various changes, modifications, and variations can be made therein without departing from the spirit and scope of the disclosure as defined in the following claims.

1. A compound of one of the following structural Formulae Ia and Ib: 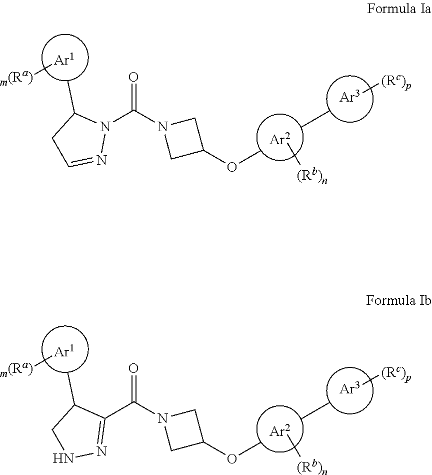
a tautomer thereof, a hydrate or stereoisomer of the compound or the tautomer, or a pharmaceutically acceptable salt of the foregoing, wherein:
Ar¹ is phenyl, C₅-C₆ cycloalkyl, 5- to 6-membered heteroaryl, or 5- to 6-membered heterocyclyl;
Ar² is phenyl, C₅-C₆ cycloalkyl, 5- to 6-membered heteroaryl, or 5- to 6-membered heterocyclyl, provided that when 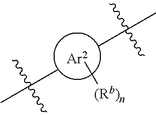
in formula Ia is 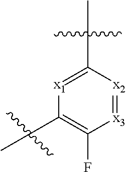
wherein X₁, X₂, and X₃ are C; or X₁ is N, X₂ and X₃ are C; or X₂ is N, X₁ and X₃ are C; or X₁ and X₂ are N, and X₃ is C; or X₁ and X₂ are C, and X₃ is N, 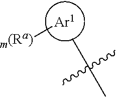
cannot be 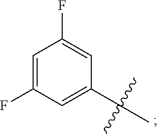 [SMILES: *c1cc(F)cc(F)c1]
Ar³ is phenyl, C₅-C₆ cycloalkyl, 5- to 6-membered heteroaryl, or 5- to 6-membered heterocyclyl;
R^{a}, for each occurrence, is independently selected from halogen, CN, C₁-C₃ alkyl, and OH;
R^{b}, for each occurrence, is independently selected from halogen, CN, C₁-C₃ alkyl, and OH;
R^{c}, for each occurrence, is independently selected from halogen, cyano, C₁-C₆ alkyl, C₃-C₆ cycloalkyl, 3- to 6-membered heterocyclyl, C₂-C₆ alkenyl, C₁-C₆ alkoxy, —C(═O)(C₁-C₆ alkyl), —C(═O)(C₃-C₆ cycloalkyl), —C(═O)(3- to 6-membered heterocyclyl), ═O, —NO₂, —C(═O)OR^{s}, —C(═O)NR^{p}R^{q}, —NR^{p}R^{q}, —NR^{p}C(═O)R^{s}, —NR^{p}C(═O)OR^{s}, —NR^{p}C(═O)NR^{q}R^{r}, —NR^{p}S(═O)_{w}R^{s}, —OR^{s}, —OC(═O)R^{s}, —OC(═O)OR^{s}, —OC(═O)NR^{p}R^{q}, —S(═O)_{w}R^{s}, and —S(═O)_{w}NR^{p}R^{q}; wherein
the C₁-C₆ alkyl, C₃-C₆ cycloalkyl, 3- to 6-membered heterocyclyl, C₂-C₆ alkenyl, and C₁-C₆ alkoxy of R^{c}, the C₁-C₆ alkyl of —C(═O)(C₁-C₆ alkyl), the C₃-C₆ cycloalkyl of —C(═O)(C₃-C₆ cycloalkyl), and the 3- to 6-membered heterocyclyl of —C(═O)(3- to 6-membered heterocyclyl) are each optionally substituted with 1 to 3 groups selected from halogen, cyano, —C(═O)R^{s}, —C(═O)OR^{s}, —C(═O)NR^{p}R^{q}, —NR^{p}R^{q}, —NR^{p}C(═O)R^{s}, —NR^{p}C(═O)OR^{s}, —NR^{p}C(═O)NR^{q}R^{r}, —NR^{p}S(═O)_{w}R^{s}, —OR^{s}, —OC(═O)R^{s}, —OC(═O)OR^{s}, —OC(═O)NR^{p}R^{q}, —S(═O)_{w}R^{s}, —S(═O)_{w}NR^{p}R^{q}, C₃-C₆ cycloalkyl, and 3- to 6-membered heterocyclyl; wherein
R^{p}, R^{q}, R^{r}, and R^{s}, for each occurrence, are each independently selected from hydrogen, OH, NH₂, C₁-C₄ alkyl, —C(═O)(C₁-C₄ alkyl), C₃-C₆ cycloalkyl, and 3- to 6-membered heterocyclyl; wherein
the C₁-C₄ alkyl, C₃-C₆ cycloalkyl, and 3- to 6-membered heterocyclyl of any one of R^{p}, R^{q}, R^{r}, and R^{s} are optionally substituted with 1 to 3 groups selected from halogen, cyano, —OH, C₁-C₆ alkyl, —O(C₁-C₆ alkyl), —C(═O)N(C₁-C₆ alkyl)(C₁-C₆ alkyl), —C(═O)NH(C₁-C₆ alkyl), —C(═O)(3- to 6-membered heterocyclyl), —C(═O)(C₃-C₆ cycloalkyl), C₃-C₆ cycloalkyl, phenyl, and 3- to 6-membered heterocyclyl; and wherein
w is an integer selected from 0, 1 and 2;
m and p are each an integer independently selected from 0, 1, 2, and 3; and
n is selected from 0, 1, and 2.

2. The compound, tautomer, hydrate, stereoisomer, or pharmaceutically acceptable salt of claim 1, wherein Ar¹ is phenyl or 5- to 6-membered heteroaryl, Ar² is phenyl or 6-membered heteroaryl, and Ar³ is 5- to 6-membered heteroaryl.

3. The compound of claim 1, wherein the compound has the following structural formula IIa: 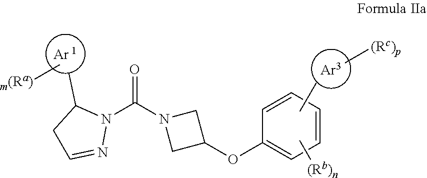
a tautomer thereof, a hydrate or stereoisomer of the compound or the tautomer, or a pharmaceutically acceptable salt of the foregoing.

4. The compound of claim 1, wherein the compound has the following structural formula IIb: 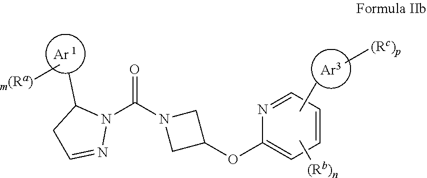
a tautomer thereof, a hydrate or stereoisomer of the compound or the tautomer, or a pharmaceutically acceptable salt of the foregoing.

5. The compound of claim 1, wherein the compound has the following structural formula IIc: 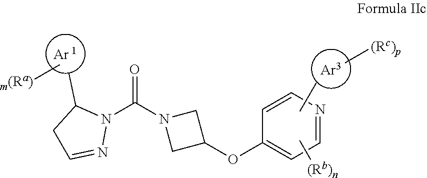
a tautomer thereof, a hydrate or stereoisomer of the compound or the tautomer, or a pharmaceutically acceptable salt of the foregoing.

6. The compound of claim 1, wherein the compound has the following structural formula IId: 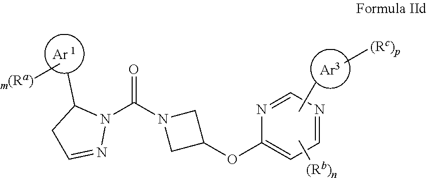
a tautomer thereof, a hydrate or stereoisomer of the compound or the tautomer, or a pharmaceutically acceptable salt of the foregoing.

7. The compound of claim 1, wherein the compound has the following structural formula IIe: 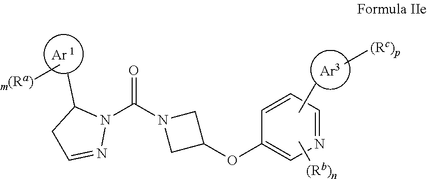
a tautomer thereof, a hydrate or stereoisomer of the compound or the tautomer, or a pharmaceutically acceptable salt of the foregoing.

8. The compound of claim 1, wherein the compound has the following structural formula IIf: 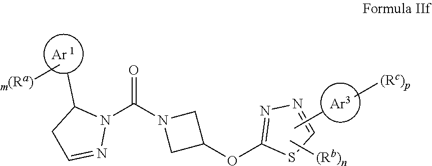
a tautomer thereof, a hydrate or stereoisomer of the compound or the tautomer, or a pharmaceutically acceptable salt of the foregoing.

9. The compound of claim 1, wherein the compound has the following structural formula IIg: 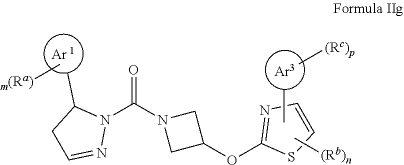
a tautomer thereof, a hydrate or stereoisomer of the compound or the tautomer, or a pharmaceutically acceptable salt of the foregoing.

10. The compound of claim 1, wherein the compound has the following structural formula IIh: 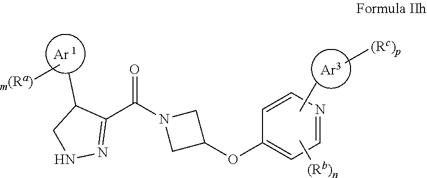
a tautomer thereof, a hydrate or stereoisomer of the compound or the tautomer, or a pharmaceutically acceptable salt of the foregoing.

11. The compound of claim 1, wherein the compound has one of the following structural formula IIIa-1 and formula IIIa-2: 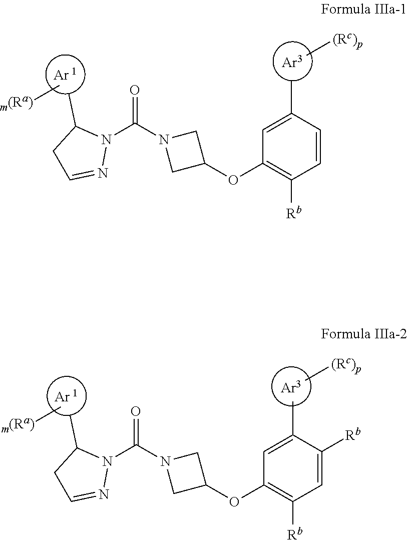
a tautomer thereof, a hydrate or stereoisomer of the compound or the tautomer, or a pharmaceutically acceptable salt of the foregoing, wherein R^{b}, for each occurrence, is independently selected from F and Cl.

12. The compound of claim 1, wherein the compound has one of the following structural formula IIIb-1 and formula IIIb-2: 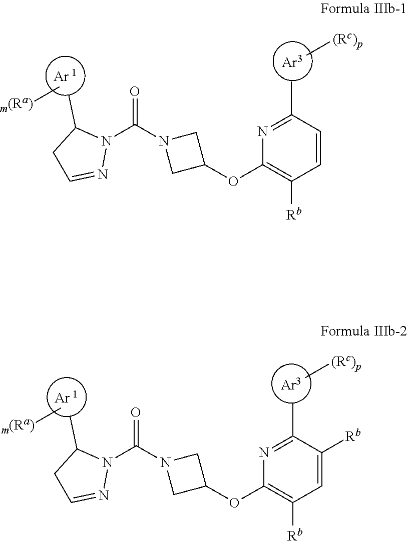
a tautomer thereof, a hydrate or stereoisomer of the compound or the tautomer, or a pharmaceutically acceptable salt of the foregoing, R^{b}, for each occurrence, is independently selected from F and Cl.

13. The compound of claim 1, wherein the compound has the following structural formula IIIc-1: 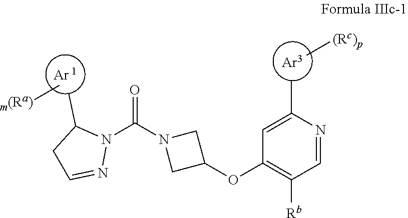
a tautomer thereof, a hydrate or stereoisomer of the compound or the tautomer, or a pharmaceutically acceptable salt of the foregoing, wherein R^{b} is selected from F and Cl.

14. The compound of claim 1, wherein the compound has the following structural formula IIId-1: 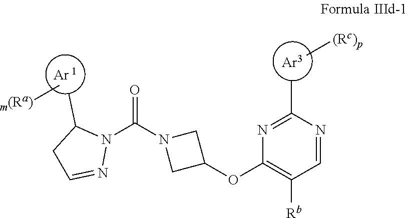
a tautomer thereof, a hydrate or stereoisomer of the compound or the tautomer, or a pharmaceutically acceptable salt of the foregoing, wherein R^{b} is selected from F and Cl.

15. The compound of claim 1, wherein the compound has the following structural formula IIIe-1: 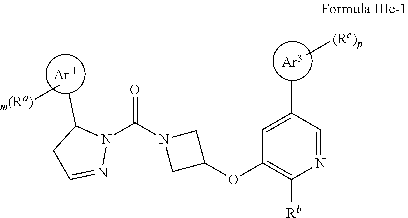
a tautomer thereof, a hydrate or stereoisomer of the compound or the tautomer, or a pharmaceutically acceptable salt of the foregoing, wherein R^{b} is selected from F and Cl.

16. The compound of claim 1, wherein the compound has the following structural formula IIIf-1: 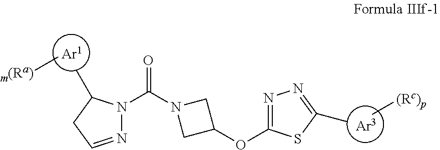
a tautomer thereof, a hydrate or stereoisomer of the compound or the tautomer, or a pharmaceutically acceptable salt of the foregoing.

17. The compound of claim 1, wherein the compound has one of the following structural formula IIIg-1 and IIIg-2: 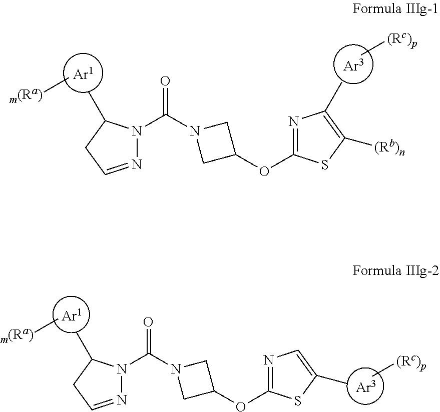
a tautomer thereof, a hydrate or stereoisomer of the compound or the tautomer, or a pharmaceutically acceptable salt of the foregoing, wherein R^{b} is selected from CN and Cl.

18. The compound of claim 1, wherein the compound has the following structural formula IIIh-1: 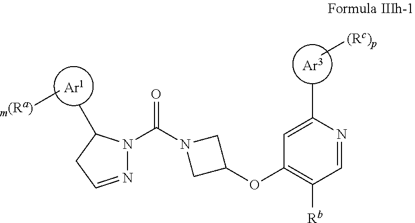
a tautomer thereof, a hydrate or stereoisomer of the compound or the tautomer, or a pharmaceutically acceptable salt of the foregoing, wherein R^{b} is selected from F and Cl.

19. The compound, tautomer, hydrate, stereoisomer, or pharmaceutically acceptable salt of claim 1, wherein

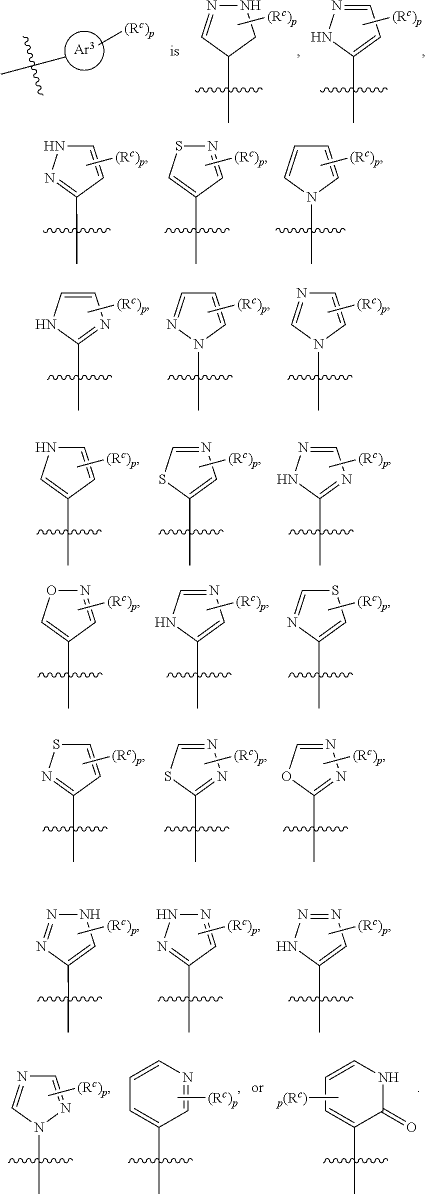

20. The compound, tautomer, hydrate, stereoisomer, or pharmaceutically acceptable salt of claim 1, wherein

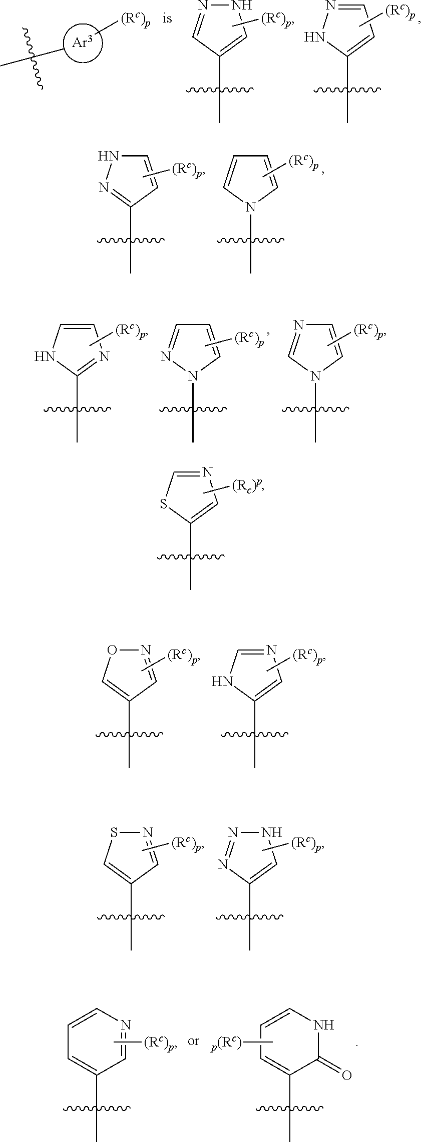

21. The compound, tautomer, hydrate, stereoisomer, or pharmaceutically acceptable salt of claim 1, wherein

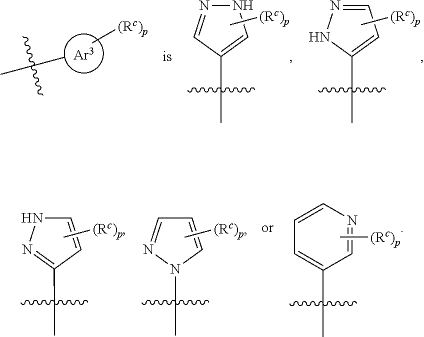

22. The compound, tautomer, hydrate, stereoisomer, or pharmaceutically acceptable salt of claim 1, wherein

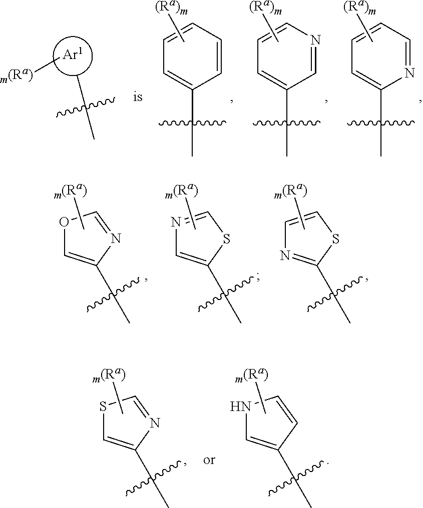

23. The compound, tautomer, hydrate, stereoisomer, or pharmaceutically acceptable salt of claim 1, wherein R^{a}, for each occurrence, is independently selected from F, Cl, CN, C₁-C₃ alkyl, and OH.

24. The compound, tautomer, hydrate, stereoisomer, or pharmaceutically acceptable salt of claim 1, wherein R^{a}, for each occurrence, is independently selected from F, Cl, CN, and methyl.

25. The compound, tautomer, hydrate, stereoisomer, or pharmaceutically acceptable salt of claim 1, wherein m is 1 or 2.

26. The compound, tautomer, hydrate, stereoisomer, or pharmaceutically acceptable salt of claim 1, wherein R^{c}, for each occurrence, is independently selected from halogen;
CN;
═O;
—C(═O)OR^{s}, wherein R^{s} is H or C₁-C₃ alkyl;
C₁-C₃ alkyl, optionally substituted with 1 to 3 groups selected from OH, NH₂, cyano, halogen, C₁-C₃ alkoxyl, 3- to 4-membered cycloalkyl;
—C(═O)NR^{p}R^{q}, wherein R^{p} and R^{q} each are independently selected from H, OH, 3- to 4-membered cycloalkyl, and 4- to 6-membered heterocyclyl;
—NR^{p}R^{q}, wherein R^{p} and R^{q} each are independently selected from H, OH, —C(═O)CH₃, and C₁-C₃ alkyl;
—S(═O)_{w}R^{s}, wherein R^{s} is selected from C₁-C₃ alkyl and wherein w is 0 or 2; and
—S(═O)_{w}NR^{p}R^{q}, wherein R^{p} and R^{q} each are independently selected from H and C₁-C₃ alkyl and wherein w is 2.

27. The compound, tautomer, hydrate, stereoisomer, or pharmaceutically acceptable salt of claim 1, wherein R^{c}, for each occurrence, is independently selected from methyl, Cl, CN, ethyl, —C(═O)NH₂, —CH₂CH₂OCH₃, —CH₂C(═O)NH₂, —NH₂, F, —S(═O)₂CH₂CH₃, 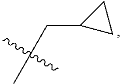 [SMILES: *CC1CC1]
—C(═O)NHCH₂CH₂OH, 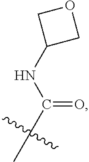 [SMILES: *C(=O)NC1COC1]
—C(═O)NHCH₃, 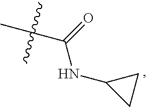 [SMILES: *C(=O)NC1CC1]
—C(═O)OH, —C(═O)NHCH₂CH₃, and —S(═O)₂NH₂.

28. The compound, tautomer, hydrate, stereoisomer, or pharmaceutically acceptable salt of claim 1, wherein R^{c}, for each occurrence, is independently selected from CN, methyl, ethyl, F, Cl, and —C(═O)NH₂.

29. The compound, tautomer, hydrate, stereoisomer, or pharmaceutically acceptable salt of claim 1, wherein p is 1, 2, or 3.

30. The compound of claim 1, wherein the compound has the following structural formula IVa: 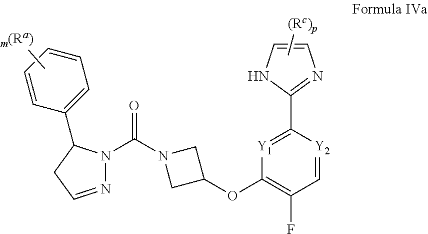
a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

31. The compound of claim 1, wherein the compound has the following structural formula IVb: 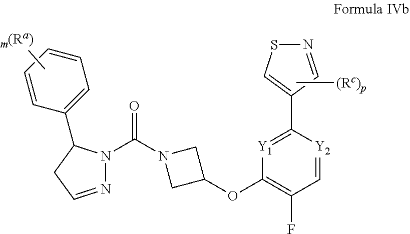
a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

32. The compound of claim 1, wherein the compound has the following structural formula IVc: 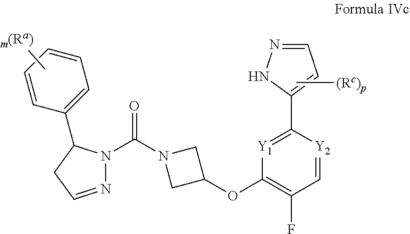
a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

33. The compound of claim 1, wherein the compound has one of the following structural formula IVd: 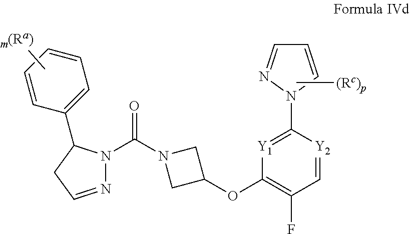
a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

34. The compound of claim 1, wherein the compound has the following structural formula IVe: 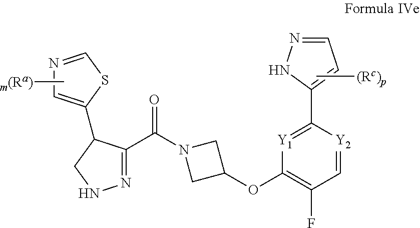
a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

35. The compound of claim 1, wherein the compound has the following structural formula IVf: 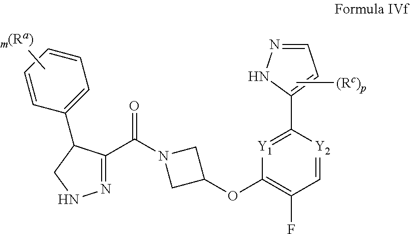
a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

36. The compound of claim 1, wherein the compound has the following structural formula IVg: 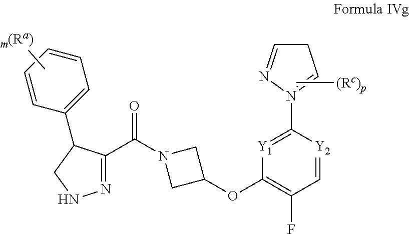
a tautomer thereof, a hydrate or stereoisomer of the compound or the tautomer, or a pharmaceutically acceptable salt of the foregoing, wherein Y₁ is N and Y₂ is C, Y₁ is C and Y₂ is N, or Y₁ and Y₂ are C; R^{a}, for each occurrence, is independently selected from F and CN; m is 1, 2, or 3; R^{c}, for each occurrence, is independently selected from methyl, F, CN, —S(═O)₂NH₂, and —C(═O)NH₂; and p is 1, 2, or 3.

37. The compound according to claim 1, wherein the compound is selected from:

| | |
|---|---|
| 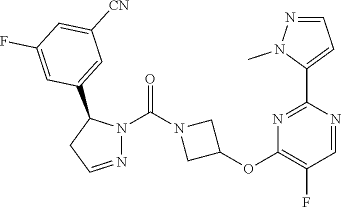 [SMILES: Cn1nccc1-c1ncc(F)c(OC2CN(C(=O)N3N=CC[C@H]3c3cc(F)cc(C#N)c3)C2)n1] | 1 |
| 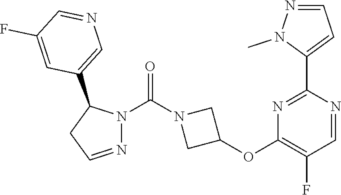 [SMILES: Cn1nccc1-c1ncc(F)c(OC2CN(C(=O)N3N=CC[C@H]3c3cncc(F)c3)C2)n1] | 2 |
| 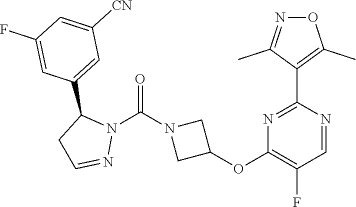 [SMILES: Cc1noc(C)c1-c1ncc(F)c(OC2CN(C(=O)N3N=CC[C@H]3c3cc(F)cc(C#N)c3)C2)n1] | 3 |
| 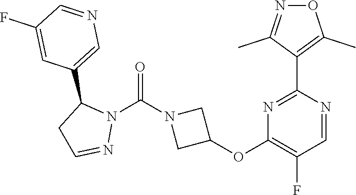 [SMILES: Cc1noc(C)c1-c1ncc(F)c(OC2CN(C(=O)N3N=CC[C@H]3c3cncc(F)c3)C2)n1] | 4 |
| 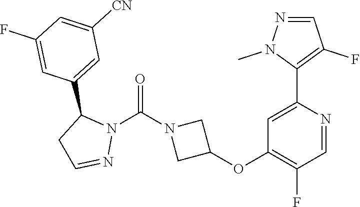 [SMILES: Cn1ncc(F)c1-c1cc(OC2CN(C(=O)N3N=CC[C@H]3c3cc(F)cc(C#N)c3)C2)c(F)cn1] | 5 |
| 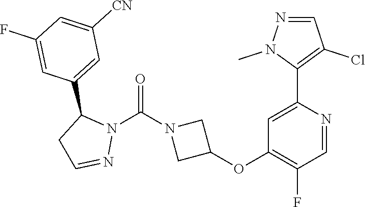 [SMILES: Cn1ncc(Cl)c1-c1cc(OC2CN(C(=O)N3N=CC[C@H]3c3cc(F)cc(C#N)c3)C2)c(F)cn1] | 6 |
| 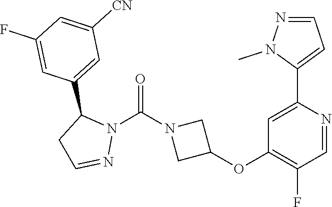 [SMILES: Cn1nccc1-c1cc(OC2CN(C(=O)N3N=CC[C@H]3c3cc(F)cc(C#N)c3)C2)c(F)cn1] | 7 |
| 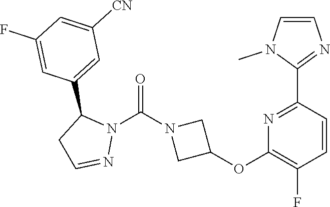 [SMILES: Cn1ccnc1-c1ccc(F)c(OC2CN(C(=O)N3N=CC[C@H]3c3cc(F)cc(C#N)c3)C2)n1] | 8 |
| 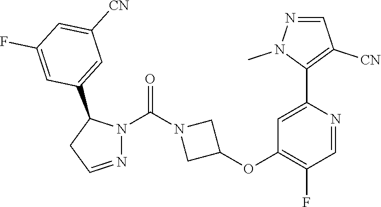 [SMILES: Cn1ncc(C#N)c1-c1cc(OC2CN(C(=O)N3N=CC[C@H]3c3cc(F)cc(C#N)c3)C2)c(F)cn1] | 9 |
| 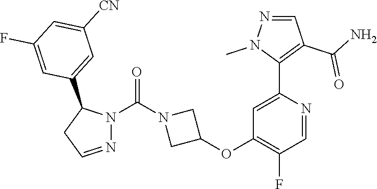 [SMILES: Cn1ncc(C(N)=O)c1-c1cc(OC2CN(C(=O)N3N=CC[C@H]3c3cc(F)cc(C#N)c3)C2)c(F)cn1] | 10 |
| 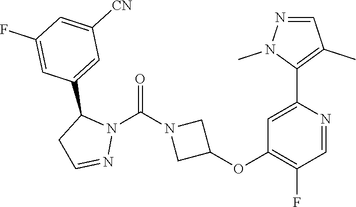 [SMILES: Cc1cnn(C)c1-c1cc(OC2CN(C(=O)N3N=CC[C@H]3c3cc(F)cc(C#N)c3)C2)c(F)cn1] | 11 |
| 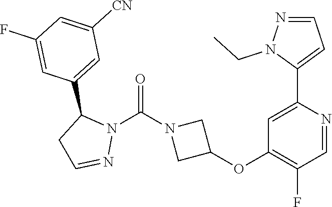 [SMILES: CCn1nccc1-c1cc(OC2CN(C(=O)N3N=CC[C@H]3c3cc(F)cc(C#N)c3)C2)c(F)cn1] | 12 |
| 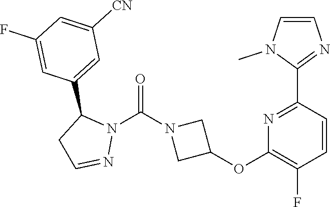 [SMILES: Cn1ccnc1-c1ccc(F)c(OC2CN(C(=O)N3N=CC[C@H]3c3cc(F)cc(C#N)c3)C2)n1] | 13 |
| 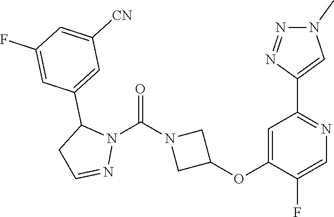 [SMILES: Cn1cc(-c2cc(OC3CN(C(=O)N4N=CCC4c4cc(F)cc(C#N)c4)C3)c(F)cn2)nn1] | 14 |
| 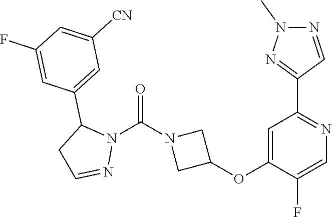 [SMILES: Cn1ncc(-c2cc(OC3CN(C(=O)N4N=CCC4c4cc(F)cc(C#N)c4)C3)c(F)cn2)n1] | 15 |
| 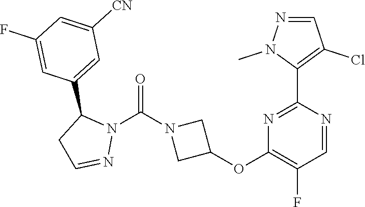 [SMILES: Cn1ncc(Cl)c1-c1ncc(F)c(OC2CN(C(=O)N3N=CC[C@H]3c3cc(F)cc(C#N)c3)C2)n1] | 16 |
| 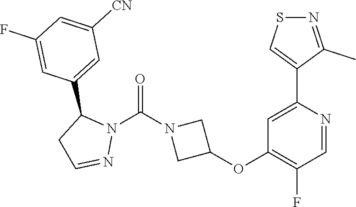 [SMILES: Cc1nscc1-c1cc(OC2CN(C(=O)N3N=CC[C@H]3c3cc(F)cc(C#N)c3)C2)c(F)cn1] | 17 |
| 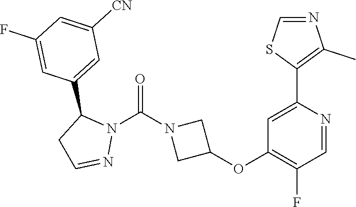 [SMILES: Cc1ncsc1-c1cc(OC2CN(C(=O)N3N=CC[C@H]3c3cc(F)cc(C#N)c3)C2)c(F)cn1] | 18 |
| 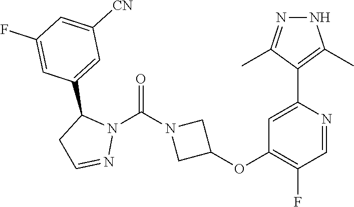 [SMILES: Cc1n[nH]c(C)c1-c1cc(OC2CN(C(=O)N3N=CC[C@H]3c3cc(F)cc(C#N)c3)C2)c(F)cn1] | 19 |
| 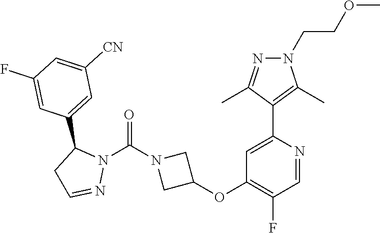 [SMILES: COCCn1nc(C)c(-c2cc(OC3CN(C(=O)N4N=CC[C@H]4c4cc(F)cc(C#N)c4)C3)c(F)cn2)c1C] | 20 |
| 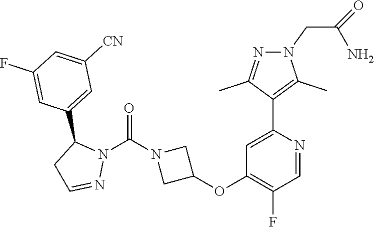 [SMILES: Cc1nn(CC(N)=O)c(C)c1-c1cc(OC2CN(C(=O)N3N=CC[C@H]3c3cc(F)cc(C#N)c3)C2)c(F)cn1] | 21 |
| 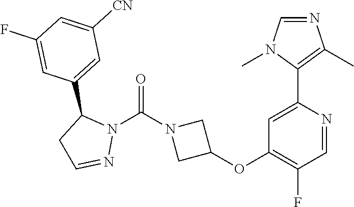 [SMILES: Cc1ncn(C)c1-c1cc(OC2CN(C(=O)N3N=CC[C@H]3c3cc(F)cc(C#N)c3)C2)c(F)cn1] | 22 |
| 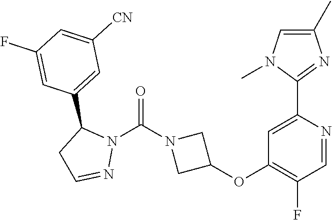 [SMILES: Cc1cn(C)c(-c2cc(OC3CN(C(=O)N4N=CC[C@H]4c4cc(F)cc(C#N)c4)C3)c(F)cn2)n1] | 23 |
| 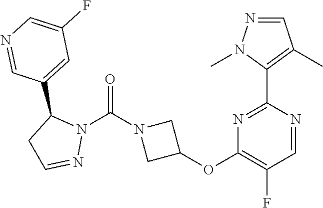 [SMILES: Cc1cnn(C)c1-c1ncc(F)c(OC2CN(C(=O)N3N=CC[C@H]3c3cncc(F)c3)C2)n1] | 24 |
| 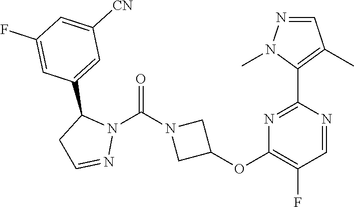 [SMILES: Cc1cnn(C)c1-c1ncc(F)c(OC2CN(C(=O)N3N=CC[C@H]3c3cc(F)cc(C#N)c3)C2)n1] | 25 |
| 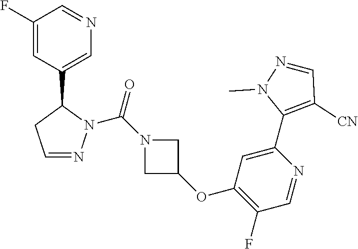 [SMILES: Cn1ncc(C#N)c1-c1cc(OC2CN(C(=O)N3N=CC[C@H]3c3cncc(F)c3)C2)c(F)cn1] | 26 |
| 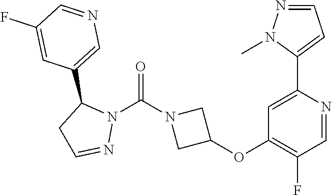 [SMILES: Cn1nccc1-c1cc(OC2CN(C(=O)N3N=CC[C@H]3c3cncc(F)c3)C2)c(F)cn1] | 27 |
| 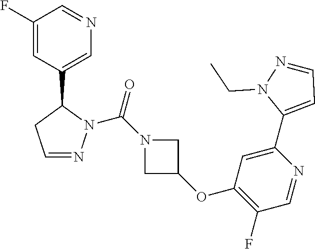 [SMILES: CCn1nccc1-c1cc(OC2CN(C(=O)N3N=CC[C@H]3c3cncc(F)c3)C2)c(F)cn1] | 28 |
| 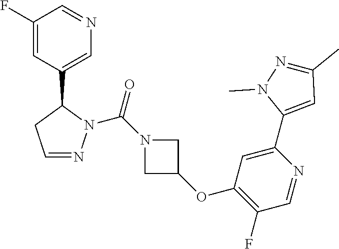 [SMILES: Cc1cc(-c2cc(OC3CN(C(=O)N4N=CC[C@H]4c4cncc(F)c4)C3)c(F)cn2)n(C)n1] | 29 |
| 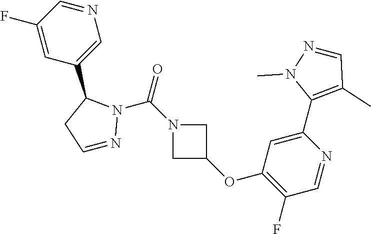 [SMILES: Cc1cnn(C)c1-c1cc(OC2CN(C(=O)N3N=CC[C@H]3c3cncc(F)c3)C2)c(F)cn1] | 30 |
| 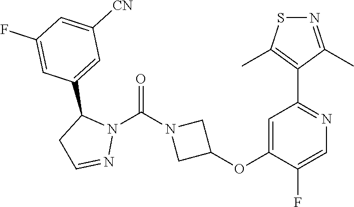 [SMILES: Cc1nsc(C)c1-c1cc(OC2CN(C(=O)N3N=CC[C@H]3c3cc(F)cc(C#N)c3)C2)c(F)cn1] | 31 |
| 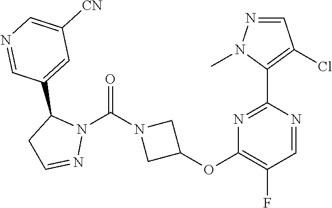 [SMILES: Cn1ncc(Cl)c1-c1ncc(F)c(OC2CN(C(=O)N3N=CC[C@H]3c3cncc(C#N)c3)C2)n1] | 32 |
| 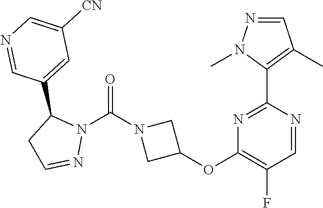 [SMILES: Cc1cnn(C)c1-c1ncc(F)c(OC2CN(C(=O)N3N=CC[C@H]3c3cncc(C#N)c3)C2)n1] | 33 |
| 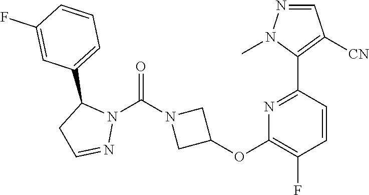 [SMILES: Cn1ncc(C#N)c1-c1ccc(F)c(OC2CN(C(=O)N3N=CC[C@H]3c3cccc(F)c3)C2)n1] | 34 |
| 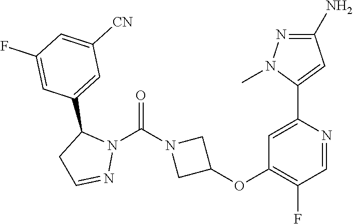 [SMILES: Cn1nc(N)cc1-c1cc(OC2CN(C(=O)N3N=CC[C@H]3c3cc(F)cc(C#N)c3)C2)c(F)cn1] | 35 |
| 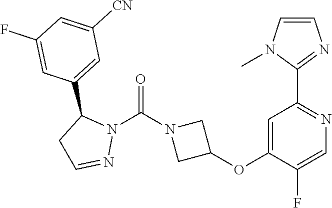 [SMILES: Cn1ccnc1-c1cc(OC2CN(C(=O)N3N=CC[C@H]3c3cc(F)cc(C#N)c3)C2)c(F)cn1] | 36 |
| 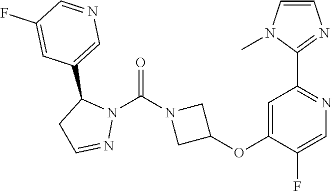 [SMILES: Cn1ccnc1-c1cc(OC2CN(C(=O)N3N=CC[C@H]3c3cncc(F)c3)C2)c(F)cn1] | 37 |
| 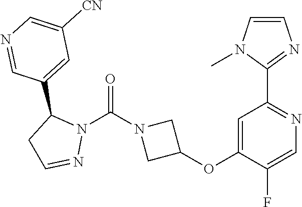 [SMILES: Cn1ccnc1-c1cc(OC2CN(C(=O)N3N=CC[C@H]3c3cncc(C#N)c3)C2)c(F)cn1] | 38 |
| 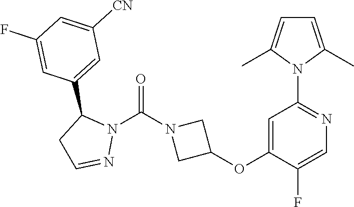 [SMILES: Cc1ccc(C)n1-c1cc(OC2CN(C(=O)N3N=CC[C@H]3c3cc(F)cc(C#N)c3)C2)c(F)cn1] | 39 |
| 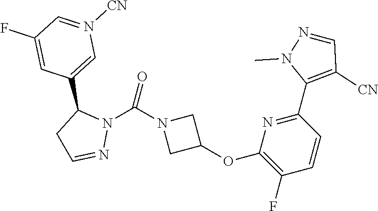 | 40 |
| 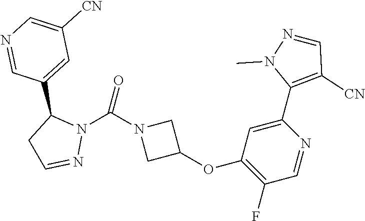 [SMILES: Cn1ncc(C#N)c1-c1cc(OC2CN(C(=O)N3N=CC[C@H]3c3cncc(C#N)c3)C2)c(F)cn1] | 41 |
| 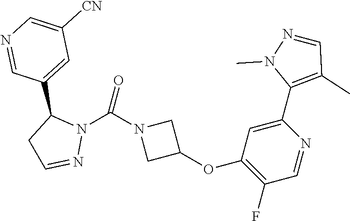 [SMILES: Cc1cnn(C)c1-c1cc(OC2CN(C(=O)N3N=CC[C@H]3c3cncc(C#N)c3)C2)c(F)cn1] | 42 |
| 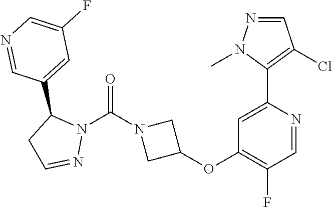 [SMILES: Cn1ncc(Cl)c1-c1cc(OC2CN(C(=O)N3N=CC[C@H]3c3cncc(F)c3)C2)c(F)cn1] | 43 |
| 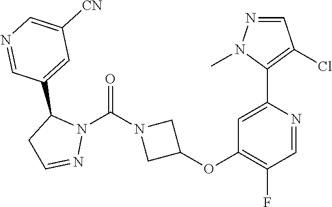 [SMILES: Cn1ncc(Cl)c1-c1cc(OC2CN(C(=O)N3N=CC[C@H]3c3cncc(C#N)c3)C2)c(F)cn1] | 44 |
| 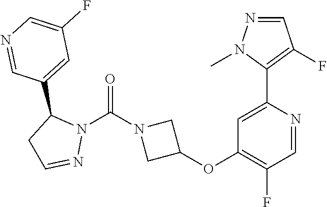 [SMILES: Cn1ncc(F)c1-c1cc(OC2CN(C(=O)N3N=CC[C@H]3c3cncc(F)c3)C2)c(F)cn1] | 45 |
| 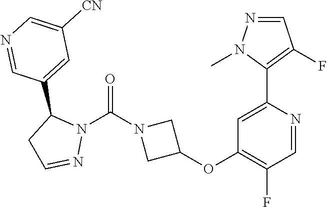 [SMILES: Cn1ncc(F)c1-c1cc(OC2CN(C(=O)N3N=CC[C@H]3c3cncc(C#N)c3)C2)c(F)cn1] | 46 |
| 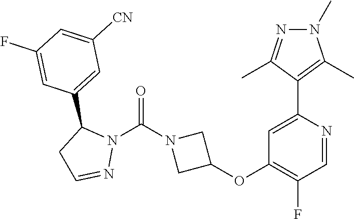 [SMILES: Cc1nn(C)c(C)c1-c1cc(OC2CN(C(=O)N3N=CC[C@H]3c3cc(F)cc(C#N)c3)C2)c(F)cn1] | 47 |
| 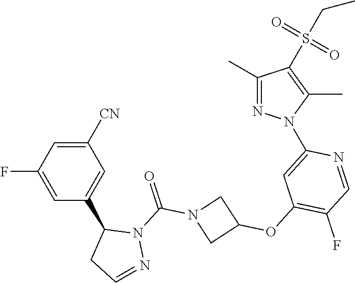 [SMILES: CCS(=O)(=O)c1c(C)nn(-c2cc(OC3CN(C(=O)N4N=CC[C@H]4c4cc(F)cc(C#N)c4)C3)c(F)cn2)c1C] | 48 |
| 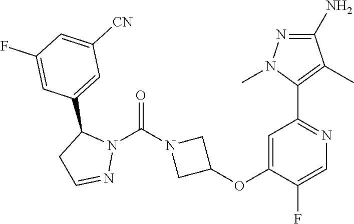 [SMILES: Cc1c(N)nn(C)c1-c1cc(OC2CN(C(=O)N3N=CC[C@H]3c3cc(F)cc(C#N)c3)C2)c(F)cn1] | 49 |
| 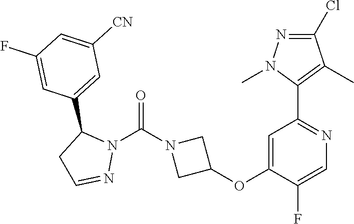 [SMILES: Cc1c(Cl)nn(C)c1-c1cc(OC2CN(C(=O)N3N=CC[C@H]3c3cc(F)cc(C#N)c3)C2)c(F)cn1] | 50 |
| 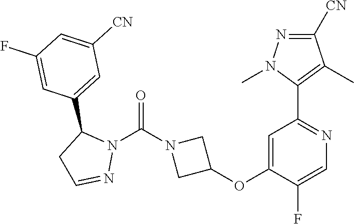 [SMILES: Cc1c(C#N)nn(C)c1-c1cc(OC2CN(C(=O)N3N=CC[C@H]3c3cc(F)cc(C#N)c3)C2)c(F)cn1] | 51 |
| 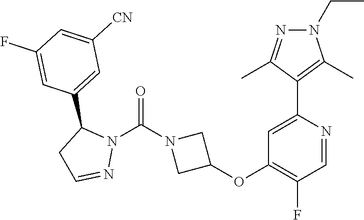 [SMILES: CCn1nc(C)c(-c2cc(OC3CN(C(=O)N4N=CC[C@H]4c4cc(F)cc(C#N)c4)C3)c(F)cn2)c1C] | 52 |
| 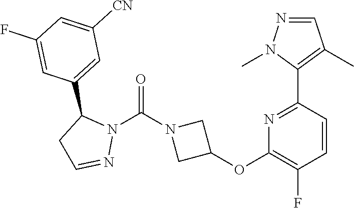 [SMILES: Cc1cnn(C)c1-c1ccc(F)c(OC2CN(C(=O)N3N=CC[C@H]3c3cc(F)cc(C#N)c3)C2)n1] | 53 |
| 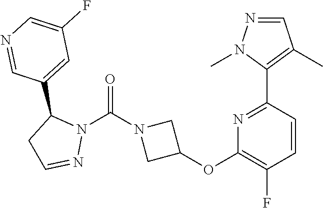 [SMILES: Cc1cnn(C)c1-c1ccc(F)c(OC2CN(C(=O)N3N=CC[C@H]3c3cncc(F)c3)C2)n1] | 54 |
| 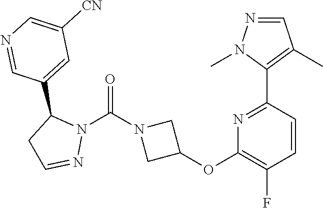 [SMILES: Cc1cnn(C)c1-c1ccc(F)c(OC2CN(C(=O)N3N=CC[C@H]3c3cncc(C#N)c3)C2)n1] | 55 |
| 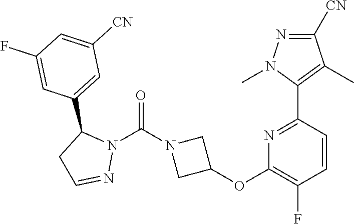 [SMILES: Cc1c(C#N)nn(C)c1-c1ccc(F)c(OC2CN(C(=O)N3N=CC[C@H]3c3cc(F)cc(C#N)c3)C2)n1] | 56 |
| 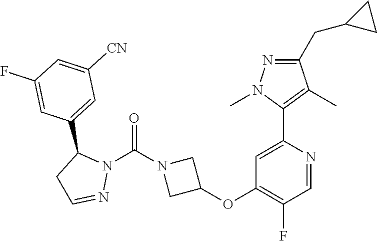 [SMILES: Cc1c(CC2CC2)nn(C)c1-c1cc(OC2CN(C(=O)N3N=CC[C@H]3c3cc(F)cc(C#N)c3)C2)c(F)cn1] | 57 |
| 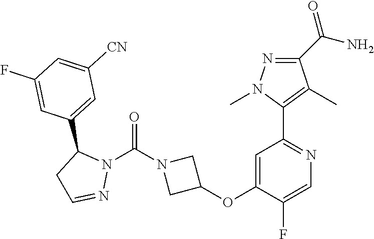 [SMILES: Cc1c(C(N)=O)nn(C)c1-c1cc(OC2CN(C(=O)N3N=CC[C@H]3c3cc(F)cc(C#N)c3)C2)c(F)cn1] | 58 |
| 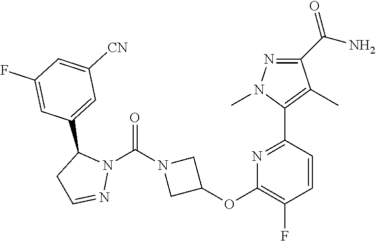 [SMILES: Cc1c(C(N)=O)nn(C)c1-c1ccc(F)c(OC2CN(C(=O)N3N=CC[C@H]3c3cc(F)cc(C#N)c3)C2)n1] | 59 |
| 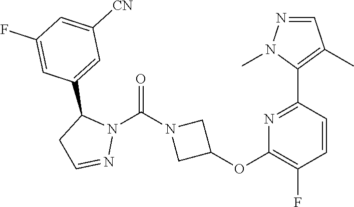 [SMILES: Cc1cnn(C)c1-c1ccc(F)c(OC2CN(C(=O)N3N=CC[C@H]3c3cc(F)cc(C#N)c3)C2)n1] | 60 |
| 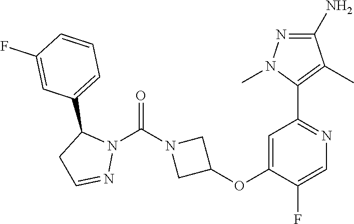 [SMILES: Cc1c(N)nn(C)c1-c1cc(OC2CN(C(=O)N3N=CC[C@H]3c3cccc(F)c3)C2)c(F)cn1] | 61 |
| 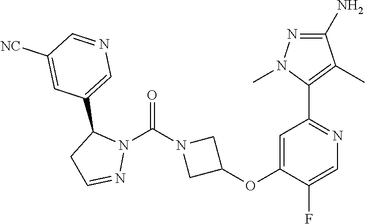 [SMILES: Cc1c(N)nn(C)c1-c1cc(OC2CN(C(=O)N3N=CC[C@H]3c3cncc(C#N)c3)C2)c(F)cn1] | 62 |
| 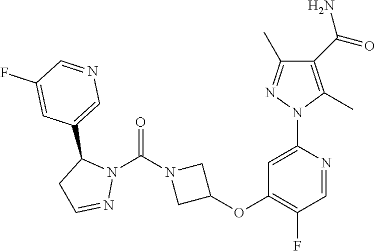 [SMILES: Cc1nn(-c2cc(OC3CN(C(=O)N4N=CC[C@H]4c4cncc(F)c4)C3)c(F)cn2)c(C)c1C(N)=O] | 63 |
| 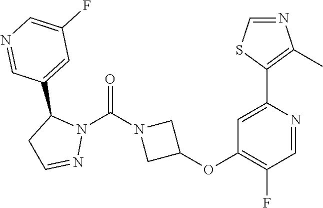 [SMILES: Cc1ncsc1-c1cc(OC2CN(C(=O)N3N=CC[C@H]3c3cncc(F)c3)C2)c(F)cn1] | 64 |
| 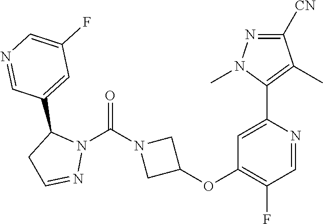 [SMILES: Cc1c(C#N)nn(C)c1-c1cc(OC2CN(C(=O)N3N=CC[C@H]3c3cncc(F)c3)C2)c(F)cn1] | 65 |
| 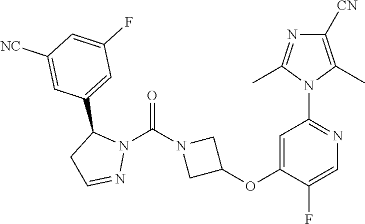 [SMILES: Cc1nc(C#N)c(C)n1-c1cc(OC2CN(C(=O)N3N=CC[C@H]3c3cc(F)cc(C#N)c3)C2)c(F)cn1] | 66 |
| 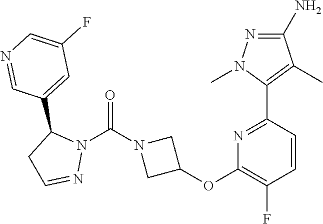 [SMILES: Cc1c(N)nn(C)c1-c1ccc(F)c(OC2CN(C(=O)N3N=CC[C@H]3c3cncc(F)c3)C2)n1] | 67 |
| 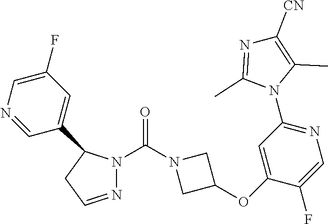 [SMILES: Cc1nc(C#N)c(C)n1-c1cc(OC2CN(C(=O)N3N=CC[C@H]3c3cncc(F)c3)C2)c(F)cn1] | 68 |
| 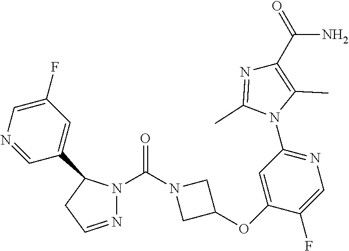 [SMILES: Cc1nc(C(N)=O)c(C)n1-c1cc(OC2CN(C(=O)N3N=CC[C@H]3c3cncc(F)c3)C2)c(F)cn1] | 69 |
| 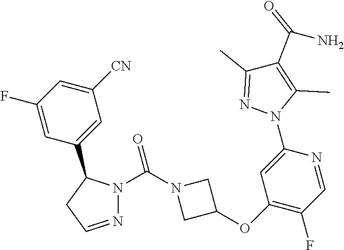 [SMILES: Cc1nn(-c2cc(OC3CN(C(=O)N4N=CC[C@H]4c4cc(F)cc(C#N)c4)C3)c(F)cn2)c(C)c1C(N)=O] | 70 |
| 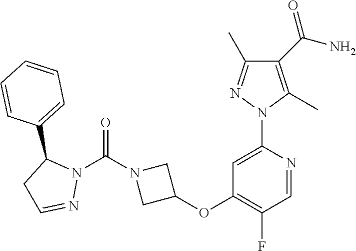 [SMILES: Cc1nn(-c2cc(OC3CN(C(=O)N4N=CC[C@H]4c4ccccc4)C3)c(F)cn2)c(C)c1C(N)=O] | 71 |
| 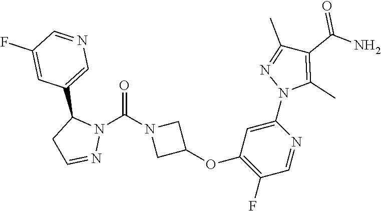 [SMILES: Cc1nn(-c2cc(OC3CN(C(=O)N4N=CC[C@H]4c4cncc(F)c4)C3)c(F)cn2)c(C)c1C(N)=O] | 72 |
| 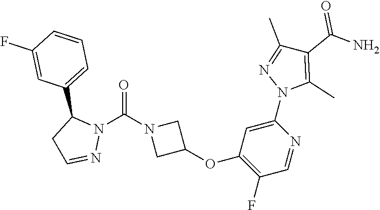 [SMILES: Cc1nn(-c2cc(OC3CN(C(=O)N4N=CC[C@H]4c4cccc(F)c4)C3)c(F)cn2)c(C)c1C(N)=O] | 73 |
| 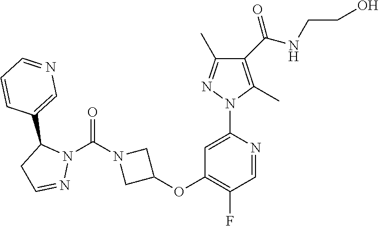 [SMILES: Cc1nn(-c2cc(OC3CN(C(=O)N4N=CC[C@H]4c4cccnc4)C3)c(F)cn2)c(C)c1C(=O)NCCO] | 74 |
| 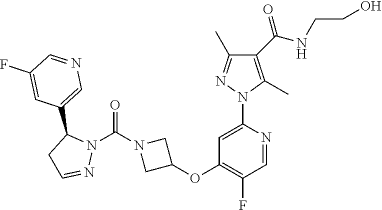 [SMILES: Cc1nn(-c2cc(OC3CN(C(=O)N4N=CC[C@H]4c4cncc(F)c4)C3)c(F)cn2)c(C)c1C(=O)NCCO] | 75 |
| 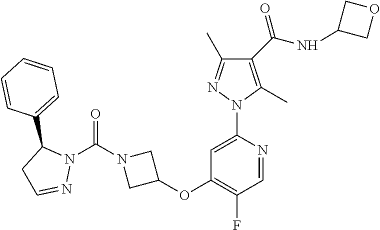 [SMILES: Cc1nn(-c2cc(OC3CN(C(=O)N4N=CC[C@H]4c4ccccc4)C3)c(F)cn2)c(C)c1C(=O)NC1COC1] | 76 |
| 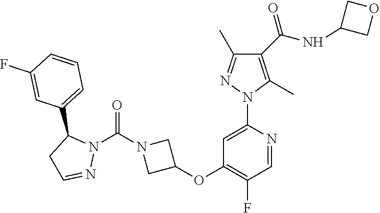 [SMILES: Cc1nn(-c2cc(OC3CN(C(=O)N4N=CC[C@H]4c4cccc(F)c4)C3)c(F)cn2)c(C)c1C(=O)NC1COC1] | 77 |
| 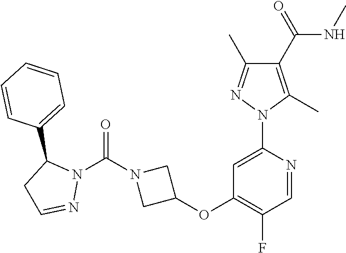 [SMILES: CNC(=O)c1c(C)nn(-c2cc(OC3CN(C(=O)N4N=CC[C@H]4c4ccccc4)C3)c(F)cn2)c1C] | 78 |
| 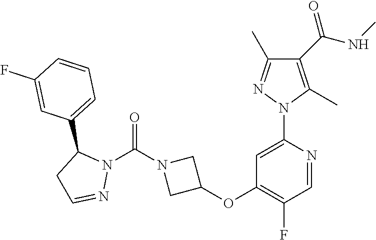 [SMILES: CNC(=O)c1c(C)nn(-c2cc(OC3CN(C(=O)N4N=CC[C@H]4c4cccc(F)c4)C3)c(F)cn2)c1C] | 79 |
| 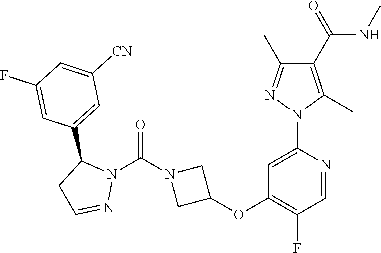 [SMILES: CNC(=O)c1c(C)nn(-c2cc(OC3CN(C(=O)N4N=CC[C@H]4c4cc(F)cc(C#N)c4)C3)c(F)cn2)c1C] | 80 |
| 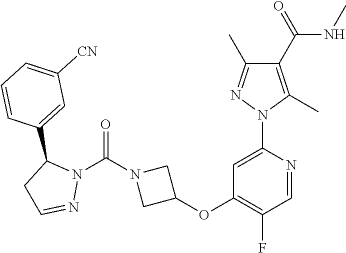 [SMILES: CNC(=O)c1c(C)nn(-c2cc(OC3CN(C(=O)N4N=CC[C@H]4c4cccc(C#N)c4)C3)c(F)cn2)c1C] | 81 |
| 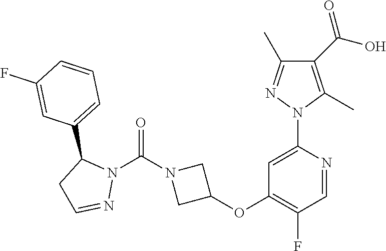 [SMILES: Cc1nn(-c2cc(OC3CN(C(=O)N4N=CC[C@H]4c4cccc(F)c4)C3)c(F)cn2)c(C)c1C(=O)O] | 82 |
| 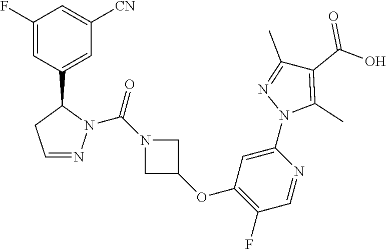 [SMILES: Cc1nn(-c2cc(OC3CN(C(=O)N4N=CC[C@H]4c4cc(F)cc(C#N)c4)C3)c(F)cn2)c(C)c1C(=O)O] | 83 |
| 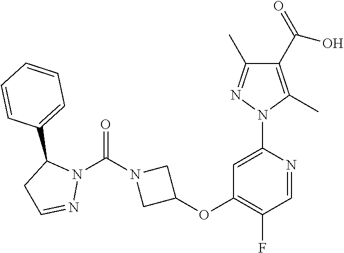 [SMILES: Cc1nn(-c2cc(OC3CN(C(=O)N4N=CC[C@H]4c4ccccc4)C3)c(F)cn2)c(C)c1C(=O)O] | 84 |
| 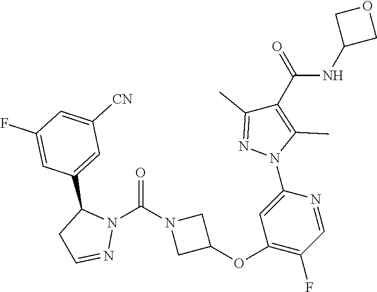 [SMILES: Cc1nn(-c2cc(OC3CN(C(=O)N4N=CC[C@H]4c4cc(F)cc(C#N)c4)C3)c(F)cn2)c(C)c1C(=O)NC1COC1] | 85 |
| 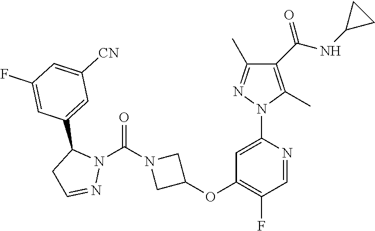 [SMILES: Cc1nn(-c2cc(OC3CN(C(=O)N4N=CC[C@H]4c4cc(F)cc(C#N)c4)C3)c(F)cn2)c(C)c1C(=O)NC1CC1] | 86 |
| 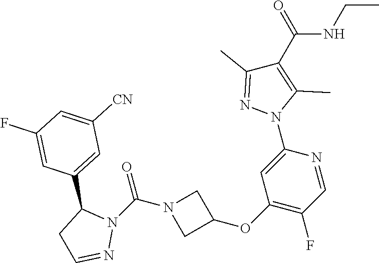 [SMILES: CCNC(=O)c1c(C)nn(-c2cc(OC3CN(C(=O)N4N=CC[C@H]4c4cc(F)cc(C#N)c4)C3)c(F)cn2)c1C] | 87 |
| 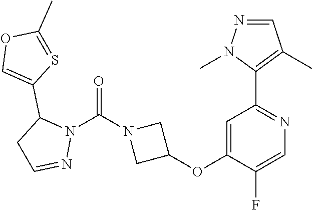 | 88 |
| 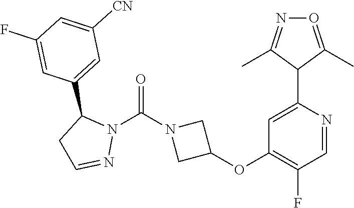 | 89 |
| 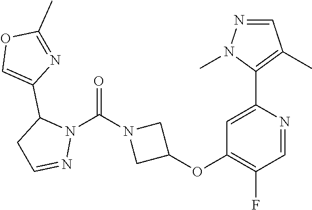 [SMILES: Cc1nc(C2CC=NN2C(=O)N2CC(Oc3cc(-c4c(C)cnn4C)ncc3F)C2)co1] | 90 |
| 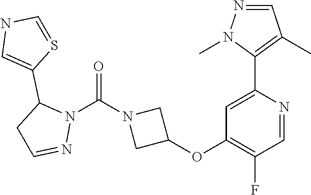 | 91 |
| 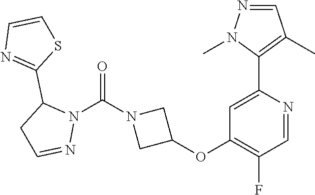 [SMILES: Cc1cnn(C)c1-c1cc(OC2CN(C(=O)N3N=CCC3c3nccs3)C2)c(F)cn1] | 92 |
| 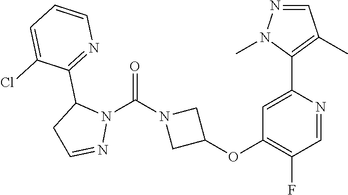 [SMILES: Cc1cnn(C)c1-c1cc(OC2CN(C(=O)N3N=CCC3c3ncccc3Cl)C2)c(F)cn1] | 93 |
| 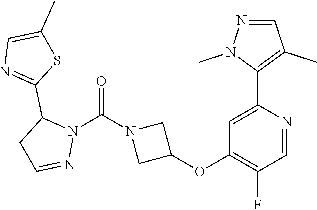 [SMILES: Cc1cnc(C2CC=NN2C(=O)N2CC(Oc3cc(-c4c(C)cnn4C)ncc3F)C2)s1] | 94 |
| 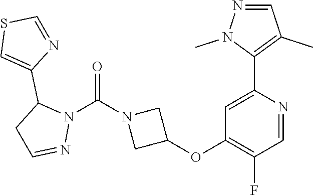 [SMILES: Cc1cnn(C)c1-c1cc(OC2CN(C(=O)N3N=CCC3c3cscn3)C2)c(F)cn1] | 95 |
| 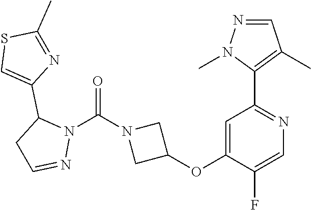 [SMILES: Cc1nc(C2CC=NN2C(=O)N2CC(Oc3cc(-c4c(C)cnn4C)ncc3F)C2)cs1] | 96 |
| 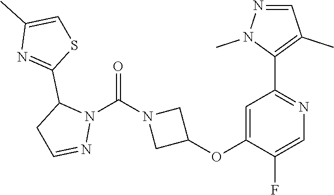 | 97 |
| 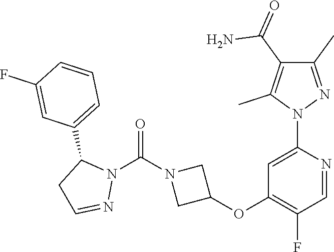 [SMILES: Cc1nn(-c2cc(OC3CN(C(=O)N4N=CC[C@@H]4c4cccc(F)c4)C3)c(F)cn2)c(C)c1C(N)=O] | 98 |
| 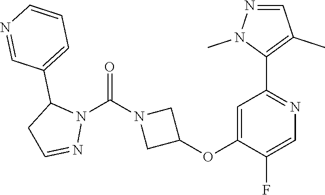 [SMILES: Cc1cnn(C)c1-c1cc(OC2CN(C(=O)N3N=CCC3c3cccnc3)C2)c(F)cn1] | 99 |
| 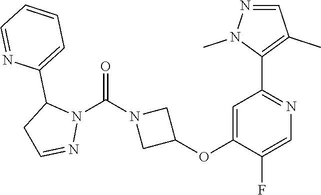 [SMILES: Cc1cnn(C)c1-c1cc(OC2CN(C(=O)N3N=CCC3c3ccccn3)C2)c(F)cn1] | 100 |
| 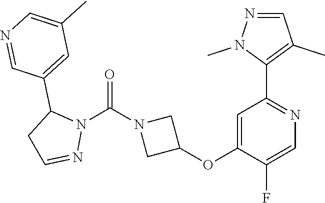 [SMILES: Cc1cncc(C2CC=NN2C(=O)N2CC(Oc3cc(-c4c(C)cnn4C)ncc3F)C2)c1] | 101 |
| 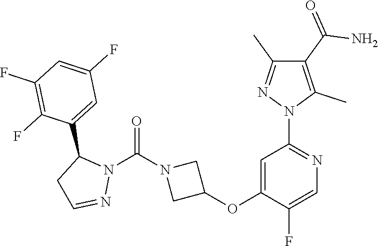 [SMILES: Cc1nn(-c2cc(OC3CN(C(=O)N4N=CC[C@H]4c4cc(F)cc(F)c4F)C3)c(F)cn2)c(C)c1C(N)=O] | 102 |
| 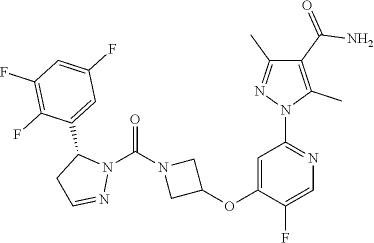 [SMILES: Cc1nn(-c2cc(OC3CN(C(=O)N4N=CC[C@@H]4c4cc(F)cc(F)c4F)C3)c(F)cn2)c(C)c1C(N)=O] | 103 |
| 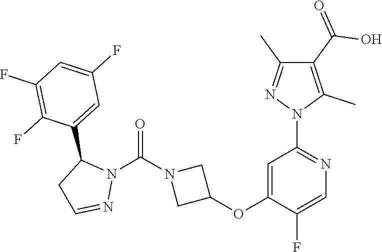 [SMILES: Cc1nn(-c2cc(OC3CN(C(=O)N4N=CC[C@H]4c4cc(F)cc(F)c4F)C3)c(F)cn2)c(C)c1C(=O)O] | 104 |
| 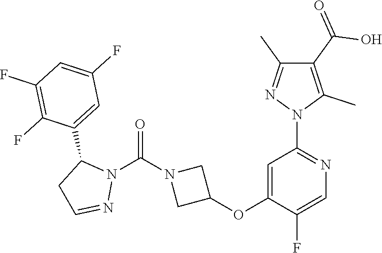 [SMILES: Cc1nn(-c2cc(OC3CN(C(=O)N4N=CC[C@@H]4c4cc(F)cc(F)c4F)C3)c(F)cn2)c(C)c1C(=O)O] | 105 |
| 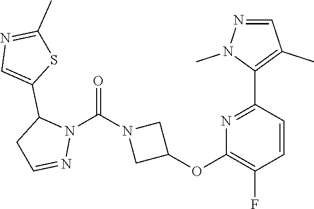 [SMILES: Cc1ncc(C2CC=NN2C(=O)N2CC(Oc3nc(-c4c(C)cnn4C)ccc3F)C2)s1] | 106 |
| 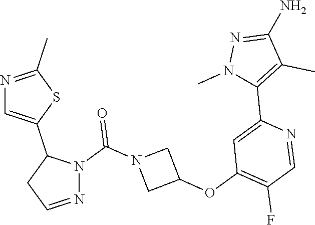 [SMILES: Cc1ncc(C2CC=NN2C(=O)N2CC(Oc3cc(-c4c(C)c(N)nn4C)ncc3F)C2)s1] | 107 |
| 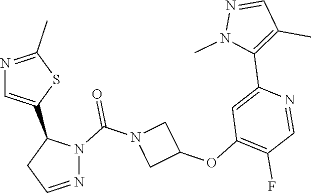 [SMILES: Cc1ncc([C@@H]2CC=NN2C(=O)N2CC(Oc3cc(-c4c(C)cnn4C)ncc3F)C2)s1] | 108 |
| 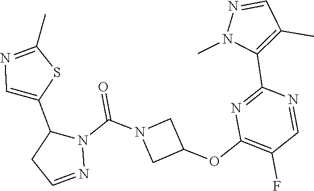 [SMILES: Cc1ncc(C2CC=NN2C(=O)N2CC(Oc3nc(-c4c(C)cnn4C)ncc3F)C2)s1] | 109 |
| 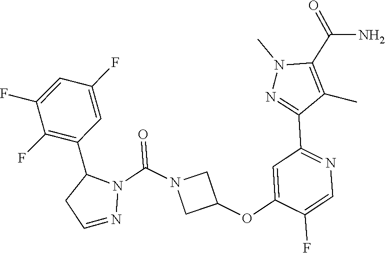 [SMILES: Cc1c(-c2cc(OC3CN(C(=O)N4N=CCC4c4cc(F)cc(F)c4F)C3)c(F)cn2)nn(C)c1C(N)=O] | 110 |
| 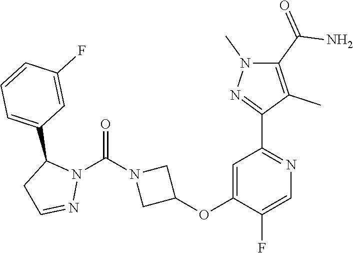 [SMILES: Cc1c(-c2cc(OC3CN(C(=O)N4N=CC[C@H]4c4cccc(F)c4)C3)c(F)cn2)nn(C)c1C(N)=O] | 111 |
| 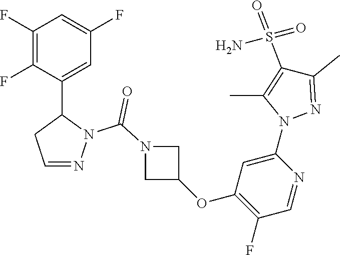 [SMILES: Cc1nn(-c2cc(OC3CN(C(=O)N4N=CCC4c4cc(F)cc(F)c4F)C3)c(F)cn2)c(C)c1S(N)(=O)=O] | 112 |
| 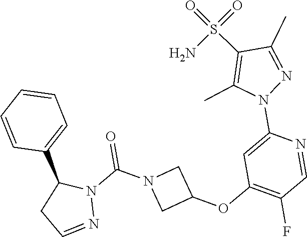 [SMILES: Cc1nn(-c2cc(OC3CN(C(=O)N4N=CC[C@H]4c4ccccc4)C3)c(F)cn2)c(C)c1S(N)(=O)=O] | 113 |
| 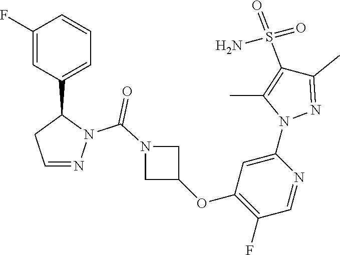 [SMILES: Cc1nn(-c2cc(OC3CN(C(=O)N4N=CC[C@H]4c4cccc(F)c4)C3)c(F)cn2)c(C)c1S(N)(=O)=O] | 114 |
| 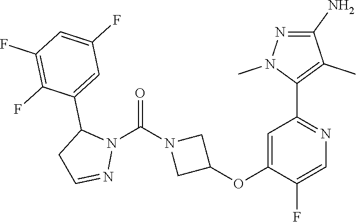 [SMILES: Cc1c(N)nn(C)c1-c1cc(OC2CN(C(=O)N3N=CCC3c3cc(F)cc(F)c3F)C2)c(F)cn1] | 115 |
| 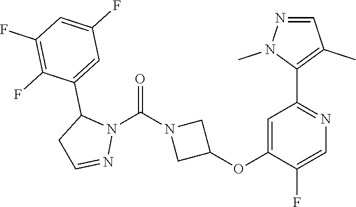 [SMILES: Cc1cnn(C)c1-c1cc(OC2CN(C(=O)N3N=CCC3c3cc(F)cc(F)c3F)C2)c(F)cn1] | 116 |
| 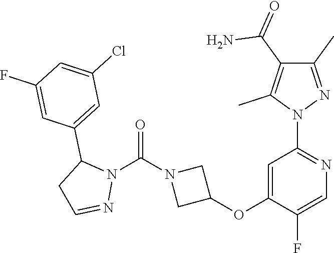 [SMILES: Cc1nn(-c2cc(OC3CN(C(=O)N4N=CCC4c4cc(F)cc(Cl)c4)C3)c(F)cn2)c(C)c1C(N)=O] | 117 |
| 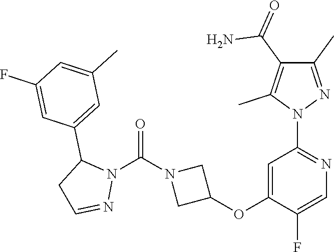 [SMILES: Cc1cc(F)cc(C2CC=NN2C(=O)N2CC(Oc3cc(-n4nc(C)c(C(N)=O)c4C)ncc3F)C2)c1] | 118 |
| 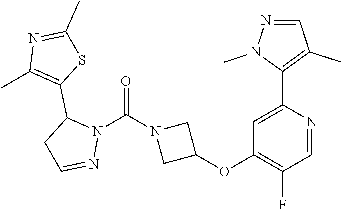 | 119 |
| 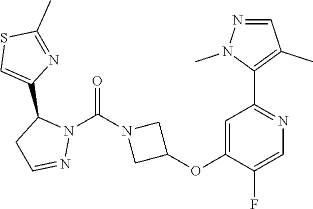 [SMILES: Cc1nc([C@@H]2CC=NN2C(=O)N2CC(Oc3cc(-c4c(C)cnn4C)ncc3F)C2)cs1] | 120 |
| 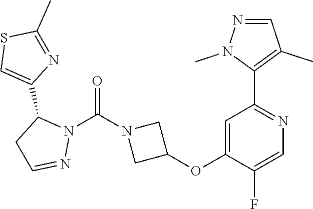 [SMILES: Cc1nc([C@H]2CC=NN2C(=O)N2CC(Oc3cc(-c4c(C)cnn4C)ncc3F)C2)cs1] | 121 |
| 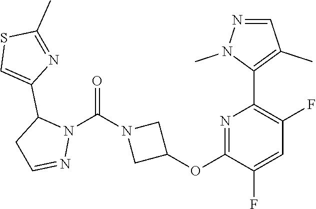 [SMILES: Cc1nc(C2CC=NN2C(=O)N2CC(Oc3nc(-c4c(C)cnn4C)c(F)cc3F)C2)cs1] | 122 |
| 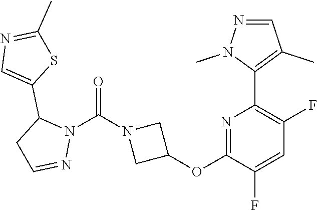 [SMILES: Cc1ncc(C2CC=NN2C(=O)N2CC(Oc3nc(-c4c(C)cnn4C)c(F)cc3F)C2)s1] | 123 |
| 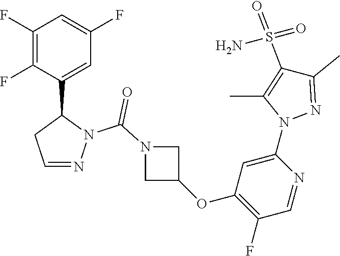 [SMILES: Cc1nn(-c2cc(OC3CN(C(=O)N4N=CC[C@H]4c4cc(F)cc(F)c4F)C3)c(F)cn2)c(C)c1S(N)(=O)=O] | 124 |
| 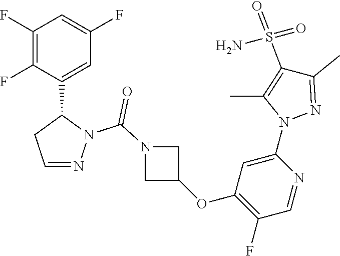 [SMILES: Cc1nn(-c2cc(OC3CN(C(=O)N4N=CC[C@@H]4c4cc(F)cc(F)c4F)C3)c(F)cn2)c(C)c1S(N)(=O)=O] | 125 |
| 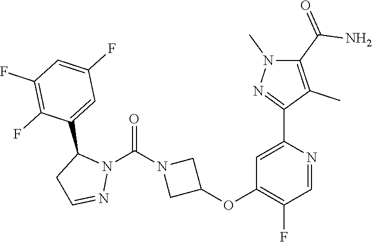 [SMILES: Cc1c(-c2cc(OC3CN(C(=O)N4N=CC[C@H]4c4cc(F)cc(F)c4F)C3)c(F)cn2)nn(C)c1C(N)=O] | 126 |
| 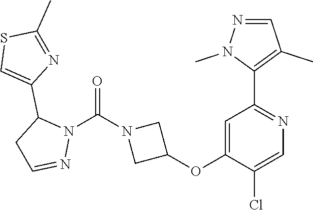 [SMILES: Cc1nc(C2CC=NN2C(=O)N2CC(Oc3cc(-c4c(C)cnn4C)ncc3Cl)C2)cs1] | 127 |
| 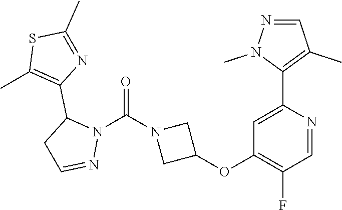 | 128 |
| 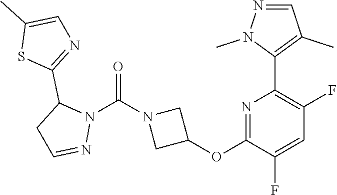 | 129 |
| 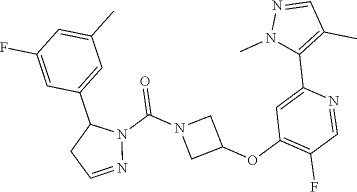 [SMILES: Cc1cc(F)cc(C2CC=NN2C(=O)N2CC(Oc3cc(-c4c(C)cnn4C)ncc3F)C2)c1] | 130 |
| 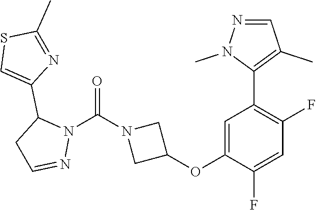 [SMILES: Cc1nc(C2CC=NN2C(=O)N2CC(Oc3cc(-c4c(C)cnn4C)c(F)cc3F)C2)cs1] | 131 |
| 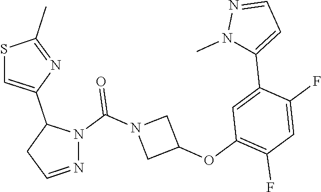 [SMILES: Cc1nc(C2CC=NN2C(=O)N2CC(Oc3cc(-c4ccnn4C)c(F)cc3F)C2)cs1] | 132 |
| 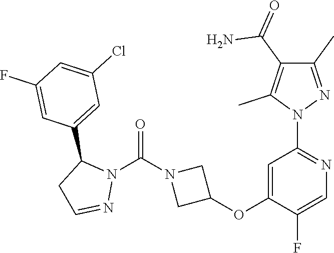 [SMILES: Cc1nn(-c2cc(OC3CN(C(=O)N4N=CC[C@H]4c4cc(F)cc(Cl)c4)C3)c(F)cn2)c(C)c1C(N)=O] | 133 |
| 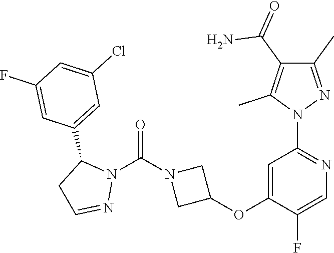 [SMILES: Cc1nn(-c2cc(OC3CN(C(=O)N4N=CC[C@@H]4c4cc(F)cc(Cl)c4)C3)c(F)cn2)c(C)c1C(N)=O] | 134 |
| 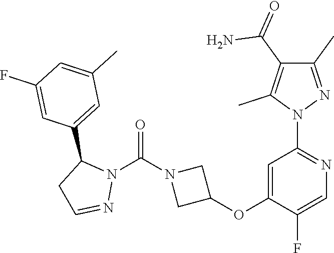 [SMILES: Cc1cc(F)cc([C@@H]2CC=NN2C(=O)N2CC(Oc3cc(-n4nc(C)c(C(N)=O)c4C)ncc3F)C2)c1] | 135 |
| 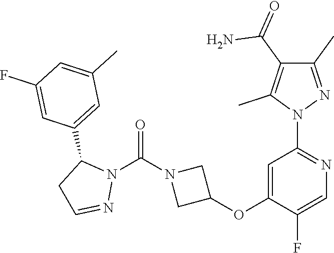 [SMILES: Cc1cc(F)cc([C@H]2CC=NN2C(=O)N2CC(Oc3cc(-n4nc(C)c(C(N)=O)c4C)ncc3F)C2)c1] | 136 |
| 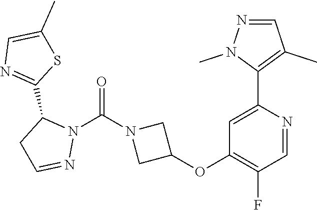 [SMILES: Cc1cnc([C@H]2CC=NN2C(=O)N2CC(Oc3cc(-c4c(C)cnn4C)ncc3F)C2)s1] | 137 |
| 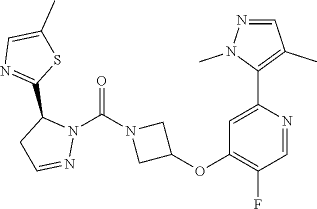 [SMILES: Cc1cnc([C@@H]2CC=NN2C(=O)N2CC(Oc3cc(-c4c(C)cnn4C)ncc3F)C2)s1] | 138 |
| 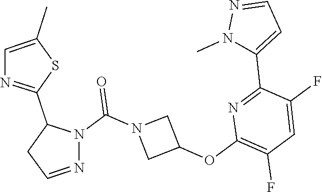 [SMILES: Cc1cnc(C2CC=NN2C(=O)N2CC(Oc3nc(-c4ccnn4C)c(F)cc3F)C2)s1] | 139 |
| 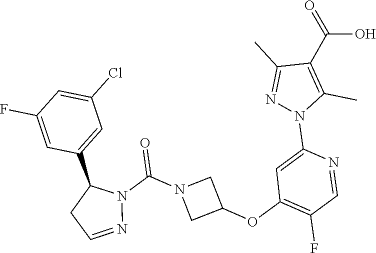 [SMILES: Cc1nn(-c2cc(OC3CN(C(=O)N4N=CC[C@H]4c4cc(F)cc(Cl)c4)C3)c(F)cn2)c(C)c1C(=O)O] | 140 |
| 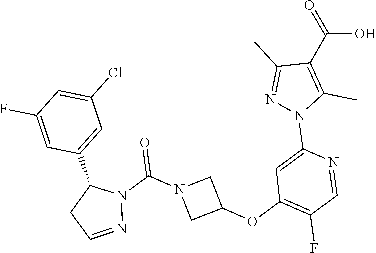 [SMILES: Cc1nn(-c2cc(OC3CN(C(=O)N4N=CC[C@@H]4c4cc(F)cc(Cl)c4)C3)c(F)cn2)c(C)c1C(=O)O] | 141 |
| 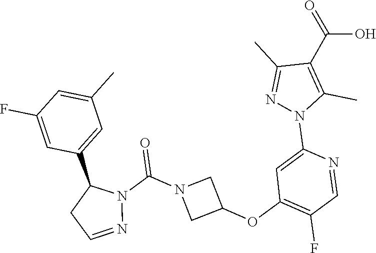 [SMILES: Cc1cc(F)cc([C@@H]2CC=NN2C(=O)N2CC(Oc3cc(-n4nc(C)c(C(=O)O)c4C)ncc3F)C2)c1] | 142 |
| 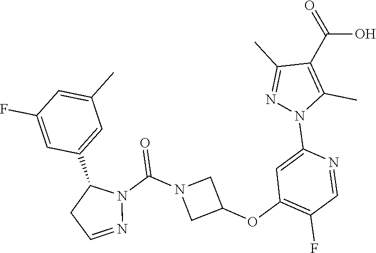 [SMILES: Cc1cc(F)cc([C@H]2CC=NN2C(=O)N2CC(Oc3cc(-n4nc(C)c(C(=O)O)c4C)ncc3F)C2)c1] | 143 |
| 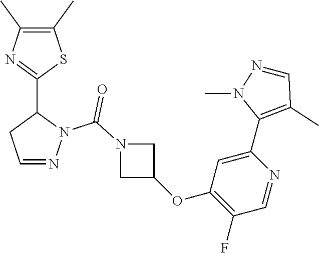 | 144 |
| 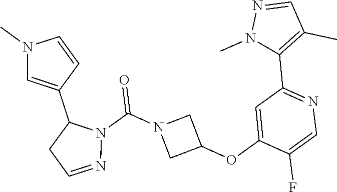 | 145 |
| 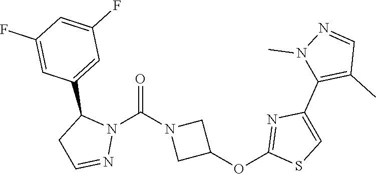 [SMILES: Cc1cnn(C)c1-c1csc(OC2CN(C(=O)N3N=CC[C@H]3c3cc(F)cc(F)c3)C2)n1] | 146 |
| 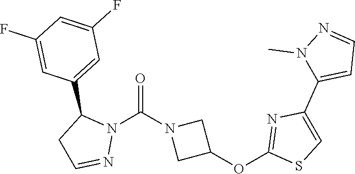 [SMILES: Cn1nccc1-c1csc(OC2CN(C(=O)N3N=CC[C@H]3c3cc(F)cc(F)c3)C2)n1] | 147 |
| 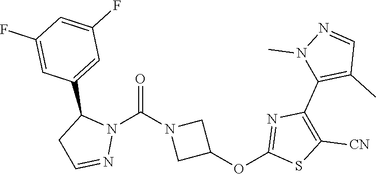 [SMILES: Cc1cnn(C)c1-c1nc(OC2CN(C(=O)N3N=CC[C@H]3c3cc(F)cc(F)c3)C2)sc1C#N] | 148 |
| 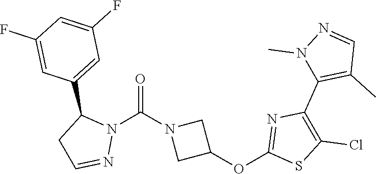 [SMILES: Cc1cnn(C)c1-c1nc(OC2CN(C(=O)N3N=CC[C@H]3c3cc(F)cc(F)c3)C2)sc1Cl] | 149 |
| 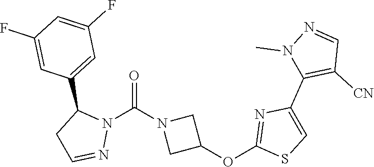 [SMILES: Cn1ncc(C#N)c1-c1csc(OC2CN(C(=O)N3N=CC[C@H]3c3cc(F)cc(F)c3)C2)n1] | 150 |
| 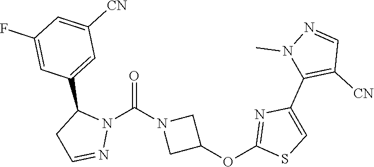 [SMILES: Cn1ncc(C#N)c1-c1csc(OC2CN(C(=O)N3N=CC[C@H]3c3cc(F)cc(C#N)c3)C2)n1] | 151 |
| 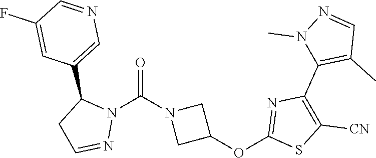 [SMILES: Cc1cnn(C)c1-c1nc(OC2CN(C(=O)N3N=CC[C@H]3c3cncc(F)c3)C2)sc1C#N] | 152 |
| 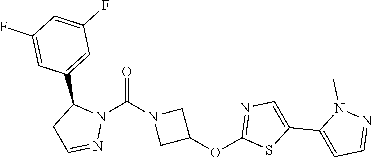 [SMILES: Cn1nccc1-c1cnc(OC2CN(C(=O)N3N=CC[C@H]3c3cc(F)cc(F)c3)C2)s1] | 153 |
| 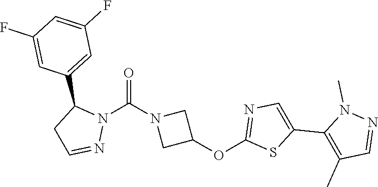 [SMILES: Cc1cnn(C)c1-c1cnc(OC2CN(C(=O)N3N=CC[C@H]3c3cc(F)cc(F)c3)C2)s1] | 154 |
| 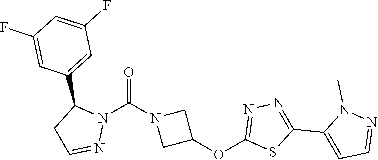 [SMILES: Cn1nccc1-c1nnc(OC2CN(C(=O)N3N=CC[C@H]3c3cc(F)cc(F)c3)C2)s1] | 155 |
| 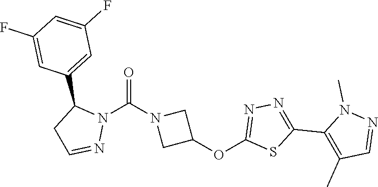 [SMILES: Cc1cnn(C)c1-c1nnc(OC2CN(C(=O)N3N=CC[C@H]3c3cc(F)cc(F)c3)C2)s1] | 156 |
| 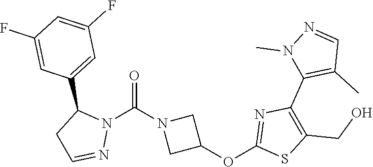 [SMILES: Cc1cnn(C)c1-c1nc(OC2CN(C(=O)N3N=CC[C@H]3c3cc(F)cc(F)c3)C2)sc1CO] | 157 |
| 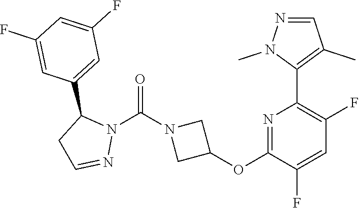 [SMILES: Cc1cnn(C)c1-c1nc(OC2CN(C(=O)N3N=CC[C@H]3c3cc(F)cc(F)c3)C2)c(F)cc1F] | 158 |
| 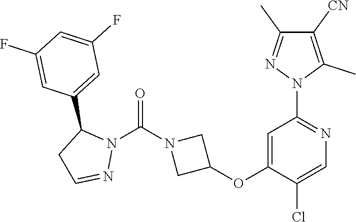 [SMILES: Cc1nn(-c2cc(OC3CN(C(=O)N4N=CC[C@H]4c4cc(F)cc(F)c4)C3)c(Cl)cn2)c(C)c1C#N] | 159 |
| 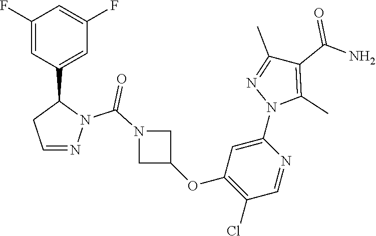 [SMILES: Cc1nn(-c2cc(OC3CN(C(=O)N4N=CC[C@H]4c4cc(F)cc(F)c4)C3)c(Cl)cn2)c(C)c1C(N)=O] | 160 |
| 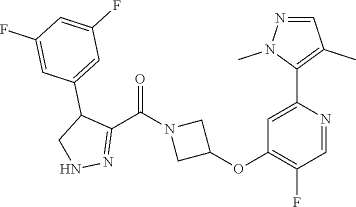 [SMILES: Cc1cnn(C)c1-c1cc(OC2CN(C(=O)C3=NNCC3c3cc(F)cc(F)c3)C2)c(F)cn1] | 161 |
| 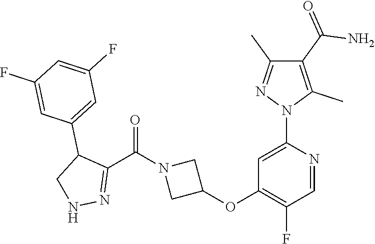 [SMILES: Cc1nn(-c2cc(OC3CN(C(=O)C4=NNCC4c4cc(F)cc(F)c4)C3)c(F)cn2)c(C)c1C(N)=O] | 162 |
| 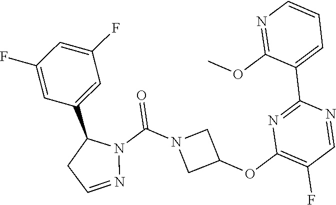 [SMILES: COc1ncccc1-c1ncc(F)c(OC2CN(C(=O)N3N=CC[C@H]3c3cc(F)cc(F)c3)C2)n1] | 163 |
| 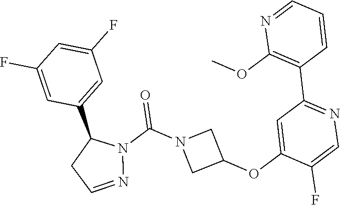 [SMILES: COc1ncccc1-c1cc(OC2CN(C(=O)N3N=CC[C@H]3c3cc(F)cc(F)c3)C2)c(F)cn1] | 164 |
| 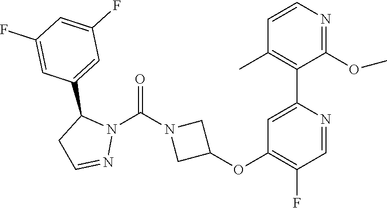 [SMILES: COc1nccc(C)c1-c1cc(OC2CN(C(=O)N3N=CC[C@H]3c3cc(F)cc(F)c3)C2)c(F)cn1] | 165 |
| 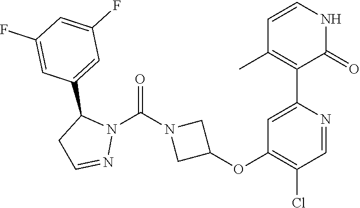 [SMILES: Cc1cc[nH]c(=O)c1-c1cc(OC2CN(C(=O)N3N=CC[C@H]3c3cc(F)cc(F)c3)C2)c(Cl)cn1] | 166 |
| 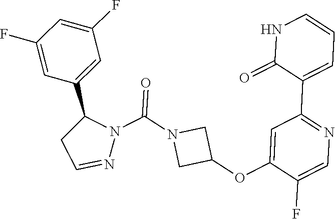 [SMILES: O=C(N1CC(Oc2cc(-c3ccc[nH]c3=O)ncc2F)C1)N1N=CC[C@H]1c1cc(F)cc(F)c1] | 167 |
| 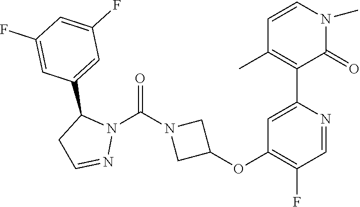 [SMILES: Cc1ccn(C)c(=O)c1-c1cc(OC2CN(C(=O)N3N=CC[C@H]3c3cc(F)cc(F)c3)C2)c(F)cn1] | 168 |
| 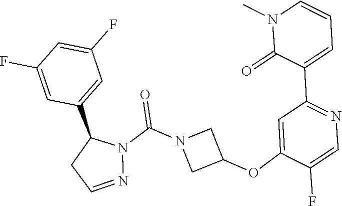 [SMILES: Cn1cccc(-c2cc(OC3CN(C(=O)N4N=CC[C@H]4c4cc(F)cc(F)c4)C3)c(F)cn2)c1=O] | 169 |

a tautomer thereof, a hydrate or stereoisomer of the compound or the tautomer, or a pharmaceutically acceptable salt of the foregoing.

38. A pharmaceutical composition comprising a compound according to claim 1, a tautomer thereof, a hydrate or stereoisomer of the compound or the tautomer, or a pharmaceutically acceptable salt of the foregoing and at least one pharmaceutically acceptable carrier.

39. A method of treating a disease or condition, comprising administering to a subject, a therapeutically effective amount of a compound according to claim 1, a tautomer thereof, a hydrate or stereoisomer of the compound or the tautomer, or a pharmaceutically acceptable salt of the foregoing wherein the disease or condition is selected from a inflammatory disease, an immune disease, an allergic disease, transplant rejection, a necrotic cell disease, a neurodegenerative disease, a central nervous system (CNS) disease, ischemic brain injury, an ocular disease, an infectious disease, and a malignancy.

40. The method according to claim 39, wherein the disease or condition is mediated by receptor-interacting protein 1 (RIP1) signaling.

41. A method of treating a disease or condition mediated by receptor-interacting protein 1 (RIP1) signaling, comprising administering to a subject, a therapeutically effective amount of a compound according to claim 1, a tautomer thereof, a hydrate or stereoisomer of the compound or the tautomer, or a pharmaceutically acceptable salt of the foregoing.

42. The method according to claim 39, wherein the disease or condition is selected from ulcerative colitis, Crohn's disease, psoriasis, rheumatoid arthritis, amyotrophic lateral sclerosis (ALS), Alzheimer's disease, and a viral infection.

43. A method of inhibiting receptor-interacting protein 1 (RIP1), comprising contacting the RIP1 protein or a fragment thereof with a compound according to claim 1, a tautomer thereof, a hydrate or stereoisomer of the compound or the tautomer, or a pharmaceutically acceptable salt of the foregoing.
